SEQUENCE LISTING (227 sequences)

SEQ ID NO: 1 - protein, 8 aa, synthetic construct
  REGION 1..8: note = Synthetic
GYTFTGYY

SEQ ID NO: 2 - protein, 8 aa, synthetic construct
  REGION 1..8: note = Synthetic
GFTFSSYT

SEQ ID NO: 3 - protein, 8 aa, synthetic construct
  REGION 1..8: note = Synthetic
GFTFSSYS

SEQ ID NO: 4 - protein, 8 aa, synthetic construct
  REGION 1..8: note = Synthetic
GFTFSSYA

SEQ ID NO: 5 - protein, 8 aa, synthetic construct
  REGION 1..8: note = Synthetic
GFTFSRFW

SEQ ID NO: 6 - protein, 8 aa, synthetic construct
  REGION 1..8: note = Synthetic
GGSISSYY

SEQ ID NO: 7 - protein, 8 aa, synthetic construct
  REGION 1..8: note = Synthetic
GYSFTRYW

SEQ ID NO: 8 - protein, 8 aa, synthetic construct
  REGION 1..8: note = Synthetic
GYTFTSYW

SEQ ID NO: 9 - protein, 8 aa, synthetic construct
  REGION 1..8: note = Synthetic
GFSLTSYG

SEQ ID NO: 10 - protein, 8 aa, synthetic construct
  REGION 1..8: note = Synthetic
GFTFSSDA

SEQ ID NO: 11 - protein, 8 aa, synthetic construct
  REGION 1..8: note = Synthetic
GFTFSSSA

SEQ ID NO: 12 - protein, 8 aa, synthetic construct
  REGION 1..8: note = Synthetic
GFTFSNCA

SEQ ID NO: 13 - protein, 10 aa, synthetic construct
  REGION 1..10: note = Synthetic
GFSLSTSDMG

SEQ ID NO: 14 - protein, 10 aa, synthetic construct
  REGION 1..10: note = Synthetic
GFSLSTSDLG

SEQ ID NO: 15 - protein, 8 aa, synthetic construct
  REGION 1..8: note = Synthetic
INPNSGGT

SEQ ID NO: 16 - protein, 8 aa, synthetic construct
  REGION 1..8: note = Synthetic
ISIVGNYI

SEQ ID NO: 17 - protein, 8 aa, synthetic construct
  REGION 1..8: note = Synthetic
ISTSSSYI

SEQ ID NO: 18 - protein, 8 aa, synthetic construct
  REGION 1..8: note = Synthetic
ISDSGGIA

SEQ ID NO: 19 - protein, 8 aa, synthetic construct
  REGION 1..8: note = Synthetic
IHYSGGTT

SEQ ID NO: 20 - protein, 8 aa, synthetic construct
  REGION 1..8: note = Synthetic
TKQDGSEK

SEQ ID NO: 21 - protein, 7 aa, synthetic construct
  REGION 1..7: note = Synthetic
IYTRGST

SEQ ID NO: 22 - protein, 8 aa, synthetic construct
  REGION 1..8: note = Synthetic
IDPSDSET

SEQ ID NO: 23 - protein, 8 aa, synthetic construct
  REGION 1..8: note = Synthetic
IYPSDSYT

SEQ ID NO: 24 - protein, 7 aa, synthetic construct
  REGION 1..7: note = Synthetic
IWVGGIT

SEQ ID NO: 25 - protein, 7 aa, synthetic construct
  REGION 1..7: note = Synthetic
IWAGGIT

SEQ ID NO: 26 - protein, 7 aa, synthetic construct
  REGION 1..7: note = Synthetic
ISIGGSA

SEQ ID NO: 27 - protein, 7 aa, synthetic construct
  REGION 1..7: note = Synthetic
ISVGGST

SEQ ID NO: 28 - protein, 7 aa, synthetic construct
  REGION 1..7: note = Synthetic
ISIGGST

SEQ ID NO: 29 - protein, 7 aa, synthetic construct
  REGION 1..7: note = Synthetic
ISVGGSI

SEQ ID NO: 30 - protein, 7 aa, synthetic construct
  REGION 1..7: note = Synthetic
IYWDDDK

SEQ ID NO: 31 - protein, 16 aa, synthetic construct
  REGION 1..16: note = Synthetic
AREGANWDYYYYGMDV

SEQ ID NO: 32 - protein, 12 aa, synthetic construct
  REGION 1..12: note = Synthetic
ARRKWEHDAFDI

SEQ ID NO: 33 - protein, 12 aa, synthetic construct
  REGION 1..12: note = Synthetic
ARRAWESDAFAI

SEQ ID NO: 34 - protein, 5 aa, synthetic construct
  REGION 1..5: note = Synthetic
VRDGY

SEQ ID NO: 35 - protein, 5 aa, synthetic construct
  REGION 1..5: note = Synthetic
VKDGY

SEQ ID NO: 36 - protein, 13 aa, synthetic construct
  REGION 1..13: note = Synthetic
AREGSGAYWAFDI

SEQ ID NO: 37 - protein, 15 aa, synthetic construct
  REGION 1..15: note = Synthetic
ARVFGGNYRFDAFHI

SEQ ID NO: 38 - protein, 14 aa, synthetic construct
  REGION 1..14: note = Synthetic
ARGGYFGEGWYFDV

SEQ ID NO: 39 - protein, 12 aa, synthetic construct
  REGION 1..12: note = Synthetic
TRRANYVGAMDY

SEQ ID NO: 40 - protein, 11 aa, synthetic construct
  REGION 1..11: note = Synthetic
ARDGYYGDFDV

SEQ ID NO: 41 - protein, 11 aa, synthetic construct
  REGION 1..11: note = Synthetic
ARDGYYGHFDV

SEQ ID NO: 42 - protein, 11 aa, synthetic construct
  REGION 1..11: note = Synthetic
ARGGDYDAMDY

SEQ ID NO: 43 - protein, 11 aa, synthetic construct
  REGION 1..11: note = Synthetic
ARGGDYDPMDY

SEQ ID NO: 44 - protein, 11 aa, synthetic construct
  REGION 1..11: note = Synthetic
TRGGDYDAMDY

SEQ ID NO: 45 - protein, 15 aa, synthetic construct
  REGION 1..15: note = Synthetic
VRGTAVVAFYWYFDV

SEQ ID NO: 46 - protein, 8 aa, synthetic construct
  REGION 1..8: note = Synthetic
SSNIGSKT

SEQ ID NO: 47 - protein, 6 aa, synthetic construct
  REGION 1..6: note = Synthetic
SLRSYY

SEQ ID NO: 48 - protein, 12 aa, synthetic construct
  REGION 1..12: note = Synthetic
QSLLDSDDGYTY

SEQ ID NO: 49 - protein, 11 aa, synthetic construct
  REGION 1..11: note = Synthetic
QSLVHNDGNTY

SEQ ID NO: 50 - protein, 9 aa, synthetic construct
  REGION 1..9: note = Synthetic
SSNIGAGYD

SEQ ID NO: 51 - protein, 11 aa, synthetic construct
  REGION 1..11: note = Synthetic
QSLVHRNGNTY

SEQ ID NO: 52 - protein, 5 aa, synthetic construct
  REGION 1..5: note = Synthetic
SSVSY

SEQ ID NO: 53 - protein, 6 aa, synthetic construct
  REGION 1..6: note = Synthetic
QDINKF

SEQ ID NO: 54 - protein, 6 aa, synthetic construct
  REGION 1..6: note = Synthetic
QDINKY

SEQ ID NO: 55 - protein, 6 aa, synthetic construct
  REGION 1..6: note = Synthetic
QSISDL

SEQ ID NO: 56 - protein, 6 aa, synthetic construct
  REGION 1..6: note = Synthetic
QSISDY

SEQ ID NO: 57 - protein, 6 aa, synthetic construct
  REGION 1..6: note = Synthetic
QDINNY

SEQ ID NO: 58-68 - skipped (11 entries)

SEQ ID NO: 69 - protein, 11 aa, synthetic construct
  REGION 1..11: note = Synthetic
AAWDDSLNGPV

SEQ ID NO: 70 - protein, 11 aa, synthetic construct
  REGION 1..11: note = Synthetic
TSRDNSGNHVV

SEQ ID NO: 71 - protein, 11 aa, synthetic construct
  REGION 1..11: note = Synthetic
NSRDSSGNHVV

SEQ ID NO: 72 - protein, 9 aa, synthetic construct
  REGION 1..9: note = Synthetic
MQRIEFPLT

SEQ ID NO: 73 - protein, 9 aa, synthetic construct
  REGION 1..9: note = Synthetic
LQATQFPLT

SEQ ID NO: 74 - protein, 11 aa, synthetic construct
  REGION 1..11: note = Synthetic
QSYDSSLSGYV

SEQ ID NO: 75 - protein, 9 aa, synthetic construct
  REGION 1..9: note = Synthetic
SQSTHVPWT

SEQ ID NO: 76 - protein, 9 aa, synthetic construct
  REGION 1..9: note = Synthetic
QQWSSNPLT

SEQ ID NO: 77 - protein, 8 aa, synthetic construct
  REGION 1..8: note = Synthetic
LQYDNLRT

SEQ ID NO: 78 - protein, 8 aa, synthetic construct
  REGION 1..8: note = Synthetic
LQYDNLRA

SEQ ID NO: 79 - protein, 9 aa, synthetic construct
  REGION 1..9: note = Synthetic
QNGHNFPFT

SEQ ID NO: 80 - protein, 9 aa, synthetic construct
  REGION 1..9: note = Synthetic
QNGHSFPFT

SEQ ID NO: 81 - protein, 9 aa, synthetic construct
  REGION 1..9: note = Synthetic
LHYDDFPLT

SEQ ID NO: 82 - protein, 9 aa, synthetic construct
  REGION 1..9: note = Synthetic
LQYDDFPLT

SEQ ID NO: 83-100 - skipped (18 entries)

SEQ ID NO: 101 - protein, 8 aa, synthetic construct
  REGION 1..8: note = Synthetic
GYSFTRYW

SEQ ID NO: 102 - protein, 8 aa, synthetic construct
  REGION 1..8: note = Synthetic
IDPSDSET

SEQ ID NO: 103 - protein, 14 aa, synthetic construct
  REGION 1..14: note = Synthetic
ARGGYFGREWYYDV

SEQ ID NO: 104 - protein, 14 aa, synthetic construct
  REGION 1..14: note = Synthetic
ARGGYFGEEWYHDV

SEQ ID NO: 105 - protein, 11 aa, synthetic construct
  REGION 1..11: note = Synthetic
QSLVHRNGNTY

SEQ ID NO: 106 - skipped

SEQ ID NO: 107 - protein, 9 aa, synthetic construct
  REGION 1..9: note = Synthetic
AQSTQLPYT

SEQ ID NO: 108 - protein, 121 aa, synthetic construct
  REGION 1..121: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGREWYYDVWGQGTLVTVSS

SEQ ID NO: 109 - protein, 112 aa, synthetic construct
  REGION 1..112: note = Synthetic
DIVMTQSPLSLPVTPGEPASISCRSSQSLVHRNGNTYLHWYLQKPGQSPQLLIYEVSNRAAGVPDRFSGSGSGTDFTLKISRVEAEDVGVYFCSQSTHVPWTFGGGTKLEIK

SEQ ID NO: 110 - protein, 121 aa, synthetic construct
  REGION 1..121: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGEEWYHDVWGQGTLVTVSS

SEQ ID NO: 111 - protein, 112 aa, synthetic construct
  REGION 1..112: note = Synthetic
DIVMTQSPLSLPVTPGEPASISCRSSQSLVHRNGNTYLHWYLQKPGQSPQLLIYEVSNRFTGVPDRFSGSGSGTDFTLKISRVEAEDVGVYFCAQSTQLPYTFGGGTKLEIK

SEQ ID NO: 112 - protein, 450 aa, synthetic construct
  REGION 1..450: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGREWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 113 - protein, 219 aa, synthetic construct
  REGION 1..219: note = Synthetic
DIVMTQSPLSLPVTPGEPASISCRSSQSLVHRNGNTYLHWYLQKPGQSPQLLIYEVSNRAAGVPDRFSGSGSGTDFTLKISRVEAEDVGVYFCSQSTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 450 aa, synthetic construct
  REGION 1..450: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGREWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 450 aa, synthetic construct
  REGION 1..450: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGEEWYHDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 116 - protein, 219 aa, synthetic construct
  REGION 1..219: note = Synthetic
DIVMTQSPLSLPVTPGEPASISCRSSQSLVHRNGNTYLHWYLQKPGQSPQLLIYEVSNRFTGVPDRFSGSGSGTDFTLKISRVEAEDVGVYFCAQSTQLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 450 aa, synthetic construct
  REGION 1..450: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGEEWYHDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 448 aa, synthetic construct
  REGION 1..448: note = Synthetic
EVQLVESGGGLVKPGGSLRLSCAASGFTFSSYTMNWVRQAPGKGLEWVSSISIVGNYIYYADSLKGRFTISRDNAKNSLYLQMNSLRAEDTAVYYCARRKWEHDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 119 - protein, 214 aa, synthetic construct
  REGION 1..214: note = Synthetic
SSELTQDPAVSVALGQTVRITCQGDSLRSYYASWYQQKPGQAPVLVIYGKNNRPSGIPDRFSGSSSGNTASLTITGAQAEDEADFYCTSRDNSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 120 - protein, 448 aa, synthetic construct
  REGION 1..448: note = Synthetic
EVQLVESGGGLVKPGGSLRLSCAASGFTFSSYTMNWVRQAPGKGLEWVSSISIVGNYIYYADSLKGRFTISRDNAKNSLYLQMNSLRAEDTAVYYCARRKWEHDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 214 aa, synthetic construct; identical to SEQ ID NO: 119
  REGION 1..214: note = Synthetic

SEQ ID NO: 122 - protein, 1181 aa, Homo sapiens
MAGARSRDPWGASGICYLFGSLLVELLFSRAVAFNLDVMGALRKEGEPGSLFGFSVALHRQLQPRPQSWLLVGAPQALALPGQQANRTGGLFACPLSLEETDCYRVDIDQGADMQKESKENQWLGVSVRSQGPGGKIVTCAHRYEARQRVDQILETRDMIGRCFVLSQDLAIRDELDGGEWKFCEGRPQGHEQFGFCQQGTAAAFSPDSHYLLFGAPGTYNWKGTARVELCAQGSADLAHLDDGPYEAGGEKEQDPRLIPVPANSYFGLLFVTNIDSSDPDQLVYKTLDPADRLPGPAGDLALNSYLGFSIDSGKGLVRAEELSFVAGAPRANHKGAVVILRKDSASRLVPEVMLSGERLTSGFGYSLAVADLNSDGWPDLIVGAPYFFERQEELGGAVYVYLNQGGHWAGISPLRLCGSPDSMFGISLAVLGDLNQDGFPDIAVGAPFDGDGKVFIYHGSSLGVVAKPSQVLEGEAVGIKSFGYSLSGSLDMDGNQYPDLLVGSLADTAVLFRARPILHVSHEVSIAPRSIDLEQPNCAGGHSVCVDLRVCFSYIAVPSSYSPTVALDYVLDADTDRRLRGQVPRVTFLSRNLEEPKHQASGTVWLKHQHDRVCGDAMFQLQENVKDKLRAIVVTLSYSLQTPRLRRQAPGQGLPPVAPILNAHQPSTQRAEIHFLKQGCGEDKICQSNLQLVRARFCTRVSDTEFQPLPMDVDGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIELPLSIAGMAIPQQLFFSGVVRGERAMQSERDVGSKVKYEVTVSNQGQSLRTLGSAFLNIMWPHEIANGKWLLYPMQVELEGGQGPGQKGLCSPRPNILHLDVDSRDRRRRELEPPEQQEPGERQEPSMSWWPVSSAEKKKNITLDCARGTANCVVFSCPLYSFDRAAVLHVWGRLWNSTFLEEYSAVKSLEVIVRANITVKSSIKNLMLRDASTVIPVMVYLDPMAVVAEGVPWWVILLAVLAGLLVLALLVLLLWKMGFFKRAKHPEATVPQYHAVKIPREDRQQFKEEKTGTILRNNWGSPRREGPDAHPILAADGHPELGPDGHPGPGTA

SEQ ID NO: 123 - protein, 1001 aa, Homo sapiens
FNLDVMGALRKEGEPGSLFGFSVALHRQLQPRPQSWLLVGAPQALALPGQQANRTGGLFACPLSLEETDCYRVDIDQGADMQKESKENQWLGVSVRSQGPGGKIVTCAHRYEARQRVDQILETRDMIGRCFVLSQDLAIRDELDGGEWKFCEGRPQGHEQFGFCQQGTAAAFSPDSHYLLFGAPGTYNWKGLLFVTNIDSSDPDQLVYKTLDPADRLPGPAGDLALNSYLGFSIDSGKGLVRAEELSFVAGAPRANHKGAVVILRKDSASRLVPEVMLSGERLTSGFGYSLAVADLNSDGWPDLIVGAPYFFERQEELGGAVYVYLNQGGHWAGISPLRLCGSPDSMFGISLAVLGDLNQDGFPDIAVGAPFDGDGKVFIYHGSSLGVVAKPSQVLEGEAVGIKSFGYSLSGSLDMDGNQYPDLLVGSLADTAVLFRARPILHVSHEVSIAPRSIDLEQPNCAGGHSVCVDLRVCFSYIAVPSSYSPTVALDYVLDADTDRRLRGQVPRVTFLSRNLEEPKHQASGTVWLKHQHDRVCGDAMFQLQENVKDKLRAIVVTLSYSLQTPRLRRQAPGQGLPPVAPILNAHQPSTQRAEIHFLKQGCGEDKICQSNLQLVRARFCTRVSDTEFQPLPMDVDGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIELPLSIAGMAIPQQLFFSGVVRGERAMQSERDVGSKVKYEVTVSNQGQSLRTLGSAFLNIMWPHEIANGKWLLYPMQVELEGGQGPGQKGLCSPRPNILHLDVDSRDRRRRELEPPEQQEPGERQEPSMSWWPVSSAEKKKNITLDCARGTANCVVFSCPLYSFDRAAVLHVWGRLWNSTFLEEYSAVKSLEVIVRANITVKSSIKNLMLRDASTVIPVMVYLDPMAVVAE

SEQ ID NO: 124 - protein, 1005 aa, Homo sapiens
FNLDVMGALRKEGEPGSLFGFSVALHRQLQPRPQSWLLVGAPQALALPGQQANRTGGLFACPLSLEETDCYRVDIDQGADMQKESKENQWLGVSVRSQGPGGKIVTCAHRYEARQRVDQILETRDMIGRCFVLSQDLAIRDELDGGEWKFCEGRPQGHEQFGFCQQGTAAAFSPDSHYLLFGAPGTYNWKGTARVELCAQGSADLAHLDDGPYEAGGEKEQDPRLIPVPANSYFGFSIDSGKGLVRAEELSFVAGAPRANHKGAVVILRKDSASRLVPEVMLSGERLTSGFGYSLAVADLNSDGWPDLIVGAPYFFERQEELGGAVYVYLNQGGHWAGISPLRLCGSPDSMFGISLAVLGDLNQDGFPDIAVGAPFDGDGKVFIYHGSSLGVVAKPSQVLEGEAVGIKSFGYSLSGSLDMDGNQYPDLLVGSLADTAVLFRARPILHVSHEVSIAPRSIDLEQPNCAGGHSVCVDLRVCFSYIAVPSSYSPTVALDYVLDADTDRRLRGQVPRVTFLSRNLEEPKHQASGTVWLKHQHDRVCGDAMFQLQENVKDKLRAIVVTLSYSLQTPRLRRQAPGQGLPPVAPILNAHQPSTQRAEIHFLKQGCGEDKICQSNLQLVRARFCTRVSDTEFQPLPMDVDGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIELPLSIAGMAIPQQLFFSGVVRGERAMQSERDVGSKVKYEVTVSNQGQSLRTLGSAFLNIMWPHEIANGKWLLYPMQVELEGGQGPGQKGLCSPRPNILHLDVDSRDRRRRELEPPEQQEPGERQEPSMSWWPVSSAEKKKNITLDCARGTANCVVFSCPLYSFDRAAVLHVWGRLWNSTFLEEYSAVKSLEVIVRANITVKSSIKNLMLRDASTVIPVMVYLDPMAVVAE

SEQ ID NO: 125 - protein, 400 aa, Homo sapiens
QGCGEDKICQSNLQLVRARFCTRVSDTEFQPLPMDVDGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIELPLSIAGMAIPQQLFFSGVVRGERAMQSERDVGSKVKYEVTVSNQGQSLRTLGSAFLNIMWPHEIANGKWLLYPMQVELEGGQGPGQKGLCSPRPNILHLDVDSRDRRRRELEPPEQQEPGERQEPSMSWWPVSSAEKKKNITLDCARGTANCVVFSCPLYSFDRAAVLHVWGRLWNSTFLEEYSAVKSLEVIVRANITVKSSIKNLMLRDASTVIPVMVYLDPMAVVAE

SEQ ID NO: 126 - protein, 161 aa, Homo sapiens
QSNLQLVRARFCTRVSDTEFQPLPMDVDGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIE

SEQ ID NO: 127 - protein, 230 aa, Homo sapiens
LPLSIAGMAIPQQLFFSGVVRGERAMQSERDVGSKVKYEVTVSNQGQSLRTLGSAFLNIMWPHEIANGKWLLYPMQVELEGGQGPGQKGLCSPRPNILHLDVDSRDRRRRELEPPEQQEPGERQEPSMSWWPVSSAEKKKNITLDCARGTANCVVFSCPLYSFDRAAVLHVWGRLWNSTFLEEYSAVKSLEVIVRANITVKSSIKNLMLRDASTVIPVMVYLDPMAVVAE

SEQ ID NO: 128 - protein, 123 aa, synthetic construct
  REGION 1..123: note = Synthetic
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREGANWDYYYYGMDVWGQGTTVTVSS

SEQ ID NO: 129 - protein, 110 aa, synthetic construct
  REGION 1..110: note = Synthetic
QSVLTQPPSASGTPGQRVTISCSGSSSNIGSKTVNWYQQLPGTAPKLLIYSNNQRPSGVPDRFSGSKSGTSASLAISGLQSEDEADYYCAAWDDSLNGPVFGGGTKLTVL

SEQ ID NO: 130 - protein, 119 aa, synthetic construct
  REGION 1..119: note = Synthetic
EVQLVESGGGLVKPGGSLRLSCAASGFTFSSYTMNWVRQAPGKGLEWVSSISIVGNYIYYADSLKGRFTISRDNAKNSLYLQMNSLRAEDTAVYYCARRKWEHDAFDIWGQGTMVTVSS

SEQ ID NO: 131 - protein, 108 aa, synthetic construct
  REGION 1..108: note = Synthetic
SSELTQDPAVSVALGQTVRITCQGDSLRSYYASWYQQKPGQAPVLVIYGKNNRPSGIPDRFSGSSSGNTASLTITGAQAEDEADFYCTSRDNSGNHVVFGGGTKLTVL

SEQ ID NO: 132 - protein, 119 aa, synthetic construct
  REGION 1..119: note = Synthetic
EVQLVESGGGLVKPGGSLRLSCAASGFTFSSYSMNWVRQAPGKGLEWVSSISTSSSYIYYADSVKGRFTISRDNAKNSLYLQMNSLRAEDTAVYYCARRAWESDAFAIWGQGTMVTVSS

SEQ ID NO: 133 - protein, 108 aa, synthetic construct
  REGION 1..108: note = Synthetic
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 134 - protein, 112 aa, synthetic construct
  REGION 1..112: note = Synthetic
EVQLLESGGGLEQPGGSLRLSCAASGFTFSSYAMNWVRQGLGKGLEWVSAISDSGGIAYYADSVKGRFTISRDNSKNTLYLQMDSLRGEDTAVYYCVRDGYWGQGTLVTVSS

SEQ ID NO: 135 - protein, 113 aa, synthetic construct
  REGION 1..113: note = Synthetic
DIVMTQTPLSLPVTPGEPASISCRSSQSLLDSDDGYTYLDWYLQKPGQSPQLLIYTLSYRASGVPDRFSGSGSGTDFSLKISRVEAEDAGIYYCMQRIEFPLTFGGGTKVEIK

SEQ ID NO: 136 - protein, 112 aa, synthetic construct
  REGION 1..112: note = Synthetic
EVQLLESGGGLVQPGGSLRLSCVASGFTFSSYAMTWVRQAPGKGLEWVSAIHYSGGTTNYAASVKGRFTISRDNSKNTLYLQMNSLRAEDTAVYYCVKDGYWGQGTLVTVSS

SEQ ID NO: 137 - protein, 113 aa, synthetic construct
  REGION 1..113: note = Synthetic
DIMMTQTPLSLPVSPGEPASISCRSSQSLLDSDDGYTYLDWYLQKPGQSPQLLIYTLSYRASGVPDRFSGSGSGTDFTLKISRVEAEDVGVYYCMQRIEFPLTFGGGTKVEIK

SEQ ID NO: 138 - protein, 120 aa, synthetic construct
  REGION 1..120: note = Synthetic
EVQLVESGGGLVQPGGSLRLSCAASGFTFSRFWMSWVRQAPGKGLEWVANTKQDGSEKYYVDSVKGRFTISRDNAKNSLYLQMNSLRADDTAVYYCAREGSGAYWAFDIWGQGTMVTVSS

SEQ ID NO: 139 - protein, 112 aa, synthetic construct
  REGION 1..112: note = Synthetic
DIVMTQTPLSSPVTLGQPASISCRSSQSLVHNDGNTYLSWLQQRPGQPPRLLIYKISNRFSGVPDRFSGSGAGTDFTLKISRVEAEDVGVYYCLQATQFPLTFGGGTKVEIK

SEQ ID NO: 140 - protein, 121 aa, synthetic construct
  REGION 1..121: note = Synthetic
QVQLQESGPGLVKPSETLSLTCTVSGGSISSYYWSWIRQPAGKGLEWIGRIYTRGSTNYNPSLKSRVTMSVDTSKNQFSLKLSSVTAADTAVYYCARVFGGNYRFDAFHIWSQGTMVTVSS

SEQ ID NO: 141 - protein, 111 aa, synthetic construct
  REGION 1..111: note = Synthetic
QSVLTQPPSVSGAPGQRVTISCTGSSSNIGAGYDVHWYQQLPGTAPKLLIYGNNNRPSGVPDRFSGSKSGTSASLAITGLQAEDEADYYCQSYDSSLSGYVFGTGTKVTVL

SEQ ID NO: 142 - protein, 121 aa, synthetic construct
  REGION 1..121: note = Synthetic
QVPLQQSGPQLVRPGASVKISCKASGYSFTRYWMHWVKQRPGQGLDWIGKIDPSDSETRLNQEFRDKATLTVDKSSSTAYLQLSSPTSEDSAVYYCARGGYFGEGWYFDVWGAGTTVTVSS

SEQ ID NO: 143 - protein, 112 aa, synthetic construct
  REGION 1..112: note = Synthetic
DVVMTQIPLSLPVSLGDQASISCRSSQSLVHRNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 144 - protein, 123 aa, synthetic construct
  REGION 1..123: note = Synthetic
QVTLKESGPGILQPSQTLSLTCSFSGFSLSTSDMGVSWIRQPSGKGLEWLAHIYWDDDKRYSPSLKSRLTISKDTSRNQVFLKITSVDTADTATYYCVRGTAVVAFYWYFDVWGAGTTVTVSS

SEQ ID NO: 145 - protein, 107 aa, synthetic construct
  REGION 1..107: note = Synthetic
DIKMTQSPSSMYASLGERVTITCKASQDINNYLNWFQQKPGKSPKTLIYRANRLVDGVPSRFSGSGSGQDYSLTISSLEYEDMGIYYCLHYDDFPLTFGAGTKVELK

SEQ ID NO: 146 - protein, 123 aa, synthetic construct
  REGION 1..123: note = Synthetic
QVTLKESGPGILQPSQTLSLTCSCSGFSLSTSDLGVSWIRQPSGKGLEWLAHIYWDDDKRYSPSLKSRLTISKDTSRNQVFLKITSVDTADTATYYCVRGTAVVAFYWYFDVWGAGTTVTVSS

SEQ ID NO: 147 - protein, 107 aa, synthetic construct
  REGION 1..107: note = Synthetic
DIKMTQSPSSMYASLGERVTITCKASQDINNYLNWFQQKPGKSPKTLIYRANRLVDGVPSRFSGSGSGQDYSLTISSLEYEDMGIYYCLQYDDFPLTFGAGTKVELK

SEQ ID NO: 148 - protein, 117 aa, synthetic construct
  REGION 1..117: note = Synthetic
QVQLKESGPGLVAPSQSLSITCTVSGFSLTSYGVHWVRQPPGKGLEWLGVIWVGGITHYNSALMSRLSISKDNSKSQVFLKMNSLQTDDIAMYYCARDGYYGDFDVWGAGTTVTVSS

SEQ ID NO: 149 - protein, 106 aa, synthetic construct
  REGION 1..106: note = Synthetic
DIQMTQSPSSLSASLGGKVTITCTASQDINKFIAWYQHKPGKGPRLLIHYTSTLQPGIPSRFSGSGSGRDYSFSISNLEPEDIATYYCLQYDNLRTFGGGTKLEIK

SEQ ID NO: 150 - protein, 117 aa, synthetic construct
  REGION 1..117: note = Synthetic
QVQLKESGPGLVAPSQSLSITCTVSGFSLTSYGVHWVRQPPGKGLEWLGVIWAGGITHYNSALMSRLSISKDNSKSQVFLKMNSPQTDDIAMYYCARDGYYGDFDVWGAGTTVTVSS

SEQ ID NO: 151 - protein, 106 aa, synthetic construct
  REGION 1..106: note = Synthetic
DIQMTQSPSSLSASLGGKVTITCKASQDINKYIAWYQHKPGKGPRLLIHYTSTLQPGIPSRFSGSGSGRDYSFSISNLVPEDIATYYCLQYDNLRTFGGGTKLEIK

SEQ ID NO: 152 - protein, 117 aa, synthetic construct
  REGION 1..117: note = Synthetic
QVQLKESGPGLVAPSQSLSITCTVSGFSLTSYGVHWVRQPPGKGLEWLGVIWAGGITHYNSALMSRLSISKDNSKSQVFLKMNSLQTDDTAMFYCARDGYYGHFDVWGAGTTVTVSS

SEQ ID NO: 153 - protein, 106 aa, synthetic construct
  REGION 1..106: note = Synthetic
DIQMTQSPSSLSASLGGKVTITCKASQDINKYIAWYQHKPGQGPRLLIDYTSTLQPGIPSRFSGSGSGRDYSFTISNLEPEDIATYFCLQYDNLRAFGGGTKLEIK

SEQ ID NO: 154 - protein, 119 aa, synthetic construct
  REGION 1..119: note = Synthetic
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRRANYVGAMDYWGQGTSVTVSS

SEQ ID NO: 155 - protein, 106 aa, synthetic construct
  REGION 1..106: note = Synthetic
QIVLSQSPAILSASPGEKVTMTCRASSSVSYMYWYQQKPGSSPKPWIYATSNLASGVPARFSGSGSGTSYSLTISRVEAEDAATYYCQQWSSNPLTFGAGTKLELK

SEQ ID NO: 156 - protein, 117 aa, synthetic construct
  REGION 1..117: note = Synthetic
EVKLVESGGGLVKPGGSLKLSCAASGFTFSSDAMSWVRQTPEKSLEWVASISIGGSAFYPESVRGRFTISRDNARNILYLQMSSLRSEDTAMYYCARGGDYDAMDYWGQGTSVTVSS

SEQ ID NO: 157 - protein, 107 aa, synthetic construct
  REGION 1..107: note = Synthetic
DIVMTQSPATLSVTPGDRVSLSCRASQSISDLLHWYQQKSHESPRLLIKYASQSISGIPSRFSGSGSGSDFTLSINSVEPEDVGVYFCQNGHNFPFTFGSGTKLEIK

SEQ ID NO: 158 - protein, 117 aa, synthetic construct
  REGION 1..117: note = Synthetic
EVKLVESGGGLVKPGGSLKLSCAASGFTFSSSAMSWVRQTPEKRLEWVASISVGGSTFYPDSVKGRFTISRDNARKILYLQMSSLRSEDTAMYYCARGGDYDAMDYWGQGTSVTVSS

SEQ ID NO: 159 - protein, 107 aa, synthetic construct
  REGION 1..107: note = Synthetic
DIVMTQSPATLSVTPGDRVSLSCRASQSISDYLHWFQQKSHESPRLLIKYASQSISGIPSRFSGSGSGSDFTLSINSVEPEDVGVYYCQNGHSFPFTFGSGTKLEIK

SEQ ID NO: 160 - protein, 117 aa, synthetic construct
  REGION 1..117: note = Synthetic
EVKLVESGGGLVKPGGSLKLSCAASGFTFSSSAMSWVRQTPEKRLEWVASISVGGSTFYPDSVKGRFTISRDNARNILYLQMSSLRSEDTAMYYCARGGDYDAMDYWGQGTSVTVSS

SEQ ID NO: 161 - protein, 107 aa, synthetic construct
  REGION 1..107: note = Synthetic
DIVMTQSPATLSVTPGDRVSLSCRASQSISDYLHWFQQKSHESPRLLIKYSSQSISGIPSRFSGSGSGSDFTLSINSVEPEDVGVYYCQNGHSFPFTFGSGTKLEIK

SEQ ID NO: 162 - protein, 117 aa, synthetic construct
  REGION 1..117: note = Synthetic
EVKLVESGGGLVKPGGSLKLSCAASGFTFSSSAMSWVRQTPEKRLEWVASISVGGSTFYADSVKGRFTISRDNARNILYLQMSSLRSEDTAMYYCARGGDYDPMDYWGQGTSVTVSS

SEQ ID NO: 163 - protein, 107 aa, synthetic construct; identical to SEQ ID NO: 159
  REGION 1..107: note = Synthetic

SEQ ID NO: 164 - protein, 117 aa, synthetic construct
  REGION 1..117: note = Synthetic
EVKLVESGGGLVKPGGSLKLSCAASGFTFSSDAMSWVRQTPEKRLEWVASISIGGSTFYPESVKGRFTISRDNARNILYLQMSSLRSEDTAMYYCARGGDYDAMDYWGQGTSVTVSS

SEQ ID NO: 165 - protein, 107 aa, synthetic construct; identical to SEQ ID NO: 157
  REGION 1..107: note = Synthetic

SEQ ID NO: 166 - protein, 117 aa, synthetic construct
  REGION 1..117: note = Synthetic
EVKVVESGGGLVKPGGSLKLSCATSGFTFSSSAMSWVRQTPERRLEWVASISVGGSIFYPDSVKGRFTISRENARNILYLQMSSLRSEDTAMYYCARGGDYDAMDYWGQGTSVTVSS

SEQ ID NO: 167 - protein, 107 aa, synthetic construct; identical to SEQ ID NO: 159
  REGION 1..107: note = Synthetic

SEQ ID NO: 168-169 - skipped (2 entries)

SEQ ID NO: 170 - protein, 117 aa, synthetic construct
  REGION 1..117: note = Synthetic
EVKLVESGGGLVKPGGSLKLSCAASGFTFSNCAMSWVRQTPEKRLEWVASISIGGSTFYPDSVKGRFTISRDNGRNILYLQMSSLRSEDTALYYCARGGDYDAMDYWGQGTSVTVSS

SEQ ID NO: 171 - protein, 107 aa, synthetic construct
  REGION 1..107: note = Synthetic
DIVMTQFPDTLSVTPGDRVSLSCRASQSISDLLHWYQQKSHESPRLLIKYASQSISEIPSRFSGSGSGSDFTLSINSVEPEDVGVYYCQNGHSFPFTFGSGTKLEIK

SEQ ID NO: 172 - protein, 117 aa, synthetic construct
  REGION 1..117: note = Synthetic
EVKLVESGGGLVKPGGSLKLSCEASGFTFSSDAMSWVRQTPEKRLEWVASISIGGSTFYPESVKGRFTISRDNARNILYLQMSSLRSEDTAMYYCTRGGDYDAMDYWGQGTSVTVSS

SEQ ID NO: 173 - protein, 107 aa, synthetic construct; identical to SEQ ID NO: 157
  REGION 1..107: note = Synthetic

SEQ ID NO: 174-177 - skipped (4 entries)

SEQ ID NO: 178 - DNA, 11 bp, synthetic construct
  source 1..11: mol_type = other DNA
arggdydamdy

SEQ ID NO: 179 - skipped

SEQ ID NO: 180 - protein, 402 aa, synthetic construct
  REGION 1..402: note = Synthetic
KQGCGEDKICQSNLQLVRARFCTRVSDTEFQPLPMDVDGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIELPLSIAGMAIPQQLFFSGVVRGERAMQSERDVGSKVKYEVTVSNQGQSLRTLGSAFLNIMWPHEIANGKWLLYPMQVELEGGQGPGQKGLCSPRPNILHLDVDSRDRRRRELEPPEQQEPGERQEPSMSWWPVSSAEKKKNITLDCARGTANCVVFSCPLYSFDRAAVLHVWGRLWNSTFLEEYSAVKSLEVIVRANITVKSSIKNLMLRDASTVIPVMVYLDPMAVVAEA

SEQ ID NO: 181 - protein, 7 aa, synthetic construct
  REGION 1..7: note = Synthetic
MAIPQQL

SEQ ID NO: 182 - protein, 23 aa, synthetic construct
  REGION 1..23: note = Synthetic
VVRGERAMQSERDVGSKVKYEVT

SEQ ID NO: 183 - protein, 7 aa, synthetic construct
  REGION 1..7: note = Synthetic
FDRAAVL

SEQ ID NO: 184 - protein, 9 aa, synthetic construct
  REGION 1..9: note = Synthetic
LRTLGSAFL

SEQ ID NO: 185 - protein, 9 aa, synthetic construct
  REGION 1..9: note = Synthetic
VVFSCPLYS

SEQ ID NO: 186 - protein, 121 aa, synthetic construct
  REGION 1..121: note = Synthetic
QVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGEGWYFDVWGQGTLVTVSS

SEQ ID NO: 187 - protein, 112 aa, synthetic construct
  REGION 1..112: note = Synthetic
DVVMTQSPLSLPVTPGEPASISCRSSQSLVHRNGNTYLHWYLQKPGQSPQLLIYKVSNRFSGVPDRFSGSGSGTDFTLKISRVEAEDVGVYFCSQSTHVPWTFGGGTKLEIK

SEQ ID NO: 188 - protein, 14 aa, synthetic construct
  REGION 1..14: note = Synthetic
ARGGYFGEGWYHDV

SEQ ID NO: 189 - protein, 14 aa, synthetic construct
  REGION 1..14: note = Synthetic
ARGGYFGRQWYFDV

SEQ ID NO: 190 - protein, 14 aa, synthetic construct
  REGION 1..14: note = Synthetic
ARGGYFGEQWYHDV

SEQ ID NO: 191 - protein, 14 aa, synthetic construct
  REGION 1..14: note = Synthetic
ARGGYFGVGWYNDV

SEQ ID NO: 192 - protein, 14 aa, synthetic construct
  REGION 1..14: note = Synthetic
ARGGYFGETWYHDV

SEQ ID NO: 193 - protein, 14 aa, synthetic construct
  REGION 1..14: note = Synthetic
ARGGYFGENWYHDV

SEQ ID NO: 194 - protein, 14 aa, synthetic construct
  REGION 1..14: note = Synthetic
ARGGYFGEKWYHDV

SEQ ID NO: 195 - protein, 14 aa, synthetic construct
  REGION 1..14: note = Synthetic
ARGGYFGEEWYNDV

SEQ ID NO: 196-197 - skipped (2 entries)

SEQ ID NO: 198 - protein, 9 aa, synthetic construct
  REGION 1..9: note = Synthetic
AQSIQVPWT

SEQ ID NO: 199 - protein, 121 aa, synthetic construct
  REGION 1..121: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGEGWYHDVWGQGTLVTVSS

SEQ ID NO: 200 - protein, 112 aa, synthetic construct
  REGION 1..112: note = Synthetic
DIVMTQSPLSLPVTPGEPASISCRSSQSLVHRNGNTYLHWYLQKPGQSPQLLIYEVSKRASGVPDRFSGSGSGTDFTLKISRVEAEDVGVYFCAQSIQVPWTFGGGTKLEIK

SEQ ID NO: 201 - protein, 121 aa, synthetic construct
  REGION 1..121: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGRQWYFDVWGQGTLVTVSS

SEQ ID NO: 202 - protein, 112 aa, synthetic construct
  REGION 1..112: note = Synthetic
DIVMTQSPLSLPVTPGEPASISCRSSQSLVHRNGNTYLHWYLQKPGQSPQLLIYEVSNRASGVPDRFSGSGSGTDFTLKISRVEAEDVGVYFCSQSTHVPWTFGGGTKLEIK

SEQ ID NO: 203 - protein, 121 aa, synthetic construct
  REGION 1..121: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGEQWYHDVWGQGTLVTVSS

SEQ ID NO: 204 - protein, 112 aa, synthetic construct; identical to SEQ ID NO: 202
  REGION 1..112: note = Synthetic

SEQ ID NO: 205 - protein, 121 aa, synthetic construct
  REGION 1..121: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGVGWYNDVWGQGTLVTVSS

SEQ ID NO: 206 - protein, 112 aa, synthetic construct
  REGION 1..112: note = Synthetic
DIVMTQSPLSLPVTPGEPASISCRSSQSLVHRNGNTYLHWYLQKPGQSPQLLIYEVSNRVPGVPDRFSGSGSGTDFTLKISRVEAEDVGVYFCSQSTHVPWTFGGGTKLEIK

SEQ ID NO: 207 - protein, 121 aa, synthetic construct
  REGION 1..121: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGETWYHDVWGQGTLVTVSS

SEQ ID NO: 208 - protein, 112 aa, synthetic construct
  REGION 1..112: note = Synthetic
DIVMTQSPLSLPVTPGEPASISCRSSQSLVHRNGNTYLHWYLQKPGQSPQLLIYEVSHRASGVPDRFSGSGSGTDFTLKISRVEAEDVGVYFCSQSTHVPWTFGGGTKLEIK

SEQ ID NO: 209 - protein, 121 aa, synthetic construct
  REGION 1..121: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGENWYHDVWGQGTLVTVSS

SEQ ID NO: 210 - protein, 112 aa, synthetic construct
  REGION 1..112: note = Synthetic
DIVMTQSPLSLPVTPGEPASISCRSSQSLVHRNGNTYLHWYLQKPGQSPQLLIYEVSNRYSGVPDRFSGSGSGTDFTLKISRVEAEDVGVYFCAQSIQVPWTFGGGTKLEIK

SEQ ID NO: 211 - protein, 121 aa, synthetic construct
  REGION 1..121: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGEKWYHDVWGQGTLVTVSS

SEQ ID NO: 212 - protein, 112 aa, synthetic construct
  REGION 1..112: note = Synthetic
DIVMTQSPLSLPVTPGEPASISCRSSQSLVHRNGNTYLHWYLQKPGQSPQLLIYEVSHRFPGVPDRFSGSGSGTDFTLKISRVEAEDVGVYFCAQSTQLPYTFGGGTKLEIK

SEQ ID NO: 213 - protein, 121 aa, synthetic construct; identical to SEQ ID NO: 211
  REGION 1..121: note = Synthetic

SEQ ID NO: 214 - protein, 112 aa, synthetic construct
  REGION 1..112: note = Synthetic
DIVMTQSPLSLPVTPGEPASISCRSSQSLVHRNGNTYLHWYLQKPGQSPQLLIYGVSNRFAGVPDRFSGSGSGTDFTLKISRVEAEDVGVYFCAQSTQLPYTFGGGTKLEIK

SEQ ID NO: 215 - protein, 121 aa, synthetic construct
  REGION 1..121: note = Synthetic
EVQLVQSGAEVKKPGSSVKVSCKASGYSFTRYWMHWVRQAPGQGLEWIGKIDPSDSETRLNQEFRDRATLTVDKSTSTAYMELSSLRSEDTAVYYCARGGYFGEEWYNDVWGQGTLVTVSS

SEQ ID NO: 216 - protein, 112 aa, synthetic construct
  REGION 1..112: note = Synthetic
DIVMTQSPLSLPVTPGEPASISCRSSQSLVHRNGNTYLHWYLQKPGQSPQLLIYKVSNRASGVPDRFSGSGSGTDFTLKISRVEAEDVGVYFCSQSTHVPWTFGGGTKLEIK

SEQ ID NO: 217 - protein, 121 aa, synthetic construct; identical to SEQ ID NO: 110
  REGION 1..121: note = Synthetic

SEQ ID NO: 218 - protein, 112 aa, synthetic construct; identical to SEQ ID NO: 216
  REGION 1..112: note = Synthetic

SEQ ID NO: 219 - protein, 121 aa, synthetic construct; identical to SEQ ID NO: 110
  REGION 1..121: note = Synthetic

SEQ ID NO: 220 - protein, 112 aa, synthetic construct
  REGION 1..112: note = Synthetic
DIVMTQSPLSLPVTPGEPASISCRSSQSLVHRNGNTYLHWYLQKPGQSPQLLIYEGSKRFSGVPDRFSGSGSGTDFTLKISRVEAEDVGVYFCSQSTHVPWTFGGGTKLEIK

SEQ ID NO: 221 - protein, 121 aa, synthetic construct; identical to SEQ ID NO: 203
  REGION 1..121: note = Synthetic

SEQ ID NO: 222 - protein, 112 aa, synthetic construct
  REGION 1..112: note = Synthetic
DIVMTQSPLSLPVTPGEPASISCRSSQSLVHRNGNTYLHWYLQKPGQSPQLLIYEVSKRFSGVPDRFSGSGSGTDFTLKISRVEAEDVGVYFCAQSIQVPWTFGGGTKLEIK

SEQ ID NO: 223 - protein, 121 aa, synthetic construct; identical to SEQ ID NO: 201
  REGION 1..121: note = Synthetic

SEQ ID NO: 224 - protein, 112 aa, synthetic construct
  REGION 1..112: note = Synthetic
DIVMTQSPLSLPVTPGEPASISCRSSQSLVHRNGNTYLHWYLQKPGQSPQLLIYEGSKRFSGVPDRFSGSGSGTDFTLKISRVEAEDVGVYFCAQSTQLPYTFGGGTKLEIK

SEQ ID NO: 225 - protein, 172 aa, Homo sapiens
QGCGEDKICQSNLQLVRARFCTRVSDTEFQPLPMDVDGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIELP

SEQ ID NO: 226 - protein, 172 aa, Macaca fascicularis
QGCGEDKICQSNLQLVRARFCARVSDTEFQPLPMDADGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIELP

SEQ ID NO: 227 - protein, 172 aa, Mus musculus
QGCGQDKICQSNLQLERYQFCSRISDTEFQALPMDLDGRTALFALSGQPFIGLELTVTNLPSDPSRPQADGDDAHEAQLLVTLPASLRYSGVRALDSVEKPLCLSNDSASHVECELGNPMKRGAQVTFYLILSTSGITIETTELEVKLLLATISEQELDPVSVRAHVFIELP

(19) United States
(12) Patent Application Publication
(10) Pub. No.: US 2025/0289894 A1
(43) Pub. Date: Sep. 18, 2025

(54) INTEGRIN ALPHA-7 BETA-1 ANTIBODIES AND USES THEREOF

(71) Applicant: Morphic Therapeutic, Inc., Waltham, MA (US)

(72) Inventors: Hua Wang, Waltham, MA (US); Fu-Yang Lin, Sudbury, MA (US); Xinqiang Huang, Waltham, MA (US); Ziad Mahmassani, Waltham, MA (US); Kerim Babaoglu, Sudbury, MA (US); Terence Moy, Waltham, MA (US); Jessica Pondish, Watertown, MA (US)

(21) Appl. No.: 19/080,336

(22) Filed: Mar. 14, 2025

Related U.S. Application Data
(60) Provisional application No. 63/566,145, filed on Mar. 15, 2024.
(60) Provisional application No. 63/667,476, filed on Jul. 3, 2024.

(51) Int. Cl. C07K 16/28; A61K 39/00; A61P 21/00

(52) U.S. Cl. CPC C07K 16/2842; A61P 21/00; A61K 2039/505; C07K 2317/24; C07K 2317/34; C07K 2317/55; C07K 2317/75; C07K 2317/92

(57) ABSTRACT

The present disclosure provides, among other things, a class of antibodies or an antigen-binding fragments that bind to calf-1 or calf-2 domain of α7β1 integrin. The anti-α7β1 antibodies of the present disclosure are effective in stabilizing the α7β1 integrin in the extended-closed and extended-open conformations, which enhances the binding to laminins.

### CROSS-REFERENCE TO RELATED APPLICATIONS

This application claims the benefit of priority to U.S. Provisional Application Nos. 63/667,476, filed Jul. 3, 2024; and 63/566,145, filed Mar. 15, 2024; the contents of each of which are hereby incorporated by reference in their entireties.

### REFERENCE TO ELECTRONIC SEQUENCE LISTING

This application contains a Sequence Listing which has been submitted electronically in .XML format and is hereby incorporated by reference in its entirety. Said .XML copy, created on May 8, 2025, is named “MTX_04201.XML” and is 253,749 bytes in size. The sequence listing contained in this .xml file is part of the specification and is hereby incorporated by reference herein in its entirety.

### BACKGROUND

Integrin α7β1 is a major laminin receptor in skeletal and cardiac muscle. It serves as a transmembrane linkage between basal lamina and muscle cell membrane to maintain functional integrity of muscle cells. Loss of function mutations of ITGA7 in humans results in congenital myopathies and adult-onset cardiomyopathy. In contrast, overexpression of ITGA7 in the skeletal muscle ameliorated the development of muscular dystrophy and overexpression of ITGA7 in the cardiomyocyte protects cardiomyocyte from ischemia/reperfusion injury.

Muscular dystrophies, such as Duchenne Muscular Dystrophy (DMD), are a category of genetically inherited degenerative disorders characterized by progressive deterioration of muscles and loss of muscle related functions throughout the body. Existing treatments for muscular dystrophy disorders, including dystrophinopathies, simply focus on mitigating the effects of the disease and improving quality of life, often through glucocorticoids, physical therapy or use of orthopedic devices. There is a need for new approaches to treat dystrophinopathies, including DMD.

### SUMMARY OF THE INVENTION

The present disclosure provides, among other things, integrin α7β1 antibodies and therapeutic uses of such antibodies in effectively treating diseases or disorders associated with muscle membrane integrity, such as Duchenne Muscular Dystrophy (DMD). As described herein, the present disclosure is based, in part, on identification of integrin α7β1 antibodies that are effective in stabilizing the α7β1 integrin in the extended-closed and extended-open conformations and therefore enhancing the binding to laminin ligands. As a result, the anti-α7β1 antibodies of the present disclosure potently increase adhesion of muscle membranes (sarcolemma) to the extracellular matrix, restoring some or all of the sarcolemma damages due to the absence of functional dystrophin and ultimately improving locomotive ability, increasing muscle force, and reducing fibrosis in patients with dystrophinopathies.

In one aspect, the present disclosure provides an antibody or antigen-binding fragment thereof that binds to the calf-2 domain of α7β1 integrin but does not compete with the 20TFT antibody, wherein the antibody stabilizes the extended conformations of α7β1 integrin.

In one aspect, the present disclosure provides an antibody or antigen-binding fragment thereof that binds to α7β1 integrin and competes with a reference antibody for binding to α7β1, wherein the reference antibody comprises an HCDR1 of sequence GFTFSSYT (SEQ ID NO: 2, HCDR1B), an HCDR2 of sequence ISIVGNYI (SEQ ID NO: 16, HCDR2B), an HCDR3 of sequence ARRKWEHDAFDI (SEQ ID NO: 32, HCDR3B), an LCDR1 of sequence SLRSYY (SEQ ID NO: 47, LCDR1B), an LCDR2 of sequence GKN (SEQ ID NO: 59, LCDR2B), and an LCDR3 of sequence TSRDNSGNHVV (SEQ ID NO: 70, LCDR3B).

In one aspect, the present disclosure provides an antibody or antigen-binding fragment thereof that binds to α7β1 integrin, wherein the antibody or antigen-binding fragment thereof comprises (a) a means for stabilizing the extended conformations of the α7β1 integrin and, optionally, (b) an Fc domain. In some embodiments, the antibody is not the 20TFT antibody.

In one aspect, the present disclosure provides an antibody or antigen-binding fragment thereof that binds to α7β1, wherein the antibody or antigen-binding fragment thereof binds to a region comprising positions 856-862, 867-889, 1011-1017, 897-905, or 1002-1010 of the amino acid sequence of integrin α7, and wherein the antibody or antigen-binding fragment thereof stabilizes the extended conformations of α7β1 integrin.

In some embodiments, the antibody or antigen-binding fragment thereof binds to a region comprising positions 856-862, 867-889, or 1011-1017 of the amino acid sequence of integrin α7. In some embodiments, the antibody or antigen-binding fragment thereof binds to a region comprising positions 856-862 of the amino acid sequence of integrin α7. In some embodiments, the antibody or antigen-binding fragment thereof binds to a region comprising positions 867-889 of the amino acid sequence of integrin α7. In some embodiments, the antibody or antigen-binding fragment thereof binds to a region comprising positions 1011-1017 of the amino acid sequence of integrin α7.

In some embodiments, the antibody or antigen-binding fragment thereof binds to a region comprising residues M856, A857, 1858, P859, Q860, Q861, and L862 of integrin α7. In some embodiments, the antibody or antigen-binding fragment thereof binds to a region comprising residues V867, V868, R869, G870, E871, R872, A873, M874, Q875, S876, E877, R878, D879, V880, G881, S882, K883, V884, K885, Y886, E887, V888, and T889 of integrin α7. In some embodiments, the antibody or antigen-binding fragment thereof binds to a region comprising residues F1011, D1102, R1103, A1104, A1105, V1106, and L1107 of integrin α7.

In some embodiments, the antibody or antigen-binding fragment thereof binds to a region comprising positions 897-905 or 1002-1010 of the amino acid sequence of integrin α7. In some embodiments, the antibody or antigen-binding fragment thereof binds to a region comprising positions 897-905 of the amino acid sequence of integrin α7. In some embodiments, the antibody or antigen-binding fragment thereof binds to a region comprising positions 1002-1010 of the amino acid sequence of integrin α7.

In some embodiments, the antibody or antigen-binding fragment thereof binds to a region comprising positions L897, R898, T899, L900, G901, S902, A903, F904, and L905 of integrin α7. In some embodiments, the antibody or antigen-binding fragment thereof binds to a region comprising positions V1002, V1003, F1004, S1005, C1006, P1007, L1008, Y1009, and S1100 of integrin α7.

In one aspect, the present disclosure provides an antibody or antigen-binding fragment thereof that binds to the calf-1 domain of α7β1 integrin and stabilizes the extended conformations of α7β1 integrin.

In one aspect, the present disclosure provides an antibody or antigen-binding fragment that binds to α7β1 integrin and competes with a reference antibody for binding to α7β1 antibody, wherein the reference antibody comprises a HCDR1 of sequence GYSFTRYW (SEQ ID NO: 7, HCDR1G), a HCDR2 of sequence IDPSDSET (SEQ ID NO: 22, HCDR2H), a HCDR3 of sequence ARGGYFGEGWYFDV (SEQ ID NO: 38, HCDR3H), a LCDR1 of sequence QSLVHRNGNTY (SEQ ID NO: 51, LCDR1F), a LCDR2 of sequence KVS (SEQ ID NO: 63, LCDR2F), and a LCDR3 of sequence SQSTHVPWT (SEQ ID NO: 75, LCDR3G).

In one aspect, the present disclosure provides an antibody or antigen-binding fragment that binds to α7β1 integrin and competes with a reference antibody for binding to α7β1 antibody, wherein the reference antibody comprises a HCDR1 of sequence GFSLTSYG (SEQ ID NO: 9, HCDR1I), a HCDR2 of sequence IWVGGIT (SEQ ID NO: 24, HCDR2J), a HCDR3 of sequence ARDGYYGDFDV (SEQ ID NO: 40, HCDR3J), a LCDR1 of sequence QDINKF (SEQ ID NO: 53, LCDR1H), a LCDR2 of sequence YTS (SEQ ID NO: 65, LCDR2H), and a LCDR3 of sequence LQYDNLRT (SEQ ID NO: 77 LCDR3I).

In one aspect, the present disclosure provides an antibody or an antigen-binding fragment that binds to α7β1 integrin comprising (a) a means for binding to calf-1 domain of α7β1 integrin and (b) an Fc domain.

In one aspect, the present disclosure provides an antibody or antigen-binding fragment that binds to α7β1 integrin, wherein the antibody binds to a region comprising positions 771-778, 793-779, or 803-805 of integrin α7, wherein the antibody stabilizes the extended conformations of α7β1 integrin. In some embodiments, the region comprises amino acids R771, A772, L773, D774, A776, E777, K778, E793, N796, K799, Q803, and/or T805 according to human α7 amino acid numbering.

In one aspect, the present disclosure provides an antibody or antigen-binding fragment that binds to α7β1 integrin, wherein the antibody comprises a HCDR1 of sequence GFTFSSYT (SEQ ID NO: 2, HCDR1B), a HCDR2 of sequence ISIVGNYI (SEQ ID NO: 16, HCDR2B), a HCDR3 of sequence ARRKWEHDAFDI (SEQ ID NO: 32, HCDR3B), a LCDR1 of sequence SLRSYY (SEQ ID NO: 47, LCDR1B), a LCDR2 of sequence GKN (SEQ ID NO: 59, LCDR2B), and a LCDR3 of sequence TSRDNSGNHVV (SEQ ID NO: 70, LCDR3B).

In one aspect, the present disclosure provides an antibody or antigen-binding fragment that binds to α7β1 integrin, wherein the antibody comprises a HCDR1 of sequence GYTFTGYY (SEQ ID NO:1, HCDR1A), a HCDR2 of sequence INPNSGGT (SEQ ID NO: 15, HCDR2A), a HCDR3 of sequence AREGANWDYYYYGMDV (SEQ ID NO: 31, HCDR3A), a LCDR1 of sequence SSNIGSKT (SEQ ID NO: 46, LCDR1A), a LCDR2 of sequence SNN (SEQ ID NO: 58, LCDR2A), and a LCDR3 of sequence AAWDDSLNGPV (SEQ ID NO: 69, LCDR3A).

In one aspect, the present disclosure provides an antibody or antigen-binding fragment that binds to α7β1 integrin, wherein the antibody comprises a HCDR1 of sequence GFTFSSYS (SEQ ID NO:3, HCDR1C), a HCDR2 of sequence ISTSSSYI (SEQ ID NO: 17, HCDR2C), a HCDR3 of sequence ARRAWESDAFAI (SEQ ID NO: 33, HCDR3C), a LCDR1 of sequence SLRSYY (SEQ ID NO: 47, LCDR1B), a LCDR2 of sequence GKN (SEQ ID NO: 59, LCDR2B), and a LCDR3 of sequence NSRDSSGNHVV (SEQ ID NO: 71, LCDR3C).

In one aspect, the present disclosure provides an antibody or antigen-binding fragment that binds to α7β1 integrin, wherein the antibody comprises a HCDR1 of sequence GFTFSSYA (SEQ ID NO: 4, HCDR1D), a HCDR2 of sequence ISDSGGIA (SEQ ID NO: 18, HCDR2D), a HCDR3 of sequence VRDGY (SEQ ID NO: 34, HCDR3D), a LCDR1 of sequence QSLLDSDDGYTY (SEQ ID NO: 48, LCDR1C), a LCDR2 of sequence TLS (SEQ ID NO: 60, LCDR2C), and a LCDR3 of sequence MQRIEFPLT (SEQ ID NO: 72, LCDR3D).

In one aspect, the present disclosure provides an antibody or antigen-binding fragment that binds to α7β1 integrin, wherein the antibody comprises a HCDR1 of sequence GFTFSSYA (SEQ ID NO: 4, HCDR1D), a HCDR2 of sequence IHYSGGTT (SEQ ID NO: 19, HCDR2E), a HCDR3 of sequence VKDGY (SEQ ID NO: 35, HCDR3E), a LCDR1 of sequence QSLLDSDDGYTY (SEQ ID NO: 48, LCDR1C), a LCDR2 of sequence TLS (SEQ ID NO: 60, LCDR2C), and a LCDR3 of sequence MQRIEFPLT (SEQ ID NO: 72, LCDR3D).

In one aspect, the present disclosure provides an antibody or antigen-binding fragment that binds to α7β1 integrin, wherein the antibody comprises a HCDR1 of sequence GFTFSRFW (SEQ ID NO: 5, HCDR1E), a HCDR2 of sequence TKQDGSEK (SEQ ID NO: 20, HCDR2F), a HCDR3 of sequence AREGSGAYWAFDI (SEQ ID NO: 36, HCDR3F), a LCDR1 of sequence QSLVHNDGNTY (SEQ ID NO: 49, LCDR1D), a LCDR2 of sequence KIS (SEQ ID NO: 49, LCDR1D), and a LCDR3 of sequence LQATQFPLT (SEQ ID NO: 73, LCDR3E).

In one aspect, the present disclosure provides an antibody or antigen-binding fragment that binds to α7β1 integrin, wherein the antibody comprises a HCDR1 of sequence GGSISSYY (SEQ ID NO: 6, HCDR1F), a HCDR2 of sequence IYTRGST (SEQ ID NO: 21, HCDR2G), a HCDR3 of sequence ARVFGGNYRFDAFHI (SEQ ID NO: 37, HCDR3G), a LCDR1 of sequence SSNIGAGYD (SEQ ID NO: 50, LCDR1E), a LCDR2 of sequence GNN (SEQ ID NO: 62, LCDR2E), and a LCDR3 of sequence QSYDSSLSGYV (SEQ ID NO: 74, LCDR3F).

In one aspect, the present disclosure provides an antibody or antigen-binding fragment that binds to α7β1 integrin, wherein the antibody comprises a HCDR1 of sequence GYSFTRYW (SEQ ID NO: 7, HCDR1G), a HCDR2 of sequence IDPSDSET (SEQ ID NO: 22, HCDR2H), a HCDR3 of sequence ARGGYFGEGWYFDV (SEQ ID NO: 38, HCDR3H), a LCDR1 of sequence QSLVHRNGNTY (SEQ ID NO: 51, LCDRIF), a LCDR2 of sequence KVS (SEQ ID NO: 63, LCDR2F), and a LCDR3 of sequence SQSTHVPWT (SEQ ID NO: 75, LCDR3G).

In one aspect, the present disclosure provides an antibody or antigen-binding fragment that binds to α7β1 integrin, wherein the antibody comprises a HCDR1 of sequence GYTFTSYW (SEQ ID NO: 8, HCDR1H), a HCDR2 of sequence IYPSDSYT (SEQ ID NO: 23, HCDR2I), a HCDR3 of sequence TRRANYVGAMDY (SEQ ID NO: 39, HCDR3I), a LCDR1 of sequence SSVSY (SEQ ID NO: 52, LCDR1G), a LCDR2 of sequence ATS (SEQ ID NO: 64, LCDR2G), and a LCDR3 of sequence QQWSSNPLT (SEQ ID NO: 76, LCDR3H).

In one aspect, the present disclosure provides an antibody or antigen-binding fragment that binds to α7β1 integrin, wherein the antibody comprises a HCDR1 of sequence GFSLTSYG (SEQ ID NO: 9, HCDR1I), a HCDR2 of sequence IWVGGIT (SEQ ID NO: 24, HCDR2J), a HCDR3 of sequence ARDGYYGDFDV (SEQ ID NO: 40, HCDR3J), a LCDR1 of sequence QDINKF (SEQ ID NO: 53, LCDR1H), a LCDR2 of sequence YTS (SEQ ID NO: 65, LCDR2H), and a LCDR3 of sequence LQYDNLRT (SEQ ID NO: 77, LCDR3I).

In one aspect, the present disclosure provides an antibody or antigen-binding fragment that binds to α7β1 integrin, wherein the antibody comprises a HCDR1 of sequence GFSLTSYG (SEQ ID NO: 9, HCDR1I), a HCDR2 of sequence IWAGGIT (SEQ ID NO: 25, HCDR2K), a HCDR3 of sequence ARDGYYGDFDV (SEQ ID NO: 40, HCDR3J), a LCDR1 of sequence QDINKY (SEQ ID NO: 54, LCDR1I), a LCDR2 of sequence YTS (SEQ ID NO: 65, LCDR2H), and a LCDR3 of sequence LQYDNLRT (SEQ ID NO: 77, LCDR3I).

In one aspect, the present disclosure provides an antibody or antigen-binding fragment that binds to α7β1 integrin, wherein the antibody comprises a HCDR1 of sequence GFSLTSYG (SEQ ID NO: 9, HCDR1I), a HCDR2 of sequence IWAGGIT (SEQ ID NO: 25, HCDR2K), a HCDR3 of sequence ARDGYYGHFDV (SEQ ID NO: 41, HCDR3K), a LCDR1 of sequence QDINKY (SEQ ID NO: 54, LCDR1I), a LCDR2 of sequence YTS (SEQ ID NO: 65, LCDR2H), and a LCDR3 of sequence LQYDNLRA (SEQ ID NO: 78, LCDR3J).

In one aspect, the present disclosure provides an antibody or antigen-binding fragment that binds to α7β1 integrin, wherein the antibody comprises a HCDR1 of sequence GFTFSSDA (SEQ ID NO: 10, HCDR1J), a HCDR2 of sequence ISIGGSA (SEQ ID NO: 26, HCDR2L), a HCDR3 of sequence ARGGDYDAMDY (SEQ ID NO: 42, HCDR3L), a LCDR1 of sequence QSISDL (SEQ ID NO: 55, LCDR1J), a LCDR2 of sequence YAS (SEQ ID NO: 66, LCDR2I), and a LCDR3 of sequence QNGHNFPFT (SEQ ID NO: 79, LCDR3K).

In one aspect, the present disclosure provides an antibody or antigen-binding fragment that binds to α7β1 integrin, wherein the antibody comprises a HCDR1 of sequence GFTFSSSA (SEQ ID NO: 11, HCDR1K), a HCDR2 of sequence ISVGGST (SEQ ID NO: 27, HCDR2M), a HCDR3 of sequence ARGGDYDAMDY (SEQ ID NO: 42, HCDR3L), a LCDR1 of sequence QSISDY (SEQ ID NO: 56, LCDR1K), a LCDR2 of sequence YAS (SEQ ID NO: 66, LCDR2I), and a LCDR3 of sequence QNGHSFPFT (SEQ ID NO: 80, LCDR3L).

In one aspect, the present disclosure provides an antibody or antigen-binding fragment that binds to α7β1 integrin, wherein the antibody comprises a HCDR1 of sequence GFTFSSSA (SEQ ID NO: 11, HCDR1K), a HCDR2 of sequence ISVGGST (SEQ ID NO: 27, HCDR2M), a HCDR3 of sequence ARGGDYDAMDY (SEQ ID NO: 42, HCDR3L), a LCDR1 of sequence QSISDY (SEQ ID NO: 56, LCDR1K), a LCDR2 of sequence YSS (SEQ ID NO: 67, LCDR2J), and a LCDR3 of sequence QNGHSFPFT (SEQ ID NO: 80, LCDR3L).

In one aspect, the present disclosure provides an antibody or antigen-binding fragment that binds to α7β1 integrin, wherein the antibody comprises a HCDR1 of sequence GFTFSSSA (SEQ ID NO: 11, HCDR1K), a HCDR2 of sequence ISVGGST (SEQ ID NO: 27, HCDR2M), a HCDR3 of sequence ARGGDYDPMDY (SEQ ID NO: 43, HCDR3M), a LCDR1 of sequence QSISDY (SEQ ID NO: 56, LCDR1K), a LCDR2 of sequence YAS (SEQ ID NO: 66, LCDR2I), and a LCDR3 of sequence QNGHSFPFT (SEQ ID NO: 80, LCDR3L).

In one aspect, the present disclosure provides an antibody or antigen-binding fragment that binds to α7β1 integrin, wherein the antibody comprises a HCDR1 of sequence GFTFSSDA (SEQ ID NO: 10, HCDR1J), a HCDR2 of sequence ISIGGST (SEQ ID NO: 28, HCDR2N), a HCDR3 of sequence ARGGDYDAMDY (SEQ ID NO: 42, HCDR3L), a LCDR1 of sequence QSISDL (SEQ ID NO: 55, LCDR1J), a LCDR2 of sequence YAS (SEQ ID NO: 66, LCDR2I), and a LCDR3 of sequence QNGHNFPFT (SEQ ID NO: 79, LCDR3K).

In one aspect, the present disclosure provides an antibody or antigen-binding fragment that binds to α7β1 integrin, wherein the antibody comprises a HCDR1 of sequence GFTFSSSA (SEQ ID NO: 11, HCDR1K), a HCDR2 of sequence ISVGGSI (SEQ ID NO: 29, HCDR2O), a HCDR3 of sequence ARGGDYDAMDY (SEQ ID NO: 42, HCDR3L), a LCDR1 of sequence QSISDY (SEQ ID NO: 56, LCDR1K), a LCDR2 of sequence YAS (SEQ ID NO: 66, LCDR2I), and a LCDR3 of sequence QNGHSFPFT (SEQ ID NO: 80, LCDR3L).

In one aspect, the present disclosure provides an antibody or antigen-binding fragment that binds to α7β1 integrin, wherein the antibody comprises a HCDR1 of sequence GFTFSSDA (SEQ ID NO: 10, HCDR1J), a HCDR2 of sequence ISIGGST (SEQ ID NO: 28, HCDR2N), a HCDR3 of sequence ARGGDYDAMDY (SEQ ID NO: 42, HCDR3L), a LCDR1 of sequence QSISDL (SEQ ID NO: 55, LCDR1J), a LCDR2 of sequence YAS (SEQ ID NO: 66, LCDR2I), and a LCDR3 of sequence QNGHNFPFT (SEQ ID NO: 79, LCDR3K).

In one aspect, the present disclosure provides an antibody or antigen-binding fragment that binds to α7β1 integrin, wherein the antibody comprises a HCDR1 of sequence GFTFSNCA (SEQ ID NO: 12, HCDR1L), a HCDR2 of sequence ISIGGST (SEQ ID NO: 28, HCDR2N), a HCDR3 of sequence ARGGDYDAMDY (SEQ ID NO: 42, HCDR3L), a LCDR1 of sequence QSISDL (SEQ ID NO: 55, LCDR1J), a LCDR2 of sequence YAS (SEQ ID NO: 66, LCDR2I), and a LCDR3 of sequence QNGHSFPFT (SEQ ID NO: 80, LCDR3L).

In one aspect, the present disclosure provides an antibody or antigen-binding fragment that binds to α7β1 integrin, wherein the antibody comprises a HCDR1 of sequence GFTFSSDA (SEQ ID NO: 10, HCDR1J), a HCDR2 of sequence ISIGGST (SEQ ID NO: 28, HCDR2N), a HCDR3 of sequence TRGGDYDAMDY (SEQ ID NO: 44, HCDR3N), a LCDR1 of sequence QSISDL (SEQ ID NO: 55, LCDR1J), a LCDR2 of sequence YAS (SEQ ID NO: 66, LCDR2I), and a LCDR3 of sequence QNGHNFPFT (SEQ ID NO: 79, LCDR3K).

In one aspect, the present disclosure provides an antibody or antigen-binding fragment that binds to α7β1 integrin, wherein the antibody comprises a HCDR1 of sequence GFSLSTSDMG (SEQ ID NO: 13, HCDR1M), a HCDR2 of sequence IYWDDDK (SEQ ID NO: 30, HCDR2P), a HCDR3 of sequence VRGTAVVAFYWYFDV (SEQ ID NO: 45, HCDR30), a LCDR1 of sequence QDINNY (SEQ ID NO: 57, LCDR1L), a LCDR2 of sequence RAN (SEQ ID NO: 68, LCDR2K), and a LCDR3 of sequence LHYDDFPLT (SEQ ID NO: 81, LCDR3M).

In one aspect, the present disclosure provides an antibody or antigen-binding fragment that binds to α7β1 integrin, wherein the antibody comprises a HCDR1 of sequence GFSLSTSDLG (SEQ ID NO: 14, HCDR1N), a HCDR2 of sequence IYWDDDK (SEQ ID NO: 30, HCDR2P), a HCDR3 of sequence VRGTAVVAFYWYFDV (SEQ ID NO: 45, HCDR30), a LCDR1 of sequence QDINNY (SEQ ID NO: 57, LCDR1L), a LCDR2 of sequence RAN (SEQ ID NO: 68, LCDR2K), and a LCDR3 of sequence LQYDDFPLT (SEQ ID NO: 82, LCDR3N).

In one aspect, the present disclosure provides an antibody or an antigen-binding fragment that binds α7β1 integrin, wherein the antibody comprises a HCDR1 of sequence GYSFTRYW (SEQ ID NO: 101), a HCDR2 of sequence IDPSDSET (SEQ ID NO: 27), and a HCDR3 of sequence ARGGYFGEGWYFDV (SEQ ID NO: 38); and a LCDR1 of sequence QSLVHRNGNTY (SEQ ID NO: 105), a LCDR2 of sequence KVS (SEQ ID NO: 63), and a LCDR3 of sequence SQSTHVPWT (SEQ ID NO: 75).

In one aspect, the present disclosure provides an antibody or an antigen-binding fragment that binds α7β1 integrin, wherein the antibody comprises a HCDR1 of sequence GYSFTRYW (SEQ ID NO: 101), a HCDR2 of sequence IDPSDSET (SEQ ID NO: 102), and a HCDR3 of sequence ARGGYFGREWYYDV (SEQ ID NO: 103); and a LCDR1 of sequence QSLVHRNGNTY (SEQ ID NO: 105), a LCDR2 of sequence EVS (SEQ ID NO: 106), and a LCDR3 of sequence SQSTHVPWT (SEQ ID NO: 75).

In one aspect, the present disclosure provides an antibody or an antigen-binding fragment that binds α7β1 integrin, wherein the antibody comprises a HCDR1 of sequence GYSFTRYW (SEQ ID NO: 101), a HCDR2 of sequence IDPSDSET (SEQ ID NO: 102), and a HCDR3 of sequence ARGGYFGEEWYHDV (SEQ ID NO: 104); and a LCDR1 of sequence QSLVHRNGNTY (SEQ ID NO: 105), a LCDR2 of sequence EVS (SEQ ID NO: 106), and a LCDR3 of sequence AQSTQLPYT (SEQ ID NO: 107).

In one aspect, the present disclosure provides an antibody or an antigen-binding fragment that binds α7β1 integrin, wherein the antibody comprises a, HCDR1 of SEQ ID NO: 101, an HCDR2 of SEQ ID NO: 102, an HCDR3 of SEQ ID NO: 188, an LCDR1 of SEQ ID NO: 105, an LCDR2 of SEQ ID NO: 106, and an LCDR3 of SEQ ID NO: 198.

In one aspect, the present disclosure provides an antibody or an antigen-binding fragment that binds α7β1 integrin, wherein the antibody comprises an HCDR1 of SEQ ID NO: 101, an HCDR2 of SEQ ID NO: 102, an HCDR3 of SEQ ID NO: 189, an LCDR1 of SEQ ID NO: 105, an LCDR2 of SEQ ID NO: 106, and an LCDR3 of SEQ ID NO: 75.

In one aspect, the present disclosure provides an antibody or an antigen-binding fragment that binds α7β1 integrin, wherein the antibody comprises an HCDR1 of SEQ ID NO: 101, an HCDR2 of SEQ ID NO: 102, an HCDR3 of SEQ ID NO: 190, an LCDR1 of SEQ ID NO: 105, an LCDR2 of SEQ ID NO: 106, and an LCDR3 of SEQ ID NO: 75.

In one aspect, the present disclosure provides an antibody or an antigen-binding fragment that binds α7β1 integrin, wherein the antibody comprises an HCDR1 of SEQ ID NO: 101, an HCDR2 of SEQ ID NO: 102, an HCDR3 of SEQ ID NO: 191, an LCDR1 of SEQ ID NO: 105, an LCDR2 of SEQ ID NO: 106, and an LCDR3 of SEQ ID NO: 75.

In one aspect, the present disclosure provides an antibody or an antigen-binding fragment that binds α7β1 integrin, wherein the antibody comprises an HCDR1 of SEQ ID NO: 101, an HCDR2 of SEQ ID NO: 102, an HCDR3 of SEQ ID NO: 192, an LCDR1 of SEQ ID NO: 105, an LCDR2 of SEQ ID NO: 106, and an LCDR3 of SEQ ID NO: 75.

In one aspect, the present disclosure provides an antibody or an antigen-binding fragment that binds α7β1 integrin, wherein the antibody comprises an HCDR1 of SEQ ID NO: 101, an HCDR2 of SEQ ID NO: 102, an HCDR3 of SEQ ID NO: 193, an LCDR1 of SEQ ID NO: 105, an LCDR2 of SEQ ID NO: 106, and an LCDR3 of SEQ ID NO: 198.

In one aspect, the present disclosure provides an antibody or an antigen-binding fragment that binds α7β1 integrin, wherein the antibody comprises an HCDR1 of SEQ ID NO: 101, an HCDR2 of SEQ ID NO: 102, an HCDR3 of SEQ ID NO: 194, an LCDR1 of SEQ ID NO: 105, an LCDR2 of SEQ ID NO: 106, and an LCDR3 of SEQ ID NO: 107.

In one aspect, the present disclosure provides an antibody or an antigen-binding fragment that binds α7β1 integrin, wherein the antibody comprises an HCDR1 of SEQ ID NO: 101, an HCDR2 of SEQ ID NO: 102, an HCDR3 of SEQ ID NO: 194, an LCDR1 of SEQ ID NO: 105, an LCDR2 of SEQ ID NO: 196, and an LCDR3 of SEQ ID NO: 107.

In one aspect, the present disclosure provides an antibody or an antigen-binding fragment that binds α7β1 integrin, wherein the antibody comprises an HCDR1 of SEQ ID NO: 101, an HCDR2 of SEQ ID NO: 102, an HCDR3 of SEQ ID NO: 195, an LCDR1 of SEQ ID NO: 105, an LCDR2 of SEQ ID NO: 63, and an LCDR3 of SEQ ID NO: 75.

In one aspect, the present disclosure provides an antibody or an antigen-binding fragment that binds α7β1 integrin, wherein the antibody comprises an HCDR1 of SEQ ID NO: 101, an HCDR2 of SEQ ID NO: 102, an HCDR3 of SEQ ID NO: 104, an LCDR1 of SEQ ID NO: 105, an LCDR2 of SEQ ID NO: 63, and an LCDR3 of SEQ ID NO: 75.

In one aspect, the present disclosure provides an antibody or an antigen-binding fragment that binds α7β1 integrin, wherein the antibody comprises an HCDR1 of SEQ ID NO: 101, an HCDR2 of SEQ ID NO: 102, an HCDR3 of SEQ ID NO: 104, an LCDR1 of SEQ ID NO: 105, an LCDR2 of SEQ ID NO: 197, and an LCDR3 of SEQ ID NO: 75.

In one aspect, the present disclosure provides an antibody or an antigen-binding fragment that binds α7β1 integrin, wherein the antibody comprises an HCDR1 of SEQ ID NO: 101, an HCDR2 of SEQ ID NO: 102, an HCDR3 of SEQ ID NO: 190, an LCDR1 of SEQ ID NO: 105, an LCDR2 of SEQ ID NO: 106, and an LCDR3 of SEQ ID NO: 198.

In one aspect, the present disclosure provides an antibody or an antigen-binding fragment that binds α7β1 integrin, wherein the antibody comprises an HCDR1 of SEQ ID NO: 101, an HCDR2 of SEQ ID NO: 102, an HCDR3 of SEQ ID NO: 189, an LCDR1 of SEQ ID NO: 105, an LCDR2 of SEQ ID NO: 197, and an LCDR3 of SEQ ID NO: 107.

In one aspect, the present disclosure provides an antibody or an antigen-binding fragment that binds α7β1 integrin, wherein the antibody or antigen binding fragment comprises:
i) a variable heavy region comprising an amino acid sequence of SEQ ID NO: 128, and a variable light chain region comprising an amino acid sequence of SEQ ID NO: 129;
ii) a variable heavy region comprising an amino acid sequence of SEQ ID NO: 130, and a variable light chain region comprising an amino acid sequence of SEQ ID NO: 131;
iii) a variable heavy region comprising an amino acid sequence of SEQ ID NO: 132, and a variable light chain region comprising an amino acid sequence of SEQ ID NO: 133;
iv) a variable heavy region comprising an amino acid sequence of SEQ ID NO: 134, and a variable light chain region comprising an amino acid sequence of SEQ ID NO: 135;
v) a variable heavy region comprising an amino acid sequence of SEQ ID NO: 136, and a variable light chain region comprising an amino acid sequence of SEQ ID NO: 137;
vi) a variable heavy region comprising an amino acid sequence of SEQ ID NO: 138, and a variable light chain region comprising an amino acid sequence of SEQ ID NO: 139; or
vii) a variable heavy region comprising an amino acid sequence of SEQ ID NO: 140, and a variable light chain region comprising an amino acid sequence of SEQ ID NO: 141.

In one aspect, the present disclosure provides an antibody or an antigen-binding fragment that binds α7β1 integrin, wherein the antibody or antigen binding fragment comprises:
i) a heavy chain comprising an amino acid sequence of SEQ ID NO: 118, and a light chain comprising an amino acid sequence of SEQ ID NO: 119; a heavy chain comprising an amino acid sequence of SEQ ID NO: 120, and a light chain comprising an amino acid sequence of SEQ ID NO: 121;
ii) a variable heavy region comprising an amino acid sequence of SEQ ID NO: 199, and a variable light chain region comprising an amino acid sequence of SEQ ID NO: 200;
iii) a variable heavy region comprising an amino acid sequence of SEQ ID NO: 201, and a variable light chain region comprising an amino acid sequence of SEQ ID NO: 202;
iv) a variable heavy region comprising an amino acid sequence of SEQ ID NO: 203, and a variable light chain region comprising an amino acid sequence of SEQ ID NO: 204;
v) a variable heavy region comprising an amino acid sequence of SEQ ID NO: 205, and a variable light chain region comprising an amino acid sequence of SEQ ID NO: 206;
vi) a variable heavy region comprising an amino acid sequence of SEQ ID NO: 207, and a variable light chain region comprising an amino acid sequence of SEQ ID NO: 208;
vii) a variable heavy region comprising an amino acid sequence of SEQ ID NO: 209, and a variable light chain region comprising an amino acid sequence of SEQ ID NO: 210;
viii) a variable heavy region comprising an amino acid sequence of SEQ ID NO: 211, and a variable light chain region comprising an amino acid sequence of SEQ ID NO: 212;
ix) a variable heavy region comprising an amino acid sequence of SEQ ID NO: 213, and a variable light chain region comprising an amino acid sequence of SEQ ID NO: 214;
x) a variable heavy region comprising an amino acid sequence of SEQ ID NO: 215, and a variable light chain region comprising an amino acid sequence of SEQ ID NO: 216;
xi) a variable heavy region comprising an amino acid sequence of SEQ ID NO: 217, and a variable light chain region comprising an amino acid sequence of SEQ ID NO: 218;
xii) a variable heavy region comprising an amino acid sequence of SEQ ID NO: 219, and a variable light chain region comprising an amino acid sequence of SEQ ID NO: 220;
xiii) a variable heavy region comprising an amino acid sequence of SEQ ID NO: 221, and a variable light chain region comprising an amino acid sequence of SEQ ID NO: 222; or
xiv) a variable heavy region comprising an amino acid sequence of SEQ ID NO: 223, and a variable light chain region comprising an amino acid sequence of SEQ ID NO: 224.

In one aspect, the present disclosure provides an antibody or an antigen-binding fragment that binds α7β1 integrin, wherein the antibody or antigen binding fragment comprises:
i) a variable heavy region comprising an amino acid sequence of SEQ ID NO: 142, and a variable light chain region comprising an amino acid sequence of SEQ ID NO: 143;
ii) a variable heavy region comprising an amino acid sequence of SEQ ID NO: 144, and a variable light chain region comprising an amino acid sequence of SEQ ID NO: 145;
iii) a variable heavy region comprising an amino acid sequence of SEQ ID NO: 146, and a variable light chain region comprising an amino acid sequence of SEQ ID NO: 147;
iv) a variable heavy region comprising an amino acid sequence of SEQ ID NO: 148, and a variable light chain region comprising an amino acid sequence of SEQ ID NO: 149;
v) a variable heavy region comprising an amino acid sequence of SEQ ID NO: 150, and a variable light chain region comprising an amino acid sequence of SEQ ID NO: 151;
vi) a variable heavy region comprising an amino acid sequence of SEQ ID NO: 152, and a variable light chain region comprising an amino acid sequence of SEQ ID NO: 153;
vii) a variable heavy region comprising an amino acid sequence of SEQ ID NO: 154, and a variable light chain region comprising an amino acid sequence of SEQ ID NO: 155;
viii) a variable heavy region comprising an amino acid sequence of SEQ ID NO: 156, and a variable light chain region comprising an amino acid sequence of SEQ ID NO: 157;
ix) a variable heavy region comprising an amino acid sequence of SEQ ID NO: 158, and a variable light chain region comprising an amino acid sequence of SEQ ID NO: 159;
x) a variable heavy region comprising an amino acid sequence of SEQ ID NO: 160, and a variable light chain region comprising an amino acid sequence of SEQ ID NO: 161;
xi) a variable heavy region comprising an amino acid sequence of SEQ ID NO: 162, and a variable light chain region comprising an amino acid sequence of SEQ ID NO: 163;
xii) a variable heavy region comprising an amino acid sequence of SEQ ID NO: 164, and a variable light chain region comprising an amino acid sequence of SEQ ID NO: 165;
xiii) a variable heavy region comprising an amino acid sequence of SEQ ID NO: 166, and a variable light chain region comprising an amino acid sequence of SEQ ID NO: 167;
xiv) a variable heavy region comprising an amino acid sequence of SEQ ID NO: 170, and a variable light chain region comprising an amino acid sequence of SEQ ID NO: 171;
xv) a variable heavy region comprising an amino acid sequence of SEQ ID NO: 172, and a variable light chain region comprising an amino acid sequence of SEQ ID NO: 173; or
xvi) a variable heavy region comprising an amino acid sequence of SEQ ID NO: 186, and a variable light chain region comprising an amino acid sequence of SEQ ID NO: 187.

In one aspect, the present disclosure provides an antibody or an antigen-binding fragment that binds α7β1 integrin, wherein the antibody or antigen binding fragment comprises:
i) a variable heavy region comprising an amino acid sequence of SEQ ID NO: 108, and a variable light chain region comprising an amino acid sequence of SEQ ID NO: 109;
ii) a variable heavy region comprising an amino acid sequence of SEQ ID NO: 110, and a variable light chain region comprising an amino acid sequence of SEQ ID NO: 111.

In one aspect, the present disclosure provides an antibody or an antigen-binding fragment that binds α7β1 integrin, wherein the antibody or antigen binding fragment comprises:
i) a heavy chain comprising an amino acid sequence of SEQ ID NO: 112, and a light chain comprising an amino acid sequence of SEQ ID NO: 113;
ii) a heavy chain comprising an amino acid sequence of SEQ ID NO: 114, and a light chain comprising an amino acid sequence of SEQ ID NO: 113;
iii) a heavy chain comprising an amino acid sequence of SEQ ID NO: 115, and a light chain comprising an amino acid sequence of SEQ ID NO: 116; or
iv) a heavy chain comprising an amino acid sequence of SEQ ID NO: 117, and a light chain comprising an amino acid sequence of SEQ ID NO: 116.

In some embodiments, the antibody or antigen-binding fragment has an affinity (Kd) to α7β1 integrin or a fragment thereof of between about 0.1 to 30 nM. In some embodiments, the antibody or antigen-binding fragment has an affinity (Kd) to α7β1 integrin or a fragment thereof of between about 0.5 to 30 nM. In some embodiments, the α7β1 integrin or fragment thereof comprises α7β1 ectodomain, B1 ectodomain, a7 leg, calf-1 domain, calf-2 domain, or calf-1 and calf-2 domains. In some embodiments, the α7β1 integrin or fragment thereof comprises α7β1 ectodomain. In some embodiments, the α7β1 integrin or fragment thereof comprises B1 ectodomain. In some embodiments, the α7β1 integrin or fragment thereof comprises a7 leg. In some embodiments, the α7β1 integrin or fragment thereof comprises calf-1 domain. In some embodiments, the α7β1 integrin or fragment thereof comprises calf-2 domain. In some embodiments, the α7β1 integrin or fragment thereof comprises calf-1 and calf-2 domains.

In some embodiments, the α7β1 integrin comprises human α7β1 integrin. In some embodiments, the antibody or antigen-binding fragment has an affinity to a human α7β1 ectodomain of about 3 to 6 nM.

In some embodiments, the antibody or antigen-binding fragment activates α7β1 integrin with a maximal efficacy (Emax) of between 100% to 300% as compared to the 20TFT antibody. In some embodiments, the antibody or antigen-binding fragment activates α7β1 integrin with a maximal efficacy (Emax) of between 100% to 300% as compared to the TS2/16 antibody.

In some embodiments, the antibody or antigen-binding fragment has an EC50 value of between 0.3 nM to 50 nM. In some embodiments, the antibody or antigen-binding fragment has an EC50 value of about 0.3 nM. In some embodiments, the antibody or antigen-binding fragment has an EC50 value of between 0.3 nM and 1 nM. In some embodiments, the antibody or antigen-binding fragment has an EC50 value of between 0.3 nM and 5 nM. In some embodiments, the antibody or antigen-binding fragment has an EC50 value of between 0.3 nM and 10 nM. In some embodiments, the antibody or antigen-binding fragment has an EC50 value of between 1 nM and 5 nM. In some embodiments, the antibody or antigen-binding fragment has an EC50 value of between 1 nM and 10 nM. In some embodiments, the antibody or antigen-binding fragment has an EC50 value of between 5 nM and 10 nM. In some embodiments, the antibody or antigen-binding fragment has an EC50 value of between 5 nM and 50 nM. In some embodiments, the antibody or antigen-binding fragment has an EC50 value of between 10 nM and 50 nM.

In some embodiments, the antibody or antigen-binding fragment comprises a Fab, a single chain Fv (scFv), a single domain antibody (sdAb), a VHH, one or more CDRs, a variable heavy chain (VH), a variable light chain (VL), a Fab-like bispecific antibody (bsFab), a single-domain antibody-linked Fab (s-Fab), an antibody, or a combination thereof. In some embodiments, the antibody or antigen-binding fragment comprises a Fab. In some embodiments, the antibody or antigen-binding fragment comprises a single chain Fv (scFv). In some embodiments, the antibody or antigen-binding fragment comprises a single domain antibody (sdAb). In some embodiments, the antibody or antigen-binding fragment comprises a VHH. In some embodiments, the antibody or antigen-binding fragment comprises one or more CDRs. In some embodiments, the antibody or antigen-binding fragment comprises a variable heavy chain (VH). In some embodiments, the antibody or antigen-binding fragment comprises a variable light chain (VL). In some embodiments, the antibody or antigen-binding fragment comprises a Fab-like bispecific antibody (bsFab). In some embodiments, the antibody or antigen-binding fragment comprises a single-domain antibody-linked Fab (s-Fab). In some embodiments, the antibody or antigen-binding fragment comprises an antibody.

In some embodiments, the antibody or antigen-binding fragment is a humanized antibody. In some embodiments, the antibody or antigen-binding fragment is a human antibody. In some embodiments, the antibody or antigen-binding fragment is a chimeric antibody.

In some embodiments, the antibody or antigen-binding fragment is derived from IgG. In some embodiments, the antibody or antigen-binding fragment is derived from IgGI, IgG2, IgG3, or IgG4. In some embodiments, the antibody or antigen-binding fragment is derived from IgG1. In some embodiments, the antibody or antigen-binding fragment is derived from IgG2. In some embodiments, the antibody or antigen-binding fragment is derived from IgG3. In some embodiments, the antibody or antigen-binding fragment is derived from IgG4.

In some embodiments, the antibody or antigen-binding fragment comprises a Fc region. In some embodiments, the Fc region contains one or more mutations.

In some embodiments, the present disclosure provides a method of treating a disease or disorder in a subject associated with an α7β1 integrin defect. In some embodiments, the method comprises administering to the subject an antibody or antigen-binding fragment of the instant disclosure.

In some embodiments, the disease or disorder associated with an α7β1 integrin defect comprises cardiomyopathy, congestive heart failure, muscular dystrophy, muscular atrophy and GLP-1 receptor, GIP/GLP-1 receptor agonism-induced lean muscle loss in obesity patients. In some embodiments, the disease or disorder associated with an α7β1 integrin defect comprises cardiomyopathy. In some embodiments, the disease or disorder associated with an α7β1 integrin defect comprises congestive heart failure. In some embodiments, the disease or disorder associated with an α7β1 integrin defect comprises muscular dystrophy. In some embodiments, the disease or disorder associated with an α7β1 integrin defect comprises muscular atrophy. In some embodiments, the disease or disorder associated with an α7β1 integrin defect comprises sarcopenia.

In some embodiments, the administration of the antibody or antigen-binding fragment to the subject results in amelioration of one or more symptoms of the muscular dystrophy.

In some embodiments, administration reduced one or more serum biomarkers of muscle fiber damage. In some embodiments, the one or more serum biomarkers comprises creatine kinase, LDH cytotox, and/or IL-6. In some embodiments, the serum biomarker comprises creatine kinase. In some embodiments, the serum biomarker comprises LDH cytotox. In some embodiments, the serum biomarker comprises IL-6.

In some embodiments, the administration reduces muscle damage, inflammation, fibrosis, necrosis, and/or atrophy. In some embodiments, the administration reduces muscle damage. In some embodiments, the administration reduces inflammation. In some embodiments, the administration reduces fibrosis. In some embodiments, the administration reduces necrosis. In some embodiments, the administration reduces atrophy.

In some embodiments, the administration of the antibody or antigen-binding fragment is to a muscle cell. In some embodiments, the muscle cell comprises a mutation in a dystrophin gene. In some embodiments, the muscle cell lacks dystrophin. In some embodiments, the muscle cell comprises a non-functional dystrophin.

In some embodiments, the muscular dystrophy comprises Duchenne muscular dystrophy, Becker muscular dystrophy, limb girdle muscular dystrophy, Fukuyama congenital muscular dystrophy (FCMD) or merosin deficient congenital muscular dystrophy type 1A (MDC1A). In some embodiments, the muscular dystrophy comprises Duchenne muscular dystrophy. In some embodiments, the muscular dystrophy comprises Becker muscular dystrophy. In some embodiments, the muscular dystrophy comprises limb girdle muscular dystrophy. In some embodiments, the muscular dystrophy comprises Fukuyama congenital muscular dystrophy (FCMD). In some embodiments, the muscular dystrophy comprises merosin deficient congenital muscular dystrophy type 1A (MDC1A).

In some embodiments, the muscle atrophy is due to aging, inactivity, denervation or other underlying diseases including chronic obstructive pulmonary disorder, cancer-associated cachexia, diabetes, renal failure, cardiac failure. In some embodiments, muscle atrophy is due to aging. In some embodiments, muscle atrophy is due to inactivity. In some embodiments, muscle atrophy is due to denervation. In some embodiments, muscle atrophy is due to other underlying diseases.

In some embodiments, the loss of lean muscle mass in obese patient who is treated with glucagon-like peptide 1 receptor agonist.

In some embodiments, the heart failure due to genetically inherited cardiomyopathies. In some embodiments, the heart failure due to ischemic heart disease.

In some embodiments, the present disclosure provides a pharmaceutical composition comprising the antibody or antigen-binding fragment and a pharmaceutically acceptable carrier. In some embodiments, the antibody or antigen-binding fragment is administered parenterally. In some embodiments, the antibody or antigen-binding fragment is administered orally.

In some embodiments, the antibody or antigen-binding fragment is administered intravenously. In some embodiments, the antibody or antigen-binding fragment is administered intradermally. In some embodiments, the antibody or antigen-binding fragment is administered transdermally. In some embodiments, the antibody or antigen-binding fragment is administered intraocularly. In some embodiments, the antibody or antigen-binding fragment is administered intranasally. In some embodiments, the antibody or antigen-binding fragment is administered intramuscularly. In some embodiments, the antibody or antigen-binding fragment is administered subcutaneously. In some embodiments, the antibody or antigen-binding fragment is administered transmucosally.

In some embodiments, the present disclosure provides a method of treating cardiomyopathy, congestive heart failure, or other cardiac muscle disorder, by administering to a subject the antibody or antigen-binding fragment.

In one aspect, the present disclosure provides, among other things, a method of treating a muscular dystrophy in a subject, comprising administering to a subject in need thereof a pharmaceutical composition comprising: an antibody means for facilitating the extended-open conformation of the α7β1 integrin; and one or more physiologically acceptable carriers, excipients or diluents.

In one aspect, the present disclosure provides, among other things, a method of treating a muscle atrophy in a subject, comprising administering to a subject in need thereof a therapeutic agent to increase expression of α7, wherein the increase in expression of α7 results in amelioration of one or more symptoms of the muscle atrophy. In some embodiments, the therapeutic agent comprises an antibody or antigen-binding fragment of the instant disclosure.

In one aspect, the present disclosure provides, among other things, a method of treating a muscle atrophy in a subject, comprising administering to a subject in need thereof a pharmaceutical composition comprising an antibody or antigen-binding fragment of the instant disclosure, wherein the administration results in amelioration of one or more symptoms of the muscle atrophy.

In some embodiments, the subject has reduced muscle loss as compared to a control upon administration of the antibody or the binding fragment thereof.

In some embodiments, the subject has less than 40% muscle weight change as compared to a control upon administration of the antibody or the binding fragment thereof. In some embodiments, the subject has less than 35% muscle weight change as compared to a control upon administration of the antibody or the binding fragment thereof. In some embodiments, the subject has less than 30% muscle weight change as compared to a control upon administration of the antibody or the binding fragment thereof. In some embodiments, the subject has less than 28% muscle weight change as compared to a control upon administration of the antibody or the binding fragment thereof. In some embodiments, the subject has less than 27% muscle weight change as compared to a control upon administration of the antibody or the binding fragment thereof. In some embodiments, the subject has less than 26% muscle weight change as compared to a control upon administration of the antibody or the binding fragment thereof. In some embodiments, the subject has less than 25% muscle weight change as compared to a control upon administration of the antibody or the binding fragment thereof. In some embodiments, the subject has less than 24% muscle weight change as compared to a control upon administration of the antibody or the binding fragment thereof. In some embodiments, the subject has less than 23% muscle weight change as compared to a control upon administration of the antibody or the binding fragment thereof. In some embodiments, the subject has less than 22% muscle weight change as compared to a control upon administration of the antibody or the binding fragment thereof. In some embodiments, the subject has less than 21% muscle weight change as compared to a control upon administration of the antibody or the binding fragment thereof. In some embodiments, the subject has less than 20% muscle weight change as compared to a control upon administration of the antibody or the binding fragment thereof. In some embodiments, the subject has less than 19% muscle weight change as compared to a control upon administration of the antibody or the binding fragment thereof. In some embodiments, the subject has less than 18% muscle weight change as compared to a control upon administration of the antibody or the binding fragment thereof. In some embodiments, the subject has less than 17% muscle weight change as compared to a control upon administration of the antibody or the binding fragment thereof. In some embodiments, the subject has less than 16% muscle weight change as compared to a control upon administration of the antibody or the binding fragment thereof. In some embodiments, the subject has less than 15% muscle weight change as compared to a control upon administration of the antibody or the binding fragment thereof. In some embodiments, the subject has less than 14% muscle weight change as compared to a control upon administration of the antibody or the binding fragment thereof. In some embodiments, the subject has less than 13% muscle weight change as compared to a control upon administration of the antibody or the binding fragment thereof. In some embodiments, the subject has less than 12% muscle weight change as compared to a control upon administration of the antibody or the binding fragment thereof. In some embodiments, the subject has less than 11% muscle weight change as compared to a control upon administration of the antibody or the binding fragment thereof. In some embodiments, the subject has less than 10% muscle weight change as compared to a control upon administration of the antibody or the binding fragment thereof. In some embodiments, the subject has less than 9% muscle weight change as compared to a control upon administration of the antibody or the binding fragment thereof. In some embodiments, the subject has less than 8% muscle weight change as compared to a control upon administration of the antibody or the binding fragment thereof. In some embodiments, the subject has less than 7% muscle weight change as compared to a control upon administration of the antibody or the binding fragment thereof. In some embodiments, the subject has less than 6% muscle weight change as compared to a control upon administration of the antibody or the binding fragment thereof. In some embodiments, the subject has less than 5% muscle weight change as compared to a control upon administration of the antibody or the binding fragment thereof. In some embodiments, the subject has less than 4% muscle weight change as compared to a control upon administration of the antibody or the binding fragment thereof. In some embodiments, the subject has less than 3% muscle weight change as compared to a control upon administration of the antibody or the binding fragment thereof. In some embodiments, the subject has less than 2% muscle weight change as compared to a control upon administration of the antibody or the binding fragment thereof. In some embodiments, the subject has less than 1% muscle weight change as compared to a control upon administration of the antibody or the binding fragment thereof.

In some embodiments, the control is a baseline value prior to administration of the antibody or the binding fragment thereof.

In some embodiments, the control is historical data.

In some embodiments, the control is a comparable subject without the administration of the antibody or antigen binding fragment thereof.

In one aspect, the present disclosure provides, among other things, a method of treating a muscle atrophy in a subject, comprising administering to a subject in need thereof a pharmaceutical composition comprising (a) an antibody means for stabilizing the extended conformations of the α7β1 integrin and (b) one or more physiologically acceptable carriers. In some embodiments, the antibody means for stabilizing the extended conformations of the α7β1 integrin comprises an antibody or antigen-binding fragment of the instant disclosure.

In one aspect, the present disclosure provides, among other things, a method of treating a muscle atrophy in a subject, comprising administering to a subject in need thereof a pharmaceutical composition comprising (a) an antibody means for increasing α7 expression and (b) one or more physiologically acceptable carriers. In some embodiments, the antibody means for increasing α7 expression comprises an antibody or antigen-binding fragment of the instant disclosure.

### BRIEF DESCRIPTION OF DRAWINGS

Drawings are for illustration purposes only, not for limitation.

FIG. 1A shows α7β1 integrin domains used to generate antibodies of the present disclosure. FIGS. 1B-1D are series of exemplary graphs showing binding affinity of exemplary anti-α7β1 antibodies to different α7β1 domains as determined by ELISA assays. In particular, exemplary antibodies Ab2, Ab8, and RefAbl were tested for binding to the α7β1 ecto domain, the leg domain, and the calf-1 domain.

FIG. 2 shows exemplary competitive ELISA assay results for binding of Ab2 and RefAbl to the human α7β1 ecto domain.

FIG. 3 is a series of exemplary graphs illustrating the results of competitive ELISA assays to determine if exemplary human anti-α7β1 antibodies have overlapping epitopes with Ab2, Ab8, or RefAb1.

FIG. 4 shows the results of competitive ELISA assays to determine if exemplary mouse anti-α7β1 antibodies have overlapping epitopes with Ab8 or RefAb1.

FIG. 5A is an exemplary schematic showing a bent-closed conformation of integrin α7β1, which has a low binding affinity to laminin. FIG. 5B is an exemplary schematic showing the extended-closed conformation of integrin α7β1, which has a low binding affinity to laminin and how an exemplary anti-α7β1 and antibody of the present disclosure could bind to integrin α7β1 to stabilize that conformation. FIG. 5C is an exemplary schematic showing the extended-open conformation of integrin α7β, which has a high binding affinity to laminin and how an exemplary anti-α7β1 and antibody of the present disclosure could bind to integrin α7β1 to stabilize that conformation.

FIG. 6 is an exemplary crystal structure of α7 leg and epitopes of different antibodies disclosed in the present disclosure. In particular, Ab8 binds to Calf-1, and Ab2 binds to a region in Calf-2 domain that is distinct from the epitope of RefAb1 (20TFT) antibody.

FIG. 7 is a series of electron microscopy images of exemplary anti-α7β1 antibodies binding to integrin α7β1, and their cartoon representations.

FIG. 8A is an exemplary crystal structure of the Ab2 heavy chain (orange) and light chain (yellow) in complex with α7 calf-1 (blue). FIG. 8B displays the biomolecular interactions within Ab2 Fab-α7 calf-1 complex. The calf-1 protein is blue and is shown with the Ab2 HCDR1 (pink), HCDR2 (orange), HCDR3 (green), and LCDR1 (yellow). FIG. 8C shows amino acid sequences of α7 protein, with epitope residues highlighted.

FIG. 9 is an exemplary figure depicting α7β1 structural model of α7 leg with epitope of Ab2 antibody possible epitope in spheres. The corresponding amino acid residues are highlighted in SEQ ID NO: 180. Residues highlighted in magenta (MAIPQQL, SEQ ID NO: 181; VVRGERAMQSERDVGSKVKYEVT, SEQ ID NO: 182; FDRAAVL, SEQ ID NO:183) illustrates one possible epitope, and the residues highlighted in red (LRTLGSAFL, SEQ ID NO: 184; VVFSCPLYS, SEQ ID NO: 185) represents another possible epitope. The epitope residues are determined by hydrogen/deuterium exchange mass spectrometry.

FIG. 10 is an exemplary figure showing the effect of activating conditions (i.e., conditions that stabilize the open conformation of integrin α7β1) on the cell binding of exemplary anti-α7β1 antibodies of the present disclosure to human DMD myoblast cells.

FIG. 11 is an exemplary figure showing the effect of activating conditions on the cell binding of exemplary mouse anti-α7β1 compared to RefAb1.

FIG. 12 is an exemplary bar graph showing the levels of creatine kinase (CKM), an indicator of muscle damage, after acute treadmill running in a mouse mdx model with and without application of an exemplary anti-α7β1 antibody.

FIG. 13 is an exemplary figure showing the dose response of CKM and LDH to application of exemplary anti-α7β1 antibody Ab8.

FIG. 14 displays the biological processes upregulated by application of an exemplary anti-α7β1 antibody.

FIG. 15 displays the biological processes downregulated by application of an exemplary anti-α7β1 antibody.

FIGS. 16A and 16B are series of exemplary graphs showing the increases in gene expression caused by application of exemplary anti-α7β1 antibodies.

FIG. 17 is an exemplary figure showing the running scores and total distance run for B10.mdx mice after administration of exemplary anti-α7β1 antibodies.

FIG. 18 is an exemplary figure showing the distance traveled, resting time, and horizontal beam break counts for MDX mice administered an exemplary anti-α7β1 antibody.

FIG. 19A is an exemplary figure showing the specific tetanus in the extensor digitorum longus of MDX mice treated with an exemplary α7β1 antibody and controls. FIG. 19B shows the specific tetanus in the diaphragm of MDX mice treated with an exemplary α7β1 antibody and controls.

FIG. 20 is an exemplary figure showing the results tracking bodyweight of MDX mice with or without administration of an exemplary anti-α7β1 antibody.

FIG. 21 is an exemplary figure showing the muscle fiber size from MDX mice with and without treatment with an exemplary anti-α7β1 antibody.

FIG. 22A is a series of exemplary graphs showing qPCR results tracking fibrosis markers in MDX mice after administration of an exemplary anti-α7β1 antibody. FIG. 22B provides exemplary collagen I/III immunohistochemistry images of mice treated with PBS or Ab8 and quantitative positive collagen I/III area. Lower collagen is sign of less scar tissue and fibrosis, a visible difference that can be seen in the histology images.

FIG. 23 is an exemplary schematic of a study design to assess attenuation of muscle atrophy by administration of an exemplary anti-α7β1 antibody.

FIG. 24 is a series of exemplary graphs showing the muscle weight of the gastrocnemius-soleus complex (Gastroc) after administration of an exemplary anti-α7β1 antibody to C57BL/6j mice in VIM. Disuse atrophy caused by VIM results in a loss of muscle weight in the Gastroc both absolutely and relative to bodyweight.

FIG. 25 is a series of exemplary graphs showing expression of α7 after administration of an exemplary anti-α7β1 antibody to C57BL/6j mice in VIM as compared to an ambulatory control.

### DEFINITIONS

In order for the present invention to be more readily understood, certain terms are first defined below. Additional definitions for the following terms and other terms are set forth throughout the specification.

“Alleviate” and “ameliorate” means a process by which the severity of a sign or symptom of a disorder is decreased. Importantly, a sign or symptom can be alleviated without being eliminated. Therapeutically effective dosages are expected to decrease the severity of, and so alleviate and ameliorate, a sign or symptom of disease.

As used herein, the term “affinity” refers to the characteristics of a binding interaction between a binding moiety (e.g., integrin α7β1 antibody) and a target (e.g., α7β1) and that indicates the strength of the binding interaction. In some embodiments, the measure of affinity is expressed as a dissociation constant (KD).

As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. An antibody typically includes three complementarity determining regions (abbr. CDRs) in the light chain of the immunoglobulin and three complementarity determining regions (CDRs) in the heavy chain of the immunoglobulin. The three CDRs in the light chain of the immunoglobulin are called, from the N-terminal side, CDR1, CDR2 and CDR3, respectively. The three CDRs in the heavy chain of the immunoglobulin are also called, from the N-terminal side, CDR1, CDR2 and CDR3, respectively. A “CDR” may be identified in accordance with the definitions of the Kabat, Chothia, the accumulation of both Kabat and Chothia, AbM, contact, and/or conformational definitions or any method of CDR determination well known in the art. The term “antibody” encompasses antigen-binding fragments of antibodies (e.g., single chain antibodies, Fab, F(ab′)2, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda.

As used herein, the term “M38 antibody” refers to a mouse anti-α7β1 antibody that binds to S977, W978, W979 and P980 on α7 integrin, as described in described in WO2022240833A1, which is hereby incorporated by reference. In the present application “M38” is used interchangeably with “RefAb2.”

As used herein, the term “20TFT antibody” refers to an anti-α7β1 antibody that binds to S977, W978, W979 and P980 on α7 integrin, as described in described in WO 2021/097338 A1, which is hereby incorporated by reference. 20TFT antibody is humanized antibody of M38. In the present application “20TFT” is used interchangeably with “RefAb1.”

As used herein, the term “EC50” refers to a half maximal effective concentration. The term EC50 refers to the concentration of a drug, antibody or toxicant which induces a response halfway between the baseline and maximum after a specified exposure time. More simply, EC50 can be defined as the concentration required to obtain a 50% of the desired effect.

As used herein, the term “Fab” refers to an antibody fragment comprising a portion of an intact antibody, comprising the antigen-binding or variable region thereof.

As used herein, the term “Fc region” refers to a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

As used herein, the term “EC50” refers to the concentration needed to inhibit half of the maximum biological response of the ligand binding, and can be determined by cell-adhesion assay (CAA) or solid phase assay (SP).

As used herein, the term “identity” refers a relationship between the sequences of two or more polypeptide molecules or two or more nucleic acid molecules as known in the art, comparing the sequences of these molecules. The relationship determined by doing. In the art, “identity” also means the degree of sequence relatedness between nucleic acid molecules or polypeptides, and in some cases more than one nucleotide sequence or more than one. It may be determined by a match between amino acid sequence strings. “Identity” means between a gap alignment (if any) addressed by a particular mathematical model or computer program (i.e., an “algorithm”) and a smaller sequence of two or more sequences. Measure the percent identity match.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

“Integrin activator” or “integrin α7β1 activator” refers to molecule that can bind to integrin α7β1 in a way that increases one or more activities associated with the integrin. For example, and without limitation, an integrin α7β1 activator may increase integrin α7β1 binding with laminin. In some embodiments, an integrin α7β1 activator is an anti-α7β1 antibody.

As used herein, the term “treat,” “treatment,” or “treating” refers to any method used to partially or completely alleviate, ameliorate, relieve, inhibit, prevent, delay onset of reduce severity of and/or reduce incidence of one or more symptoms of features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present disclosure provides, among other things, integrin α7β1 antibodies and therapeutic uses of such antibodies in effectively treating diseases or disorders associated with loss of dystrophin, such as Duchenne Muscular Dystrophy (DMD). As described herein, the present disclosure is based, in part, on identification of integrin α7β1 antibodies that are effective in stabilizing the α7β1 integrin in an extended-closed or extended-open conformation. As a result, the anti-α7β1 antibodies of the present disclosure effectively increase adhesion of muscle fibers to the extracellular matrix, restoring some or all the muscle adhesion lost by the absence of functional dystrophin and ultimately improving locomotive ability, increasing muscle force, and reducing fibrosis in patients with dystrophinopathies.

### Integrin α7β1

The dystrophin-glycoprotein complex maintains the integrity of skeletal muscle by associating laminin in the extracellular matrix with the actin cytoskeleton. Several human muscular dystrophies arise from defects in the components of this complex. The α7β1 integrin also binds laminin and links the extracellular matrix with the cytoskeleton. The α7β1 integrin complex clearly plays a role in the development of striated muscle and smooth muscle. α7β1 integrin promotes the adhesion and motility of myoblasts, and it is likely important in the recruitment of myogenic precursors during muscle differentiation.

Dystrophin-associated protein complex (DAPC) and α7β1 integrin maintains sarcolemma integrity. Repeated contraction and relaxation of sarcomere requires efficient handling of mechanical stress by sarcolemma. Disruption of DAPC or α7β1 linkage results in compromised association of cell membrane with ECM, leading to membrane damage due to excess load of force. Additionally, loss of function mutation (LOF) of dystrophin causes Duchenne muscular dystrophy (DMD) while LOF mutation of ITGA7 (gene encoding α7β1 integrin) leads to congenital muscular dystrophy.

### Structure and Confirmation of Integrin α7β1

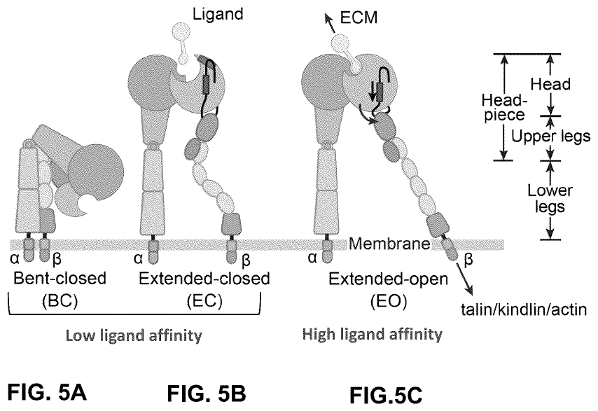

Integrin structure and conformation are key to each part of its functionality, from adhesion to signaling. α7β1 integrins can adopt a range of conformation structures from (FIG. 5A) bent closed conformation and extended closed conformation, that are inactive with a low affinity for laminin (FIG. 5B), and extended open conformation (FIG. 5C) that is active with a high affinity for laminin. α7 subunit has β-propeller, thigh, calf-1, and calf-2 domains as shown in FIG. 5C.

The present disclosure provides, among other things, antibodies that bind to an α7β1 integrin in an extended-open or in an extended-closed conformation, enhancing α7β1 integrin binding to laminin. In some embodiments, anti-α7β1 integrin antibody binds to an α7β1 integrin in an extended-closed conformation. In some embodiments, anti-α7β1 integrin antibody binds to an α7β1 integrin in an extended-open conformation. In some embodiments, anti-α7β1 integrin antibody maintains an α7β1 integrin in an extended-closed conformation. In some embodiments, anti-α7β1 integrin antibody maintains an α7β1 integrin in an extended-open conformation.

Human integrin α7 ectodomain can be present in X1 or X2 isoform.

In some embodiments, a human integrin α7 ectodomain has the following amino acid sequence (signal peptide underlined):

(SEQ ID NO: 122, a7)
MAGARSRDPWGASGICYLFGSLLVELLESRAVAFNLDVMGALRKE
GEPGSLFGFSVALHRQLQPRPQSWLLVGAPQALALPGQQANRTGG
LFACPLSLEETDCYRVDIDQGADMQKESKENQWLGVSVRSQGPGG
KIVTCAHRYEARQRVDQILETRDMIGRCFVLSQDLAIRDELDGGE
WKFCEGRPQGHEQFGFCQQGTAAAFSPDSHYLLFGAPGTYNWKGT
ARVELCAQGSADLAHLDDGPYEAGGEKEQDPRLIPVPANSYFGLL
FVTNIDSSDPDQLVYKTLDPADRLPGPAGDLALNSYLGFSIDSGK
GLVRAEELSFVAGAPRANHKGAVVILRKDSASRLVPEVMLSGERL
TSGFGYSLAVADLNSDGWPDLIVGAPYFFERQEELGGAVYVYLNQ
GGHWAGISPLRLCGSPDSMFGISLAVLGDLNQDGFPDIAVGAPFD
GDGKVFIYHGSSLGVVAKPSQVLEGEAVGIKSFGYSLSGSLDMDG
NQYPDLLVGSLADTAVLFRARPILHVSHEVSIAPRSIDLEQPNCA
GGHSVCVDLRVCFSYIAVPSSYSPTVALDYVLDADTDRRLRGQVP
RVTFLSRNLEEPKHQASGTVWLKHQHDRVCGDAMFQLQENVKDKL
RAIVVTLSYSLQTPRLRRQAPGQGLPPVAPILNAHQPSTQRAEIH
FLKQGCGEDKICQSNLQLVRARFCTRVSDTEFQPLPMDVDGTTAL
FALSGQPVIGLELMVTNLPSDPAQPQADGDDAHEAQLLVMLPDSL
HYSGVRALDPAEKPLCLSNENASHVECELGNPMKRGAQVTFYLIL
STSGISIETTELEVELLLATISEQELHPVSARARVFIELPLSIAG
MAIPQQLFFSGVVRGERAMQSERDVGSKVKYEVTVSNQGQSLRTL
GSAFLNIMWPHEIANGKWLLYPMQVELEGGQGPGQKGLCSPRPNI
LHLDVDSRDRRRRELEPPEQQEPGERQEPSMSWWPVSSAEKKKNI
TLDCARGTANCVVFSCPLYSFDRAAVLHVWGRLWNSTFLEEYSAV
KSLEVIVRANITVKSSIKNLMLRDASTVIPVMVYLDPMAVVAEGV
PWWVILLAVLAGLLVLALLVLLLWKMGFFKRAKHPEATVPQYHAV
KIPREDRQQFKEEKTGTILRNNWGSPRREGPDAHPILAADGHPEL
GPDGHPGPGTA

In some embodiments, a human integrin α7 ectodomain has the following amino acid sequence:

(SEQ ID NO: 123, ITGA7 X2 ectodomain)
FNLDVMGALRKEGEPGSLFGFSVALHRQLQPRPQSWLLVGAPQAL
ALPGQQANRTGGLFACPLSLEETDCYRVDIDQGADMQKESKENQW
LGVSVRSQGPGGKIVTCAHRYEARQRVDQILETRDMIGRCFVLSQ
DLAIRDELDGGEWKFCEGRPQGHEQFGFCQQGTAAAFSPDSHYLL
FGAPGTYNWKGLLFVTNIDSSDPDQLVYKTLDPADRLPGPAGDLA
LNSYLGFSIDSGKGLVRAEELSFVAGAPRANHKGAVVILRKDSAS
RLVPEVMLSGERLTSGFGYSLAVADLNSDGWPDLIVGAPYFFERQ
EELGGAVYVYLNQGGHWAGISPLRLCGSPDSMFGISLAVLGDLNQ
DGFPDIAVGAPFDGDGKVFIYHGSSLGVVAKPSQVLEGEAVGIKS
FGYSLSGSLDMDGNQYPDLLVGSLADTAVLFRARPILHVSHEVSI
APRSIDLEQPNCAGGHSVCVDLRVCFSYIAVPSSYSPTVALDYVL
DADTDRRLRGQVPRVTFLSRNLEEPKHQASGTVWLKHQHDRVCGD
AMFQLQENVKDKLRAIVVTLSYSLQTPRLRRQAPGQGLPPVAPIL
NAHQPSTQRAEIHFLKQGCGEDKICQSNLQLVRARFCTRVSDTEF
QPLPMDVDGTTALFALSGQPVIGLELMVTNLPSDPAQPQADGDDA
HEAQLLVMLPDSLHYSGVRALDPAEKPLCLSNENASHVECELGNP
MKRGAQVTFYLILSTSGISIETTELEVELLLATISEQELHPVSAR
ARVFIELPLSIAGMAIPQQLFFSGVVRGERAMQSERDVGSKVKYE
VTVSNQGQSLRTLGSAFLNIMWPHEIANGKWLLYPMQVELEGGQG
PGQKGLCSPRPNILHLDVDSRDRRRRELEPPEQQEPGERQEPSMS
WWPVSSAEKKKNITLDCARGTANCVVFSCPLYSFDRAAVLHVWGR
LWNSTFLEEYSAVKSLEVIVRANITVKSSIKNLMLRDASTVIPVM
VYLDPMAVVAE

In some embodiments, a human integrin α7 ectodomain has the following amino acid sequence:

(SEQ ID NO: 124, ITGA7 X1 ectodomain)
FNLDVMGALRKEGEPGSLFGFSVALHRQLQPRPQSWLLVGAPQAL
ALPGQQANRTGGLFACPLSLEETDCYRVDIDQGADMQKESKENQW
LGVSVRSQGPGGKIVTCAHRYEARQRVDQILETRDMIGRCFVLSQ
DLAIRDELDGGEWKFCEGRPQGHEQFGFCQQGTAAAFSPDSHYLL
FGAPGTYNWKGTARVELCAQGSADLAHLDDGPYEAGGEKEQDPRL
IPVPANSYFGFSIDSGKGLVRAEELSFVAGAPRANHKGAVVILRK
DSASRLVPEVMLSGERLTSGFGYSLAVADLNSDGWPDLIVGAPYF
FERQEELGGAVYVYLNQGGHWAGISPLRLCGSPDSMFGISLAVLG
DLNQDGFPDIAVGAPFDGDGKVFIYHGSSLGVVAKPSQVLEGEAV
GIKSFGYSLSGSLDMDGNQYPDLLVGSLADTAVLFRARPILHVSH
EVSIAPRSIDLEQPNCAGGHSVCVDLRVCFSYIAVPSSYSPTVAL
DYVLDADTDRRLRGQVPRVTFLSRNLEEPKHQASGTVWLKHQHDR
VCGDAMFQLQENVKDKLRAIVVTLSYSLQTPRLRRQAPGQGLPPV
APILNAHQPSTQRAEIHFLKQGCGEDKICQSNLQLVRARFCTRVS
DTEFQPLPMDVDGTTALFALSGQPVIGLELMVTNLPSDPAQPQAD
GDDAHEAQLLVMLPDSLHYSGVRALDPAEKPLCLSNENASHVECE
LGNPMKRGAQVTFYLILSTSGISIETTELEVELLLATISEQELHP
VSARARVFIELPLSIAGMAIPQQLFFSGVVRGERAMQSERDVGSK
VKYEVTVSNQGQSLRTLGSAFLNIMWPHEIANGKWLLYPMQVELE
GGQGPGQKGLCSPRPNILHLDVDSRDRRRRELEPPEQQEPGERQE
PSMSWWPVSSAEKKKNITLDCARGTANCVVFSCPLYSFDRAAVLH
VWGRLWNSTFLEEYSAVKSLEVIVRANITVKSSIKNLMLRDASTV
IPVMVYLDPMAVVAE

Human α7 leg has the following amino acid sequence:

(SEQ ID NO: 125, a7leg)
QGCGEDKICQSNLQLVRARFCTRVSDTEFQPLPMDVDGTTALFAL
SGQPVIGLELMVTNLPSDPAQPQADGDDAHEAQLLVMLPDSLHYS
GVRALDPAEKPLCLSNENASHVECELGNPMKRGAQVTFYLILSTS
GISIETTELEVELLLATISEQELHPVSARARVFIELPLSIAGMAI
PQQLFFSGVVRGERAMQSERDVGSKVKYEVTVSNQGQSLRTLGSA
FLNIMWPHEIANGKWLLYPMQVELEGGQGPGQKGLCSPRPNILHL
DVDSRDRRRRELEPPEQQEPGERQEPSMSWWPVSSAEKKKNITLD
CARGTANCVVFSCPLYSFDRAAVLHVWGRLWNSTFLEEYSAVKSL
EVIVRANITVKSSIKNLMLRDASTVIPVMVYLDPMAVVAE

Calf-1 domain of human α7 has the following amino acid sequence:

(SEQ ID NO: 126, calf1)
QSNLQLVRARFCTRVSDTEFQPLPMDVDGTTALFALSGQPVIGLE
LMVTNLPSDPAQPQADGDDAHEAQLLVMLPDSLHYSGVRALDPAE
KPLCLSNENASHVECELGNPMKRGAQVTFYLILSTSGISIETTEL
EVELLLATISEQELHPVSARARVFIE

Calf-2 domain of human α7 has the following amino acid sequence:

(SEQ ID NO: 127, calf2)
LPLSIAGMAIPQQLFFSGVVRGERAMQSERDVGSKVKYEVTVSNQ
GQSLRTLGSAFLNIMWPHEIANGKWLLYPMQVELEGGQGPGQKGL
CSPRPNILHLDVDSRDRRRRELEPPEQQEPGERQEPSMSWWPVSS
AEKKKNITLDCARGTANCVVFSCPLYSFDRAAVLHVWGRLWNSTF
LEEYSAVKSLEVIVRANITVKSSIKNLMLRDASTVIPVMVYLDPM
AVVAE

### Integrin α7β1 Antibodies

The present disclosure provides, among other things, an antibody or antigen binding fragment thereof that binds α7β1 integrin in an extended-open or an extended-closed conformation, thereby maintaining high affinity of α7β1 integrin to laminin. Furthermore, the anti-α7β1 integrin antibodies of the present disclosure are effective in promoting muscle cell adhesion to the laminin.

In some embodiments, an anti-α7β1 integrin antibody activates the α7β1 integrin. In some embodiments, an anti-α7β1 integrin antibody stabilizes an active conformation of α7β1 integrin. In some embodiments an anti-α7β1 integrin antibody increases expression of ITGA7 and/or activity of α7β1 integrin. In some embodiments an anti-α7β1 integrin antibody increases activity of α7β1 integrin.

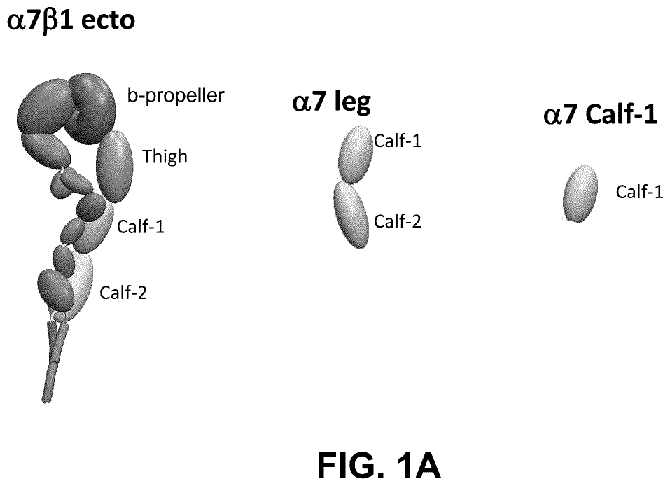

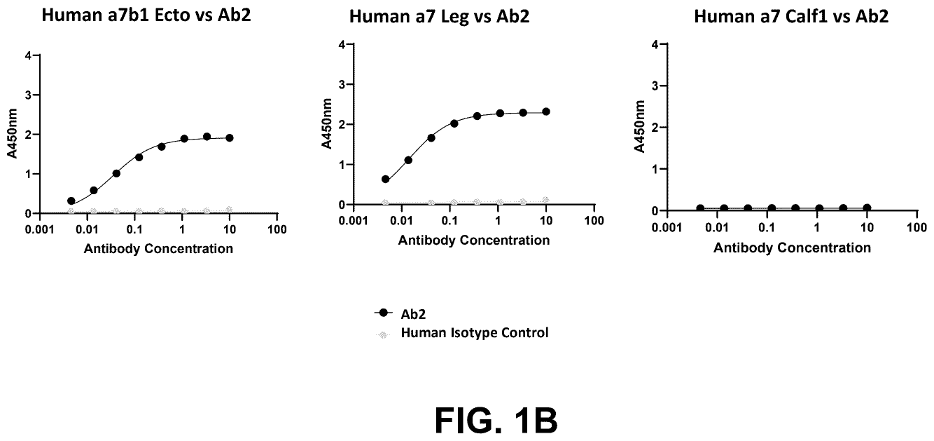

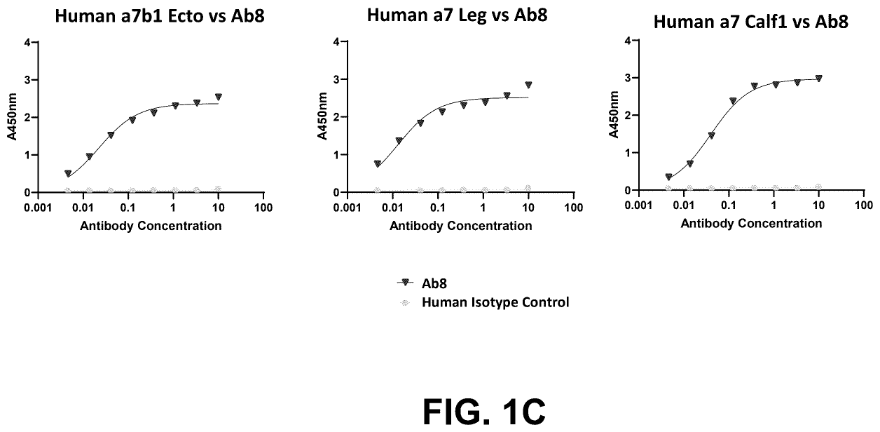

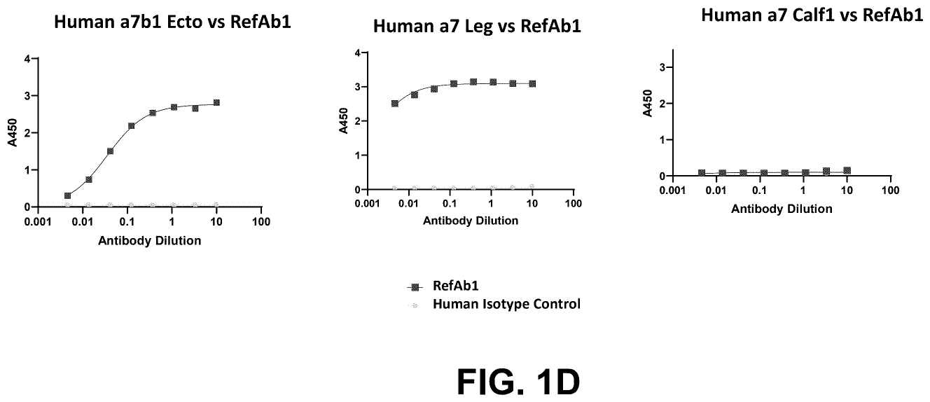

α7β1 integrin contains multiple domains as shown in FIG. 1A and FIG. 5C. In some embodiments, an anti-α7β1 integrin antibody binds to the leg of α7. In some embodiments, an anti-α7β1 integrin antibody binds to the laminin binding domain. In some embodiments, an anti-α7β1 integrin antibody binds to the calf-1 and/or calf-2 domains of α7β1 integrin. In some embodiments, an anti-α7β1 integrin antibody binds to the calf-1 domain of α7β1 integrin. In some embodiments, an anti-α7β1 integrin antibody binds to the calf-2 domain of α7β1 integrin.

### Epitope

The present disclosure is based, in part, on the identification of a new epitope of a7 integrin. The anti-α7β1 antibodies that bind to the epitope described herein are able to effectively stabilize the extended conformation of the α7β1 integrin such that the α7β1 integrin binds laminin with high affinity and remains in the active form. Therefore, the agonistic anti-α7β1 integrin antibodies of the present disclosure are effective in treating diseases associated with dystrophin such as Duchenne muscular dystrophy (DMD).

In some embodiments, an anti-α7β1 integrin antibody binds to calf-2 domain of α7β1 integrin. In some embodiments, an anti-α7β1 integrin antibody binds to a region comprising positions 856-862, 867-889, 1011-1017, 897-905 or 1002-1010 of integrin α7. In some embodiments, an anti-α7β1 integrin antibody binds to a region comprising positions 856-862 of integrin α7. In some embodiments, an anti-α7β1 integrin antibody binds to a region comprising positions 867-889 of integrin α7. In some embodiments, an anti-α7β1 integrin antibody binds to a region comprising positions 1011-1017 of integrin α7. In some embodiments, an anti-α7β1 integrin antibody binds to a region comprising positions 897-905 of integrin α7. In some embodiments, an anti-α7β1 integrin antibody binds to a region comprising positions 1002-1010 of integrin α7. In some embodiments, an anti-α7β1 integrin antibody binds to an epitope comprising amino acids M856, A857, 1858, P859, Q860, Q861, L862, V867, V868, R869, G870, E871, R872, A873, M874, Q875, S876, E877, R878, D879, V880, G881, S882, K883, V884, K885, Y886, E887, V888, T889, L897, R898, T899, L900, G901, S902, A903, F904, L905, V1002, V1003, F1004, S1005, C1006, P1007, L1008, Y1009, S1010, F1011, D1012, R1013, A1014, A1015, V1016, and/or L1017. In some embodiments, an anti-α7β1 integrin antibody binds to an epitope comprising amino acids M856, A857, 1858, P859, Q860, Q861, and/or L862. In some embodiments, an anti-α7β1 integrin antibody binds to an epitope comprising amino acids V867, V868, R869, G870, E871, R872, A873, M874, Q875, S876, E877, R878, D879, V880, G881, S882, K883, V884, K885, Y886, E887, V888, and/or T889. In some embodiments, an anti-α7β1 integrin antibody binds to an epitope comprising amino acids L897, R898, T899, L900, G901, S902, A903, F904, and/or L905. In some embodiments, an anti-α7β1 integrin antibody binds to an epitope comprising amino acids V1002, V1003, F1004, S1005, C1006, P1007, L1008, Y1009, S1010. In some embodiments, an anti-α7β1 integrin antibody binds to an epitope comprising amino acids F1011, D1012, R1013, A1014, A1015, V1016, and/or L1017.

In some embodiments, an anti-α7β1 integrin antibody binds to calf-2 domain of α7β1 integrin, but does not compete with the 20TFT antibody. In some embodiments, an anti-α7β1 integrin antibody binds to calf-2 domain of α7β1 integrin, but does not bind to an epitope comprising amino acids S977, W978, W979, and P980.

In some embodiments, an anti-α7β1 integrin antibody binds to calf-1 domain of α7β1 integrin. In some embodiments, an anti-α7β1 integrin antibody binds to a region comprising positions 771-778 or 793-805 of integrin α7. In some embodiments, an anti-α7β1 integrin antibody binds to a region comprising positions 771-778 of integrin α7. In some embodiments, an anti-α7β1 integrin antibody binds to a region comprising positions 793-805 of integrin α7. In some embodiments, an anti-α7β1 integrin antibody binds to an epitope comprising amino acids R771, A772, L773, D774, P775, A776, E777, K778, E793, L794, G795, N796, P797, M798, K799, R800, G801, A802, Q803, V804, and/or T805. In some embodiments, an anti-α7β1 integrin antibody binds to an epitope comprising amino acids R771, A772, L773, D774, A776, E777, K778, E793, N796, K799, Q803, and/or T805. In some embodiments, an anti-α7β1 integrin antibody binds to an epitope comprising amino acids R771, A772, L773, D774, A776, E777, K778, E793, N796, K799, Q803, and T805.

### Human Anti-α7β1 Integrin Antibodies

In some embodiments, the integrin α7β1 antibody or antigen binding fragment comprises at least one, two, or three complementary determining regions (CDRs) from a heavy chain variable region having an amino acid sequence as set forth in Table A, or a sequence substantially homologous thereto (e.g., a sequence at least about 85%, 90%, 95%, 99% or more identical thereto, and/or having one or more substitutions, e.g., conserved substitutions). In some embodiments, integrin α7β1 antibody or antigen binding fragment thereof comprises at least one, two, or three CDRs from a light chain variable region having an amino acid sequence as set forth in Table A, or a sequence substantially homologous thereto (e.g., a sequence at least about 85%, 90%, 95%, 99% or more identical thereto, and/or having one or more substitutions, e.g., conserved substitutions). In an embodiment, the integrin α7β1 antibody or antigen binding fragment thereof comprises at least one, two, three, four, five, or six CDRs from heavy and light chain variable regions having an amino acid sequence as set forth in Table A, or a sequence substantially homologous thereto (e.g., a sequence at least about 85%, 90%, 95%, 99% or more identical thereto, and/or having one or more substitutions, e.g., conserved substitutions).

In some embodiments, the antibody or antigen-binding fragment thereof is an anti-human α7β1 antibody. In some embodiments, the antibody or antigen-binding fragment thereof comprises CDRs selected from Table A. Unless indicated otherwise, the CDR sequences shown in the present application are in the IMGT numbering scheme.

TABLE A
Exemplary CDRs of human anti-α7β1 antibodies.

| Ab | HCDR1 | HCDR2 | HCDR3 | LCDR1 | LCDR2 | LCDR3 |
|---|---|---|---|---|---|---|
| Ab1 | GYTFTGYY | INPNSGGT | AREGANWDYYYYG | SSNIGSKT | SNN | AAWDDSLNGPV |
| | (SEQ ID | (SEQ ID | MDV | (SEQ ID | (SEQ ID | (SEQ ID NO: |
| | NO: 1, | NO: 15, | (SEQ ID NO: | NO: 46, | NO: 58, | 69, LCDR3A) |
| | HCDR1A) | HCDR2A) | 31, HCDR3A) | LCDR1A) | LCDR2A) | |
| Ab2 | GFTFSSYT | ISIVGNYI | ARRKWEHDAFDI | SLRSYY | GKN | TSRDNSGNHVV |
| | (SEQ ID | (SEQ ID | (SEQ ID NO: | (SEQ ID | (SEQ ID | (SEQ ID NO: |
| | NO: 2, | NO: 16, | 32, HCDR3B) | NO: 47, | NO: 59, | 70, LCDR3B) |
| | HCDR1B) | HCDR2B) | | LCDR1B) | LCDR2B) | |
| Ab3 | GFTFSSYS | ISTSSSYI | ARRAWESDAFAI | SLRSYY | GKN | NSRDSSGNHVV |
| | (SEQ ID | (SEQ ID | (SEQ ID NO: | (SEQ ID | (SEQ ID | (SEQ ID NO: |
| | NO: 3, | NO: 17, | 33, HCDR3C) | NO: 47, | NO: 59, | 71, LCDR3C) |
| | HCDR1C) | HCDR2C) | | LCDR1B) | LCDR2B) | |
| Ab4 | GFTFSSYA | ISDSGGIA | VRDGY | QSLLDSDD | TLS | MQRIEFPLT |
| | (SEQ ID | (SEQ ID | (SEQ ID NO: | GYTY | (SEQ ID | (SEQ ID NO: |
| | NO: 4, | NO: 18, | 34, HCDR3D) | (SEQ ID | NO: 60, | 72, LCDR3D) |
| | HCDR1D) | HCDR2D) | | NO: 48, | LCDR2C) | |
| | | | | LCDR1C) | | |
| Ab5 | GFTFSSYA | IHYSGGTT | VKDGY | QSLLDSDD | TLS | MQRIEFPLT |
| | (SEQ ID | (SEQ ID | (SEQ ID NO: | GYTY | (SEQ ID | (SEQ ID NO: |
| | NO: 4, | NO: 19, | 35, HCDR3E) | (SEQ ID | NO: 60, | 72, LCDR3D) |
| | HCDR1D) | HCDR2E) | | NO: 48, | LCDR2C) | |
| | | | | LCDR1C) | | |
| Ab6 | GFTFSRFW | TKQDGSEK | AREGSGAYWAFDI | QSLVHNDG | KIS | LQATQFPLT |
| | (SEQ ID | (SEQ ID | (SEQ ID NO: | NTY | (SEQ ID | (SEQ ID NO: |
| | NO: 5, | NO: 20, | 36, HCDR3F) | (SEQ ID | NO: 61, | 73, LCDR3E) |
| | HCDR1E) | HCDR2F) | | NO: 49, | LCDR2D) | |
| | | | | LCDR1D) | | |
| Ab7 | GGSISSYY | IYTRGST | ARVFGGNYRFDAF | SSNIGAGY | GNN | QSYDSSLSGYV |
| | (SEQ ID | (SEQ ID | HI (SEQ ID | D | (SEQ ID | (SEQ ID NO: |
| | NO: 6, | NO: 21, | NO: 37, | (SEQ ID | NO: 62, | 74, LCDR3F) |
| | HCDR1F) | HCDR2G) | HCDR3G) | NO: 50, | LCDR2E) | |
| | | | | LCDR1E) | | |

In some embodiments, the antibody or antigen-binding fragment thereof comprises an HCDR1 of SEQ ID NO: 1 (HCDR1A), an HCDR2 of SEQ ID NO: 15 (HCDR2A), and an HCDR3 of SEQ ID NO: 31 (HCDR3A). In some embodiments, the antibody or antigen-binding fragment thereof comprises an LCDR1 of SEQ ID NO: 46 (LCDR1A), an LCDR2 of SEQ ID NO: 58 (LCDR2A), and an LCDR3 of SEQ ID NO: 69, LCDR3A. In some embodiments, the antibody or antigen-binding fragment thereof comprises an HCDR1 of SEQ ID NO: 1 (HCDR1A), an HCDR2 of SEQ ID NO: 15 (HCDR2A), an HCDR3 of SEQ ID NO: 31 (HCDR3A), an LCDR1 of SEQ ID NO: 46 (LCDR1A), an LCDR2 of SEQ ID NO: 58 (LCDR2A), and an LCDR3 of SEQ ID NO: 69 (LCDR3A).

In some embodiments, the antibody or antigen-binding fragment thereof competes with a reference antibody, wherein the reference antibody comprises an HCDR1 of SEQ ID NO: 1 (HCDR1A), an HCDR2 of SEQ ID NO: 15 (HCDR2A), an HCDR3 of SEQ ID NO: 31 (HCDR3A), an LCDR1 of SEQ ID NO: 46 (LCDR1A), an LCDR2 of SEQ ID NO: 58 (LCDR2A), and an LCDR3 of SEQ ID NO: 69 (LCDR3A).

In some embodiments, the antibody or antigen-binding fragment thereof comprises an HCDR1 of SEQ ID NO: HCDR1B, an HCDR2 of SEQ ID NO: 16 (HCDR2B), and an HCDR3 of SEQ ID NO: 32(HCDR3B). In some embodiments, the antibody or antigen-binding fragment thereof comprises an LCDR1 of SEQ ID NO: 47 (LCDR1B), an LCDR2 of SEQ ID NO: 59 (LCDR2B), and an LCDR3 of SEQ ID NO: 70 (LCDR3B). In some embodiments, the antibody or antigen-binding fragment thereof comprises an HCDR1 of SEQ ID NO: HCDR1B, an HCDR2 of SEQ ID NO: 16 (HCDR2B), an HCDR3 of SEQ ID NO: 32 (HCDR3B), an LCDR1 of SEQ ID NO: 47 (LCDR1B), an LCDR2 of SEQ ID NO: 59 (LCDR2B), and an LCDR3 of SEQ ID NO: 70 (LCDR3B).

In some embodiments, the antibody or antigen-binding fragment thereof competes with a reference antibody, wherein the reference antibody comprises an HCDR1 of SEQ ID NO: HCDR1B, an HCDR2 of SEQ ID NO: 16 (HCDR2B), an HCDR3 of SEQ ID NO: 32 (HCDR3B), an LCDR1 of SEQ ID NO: 47 (LCDR1B), an LCDR2 of SEQ ID NO: 59 (LCDR2B), and an LCDR3 of SEQ ID NO: 70 (LCDR3B).

In some embodiments, the antibody or antigen-binding fragment thereof comprises an HCDR1 of SEQ ID NO: 3 (HCDR1C), an HCDR2 of SEQ ID NO: 17 (HCDR2C), and an HCDR3 of SEQ ID NO: 33 (HCDR3C). In some embodiments, the antibody or antigen-binding fragment thereof comprises an LCDR1 of SEQ ID NO: 47 (LCDR1B), an LCDR2 of SEQ ID NO: 59 (LCDR2B), and an LCDR3 of SEQ ID NO: 71 (LCDR3C). In some embodiments, the antibody or antigen-binding fragment thereof comprises an HCDR1 of SEQ ID NO: 3 (HCDR1C), an HCDR2 of SEQ ID NO: 17 (HCDR2C), an HCDR3 of SEQ ID NO: 33 (HCDR3C), an LCDR1 of SEQ ID NO: 47 (LCDR1B), an LCDR2 of SEQ ID NO: 59 (LCDR2B), and an LCDR3 of SEQ ID NO: 71 (LCDR3C).

In some embodiments, the antibody or antigen-binding fragment thereof competes with a reference antibody, wherein the reference antibody comprises an HCDR1 of SEQ ID NO: 3 (HCDR1C), an HCDR2 of SEQ ID NO: 17 (HCDR2C), an HCDR3 of SEQ ID NO: 33 (HCDR3C), an LCDR1 of SEQ ID NO: 47 (LCDR1B), an LCDR2 of SEQ ID NO: 59 (LCDR2B), and an LCDR3 of SEQ ID NO: 71 (LCDR3C).

In some embodiments, the antibody or antigen-binding fragment thereof comprises an HCDR1 of SEQ ID NO: 4 (HCDR1D), an HCDR2 of SEQ ID NO: 18 (HCDR2D), and an HCDR3 of SEQ ID NO: 34 (HCDR3D). In some embodiments, the antibody or antigen-binding fragment thereof comprises an LCDR1 of SEQ ID NO: 48 (LCDR1C), an LCDR2 of SEQ ID NO: 60 (LCDR2C), and an LCDR3 of SEQ ID NO: 72 (LCDR3D). In some embodiments, the antibody or antigen-binding fragment thereof comprises an HCDR1 of SEQ ID NO: 4 (HCDR1D), an HCDR2 of SEQ ID NO: 18 (HCDR2D), an HCDR3 of SEQ ID NO: 34 (HCDR3D), an LCDR1 of SEQ ID NO: 48 (LCDR1C), an LCDR2 of SEQ ID NO: 60 (LCDR2C), and an LCDR3 of SEQ ID NO: 72 (LCDR3D).

In some embodiments, the antibody or antigen-binding fragment thereof competes with a reference antibody, wherein the reference antibody comprises an HCDR1 of SEQ ID NO: 4 (HCDR1D), an HCDR2 of SEQ ID NO: 18 (HCDR2D), an HCDR3 of SEQ ID NO: 34 (HCDR3D), an LCDR1 of SEQ ID NO: 48 (LCDR1C), an LCDR2 of SEQ ID NO: 60 (LCDR2C), and an LCDR3 of SEQ ID NO: 72 (LCDR3D).

In some embodiments, the antibody or antigen-binding fragment thereof comprises an HCDR1 of SEQ ID NO: 4 (HCDR1D), an HCDR2 of SEQ ID NO: 19 (HCDR2E), and an HCDR3 of SEQ ID NO: 35 (HCDR3E). In some embodiments, the antibody or antigen-binding fragment thereof comprises an LCDR1 of SEQ ID NO: 48 (LCDR1C), an LCDR2 of SEQ ID NO: 60 (LCDR2C), and an LCDR3 of SEQ ID NO: 72 (LCDR3D). In some embodiments, the antibody or antigen-binding fragment thereof comprises an HCDR1 of SEQ ID NO: 4 (HCDR1D), an HCDR2 of SEQ ID NO: 19 (HCDR2E), an HCDR3 of SEQ ID NO: 35 (HCDR3E), an LCDR1 of SEQ ID NO: 48 (LCDR1C), an LCDR2 of SEQ ID NO: 60 (LCDR2C), and an LCDR3 of SEQ ID NO: 72 (LCDR3D).

In some embodiments, the antibody or antigen-binding fragment thereof competes with a reference antibody, wherein the reference antibody comprises an HCDR1 of SEQ ID NO: 4 (HCDR1D), an HCDR2 of SEQ ID NO: 19 (HCDR2E), an HCDR3 of SEQ ID NO: 35 (HCDR3E), an LCDR1 of SEQ ID NO: 48 (LCDR1C), an LCDR2 of SEQ ID NO: 60 (LCDR2C), and an LCDR3 of SEQ ID NO: 72 (LCDR3D).

In some embodiments, the antibody or antigen-binding fragment thereof comprises an HCDR1 of SEQ ID NO: 5 (HCDR1E), an HCDR2 of SEQ ID NO: 20 (HCDR2F), and an HCDR3 of SEQ ID NO: 36 (HCDR3F). In some embodiments, the antibody or antigen-binding fragment thereof comprises an LCDR1 of SEQ ID NO: 49 (LCDR1D), an LCDR2 of SEQ ID NO: 61 (LCDR2D), and an LCDR3 of SEQ ID NO: 73 (LCDR3E). In some embodiments, the antibody or antigen-binding fragment thereof comprises an HCDR1 of SEQ ID NO: 5 (HCDR1E), an HCDR2 of SEQ ID NO: 20 (HCDR2F), an HCDR3 of SEQ ID NO: 36 (HCDR3F), an LCDR1 of SEQ ID NO: 49 (LCDR1D), an LCDR2 of SEQ ID NO: 61 (LCDR2D), and an LCDR3 of SEQ ID NO: 73 (LCDR3E).

In some embodiments, the antibody or antigen-binding fragment thereof competes with a reference antibody, wherein the reference antibody comprises an HCDR1 of SEQ ID NO: 5 (HCDR1E), an HCDR2 of SEQ ID NO: 20 (HCDR2F), an HCDR3 of SEQ ID NO: 36 (HCDR3F), an LCDR1 of SEQ ID NO: 49 (LCDR1D), an LCDR2 of SEQ ID NO: 61 (LCDR2D), and an LCDR3 of SEQ ID NO: 73 (LCDR3E).

In some embodiments, the antibody or antigen-binding fragment thereof comprises an HCDR1 of SEQ ID NO: 6 (HCDR1F), an HCDR2 of SEQ ID NO: 21 (HCDR2G), and an HCDR3 of SEQ ID NO: 37 (HCDR3G). In some embodiments, the antibody or antigen-binding fragment thereof comprises an LCDR1 of SEQ ID NO: 50 (LCDR1E), an LCDR2 of SEQ ID NO: 62 (LCDR2E), and an LCDR3 of SEQ ID NO: 74 (LCDR3F). In some embodiments, the antibody or antigen-binding fragment thereof comprises an HCDR1 of SEQ ID NO: 6 (HCDR1F), an HCDR2 of SEQ ID NO: 21 (HCDR2G), and an HCDR3 of SEQ ID NO: 37 (HCDR3G), an LCDR1 of SEQ ID NO: 50 (LCDR1E), an LCDR2 of SEQ ID NO: 62 (LCDR2E), and an LCDR3 of SEQ ID NO: 74 (LCDR3F).

In some embodiments, the antibody or antigen-binding fragment thereof competes with a reference antibody, wherein the reference antibody comprises an HCDR1 of SEQ ID NO: 6 (HCDR1F), an HCDR2 of SEQ ID NO: 21 (HCDR2G), and an HCDR3 of SEQ ID NO: 37 (HCDR3G), an LCDR1 of SEQ ID NO: 50 (LCDR1E), an LCDR2 of SEQ ID NO: 62 (LCDR2E), and an LCDR3 of SEQ ID NO: 74 (LCDR3F).

In some embodiments, the integrin α7β1 antibody or antigen binding fragment thereof comprises CDRs with one or more amino acid different from CDRs shown in Table A. In some embodiments, the integrin α7β1 antibody or antigen binding fragment thereof comprises a CDR with no more than 5 amino acids different from any one of the CDRs shown in Table A. In some embodiments, the integrin α7β1 antibody or antigen binding fragment thereof comprises a CDR with no more than 4 amino acids different from any one of the CDRs shown in Table A. In some embodiments, the integrin α7β1 antibody or antigen binding fragment thereof comprises a CDR with no more than 3 amino acids different from any one of the CDRs shown in Table A. In some embodiments, the integrin α7β1 antibody or antigen binding fragment thereof comprises a CDR with no more than 2 amino acids different from any one of the CDRs shown in Table A. In some embodiments, the integrin α7β1 antibody or antigen binding fragment thereof comprises a CDR with no more than 1 amino acid different from any one of the CDRs shown in Table A.

In some embodiments, the integrin α7β1 antibody or antigen binding fragment thereof comprises a HCDR1 comprising an amino acid sequence with no greater than 2 amino acids different from GYTFTGYY (SEQ ID NO: 1, HCDR1A), a HCDR2 comprising an amino acid sequence with no greater than 1 amino acid different from INPNSGGT (SEQ ID NO: 15, HCDR2A), a HCDR3 comprising an amino acid sequence with no greater than 4 amino acids different from AREGANWDYYYYGMDV (SEQ ID NO: 31 HCDR3A), a LCDR1 comprising an amino acid sequence with no greater than 2 amino acids different from SSNIGSKT (SEQ ID NO: 46, LCDR1A), a LCDR2 comprising an amino acid sequence with no greater than 1 amino acid different from SNN (SEQ ID NO: 58, LCDR2A), and a LCDR3 comprising an amino acid sequence with no greater than 4 amino acids different from AAWDDSLNGPV (SEQ ID NO: 69, LCDR3A).

In some embodiments, the integrin α7β1 antibody or antigen binding fragment thereof comprises a HCDR1 comprising an amino acid sequence with no greater than 2 amino acids different from GFTFSSYT (SEQ ID NO: 2, HCDR1B), a HCDR2 comprising an amino acid sequence with no greater than 1 amino acid different from ISIVGNYI (SEQ ID NO: 16, HCDR2B), a HCDR3 comprising an amino acid sequence with no greater than 4 amino acids different from ARRKWEHDAFDI (SEQ ID NO: 32, HCDR3B), a LCDR1 comprising an amino acid sequence with no greater than 2 amino acids different from SLRSYY (SEQ ID NO: 47, LCDR1B), a LCDR2 comprising an amino acid sequence with no greater than 1 amino acid different from GKN (SEQ ID NO: 59, LCDR2B), and a LCDR3 comprising an amino acid sequence with no greater than 4 amino acids different from TSRDNSGNHVV (SEQ ID NO: 70, LCDR3B).

In some embodiments, the integrin α7β1 antibody or antigen binding fragment thereof comprises a HCDR1 comprising an amino acid sequence with no greater than 2 amino acids different from GFTFSSYS (SEQ ID NO: 3, HCDR1C), a HCDR2 comprising an amino acid sequence with no greater than 1 amino acid different from ISTSSSYI (SEQ ID NO: 17, HCDR2C), a HCDR3 comprising an amino acid sequence with no greater than 4 amino acids different from ARRAWESDAFAI (SEQ ID NO: 33, HCDR3C), a LCDR1 comprising an amino acid sequence with no greater than 2 amino acids different from SLRSYY (SEQ ID NO: 47, LCDR1B), a LCDR2 comprising an amino acid sequence with no greater than 1 amino acid different from GKN (SEQ ID NO: 59, LCDR2B), and a LCDR3 comprising an amino acid sequence with no greater than 4 amino acids different from NSRDSSGNHVV (SEQ ID NO: 71, LCDR3C).

In some embodiments, the integrin α7β1 antibody or antigen binding fragment thereof comprises a HCDR1 comprising an amino acid sequence with no greater than 2 amino acids different from GFTFSSYA (SEQ ID NO: 4, HCDR1D), a HCDR2 comprising an amino acid sequence with no greater than 1 amino acid different from ISDSGGIA (SEQ ID NO: 18, HCDR2D), a HCDR3 comprising an amino acid sequence with no greater than 4 amino acids different from VRDGY (SEQ ID NO: 34, HCDR3D), a LCDR1 comprising an amino acid sequence with no greater than 2 amino acids different from QSLLDSDD (SEQ ID NO: 48, LCDR1C), a LCDR2 comprising an amino acid sequence with no greater than 1 amino acid different from TLS (SEQ ID NO: 60, LCDR2C), and a LCDR3 comprising an amino acid sequence with no greater than 4 amino acids different from MQRIEFPLT (SEQ ID NO: 72, LCDR3D).

In some embodiments, the integrin α7β1 antibody or antigen binding fragment thereof comprises a HCDR1 comprising an amino acid sequence with no greater than 2 amino acids different from GFTFSSYA (SEQ ID NO: 4, HCDR1D), a HCDR2 comprising an amino acid sequence with no greater than 1 amino acid different from IHYSGGTT (SEQ ID NO: 19, HCDR2E), a HCDR3 comprising an amino acid sequence with no greater than 4 amino acids different from VKDGY (SEQ ID NO: 35, HCDR3E), a LCDR1 comprising an amino acid sequence with no greater than 2 amino acids different from QSLLDSDDGYTY (SEQ ID NO: 48, LCDR1C), a LCDR2 comprising an amino acid sequence with no greater than 1 amino acid different from TLS (SEQ ID NO: 60, LCDR2C), and a LCDR3 comprising an amino acid sequence with no greater than 4 amino acids different from MQRIEFPLT (SEQ ID NO: 72, LCDR3D).

In some embodiments, the integrin α7β1 antibody or antigen binding fragment thereof comprises a HCDR1 comprising an amino acid sequence with no greater than 2 amino acids different from GFTFSRFW (SEQ ID NO: 5, HCDR1E), a HCDR2 comprising an amino acid sequence with no greater than 1 amino acid different from TKQDGSEK (SEQ ID NO: 20, HCDR2F), a HCDR3 comprising an amino acid sequence with no greater than 4 amino acids different from AREGSGAYWAFDI (SEQ ID NO: 36, HCDR3F), a LCDR1 comprising an amino acid sequence with no greater than 2 amino acids different from QSLVHNDGNTY (SEQ ID NO: 49, LCDR1D), a LCDR2 comprising an amino acid sequence with no greater than 1 amino acid different from KIS (SEQ ID NO: 61, LCDR2D), and a LCDR3 comprising an amino acid sequence with no greater than 4 amino acids different from LQATQFPLT (SEQ ID NO: 73, LCDR3E).

In some embodiments, the integrin α7β1 antibody or antigen binding fragment thereof comprises a HCDR1 comprising an amino acid sequence with no greater than 2 amino acids different from GGSISSYY (SEQ ID NO: 6, HCDR1F), a HCDR2 comprising an amino acid sequence with no greater than 1 amino acid different from IYTRGST (SEQ ID NO: 21, HCDR2G), a HCDR3 comprising an amino acid sequence with no greater than 4 amino acids different from ARVFGGNYRFDAFHI (SEQ ID NO: 37, HCDR3G), a LCDR1 comprising an amino acid sequence with no greater than 2 amino acids different from SSNIGAGYD (SEQ ID NO: 50, LCDR1E), a LCDR2 comprising an amino acid sequence with no greater than 1 amino acid different from GNN (SEQ ID NO: 62, LCDR2E), and a LCDR3 comprising an amino acid sequence with no greater than 4 amino acids different from QSYDSSLSGYV (SEQ ID NO: 74, LCDR3F).

TABLE B
Exemplary VH and VL Sequences of Human anti-α7β1 Integrin Antibodies.

| Ab | VH Sequence | VL Sequence |
|---|---|---|
| Ab1 | QVQLVQSGAEVKKPGASVKVSCKASGYTFTGY | QSVLTQPPSASGTPGQRVTISCSGSSSNIGS |
| | YMHWVRQAPGQGLEWMGWINPNSGGTNYAQKF | KTVNWYQQLPGTAPKLLIYSNNQRPSGVPDR |
| | QGRVTMTRDTSISTAYMELSRLRSDDTAVYYC | FSGSKSGTSASLAISGLQSEDEADYYCAAWD |
| | AREGANWDYYYYGMDVWGQGTTVTVSS (SEQ | DSLNGPVFGGGTKLTVL (SEQ ID NO: |
| | ID NO: 128, VH1) | 129, VL1) |
| Ab2 | EVQLVESGGGLVKPGGSLRLSCAASGFTFSSY | SSELTQDPAVSVALGQTVRITCQGDSLRSYY |
| | TMNWVRQAPGKGLEWVSSISIVGNYIYYADSL | ASWYQQKPGQAPVLVIYGKNNRPSGIPDRES |
| | KGRFTISRDNAKNSLYLQMNSLRAEDTAVYYC | GSSSGNTASLTITGAQAEDEADFYCTSRDNS |
| | ARRKWEHDAFDIWGQGTMVTVSS (SEQ ID | GNHVVFGGGTKLTVL (SEQ ID NO: |
| | NO: 130, VH2) | 131, VL2) |
| Ab3 | EVQLVESGGGLVKPGGSLRLSCAASGFTFSSY | SSELTQDPAVSVALGQTVRITCQGDSLRSYY |
| | SMNWVRQAPGKGLEWVSSISTSSSYIYYADSV | ASWYQQKPGQAPVLVIYGKNNRPSGIPDRFS |
| | KGRFTISRDNAKNSLYLQMNSLRAEDTAVYYC | GSSSGNTASLTITGAQAEDEADYYCNSRDSS |
| | ARRAWESDAFAIWGQGTMVTVSS (SEQ ID | GNHVVFGGGTKLTVL (SEQ ID NO: |
| | NO: 132, VH3) | 133, VL3) |
| Ab4 | EVQLLESGGGLEQPGGSLRLSCAASGFTFSSY | DIVMTQTPLSLPVTPGEPASISCRSSQSLLD |
| | AMNWVRQGLGKGLEWVSAISDSGGIAYYADSV | SDDGYTYLDWYLQKPGQSPQLLIYTLSYRAS |
| | KGRFTISRDNSKNTLYLQMDSLRGEDTAVYYC | GVPDRFSGSGSGTDFSLKISRVEAEDAGIYY |
| | VRDGYWGQGTLVTVSS (SEQ ID NO: | CMQRIEFPLTFGGGTKVEIK (SEQ ID |
| | 134, VH4) | NO: 135, VL4) |
| Ab5 | EVQLLESGGGLVQPGGSLRLSCVASGFTESSY | DIMMTQTPLSLPVSPGEPASISCRSSQSLLD |
| | AMTWVRQAPGKGLEWVSAIHYSGGTTNYAASV | SDDGYTYLDWYLQKPGQSPQLLIYTLSYRAS |
| | KGRFTISRDNSKNTLYLQMNSLRAEDTAVYYC | GVPDRFSGSGSGTDFTLKISRVEAEDVGVYY |
| | VKDGYWGQGTLVTVSS (SEQ ID NO: | CMQRIEFPLTFGGGTKVEIK (SEQ ID |
| | 136, VH5) | NO: 137, VL5) |
| Ab6 | EVQLVESGGGLVQPGGSLRLSCAASGFTFSRF | DIVMTQTPLSSPVTLGQPASISCRSSQSLVH |
| | WMSWVRQAPGKGLEWVANTKQDGSEKYYVDSV | NDGNTYLSWLQQRPGQPPRLLIYKISNRFSG |
| | KGRFTISRDNAKNSLYLQMNSLRADDTAVYYC | VPDRFSGSGAGTDFTLKISRVEAEDVGVYYC |
| | AREGSGAYWAFDIWGQGTMVTVSS (SEQ ID | LQATQFPLTFGGGTKVEIK (SEQ ID NO: |
| | NO: 138, VH6) | 139, VL6) |
| Ab7 | QVQLQESGPGLVKPSETLSLTCTVSGGSISSY | QSVLTQPPSVSGAPGQRVTISCTGSSSNIGA |
| | YWSWIRQPAGKGLEWIGRIYTRGSTNYNPSLK | GYDVHWYQQLPGTAPKLLIYGNNNRPSGVPD |
| | SRVTMSVDTSKNQFSLKLSSVTAADTAVYYCA | RFSGSKSGTSASLAITGLQAEDEADYYCQSY |
| | RVFGGNYRFDAFHIWSQGTMVTVSS (SEQ | DSSLSGYVFGTGTKVTVL (SEQ ID NO: |
| | ID NO: 140, VH7) | 141, VL7) |

In some embodiments, the integrin α7β1 antibody or antigen binding fragment thereof comprises a variable heavy region comprising an amino acid sequence at least 80% identical to SEQ ID NO: 128 (VH1). In some embodiments, the integrin α7β1 antibody or antigen binding fragment thereof comprises a variable heavy region comprising an amino acid sequence at least 83% identical to SEQ ID NO: 128 (VH1). In some embodiments, the integrin α7β1 antibody or antigen binding fragment thereof comprises a variable heavy region comprising an amino acid sequence at least 85% identical to SEQ ID NO: 128 (VH1). In some embodiments, the integrin α7β1 antibody or antigen binding fragment thereof comprises a variable heavy region comprising an amino acid sequence at least 87% identical to SEQ ID NO: 128 (VH1). In some embodiments, the integrin α7β1 antibody or antigen binding fragment thereof comprises a variable heavy region comprising an amino acid sequence at least 90% identical to SEQ ID NO: 128 (VH1). In some embodiments, the integrin α7β1 antibody or antigen binding fragment thereof comprises a variable heavy region comprising an amino acid sequence at least 92% identical to SEQ ID NO: 128 (VH1). In some embodiments, the integrin α7β1 antibody or antigen binding fragment thereof comprises a variable heavy region comprising an amino acid sequence at least 93% identical to SEQ ID NO: 128 (VH1). In some embodiments, the integrin α7β1 antibody or antigen binding fragment thereof comprises a variable heavy region comprising an amino acid sequence at least 94% identical to SEQ ID NO: 128 (VH1). In some embodiments, the integrin α7β1 antibody or antigen binding fragment thereof comprises a variable heavy region comprising an amino acid sequence at least 95% identical to SEQ ID NO: 128 (VH1). In some embodiments, the integrin α7β1 antibody or antigen binding fragment thereof comprises a variable heavy region comprising an amino acid sequence at least 97% identical to SEQ ID NO: 128 (VH1). In some embodiments, the integrin α7β1 antibody or antigen binding fragment thereof comprises a variable heavy region comprising an amino acid sequence at least 98% identical to SEQ ID NO: 128 (VH1). In some embodiments, the integrin α7β1 antibody or antigen binding fragment thereof comprises a variable heavy region comprising an amino acid sequence at least 99% identical to SEQ ID NO: 128 (VH1). In some embodiments, the integrin α7β1 antibody or antigen binding fragment thereof comprises a variable heavy region comprising an amino acid sequence identical to SEQ ID NO: 128 (VH1).

In some embodiments, the integrin α7β1 antibody or antigen binding fragment thereof comprises a variable heavy region comprising an amino acid sequence at least 80% identical to SEQ ID NO: 130 (VH2). In some embodiments, the integrin α7β1 antibody or antigen binding fragment thereof comprises a variable heavy region comprising an amino acid sequence at least 83% identical to SEQ ID NO: 130 (VH2). In some embodiments, the integrin α7β1 antibody or antigen binding fragment thereof comprises a variable heavy region comprising an amino acid sequence at least 85% identical to SEQ ID NO: 130 (VH2). In some embodiments, the integrin α7β1 antibody or antigen binding fragment thereof comprises a variable heavy region comprising an amino acid sequence at least 87% identical to SEQ ID NO: 130 (VH2). In some embodiments, the integrin α7β1 antibody or antigen binding fragment thereof comprises a variable heavy region comprising an amino acid sequence at least 90% identical to SEQ ID NO: 130 (VH2). In some embodiments, the integrin α7β1 antibody or antigen binding fragment thereof comprises a variable heavy region comprising an amino acid sequence at least 92% identical to SEQ ID NO: 130 (VH2). In some embodiments, the integrin α7β1 antibody or antigen binding fragment thereof comprises a variable heavy region comprising an amino acid sequence at least 93% identical to SEQ ID NO: 130 (VH2). In some embodiments, the integrin α7β1 antibody or antigen binding fragment thereof comprises a variable heavy region comprising an amino acid sequence at least 94% identical to SEQ ID NO: 130 (VH2). In some embodiments, the integrin α7β1 antibody or antigen binding fragment thereof comprises a variable heavy region comprising an amino acid sequence at least 95% identical to SEQ ID NO: 130 (VH2). In some embodiments, the integrin α7β1 antibody or antigen binding fragment thereof comprises a variable heavy region comprising an amino acid sequence at least 97% identical to SEQ ID NO: 130 (VH2). In some embodiments, the integrin α7β1 antibody or antigen binding fragment thereof comprises a variable heavy region comprising an amino acid sequence at least 98% identical to SEQ ID NO: 130 (VH2). In some embodiments, the integrin α7β1 antibody or antigen binding fragment thereof comprises a variable heavy region comprising an amino acid sequence at least 99% identical to SEQ ID NO: 130 (VH2). In some embodiments, the integrin α7β1 antibody or antigen binding fragment thereof comprises a variable heavy region comprising an amino acid sequence identical to SEQ ID NO: 130 (VH2).

In some embodiments, the integrin α7β1 antibody or antigen binding fragment thereof comprises a variable heavy region comprising an amino acid sequence at least 80% identical to SEQ ID NO: 132 (VH3). In some embodiments, the integrin α7β1 antibody or antigen binding fragment thereof comprises a variable heavy region comprising an amino acid sequence at least 83% identical to SEQ ID NO: 132 (VH3). In some embodiments, the integrin α7β1 antibody or antigen binding fragment thereof comprises a variable heavy region comprising an amino acid sequence at least 85% identical to SEQ ID NO: 132 (VH3). In some embodiments, the integrin α7β1 antibody or antigen binding fragment thereof comprises a variable heavy region comprising an amino acid sequence at least 87% identical to SEQ ID NO: 132 (VH3). In some embodiments, the integrin α7β1 antibody or antigen binding fragment thereof comprises a variable heavy region comprising an amino acid sequence at least 90% identical to SEQ ID NO: 132 (VH3). In some embodiments, the integrin α7β1 antibody or antigen binding fragment thereof comprises a variable heavy region comprising an amino acid sequence at least 92% identical to SEQ ID NO: 132 (VH3). In some embodiments, the integrin α7β1 antibody or antigen binding fragment thereof comprises a variable heavy region comprising an amino acid sequence at least 93% identical to SEQ ID NO: 132 (VH3). In some embodiments, the integrin α7β1 antibody or antigen binding fragment thereof comprises a variable heavy region comprising an amino acid sequence at least 94% identical to SEQ ID NO: 132 (VH3). In some embodiments, the integrin α7β1 antibody or antigen binding fragment thereof comprises a variable heavy region comprising an amino acid sequence at least 95% identical to SEQ ID NO: 132 (VH3). In some embodiments, the integrin α7β1 antibody or antigen binding fragment thereof comprises a variable heavy region comprising an amino acid sequence at least 97% identical to SEQ ID NO: 132 (VH3). In some embodiments, the integrin α7β1 antibody or antigen binding fragment thereof comprises a variable heavy region comprising an amino acid sequence at least 98% identical to SEQ ID NO: 132 (VH3). In some embodiments, the integrin α7β1 antibody or antigen binding fragment thereof comprises a variable heavy region comprising an amino acid sequence at least 99% identical to SEQ ID NO: 132 (VH3). In some embodiments, the integrin α7β1 antibody or antigen binding fragment thereof comprises a variable heavy region comprising an amino acid sequence identical to SEQ ID NO: 132 (VH3).

In some embodiments, the integrin α7β1 antibody or antigen binding fragment thereof comprises a variable heavy region comprising an amino acid sequence at least 80% identical to SEQ ID NO: 134 (VH4). In some embodiments, the integrin α7β1 antibody or antigen binding fragment thereof comprises a variable heavy region comprising an amino acid sequence at least 83% identical to SEQ ID NO: 134 (VH4). In some embodiments, the integrin α7β1 antibody or antigen binding fragment thereof comprises a variable heavy region comprising an amino acid sequence at least 85% identical to SEQ ID NO: 134 (VH4). In some embodiments, the integrin α7β1 antibody or antigen binding fragment thereof comprises a variable heavy region comprising an amino acid sequence at least 87% identical to SEQ ID NO: 134 (VH4). In some embodiments, the integrin α7β1 antibody or antigen binding fragment thereof comprises a variable heavy region comprising an amino acid sequence at least 90% identical to SEQ ID NO: 134 (VH4). In some embodiments, the integrin α7β1 antibody or antigen binding fragment thereof comprises a variable heavy region comprising an amino acid sequence at least 92% identical to SEQ ID NO: 134 (VH4). In some embodiments, the integrin α7β1 antibody or antigen binding fragment thereof comprises a variable heavy region comprising an amino acid sequence at least 93% identical to SEQ ID NO: 134 (VH4). In some embodiments, the integrin α7β1 antibody or antigen binding fragment thereof comprises a variable heavy region comprising an amino acid sequence at least 94% identical to SEQ ID NO: 134 (VH4). In some embodiments, the integrin α7β1 antibody or antigen binding fragment thereof comprises a variable heavy region comprising an amino acid sequence at least 95% identical to SEQ ID NO: 134 (VH4). In some embodiments, the integrin α7β1 antibody or antigen binding fragment thereof comprises a variable heavy region comprising an amino acid sequence at least 97% identical to SEQ ID NO: 134 (VH4). In some embodiments, the integrin α7β1 antibody or antigen binding fragment thereof comprises a variable heavy region comprising an amino acid sequence at least 98% identical to SEQ ID NO: 134 (VH4). In some embodiments, the integrin α7β1 antibody or antigen binding fragment thereof comprises a variable heavy region comprising an amino acid sequence at least 99% identical to SEQ ID NO: 134 (VH4). In some embodiments, the integrin α7β1 antibody or antigen binding fragment thereof comprises a variable heavy region comprising an amino acid sequence identical to SEQ ID NO: 134 (VH4).

In some embodiments, the integrin α7β1 antibody or antigen binding fragment thereof comprises a variable heavy region comprising an amino acid sequence at least 80% identical to SEQ ID NO: 136 (VH5). In some embodiments, the integrin α7β1 antibody or antigen binding fragment thereof comprises a variable heavy region comprising an amino acid sequence at least 83% identical to SEQ ID NO: 136 (VH5). In some embodiments, the integrin α7β1 antibody or antigen binding fragment thereof comprises a variable heavy region comprising an amino acid sequence at least 85% identical to SEQ ID NO: 136 (VH5). In some embodiments, the integrin α7β1 antibody or antigen binding fragment thereof comprises a variable heavy region comprising an amino acid sequence at least 87% identical to SEQ ID NO: 136 (VH5). In some embodiments, the integrin α7β1 antibody or antigen binding fragment thereof comprises a variable heavy region comprising an amino acid sequence at least 90% identical to SEQ ID NO: 136 (VH5). In some embodiments, the integrin α7β1 antibody or antigen binding fragment thereof comprises a variable heavy region comprising an amino acid sequence at least 92% identical to SEQ ID NO: 136 (VH5). In some embodiments, the integrin α7β1 antibody or antigen binding fragment thereof comprises a variable heavy region comprising an amino acid sequence at least 93% identical to SEQ ID NO: 136 (VH5). In some embodiments, the integrin α7β1 antibody or antigen binding fragment thereof comprises a variable heavy region comprising an amino acid sequence at least 94% identical to SEQ ID NO: 136 (VH5). In some embodiments, the integrin α7β1 antibody or antigen binding fragment thereof comprises a variable heavy region comprising an amino acid sequence at least 95% identical to SEQ ID NO: 136 (VH5). In some embodiments, the integrin α7β1 antibody or antigen binding fragment thereof comprises a variable heavy region comprising an amino acid sequence at least 97% identical to SEQ ID NO: 136 (VH5). In some embodiments, the integrin α7β1 antibody or antigen binding fragment thereof comprises a variable heavy region comprising an amino acid sequence at least 98% identical to SEQ ID NO: 136 (VH5). In some embodiments, the integrin α7β1 antibody or antigen binding fragment thereof comprises a variable heavy region comprising an amino acid sequence at least 99% identical to SEQ ID NO: 136 (VH5). In some embodiments, the integrin α7β1 antibody or antigen binding fragment thereof comprises a variable heavy region comprising an amino acid sequence identical to SEQ ID NO: 136 (VH5).

In some embodiments, the integrin α7β1 antibody or antigen binding fragment thereof comprises a variable heavy region comprising an amino acid sequence at least 80% identical to SEQ ID NO: 138 (VH6). In some embodiments, the integrin α7β1 antibody or antigen binding fragment thereof comprises a variable heavy region comprising an amino acid sequence at least 83% identical to SEQ ID NO: 138 (VH6). In some embodiments, the integrin α7β1 antibody or antigen binding fragment thereof comprises a variable heavy region comprising an amino acid sequence at least 85% identical to SEQ ID NO: 138 (VH6). In some embodiments, the integrin α7β1 antibody or antigen binding fragment thereof comprises a variable heavy region comprising an amino acid sequence at least 87% identical to SEQ ID NO: 138 (VH6). In some embodiments, the integrin α7β1 antibody or antigen binding fragment thereof comprises a variable heavy region comprising an amino acid sequence at least 90% identical to SEQ ID NO: 138 (VH6). In some embodiments, the integrin α7β1 antibody or antigen binding fragment thereof comprises a variable heavy region comprising an amino acid sequence at least 92% identical to SEQ ID NO: 138 (VH6). In some embodiments, the integrin α7β1 antibody or antigen binding fragment thereof comprises a variable heavy region comprising an amino acid sequence at least 93% identical to SEQ ID NO: 138 (VH6). In some embodiments, the integrin α7β1 antibody or antigen binding fragment thereof comprises a variable heavy region comprising an amino acid sequence at least 94% identical to SEQ ID NO: 138 (VH6). In some embodiments, the integrin α7β1 antibody or antigen binding fragment thereof comprises a variable heavy region comprising an amino acid sequence at least 95% identical to SEQ ID NO: 138 (VH6). In some embodiments, the integrin α7β1 antibody or antigen binding fragment thereof comprises a variable heavy region comprising an amino acid sequence at least 97% identical to SEQ ID NO: 138 (VH6). In some embodiments, the integrin α7β1 antibody or antigen binding fragment thereof comprises a variable heavy region comprising an amino acid sequence at least 98% identical to SEQ ID NO: 138 (VH6). In some embodiments, the integrin α7β1 antibody or antigen binding fragment thereof comprises a variable heavy region comprising an amino acid sequence at least 99% identical to SEQ ID NO: 138 (VH6). In some embodiments, the integrin α7β1 antibody or antigen binding fragment thereof comprises a variable heavy region comprising an amino acid sequence identical to SEQ ID NO: 138 (VH6).

In some embodiments, the integrin α7β1 antibody or antigen binding fragment thereof comprises a variable heavy region comprising an amino acid sequence at least 80% identical to SEQ ID NO: 140 (VH7). In some embodiments, the integrin α7β1 antibody or antigen binding fragment thereof comprises a variable heavy region comprising an amino acid sequence at least 83% identical to SEQ ID NO: 140 (VH7). In some embodiments, the integrin α7β1 antibody or antigen binding fragment thereof comprises a variable heavy region comprising an amino acid sequence at least 85% identical to SEQ ID NO: 140 (VH7). In some embodiments, the integrin α7β1 antibody or antigen binding fragment thereof comprises a variable heavy region comprising an amino acid sequence at least 87% identical to SEQ ID NO: 140 (VH7). In some embodiments, the integrin α7β1 antibody or antigen binding fragment thereof comprises a variable heavy region comprising an amino acid sequence at least 90% identical to SEQ ID NO: 140 (VH7). In some embodiments, the integrin α7β1 antibody or antigen binding fragment thereof comprises a variable heavy region comprising an amino acid sequence at least 92% identical to SEQ ID NO: 140 (VH7). In some embodiments, the integrin α7β1 antibody or antigen binding fragment thereof comprises a variable heavy region comprising an amino acid sequence at least 93% identical to SEQ ID NO: 140 (VH7). In some embodiments, the integrin α7β1 antibody or antigen binding fragment thereof comprises a variable heavy region comprising an amino acid sequence at least 94% identical to SEQ ID NO: 140 (VH7). In some embodiments, the integrin α7β1 antibody or antigen binding fragment thereof comprises a variable heavy region comprising an amino acid sequence at least 95% identical to SEQ ID NO: 140 (VH7). In some embodiments, the integrin α7β1 antibody or antigen binding fragment thereof comprises a variable heavy region comprising an amino acid sequence at least 97% identical to SEQ ID NO: 140 (VH7). In some embodiments, the integrin α7β1 antibody or antigen binding fragment thereof comprises a variable heavy region comprising an amino acid sequence at least 98% identical to SEQ ID NO: 140 (VH7). In some embodiments, the integrin α7β1 antibody or antigen binding fragment thereof comprises a variable heavy region comprising an amino acid sequence at least 99% identical to SEQ ID NO: 140 (VH7). In some embodiments, the integrin α7β1 antibody or antigen binding fragment thereof comprises a variable heavy region comprising an amino acid sequence identical to SEQ ID NO: 140 (VH7).

In some embodiments, the integrin α7β1 antibody or antigen binding fragment thereof comprises a variable light region comprising an amino acid sequence at least 80% identical to SEQ ID NO: 129 (VL1). In some embodiments, the integrin α7β1 antibody or antigen binding fragment thereof comprises a variable light region comprising an amino acid sequence at least 83% identical to SEQ ID NO: 129 (VL1). In some embodiments, the integrin α7β1 antibody or antigen binding fragment thereof comprises a variable light region comprising an amino acid sequence at least 85% identical to SEQ ID NO: 129 (VL1). In some embodiments, the integrin α7β1 antibody or antigen binding fragment thereof comprises a variable light region comprising an amino acid sequence at least 87% identical to SEQ ID NO: 129 (VL1). In some embodiments, the integrin α7β1 antibody or antigen binding fragment thereof comprises a variable light region comprising an amino acid sequence at least 90% identical to SEQ ID NO: 129 (VL1). In some embodiments, the integrin α7β1 antibody or antigen binding fragment thereof comprises a variable light region comprising an amino acid sequence at least 92% identical to SEQ ID NO: 129 (VL1). In some embodiments, the integrin α7β1 antibody or antigen binding fragment thereof comprises a variable light region comprising an amino acid sequence at least 93% identical to SEQ ID NO: 129 (VL1). In some embodiments, the integrin α7β1 antibody or antigen binding fragment thereof comprises a variable light region comprising an amino acid sequence at least 94% identical to SEQ ID NO: 129 (VL1). In some embodiments, the integrin α7β1 antibody or antigen binding fragment thereof comprises a variable light region comprising an amino acid sequence at least 95% identical to SEQ ID NO: 129 (VL1). In some embodiments, the integrin α7β1 antibody or antigen binding fragment thereof comprises a variable light region comprising an amino acid sequence at least 97% identical to SEQ ID NO: 129 (VL1). In some embodiments, the integrin α7β1 antibody or antigen binding fragment thereof comprises a variable light region comprising an amino acid sequence at least 98% identical to SEQ ID NO: 129 (VL1). In some embodiments, the integrin α7β1 antibody or antigen binding fragment thereof comprises a variable light region comprising an amino acid sequence at least 99% identical to SEQ ID NO: 129 (VL1). In some embodiments, the integrin α7β1 antibody or antigen binding fragment thereof comprises a variable light region comprising an amino acid sequence identical to SEQ ID NO: 129 (VL1).

In some embodiments, the integrin α7β1 antibody or antigen binding fragment thereof comprises a variable light region comprising an amino acid sequence at least 80% identical to SEQ ID NO: 131 (VL2). In some embodiments, the integrin α7β1 antibody or antigen binding fragment thereof comprises a variable light region comprising an amino acid sequence at least 83% identical to SEQ ID NO: 131 (VL2). In some embodiments, the integrin α7β1 antibody or antigen binding fragment thereof comprises a variable light region comprising an amino acid sequence at least 85% identical to SEQ ID NO: 131 (VL2). In some embodiments, the integrin α7β1 antibody or antigen binding fragment thereof comprises a variable light region comprising an amino acid sequence at least 87% identical to SEQ ID NO: 131 (VL2). In some embodiments, the integrin α7β1 antibody or antigen binding fragment thereof comprises a variable light region comprising an amino acid sequence at least 90% identical to SEQ ID NO: 131 (VL2). In some embodiments, the integrin α7β1 antibody or antigen binding fragment thereof comprises a variable light region comprising an amino acid sequence at least 92% identical to SEQ ID NO: 131 (VL2). In some embodiments, the integrin α7β1 antibody or antigen binding fragment thereof comprises a variable light region comprising an amino acid sequence at least 93% identical to SEQ ID NO: 131 (VL2). In some embodiments, the integrin α7β1 antibody or antigen binding fragment thereof comprises a variable light region comprising an amino acid sequence at least 94% identical to SEQ ID NO: 131 (VL2). In some embodiments, the integrin α7β1 antibody or antigen binding fragment thereof comprises a variable light region comprising an amino acid sequence at least 95% identical to SEQ ID NO: 131 (VL2). In some embodiments, the integrin α7β1 antibody or antigen binding fragment thereof comprises a variable light region comprising an amino acid sequence at least 97% identical to SEQ ID NO: 131 (VL2). In some embodiments, the integrin α7β1 antibody or antigen binding fragment thereof comprises a variable light region comprising an amino acid sequence at least 98% identical to SEQ ID NO: 131 (VL2). In some embodiments, the integrin α7β1 antibody or antigen binding fragment thereof comprises a variable light region comprising an amino acid sequence at least 99% identical to SEQ ID NO: 131 (VL2). In some embodiments, the integrin α7β1 antibody or antigen binding fragment thereof comprises a variable light region comprising an amino acid sequence identical to SEQ ID NO: 131 (VL2).

In some embodiments, the integrin α7β1 antibody or antigen binding fragment thereof comprises a variable light region comprising an amino acid sequence at least 80% identical to SEQ ID NO: 133 (VL3). In some embodiments, the integrin α7β1 antibody or antigen binding fragment thereof comprises a variable light region comprising an amino acid sequence at least 83% identical to SEQ ID NO: 133 (VL3). In some embodiments, the integrin α7β1 antibody or antigen binding fragment thereof comprises a variable light region comprising an amino acid sequence at least 85% identical to SEQ ID NO: 133 (VL3). In some embodiments, the integrin α7β1 antibody or antigen binding fragment thereof comprises a variable light region comprising an amino acid sequence at least 87% identical to SEQ ID NO: 133 (VL3). In some embodiments, the integrin α7β1 antibody or antigen binding fragment thereof comprises a variable light region comprising an amino acid sequence at least 90% identical to SEQ ID NO: 133 (VL3). In some embodiments, the integrin α7β1 antibody or antigen binding fragment thereof comprises a variable light region comprising an amino acid sequence at least 92% identical to SEQ ID NO: 133 (VL3). In some embodiments, the integrin α7β1 antibody or antigen binding fragment thereof comprises a variable light region comprising an amino acid sequence at least 93% identical to SEQ ID NO: 133 (VL3). In some embodiments, the integrin α7β1 antibody or antigen binding fragment thereof comprises a variable light region comprising an amino acid sequence at least 94% identical to SEQ ID NO: 133 (VL3). In some embodiments, the integrin α7β1 antibody or antigen binding fragment thereof comprises a variable light region comprising an amino acid sequence at least 95% identical to SEQ ID NO: 133 (VL3). In some embodiments, the integrin α7β1 antibody or antigen binding fragment thereof comprises a variable light region comprising an amino acid sequence at least 97% identical to SEQ ID NO: 133 (VL3). In some embodiments, the integrin α7β1 antibody or antigen binding fragment thereof comprises a variable light region comprising an amino acid sequence at least 98% identical to SEQ ID NO: 133 (VL3). In some embodiments, the integrin α7β1 antibody or antigen binding fragment thereof comprises a variable light region comprising an amino acid sequence at least 99% identical to SEQ ID NO: 133 (VL3). In some embodiments, the integrin α7β1 antibody or antigen binding fragment thereof comprises a variable light region comprising an amino acid sequence identical to SEQ ID NO: 133 (VL3).

In some embodiments, the integrin α7β1 antibody or antigen binding fragment thereof comprises a variable light region comprising an amino acid sequence at least 80% identical to SEQ ID NO: 135 (VL4). In some embodiments, the integrin α7β1 antibody or antigen binding fragment thereof comprises a variable light region comprising an amino acid sequence at least 83% identical to SEQ ID NO: 135 (VL4). In some embodiments, the integrin α7β1 antibody or antigen binding fragment thereof comprises a variable light region comprising an amino acid sequence at least 85% identical to SEQ ID NO: 135 (VL4). In some embodiments, the integrin α7β1 antibody or antigen binding fragment thereof comprises a variable light region comprising an amino acid sequence at least 87% identical to SEQ ID NO: 135 (VL4). In some embodiments, the integrin α7β1 antibody or antigen binding fragment thereof comprises a variable light region comprising an amino acid sequence at least 90% identical to SEQ ID NO: 135 (VL4). In some embodiments, the integrin α7β1 antibody or antigen binding fragment thereof comprises a variable light region comprising an amino acid sequence at least 92% identical to SEQ ID NO: 135 (VL4). In some embodiments, the integrin α7β1 antibody or antigen binding fragment thereof comprises a variable light region comprising an amino acid sequence at least 93% identical to SEQ ID NO: 135 (VL4). In some embodiments, the integrin α7β1 antibody or antigen binding fragment thereof comprises a variable light region comprising an amino acid sequence at least 94% identical to SEQ ID NO: 135 (VL4). In some embodiments, the integrin α7β1 antibody or antigen binding fragment thereof comprises a variable light region comprising an amino acid sequence at least 95% identical to SEQ ID NO: 135 (VL4). In some embodiments, the integrin α7β1 antibody or antigen binding fragment thereof comprises a variable light region comprising an amino acid sequence at least 97% identical to SEQ ID NO: 135 (VL4). In some embodiments, the integrin α7β1 antibody or antigen binding fragment thereof comprises a variable light region comprising an amino acid sequence at least 98% identical to SEQ ID NO: 135 (VL4). In some embodiments, the integrin α7β1 antibody or antigen binding fragment thereof comprises a variable light region comprising an amino acid sequence at least 99% identical to SEQ ID NO: 135 (VL4). In some embodiments, the integrin α7β1 antibody or antigen binding fragment thereof comprises a variable light region comprising an amino acid sequence identical to SEQ ID NO: 135 (VL4).

In some embodiments, the integrin α7β1 antibody or antigen binding fragment thereof comprises a variable light region comprising an amino acid sequence at least 80% identical to SEQ ID NO: 137 (VL5). In some embodiments, the integrin α7β1 antibody or antigen binding fragment thereof comprises a variable light region comprising an amino acid sequence at least 83% identical to SEQ ID NO: 137 (VL5). In some embodiments, the integrin α7β1 antibody or antigen binding fragment thereof comprises a variable light region comprising an amino acid sequence at least 85% identical to SEQ ID NO: 137 (VL5). In some embodiments, the integrin α7β1 antibody or antigen binding fragment thereof comprises a variable light region comprising an amino acid sequence at least 87% identical to SEQ ID NO: 137 (VL5). In some embodiments, the integrin α7β1 antibody or antigen binding fragment thereof comprises a variable light region comprising an amino acid sequence at least 90% identical to SEQ ID NO: 137 (VL5). In some embodiments, the integrin α7β1 antibody or antigen binding fragment thereof comprises a variable light region comprising an amino acid sequence at least 92% identical to SEQ ID NO: 137 (VL5). In some embodiments, the integrin α7β1 antibody or antigen binding fragment thereof comprises a variable light region comprising an amino acid sequence at least 93% identical to SEQ ID NO: 137 (VL5). In some embodiments, the integrin α7β1 antibody or antigen binding fragment thereof comprises a variable light region comprising an amino acid sequence at least 94% identical to SEQ ID NO: 137 (VL5). In some embodiments, the integrin α7β1 antibody or antigen binding fragment thereof comprises a variable light region comprising an amino acid sequence at least 95% identical to SEQ ID NO: 137 (VL5). In some embodiments, the integrin α7β1 antibody or antigen binding fragment thereof comprises a variable light region comprising an amino acid sequence at least 97% identical to SEQ ID NO: 137 (VL5). In some embodiments, the integrin α7β1 antibody or antigen binding fragment thereof comprises a variable light region comprising an amino acid sequence at least 98% identical to SEQ ID NO: 137 (VL5). In some embodiments, the integrin α7β1 antibody or antigen binding fragment thereof comprises a variable light region comprising an amino acid sequence at least 99% identical to SEQ ID NO: 137 (VL5). In some embodiments, the integrin α7β1 antibody or antigen binding fragment thereof comprises a variable light region comprising an amino acid sequence identical to SEQ ID NO: 137 (VL5).

In some embodiments, the integrin α7β1 antibody or antigen binding fragment thereof comprises a variable light region comprising an amino acid sequence at least 80% identical to SEQ ID NO: 139 (VL6). In some embodiments, the integrin α7β1 antibody or antigen binding fragment thereof comprises a variable light region comprising an amino acid sequence at least 83% identical to SEQ ID NO: 139 (VL6). In some embodiments, the integrin α7β1 antibody or antigen binding fragment thereof comprises a variable light region comprising an amino acid sequence at least 85% identical to SEQ ID NO: 139 (VL6). In some embodiments, the integrin α7β1 antibody or antigen binding fragment thereof comprises a variable light region comprising an amino acid sequence at least 87% identical to SEQ ID NO: 139 (VL6). In some embodiments, the integrin α7β1 antibody or antigen binding fragment thereof comprises a variable light region comprising an amino acid sequence at least 90% identical to SEQ ID NO: 139 (VL6). In some embodiments, the integrin α7β1 antibody or antigen binding fragment thereof comprises a variable light region comprising an amino acid sequence at least 92% identical to SEQ ID NO: 139 (VL6). In some embodiments, the integrin α7β1 antibody or antigen binding fragment thereof comprises a variable light region comprising an amino acid sequence at least 93% identical to SEQ ID NO: 139 (VL6). In some embodiments, the integrin α7β1 antibody or antigen binding fragment thereof comprises a variable light region comprising an amino acid sequence at least 94% identical to SEQ ID NO: 139 (VL6). In some embodiments, the integrin α7β1 antibody or antigen binding fragment thereof comprises a variable light region comprising an amino acid sequence at least 95% identical to SEQ ID NO: 139 (VL6). In some embodiments, the integrin α7β1 antibody or antigen binding fragment thereof comprises a variable light region comprising an amino acid sequence at least 97% identical to SEQ ID NO: 139 (VL6). In some embodiments, the integrin α7β1 antibody or antigen binding fragment thereof comprises a variable light region comprising an amino acid sequence at least 98% identical to SEQ ID NO: 139 (VL6). In some embodiments, the integrin α7β1 antibody or antigen binding fragment thereof comprises a variable light region comprising an amino acid sequence at least 99% identical to SEQ ID NO: 139 (VL6). In some embodiments, the integrin α7β1 antibody or antigen binding fragment thereof comprises a variable light region comprising an amino acid sequence identical to SEQ ID NO: 139 (VL6).

In some embodiments, the integrin α7β1 antibody or antigen binding fragment thereof comprises a variable light region comprising an amino acid sequence at least 80% identical to SEQ ID NO: 141 (VL7). In some embodiments, the integrin α7β1 antibody or antigen binding fragment thereof comprises a variable light region comprising an amino acid sequence at least 83% identical to SEQ ID NO: 141 (VL7). In some embodiments, the integrin α7β1 antibody or antigen binding fragment thereof comprises a variable light region comprising an amino acid sequence at least 85% identical to SEQ ID NO: 141 (VL7). In some embodiments, the integrin α7β1 antibody or antigen binding fragment thereof comprises a variable light region comprising an amino acid sequence at least 87% identical to SEQ ID NO: 141 (VL7). In some embodiments, the integrin α7β1 antibody or antigen binding fragment thereof comprises a variable light region comprising an amino acid sequence at least 90% identical to SEQ ID NO: 141 (VL7). In some embodiments, the integrin α7β1 antibody or antigen binding fragment thereof comprises a variable light region comprising an amino acid sequence at least 92% identical to SEQ ID NO: 141 (VL7). In some embodiments, the integrin α7β1 antibody or antigen binding fragment thereof comprises a variable light region comprising an amino acid sequence at least 93% identical to SEQ ID NO: 141 (VL7). In some embodiments, the integrin α7β1 antibody or antigen binding fragment thereof comprises a variable light region comprising an amino acid sequence at least 94% identical to SEQ ID NO: 141 (VL7). In some embodiments, the integrin α7β1 antibody or antigen binding fragment thereof comprises a variable light region comprising an amino acid sequence at least 95% identical to SEQ ID NO: 141 (VL7). In some embodiments, the integrin α7β1 antibody or antigen binding fragment thereof comprises a variable light region comprising an amino acid sequence at least 97% identical to SEQ ID NO: 141 (VL7). In some embodiments, the integrin α7β1 antibody or antigen binding fragment thereof comprises a variable light region comprising an amino acid sequence at least 98% identical to SEQ ID NO: 141 (VL7). In some embodiments, the integrin α7β1 antibody or antigen binding fragment thereof comprises a variable light region comprising an amino acid sequence at least 99% identical to SEQ ID NO: 141 (VL7). In some embodiments, the integrin α7β1 antibody or antigen binding fragment thereof comprises a variable light region comprising an amino acid sequence identical to SEQ ID NO: 141 (VL7).

In some embodiments, the integrin α7β1 antibody or antigen binding fragment thereof comprises a heavy variable region comprising an amino acid sequence of SEQ ID NO: 128 (VH1) and a light variable region comprising an amino acid sequence of SEQ ID NO: 129 (VL1).

In some embodiments, the integrin α7β1 antibody or antigen binding fragment thereof comprises a heavy variable region comprising an amino acid sequence of SEQ ID NO: 130 (VH2) and a light variable region comprising an amino acid sequence of SEQ ID NO: VL2.

In some embodiments, the integrin α7β1 antibody or antigen binding fragment thereof comprises a heavy variable region comprising an amino acid sequence of SEQ ID NO: 132 (VH3) and a light variable region comprising an amino acid sequence of SEQ ID NO: 133 (VL3).

In some embodiments, the integrin α7β1 antibody or antigen binding fragment thereof comprises a heavy variable region comprising an amino acid sequence of SEQ ID NO: 134 (VH4) and a light variable region comprising an amino acid sequence of SEQ ID NO: 135 (VL4).

In some embodiments, the integrin α7β1 antibody or antigen binding fragment thereof comprises a heavy variable region comprising an amino acid sequence of SEQ ID NO: 136 (VH5) and a light variable region comprising an amino acid sequence of SEQ ID NO: 137 (VL5).

In some embodiments, the integrin α7β1 antibody or antigen binding fragment thereof comprises a heavy variable region comprising an amino acid sequence of SEQ ID NO: 138 (VH6) and a light variable region comprising an amino acid sequence of SEQ ID NO: 139 (VL6).

In some embodiments, the integrin α7β1 antibody or antigen binding fragment thereof comprises a heavy variable region comprising an amino acid sequence of SEQ ID NO: 140 (VH7) and a light variable region comprising an amino acid sequence of SEQ ID NO: 141 (VL7).

TABLE H
Exemplary Heavy Chain and Light Chain Sequences
of Human anti-781 Integrin Antibodies.

| Ab | Heavy Chain Sequence | Light Chain Sequence |
|---|---|---|
| Ab28 | EVQLVESGGGLVKPGGSLRLSCAASGFTFSS | SSELTQDPAVSVALGQTVRITCQGDSLRSYY |
| | YTMNWVRQAPGKGLEWVSSISIVGNYIYYAD | ASWYQQKPGQAPVLVIYGKNNRPSGIPDRES |
| | SLKGRFTISRDNAKNSLYLQMNSLRAEDTAV | GSSSGNTASLTITGAQAEDEADFYCTSRDNS |
| | YYCARRKWEHDAFDIWGQGTMVTVSSASTKG | GNHVVFGGGTKLTVLGQPKAAPSVTLFPPSS |
| | PSVFPLAPSSKSTSGGTAALGCLVKDYFPEP | EELQANKATLVCLISDFYPGAVTVAWKADSS |
| | VTVSWNSGALTSGVHTFPAVLQSSGLYSLSS | PVKAGVETTTPSKQSNNKYAASSYLSLTPEQ |
| | VVTVPSSSLGTQTYICNVNHKPSNTKVDKKV | WKSHKSYSCQVTHEGSTVEKTVAPTECS |
| | EPKSCDKTHTCPPCPAPEAAGGPSVFLFPPK | (SEQ ID NO: 119) |
| | PKDTLMISRTPEVTCVVVDVSHEDPEVKENW | |
| | YVDGVEVHNAKTKPREEQYNSTYRVVSVLIV | |
| | LHQDWLNGKEYKCKVSNKALAAPIEKTISKA | |
| | KGQPREPQVYTLPPSRDELTKNQVSLTCLVK | |
| | GFYPSDIAVEWESNGQPENNYKTTPPVLDSD | |
| | GSFFLYSKLTVDKSRWQQGNVFSCSVLHEAL | |
| | HSHYTQKSLSLSPG (SEQ ID NO: 118) | |
| Ab29 | EVQLVESGGGLVKPGGSLRLSCAASGFTESS | SSELTQDPAVSVALGQTVRITCQGDSLRSYY |
| | YTMNWVRQAPGKGLEWVSSISIVGNYIYYAD | ASWYQQKPGQAPVLVIYGKNNRPSGIPDRES |
| | SLKGRFTISRDNAKNSLYLQMNSLRAEDTAV | GSSSGNTASLTITGAQAEDEADFYCTSRDNS |
| | YYCARRKWEHDAFDIWGQGTMVTVSSASTKG | GNHVVFGGGTKLTVLGQPKAAPSVTLFPPSS |
| | PSVFPLAPSSKSTSGGTAALGCLVKDYFPEP | EELQANKATLVCLISDFYPGAVTVAWKADSS |
| | VTVSWNSGALTSGVHTFPAVLQSSGLYSLSS | PVKAGVETTTPSKQSNNKYAASSYLSLTPEQ |
| | VVTVPSSSLGTQTYICNVNHKPSNTKVDKKV | WKSHKSYSCQVTHEGSTVEKTVAPTECS |
| | EPKSCDKTHTCPPCPAPEAAGGPSVFLFPPK | (SEQ ID NO: 121) |
| | PKDTLYITREPEVTCVVVDVSHEDPEVKENW | |
| | YVDGVEVHNAKTKPREEQYNSTYRVVSVLIV | |
| | LHQDWLNGKEYKCKVSNKALAAPIEKTISKA | |
| | KGQPREPQVYTLPPSRDELTKNQVSLTCLVK | |
| | GFYPSDIAVEWESNGQPENNYKTTPPVLDSD | |
| | GSFFLYSKLTVDKSRWQQGNVFSCSVMHEAL | |
| | HNHYTQKSLSLSPG (SEQ ID NO: 120) | |

In some embodiments, the integrin α7β1 antibody comprises a heavy chain comprising an amino acid sequence at least 80% identical to SEQ ID NO: 118. In some embodiments, the integrin α7β1 antibody comprises a heavy chain comprising an amino acid sequence at least 83% identical to SEQ ID NO: 118. In some embodiments, the integrin α7β1 antibody comprises a heavy chain comprising an amino acid sequence at least 85% identical to SEQ ID NO: 118. In some embodiments, the integrin α7β1 antibody comprises a heavy chain comprising an amino acid sequence at least 87% identical to SEQ ID NO: 118. In some embodiments, the integrin α7β1 antibody comprises a heavy chain comprising an amino acid sequence at least 90% identical to SEQ ID NO: 118. In some embodiments, the integrin α7β1 antibody comprises a heavy chain comprising an amino acid sequence at least 92% identical to SEQ ID NO: 118. In some embodiments, the integrin α7β1 antibody comprises a heavy chain comprising an amino acid sequence at least 93% identical to SEQ ID NO: 118. In some embodiments, the integrin α7β1 antibody comprises a heavy chain comprising an amino acid sequence at least 94% identical to SEQ ID NO: 118. In some embodiments, the integrin α7β1 antibody comprises a heavy chain comprising an amino acid sequence at least 95% identical to SEQ ID NO: 118. In some embodiments, the integrin α7β1 antibody comprises a heavy chain comprising an amino acid sequence at least 97% identical to SEQ ID NO: 118. In some embodiments, the integrin α7β1 antibody comprises a heavy chain comprising an amino acid sequence at least 98% identical to SEQ ID NO: 118. In some embodiments, the integrin α7β1 antibody comprises a heavy chain comprising an amino acid sequence at least 99% identical to SEQ ID NO: 118. In some embodiments, the integrin α7β1 antibody comprises a heavy chain comprising an amino acid sequence identical to SEQ ID NO: 118.

In some embodiments, the integrin α7β1 antibody comprises a heavy chain comprising an amino acid sequence at least 80% identical to SEQ ID NO: 120. In some embodiments, the integrin α7β1 antibody comprises a heavy chain comprising an amino acid sequence at least 83% identical to SEQ ID NO: 120. In some embodiments, the integrin α7β1 antibody comprises a heavy chain comprising an amino acid sequence at least 85% identical to SEQ ID NO: 120. In some embodiments, the integrin α7β1 antibody comprises a heavy chain comprising an amino acid sequence at least 87% identical to SEQ ID NO: 120. In some embodiments, the integrin α7β1 antibody comprises a heavy chain comprising an amino acid sequence at least 90% identical to SEQ ID NO: 120. In some embodiments, the integrin α7β1 antibody comprises a heavy chain comprising an amino acid sequence at least 92% identical to SEQ ID NO: 120. In some embodiments, the integrin α7β1 antibody comprises a heavy chain comprising an amino acid sequence at least 93% identical to SEQ ID NO: 120. In some embodiments, the integrin α7β1 antibody comprises a heavy chain comprising an amino acid sequence at least 94% identical to SEQ ID NO: 120. In some embodiments, the integrin α7β1 antibody comprises a heavy chain comprising an amino acid sequence at least 95% identical to SEQ ID NO: 120. In some embodiments, the integrin α7β1 antibody comprises a heavy chain comprising an amino acid sequence at least 97% identical to SEQ ID NO: 120. In some embodiments, the integrin α7β1 antibody comprises a heavy chain comprising an amino acid sequence at least 98% identical to SEQ ID NO: 120. In some embodiments, the integrin α7β1 antibody comprises a heavy chain comprising an amino acid sequence at least 99% identical to SEQ ID NO: 120. In some embodiments, the integrin α7β1 antibody comprises a heavy chain comprising an amino acid sequence identical to SEQ ID NO: 120.

In some embodiments, the integrin α7β1 antibody comprises a light chain comprising an amino acid sequence at least 80% identical to SEQ ID NO: 119. In some embodiments, the integrin α7β1 antibody comprises a light chain comprising an amino acid sequence at least 83% identical to SEQ ID NO: 119. In some embodiments, the integrin α7β1 antibody comprises a light chain comprising an amino acid sequence at least 85% identical to SEQ ID NO: 119. In some embodiments, the integrin α7β1 antibody comprises a light chain comprising an amino acid sequence at least 87% identical to SEQ ID NO: 119. In some embodiments, the integrin α7β1 antibody comprises a light chain comprising an amino acid sequence at least 90% identical to SEQ ID NO: 119. In some embodiments, the integrin α7β1 antibody comprises a light chain comprising an amino acid sequence at least 92% identical to SEQ ID NO: 119. In some embodiments, the integrin α7β1 antibody comprises a light chain comprising an amino acid sequence at least 93% identical to SEQ ID NO: 119. In some embodiments, the integrin α7β1 antibody comprises a light chain comprising an amino acid sequence at least 94% identical to SEQ ID NO: 119. In some embodiments, the integrin α7β1 antibody comprises a light chain comprising an amino acid sequence at least 95% identical to SEQ ID NO: 119. In some embodiments, the integrin α7β1 antibody comprises a light chain comprising an amino acid sequence at least 97% identical to SEQ ID NO: 119. In some embodiments, the integrin α7β1 antibody comprises a light chain comprising an amino acid sequence at least 98% identical to SEQ ID NO: 119. In some embodiments, the integrin α7β1 antibody comprises a light chain comprising an amino acid sequence at least 99% identical to SEQ ID NO: 119. In some embodiments, the integrin α7β1 antibody comprises a light chain comprising an amino acid sequence identical to SEQ ID NO: 119.

In some embodiments, the integrin α7β1 antibody comprises a light chain comprising an amino acid sequence at least 80% identical to SEQ ID NO: 121. In some embodiments, the integrin α7β1 antibody comprises a light chain comprising an amino acid sequence at least 83% identical to SEQ ID NO: 121. In some embodiments, the integrin α7β1 antibody comprises a light chain comprising an amino acid sequence at least 85% identical to SEQ ID NO: 121. In some embodiments, the integrin α7β1 antibody comprises a light chain comprising an amino acid sequence at least 87% identical to SEQ ID NO: 121. In some embodiments, the integrin α7β1 antibody comprises a light chain comprising an amino acid sequence at least 90% identical to SEQ ID NO: 121. In some embodiments, the integrin α7β1 antibody comprises a light chain comprising an amino acid sequence at least 92% identical to SEQ ID NO: 121. In some embodiments, the integrin α7β1 antibody comprises a light chain comprising an amino acid sequence at least 93% identical to SEQ ID NO: 121. In some embodiments, the integrin α7β1 antibody comprises a light chain comprising an amino acid sequence at least 94% identical to SEQ ID NO: 121. In some embodiments, the integrin α7β1 antibody comprises a light chain comprising an amino acid sequence at least 95% identical to SEQ ID NO: 121. In some embodiments, the integrin α7β1 antibody comprises a light chain comprising an amino acid sequence at least 97% identical to SEQ ID NO: 121. In some embodiments, the integrin α7β1 antibody comprises a light chain comprising an amino acid sequence at least 98% identical to SEQ ID NO: 121. In some embodiments, the integrin α7β1 antibody comprises a light chain comprising an amino acid sequence at least 99% identical to SEQ ID NO: 121. In some embodiments, the integrin α7β1 antibody comprises a light chain comprising an amino acid sequence identical to SEQ ID NO: 121.

In some embodiments, the integrin α7β1 antibody or antigen binding fragment thereof comprises a heavy chain comprising an amino acid sequence of SEQ ID NO: 118 and a light chain comprising an amino acid sequence of SEQ ID NO: 119.

In some embodiments, the integrin α7β1 antibody or antigen binding fragment thereof comprises a heavy chain comprising an amino acid sequence of SEQ ID NO: 120 and a light chain comprising an amino acid sequence of SEQ ID NO: 121.

### Anti-Mouse α7β1 Antibodies

In some embodiments, the antibody or antigen-binding fragment thereof is an anti-mouse α7β1 antibody. In some embodiments, the antibody or antigen-binding fragment thereof comprises CDRs selected from Table C.

TABLE C
Exemplary CDRs of mouse anti-α7β1 antibodies.

| Ab | HCDR1 | HCDR2 | HCDR3 | LCDR1 | LCDR2 | LCDR3 |
|---|---|---|---|---|---|---|
| Ab8 | GYSFTRYW | IDPSDSET | ARGGYFGEGWYFD | QSLVHRNGN | KVS | SQSTHVPWT |
| | (SEQ ID | (SEQ ID | V | TY | (SEQ ID | (SEQ ID |
| | NO: 7, | NO: 22, | (SEQ ID NO: | (SEQ ID | NO: 63, | NO: 75, |
| | HCDR1G) | HCDR2H) | 38, HCDR3H) | NO: 51, | LCDR2F) | LCDR3G) |
| | | | | LCDR1F) | | |
| Ab9 | GYTFTSYW | IYPSDSYT | TRRANYVGAMDY | SSVSY | ATS | QQWSSNPLT |
| | (SEQ ID | (SEQ ID | (SEQ ID NO: | (SEQ ID | (SEQ ID | (SEQ ID |
| | NO: 8, | NO: 23, | 39, HCDR3I) | NO: 52, | NO: 64, | NO: 76, |
| | HCDR1H) | HCDR2I) | | LCDR1G) | LCDR2G) | LCDR3H) |
| Ab10 | GFSLTSYG | IWVGGIT | ARDGYYGDFDV | QDINKF | YTS | LOYDNLRT |
| | (SEQ ID | (SEQ ID | (SEQ ID NO: | (SEQ ID | (SEQ ID | (SEQ ID |
| | NO: 9, | NO: 24, | 40, HCDR3J) | NO: 53, | NO: 65, | NO: 77, |
| | HCDR1I) | HCDR2J) | | LCDR1H) | LCDR2H) | LCDR3I) |
| Ab11 | GFSLTSYG | IWAGGIT | ARDGYYGDFDV | QDINKY | YTS | LQYDNLRT |
| | (SEQ ID | (SEQ ID | (SEQ ID NO: | (SEQ ID | (SEQ ID | (SEQ ID |
| | NO: 9, | NO: 25, | 40, HCDR3J) | NO: 54, | NO: 65, | NO: 77, |
| | HCDR1I) | HCDR2K) | | LCDR1I) | LCDR2H) | LCDR3I) |
| Ab12 | GFSLTSYG | IWAGGIT | ARDGYYGHFDV | QDINKY | YTS | LOYDNLRA |
| | (SEQ ID | (SEQ ID | (SEQ ID NO: | (SEQ ID | (SEQ ID | (SEQ ID |
| | NO: 9, | NO: 25, | 41, HCDR3K) | NO: 54, | NO: 65, | NO: 78, |
| | HCDR1I) | HCDR2K) | | LCDR1I) | LCDR2H) | LCDR3J) |
| Ab13 | GFTFSSDA | ISIGGSA | ARGGDYDAMDY | QSISDL | YAS | QNGHNFPFT |
| | (SEQ ID | (SEQ ID | (SEQ ID NO: | (SEQ ID | (SEQ ID | (SEQ ID |
| | NO: 10, | NO: 26, | 42, HCDR3L) | NO: 55, | NO: 66, | NO: 79, |
| | HCDR1J) | HCDR2L) | | LCDR1J) | LCDR2I) | LCDR3K) |
| Ab14 | GFTFSSSA | ISVGGST | ARGGDYDAMDY | QSISDY | YAS | QNGHSFPFT |
| | (SEQ ID | (SEQ ID | (SEQ ID NO: | (SEQ ID | (SEQ ID | (SEQ ID |
| | NO: 11, | NO: 27, | 42, HCDR3L) | NO: 56, | NO:66, | NO: 80, |
| | HCDR1K) | HCDR2M) | | LCDR1K) | LCDR2I) | LCDR3L) |
| Ab15 | GFTFSSSA | ISVGGST | ARGGDYDAMDY | QSISDY | YSS | QNGHSFPFT |
| | (SEQ ID | (SEQ ID | (SEQ ID NO: | (SEQ ID | (SEQ ID | (SEQ ID |
| | NO: 11, | NO: 27, | 42, HCDR3L) | NO: 56, | NO: 67, | NO: 80, |
| | HCDR1K) | HCDR2M) | | LCDR1K) | LCDR2J) | LCDR3L) |
| Ab16 | GFTFSSSA | ISVGGST | ARGGDYDPMDY | QSISDY | YAS | QNGHSFPFT |
| | (SEQ ID | (SEQ ID | (SEQ ID NO: | (SEQ ID | (SEQ ID | (SEQ ID |
| | NO: 11, | NO: 27, | 43, HCDR3M) | NO: 56, | NO: 66, | NO: 80, |
| | HCDR1K) | HCDR2M) | | LCDR1K) | LCDR2I) | LCDR3L) |
| Ab17 | GFTFSSDA | ISIGGST | ARGGDYDAMDY | QSISDL | YAS | QNGHNFPFT |
| | (SEQ ID | (SEQ ID | (SEQ ID NO: | (SEQ ID | (SEQ ID | (SEQ ID |
| | NO: 10, | NO: 28, | 42, HCDR3L) | NO: 55, | NO: 66, | NO: 79, |
| | HCDR1J) | HCDR2N) | | LCDR1J) | LCDR2I) | LCDR3K) |
| Ab18 | GFTFSSSA | ISVGGSI | ARGGDYDAMDY | QSISDY | YAS | QNGHSFPFT |
| | (SEQ ID | (SEQ ID | (SEQ ID NO: | (SEQ ID | (SEQ ID | (SEQ ID |
| | NO: 11, | NO: 29, | 42, HCDR3L) | NO: 56, | NO: 66, | NO: 80, |
| | HCDR1K) | HCDR20) | | LCDR1K) | LCDR2I) | LCDR3L) |
| Ab20 | GFTFSNCA | ISIGGST | ARGGDYDAMDY | QSISDL | YAS | QNGHSFPFT |
| | (SEQ ID | (SEQ ID | (SEQ ID NO: | (SEQ ID | (SEQ ID | (SEQ ID |
| | NO: 12, | NO: 28, | 42, HCDR3L) | NO: 55, | NO: 66, | NO: 80, |
| | HCDR1L) | HCDR2N) | | LCDR1J) | LCDR2I) | LCDR3L) |
| Ab21 | GFTFSSDA | ISIGGST | TRGGDYDAMDY | QSISDL | YAS | QNGHNFPFT |
| | (SEQ ID | (SEQ ID | (SEQ ID NO: | (SEQ ID | (SEQ ID | (SEQ ID |
| | NO: 10, | NO: 28, | 44, HCDR3N) | NO: 55, | NO: 66, | NO: 79, |
| | HCDR1J) | HCDR2N) | | LCDR1J) | LCDR2I) | LCDR3K) |
| Ab22 | GFSLSTSDMG | IYWDDDK | VRGTAVVAFYWYF | QDINNY | RAN | LHYDDFPLT |
| | (SEQ ID | (SEQ ID | DV | (SEQ ID | (SEQ ID | (SEQ ID |
| | NO: 13, | NO: 30, | (SEQ ID NO: | NO: 57, | NO: 68, | NO: 81, |
| | HCDR1M) | HCDR2P) | 45, HCDR30) | LCDR1L) | LCDR2K) | LCDR3M) |
| Ab23 | GFSLSTSDLG | IYWDDDK | VRGTAVVAFYWYF | QDINNY | RAN | LQYDDFPLT |
| | (SEQ ID | (SEQ ID | DV | (SEQ ID | (SEQ ID | (SEQ ID |
| | NO: 14, | NO: 30, | (SEQ ID NO: | NO: 57, | NO: 68, | NO: 82, |
| | HCDR1N) | HCDR2P) | 45, HCDR30) | LCDR1L) | LCDR2K) | LCDR3N) |

In some embodiments, the antibody or antigen-binding fragment thereof comprises an HCDR1 of SEQ ID NO: 7 (HCDR1G), an HCDR2 of SEQ ID NO: 22 (HCDR2H), and an HCDR3 of SEQ ID NO: 38 (HCDR3H). In some embodiments, the antibody or antigen-binding fragment thereof comprises an LCDR1 of SEQ ID NO: 51 (LCDR1F), an LCDR2 of SEQ ID NO: 63 (LCDR2F), and an LCDR3 of SEQ ID NO: 75 (LCDR3G).

In some embodiments, the antibody or antigen-binding fragment thereof competes with a reference antibody, wherein the reference antibody comprises an HCDR1 of SEQ ID NO: 7 (HCDR1G), an HCDR2 of SEQ ID NO: 22 (HCDR2H), an HCDR3 of SEQ ID NO: 38 (HCDR3H), an LCDR1 of SEQ ID NO: 51 (LCDR1F), an LCDR2 of SEQ ID NO: 63 (LCDR2F), and an LCDR3 of SEQ ID NO: 75 (LCDR3G).

In some embodiments, the antibody or antigen-binding fragment thereof comprises an HCDR1 of SEQ ID NO: 8 (HCDR1H), an HCDR2 of SEQ ID NO: 23 (HCDR2I), and an HCDR3 of SEQ ID NO: 39 (HCDR3I). In some embodiments, the antibody or antigen-binding fragment thereof comprises an LCDR1 of SEQ ID NO: 52 (LCDR1G), an LCDR2 of SEQ ID NO: 64 (LCDR2G), and an LCDR3 of SEQ ID NO: 76 (LCDR3H).

In some embodiments, the antibody or antigen-binding fragment thereof competes with a reference antibody, wherein the reference antibody comprises an HCDR1 of SEQ ID NO: 8 (HCDR1H), an HCDR2 of SEQ ID NO: 23 (HCDR2I), an HCDR3 of SEQ ID NO: 39 (HCDR3I), an LCDR1 of SEQ ID NO: 52 (LCDR1G), an LCDR2 of SEQ ID NO: 64 (LCDR2G), and an LCDR3 of SEQ ID NO: 76 (LCDR3H).

In some embodiments, the antibody or antigen-binding fragment thereof comprises an HCDR1 of SEQ ID NO: 9 (HCDR1I), an HCDR2 of SEQ ID NO: 24 (HCDR2J), and an HCDR3 of SEQ ID NO: 40 (HCDR3J). In some embodiments, the antibody or antigen-binding fragment thereof comprises an LCDR1 of SEQ ID NO: 53 (LCDR1H), an LCDR2 of SEQ ID NO: 65 (LCDR2H), and an LCDR3 of SEQ ID NO: 77 (LCDR3I).

In some embodiments, the antibody or antigen-binding fragment thereof competes with a reference antibody, wherein the reference antibody comprises an HCDR1 of SEQ ID NO: 9 (HCDR1I), an HCDR2 of SEQ ID NO: 24 (HCDR2J), an HCDR3 of SEQ ID NO: 40 (HCDR3J), an LCDR1 of SEQ ID NO: 53 (LCDR1H), an LCDR2 of SEQ ID NO: 65 (LCDR2H), and an LCDR3 of SEQ ID NO: 77 (LCDR3I).

In some embodiments, the antibody or antigen-binding fragment thereof comprises an HCDR1 of SEQ ID NO: 9 (HCDR1I), an HCDR2 of SEQ ID NO: 25 (HCDR2K), and an HCDR3 of SEQ ID NO: 40 (HCDR3J). In some embodiments, the antibody or antigen-binding fragment thereof comprises an LCDR1 of SEQ ID NO: 54 (LCDR1I), an LCDR2 of SEQ ID NO: 65 (LCDR2H), and an LCDR3 of SEQ ID NO: LCDR3I.

In some embodiments, the antibody or antigen-binding fragment thereof competes with a reference antibody, wherein the reference antibody comprises an HCDR1 of SEQ ID NO: 9 (HCDR1I), an HCDR2 of SEQ ID NO: 25 (HCDR2K), an HCDR3 of SEQ ID NO: 40 (HCDR3J), an LCDR1 of SEQ ID NO: 54 (LCDR1I), an LCDR2 of SEQ ID NO: 65 (LCDR2H), and an LCDR3 of SEQ ID NO: 77 (LCDR3I).

In some embodiments, the antibody or antigen-binding fragment thereof comprises an HCDR1 of SEQ ID NO: 9 (HCDR1I), an HCDR2 of SEQ ID NO: 25 (HCDR2K), and an HCDR3 of SEQ ID NO: 41 (HCDR3K). In some embodiments, the antibody or antigen-binding fragment thereof comprises an LCDR1 of SEQ ID NO: 54 (LCDR1I), an LCDR2 of SEQ ID NO: 65 (LCDR2H), and an LCDR3 of SEQ ID NO: 78 (LCDR3J).

In some embodiments, the antibody or antigen-binding fragment thereof competes with a reference antibody, wherein the reference antibody comprises an HCDR1 of SEQ ID NO: 9 (HCDR1I), an HCDR2 of SEQ ID NO: 25 (HCDR2K), an HCDR3 of SEQ ID NO: 41 (HCDR3K), an LCDR1 of SEQ ID NO: 54 (LCDR1I), an LCDR2 of SEQ ID NO: 65 (LCDR2H), and an LCDR3 of SEQ ID NO: 78 (LCDR3J).

In some embodiments, the antibody or antigen-binding fragment thereof comprises an HCDR1 of SEQ ID NO: 10 (HCDR1J), an HCDR2 of SEQ ID NO: HCDR2L, and an HCDR3 of SEQ ID NO: 42 (HCDR3L). In some embodiments, the antibody or antigen-binding fragment thereof comprises an LCDR1 of SEQ ID NO: 55 (LCDR1J), an LCDR2 of SEQ ID NO: 66 (LCDR2I), and an LCDR3 of SEQ ID NO: 79 (LCDR3K).

In some embodiments, the antibody or antigen-binding fragment thereof competes with a reference antibody, wherein the reference antibody comprises an HCDR1 of SEQ ID NO: 10 (HCDR1J), an HCDR2 of SEQ ID NO: 26 (HCDR2L), an HCDR3 of SEQ ID NO: 42 (HCDR3L), an LCDR1 of SEQ ID NO: 55 (LCDR1J), an LCDR2 of SEQ ID NO: 66 (LCDR2I), and an LCDR3 of SEQ ID NO: 79 (LCDR3K).

In some embodiments, the antibody or antigen-binding fragment thereof comprises an HCDR1 of SEQ ID NO: 11 (HCDR1K), an HCDR2 of SEQ ID NO: 27 (HCDR2M), and an HCDR3 of SEQ ID NO: 42 (HCDR3L). In some embodiments, the antibody or antigen-binding fragment thereof comprises an LCDR1 of SEQ ID NO: 56 (LCDR1K), an LCDR2 of SEQ ID NO: 66 (LCDR2I), and an LCDR3 of SEQ ID NO: 80, LCDR3L.

In some embodiments, the antibody or antigen-binding fragment thereof competes with a reference antibody, wherein the reference antibody comprises an HCDR1 of SEQ ID NO: 11 (HCDR1K), an HCDR2 of SEQ ID NO: 27 (HCDR2M), an HCDR3 of SEQ ID NO: 42 (HCDR3L), an LCDR1 of SEQ ID NO: 56 (LCDR1K), an LCDR2 of SEQ ID NO: 66 (LCDR2I), and an LCDR3 of SEQ ID NO: 80 (LCDR3L).

In some embodiments, the antibody or antigen-binding fragment thereof comprises an HCDR1 of SEQ ID NO: 11 (HCDR1K), an HCDR2 of SEQ ID NO: 27 (HCDR2M), and an HCDR3 of SEQ ID NO: 42 (HCDR3L). In some embodiments, the antibody or antigen-binding fragment thereof comprises an LCDR1 of SEQ ID NO: 56 (LCDR1K), an LCDR2 of SEQ ID NO: 67 (LCDR2J), and an LCDR3 of SEQ ID NO: 80 (LCDR3L).

In some embodiments, the antibody or antigen-binding fragment thereof competes with a reference antibody, wherein the reference antibody comprises an HCDR1 of SEQ ID NO: 11 (HCDR1K), an HCDR2 of SEQ ID NO: 27 (HCDR2M), an HCDR3 of SEQ ID NO: 42 (HCDR3L), an LCDR1 of SEQ ID NO: 56 (LCDR1K), an LCDR2 of SEQ ID NO: 67 (LCDR2J), and an LCDR3 of SEQ ID NO: 80 (LCDR3L).

In some embodiments, the antibody or antigen-binding fragment thereof comprises an HCDR1 of SEQ ID NO: 11 (HCDR1K), an HCDR2 of SEQ ID NO: 27 (HCDR2M), and an HCDR3 of SEQ ID NO: 43 (HCDR3M). In some embodiments, the antibody or antigen-binding fragment thereof comprises an LCDR1 of SEQ ID NO: 56 (LCDR1K), an LCDR2 of SEQ ID NO: 66 (LCDR2I), and an LCDR3 of SEQ ID NO: 80 (LCDR3L).

In some embodiments, the antibody or antigen-binding fragment thereof competes with a reference antibody, wherein the reference antibody comprises an HCDR1 of SEQ ID NO: 11 (HCDR1K), an HCDR2 of SEQ ID NO: 27 (HCDR2M), an HCDR3 of SEQ ID NO: 43 (HCDR3M), an LCDR1 of SEQ ID NO: 56 (LCDR1K), an LCDR2 of SEQ ID NO: 66 (LCDR2I), and an LCDR3 of SEQ ID NO: 80 (LCDR3L).

In some embodiments, the antibody or antigen-binding fragment thereof comprises an HCDR1 of SEQ ID NO: 10 (HCDR1J), an HCDR2 of SEQ ID NO: 28 (HCDR2N), and an HCDR3 of SEQ ID NO: 42 (HCDR3L). In some embodiments, the antibody or antigen-binding fragment thereof comprises an LCDR1 of SEQ ID NO: 55 (LCDR1J), an LCDR2 of SEQ ID NO: 66 (LCDR2I), and an LCDR3 of SEQ ID NO: 79 (LCDR3K).

In some embodiments, the antibody or antigen-binding fragment thereof competes with a reference antibody, wherein the reference antibody comprises an HCDR1 of SEQ ID NO: 10 (HCDR1J), an HCDR2 of SEQ ID NO: 28 (HCDR2N), an HCDR3 of SEQ ID NO: 42 (HCDR3L), an LCDR1 of SEQ ID NO: 55 (LCDR1J), an LCDR2 of SEQ ID NO: 66 (LCDR2I), and an LCDR3 of SEQ ID NO: 79 (LCDR3K).

In some embodiments, the antibody or antigen-binding fragment thereof comprises an HCDR1 of SEQ ID NO: 11 (HCDR1K), an HCDR2 of SEQ ID NO: 29 (HCDR2O), and an HCDR3 of SEQ ID NO: 42 (HCDR3L). In some embodiments, the antibody or antigen-binding fragment thereof comprises an LCDR1 of SEQ ID NO: 56 (LCDR1K), an LCDR2 of SEQ ID NO: 66 (LCDR2I), and an LCDR3 of SEQ ID NO: 80 (LCDR3L).

In some embodiments, the antibody or antigen-binding fragment thereof competes with a reference antibody, wherein the reference antibody comprises an HCDR1 of SEQ ID NO: 11 (HCDR1K), an HCDR2 of SEQ ID NO: 29 (HCDR2O), an HCDR3 of SEQ ID NO: 42 (HCDR3L), an LCDR1 of SEQ ID NO: 56 (LCDR1K), an LCDR2 of SEQ ID NO: 66 (LCDR2I), and an LCDR3 of SEQ ID NO: 80 (LCDR3L).

In some embodiments, the antibody or antigen-binding fragment thereof comprises an HCDR1 of SEQ ID NO: 10 (HCDR1J), an HCDR2 of SEQ ID NO: 28 (HCDR2N), and an HCDR3 of SEQ ID NO: 42 (HCDR3L). In some embodiments, the antibody or antigen-binding fragment thereof comprises an LCDR1 of SEQ ID NO: 55 (LCDR1J), an LCDR2 of SEQ ID NO: 66 (LCDR2I), and an LCDR3 of SEQ ID NO: 79 (LCDR3K).

In some embodiments, the antibody or antigen-binding fragment thereof competes with a reference antibody, wherein the reference antibody comprises an HCDR1 of SEQ ID NO: 10 (HCDR1J), an HCDR2 of SEQ ID NO: 28 (HCDR2N), an HCDR3 of SEQ ID NO: 42 (HCDR3L), an LCDR1 of SEQ ID NO: 55 (LCDR1J), an LCDR2 of SEQ ID NO: 66 (LCDR2I), and an LCDR3 of SEQ ID NO: 79 (LCDR3K).

In some embodiments, the antibody or antigen-binding fragment thereof comprises an HCDR1 of SEQ ID NO: 12 (HCDR1L), an HCDR2 of SEQ ID NO: 28 (HCDR2N), and an HCDR3 of SEQ ID NO: 42 (HCDR3L). In some embodiments, the antibody or antigen-binding fragment thereof comprises an LCDR1 of SEQ ID NO: 55 (LCDR1J), an LCDR2 of SEQ ID NO: 66 (LCDR2I), and an LCDR3 of SEQ ID NO: 80 (LCDR3L).

In some embodiments, the antibody or antigen-binding fragment thereof competes with a reference antibody, wherein the reference antibody comprises an HCDR1 of SEQ ID NO: 12 (HCDR1L), an HCDR2 of SEQ ID NO: 28 (HCDR2N), an HCDR3 of SEQ ID NO: 42 (HCDR3L), an LCDR1 of SEQ ID NO: 55 (LCDR1J), an LCDR2 of SEQ ID NO: 66 (LCDR2I), and an LCDR3 of SEQ ID NO: 80 (LCDR3L).

In some embodiments, the antibody or antigen-binding fragment thereof comprises an HCDR1 of SEQ ID NO: 10 (HCDR1J), an HCDR2 of SEQ ID NO: 28 (HCDR2N), and an HCDR3 of SEQ ID NO: 44 (HCDR3N). In some embodiments, the antibody or antigen-binding fragment thereof comprises an LCDR1 of SEQ ID NO: 55 (LCDR1J), an LCDR2 of SEQ ID NO: 66 (LCDR2I), and an LCDR3 of SEQ ID NO: 79 (LCDR3K).

In some embodiments, the antibody or antigen-binding fragment thereof competes with a reference antibody, wherein the reference antibody comprises an HCDR1 of SEQ ID NO: 10 (HCDR1J), an HCDR2 of SEQ ID NO: 28 (HCDR2N), an HCDR3 of SEQ ID NO: 44 (HCDR3N), an LCDR1 of SEQ ID NO: 55 (LCDR1J), an LCDR2 of SEQ ID NO: 66 (LCDR2I), and an LCDR3 of SEQ ID NO: 79 (LCDR3K).

In some embodiments, the antibody or antigen-binding fragment thereof comprises an HCDR1 of SEQ ID NO: 13 (HCDR1M), an HCDR2 of SEQ ID NO: 30, HCDR2P, and an HCDR3 of SEQ ID NO: 45 (HCDR30). In some embodiments, the antibody or antigen-binding fragment thereof comprises an LCDR1 of SEQ ID NO: 57 (LCDR1L), an LCDR2 of SEQ ID NO: 68 (LCDR2K), and an LCDR3 of SEQ ID NO: 81 (LCDR3M).

In some embodiments, the antibody or antigen-binding fragment thereof competes with a reference antibody, wherein the reference antibody comprises an HCDR1 of SEQ ID NO: 13 (HCDR1M), an HCDR2 of SEQ ID NO: 30 (HCDR2P), an HCDR3 of SEQ ID NO: 45 (HCDR30), an LCDR1 of SEQ ID NO: 57 (LCDR1L), an LCDR2 of SEQ ID NO: 68 (LCDR2K), and an LCDR3 of SEQ ID NO: 81 (LCDR3M).

In some embodiments, the antibody or antigen-binding fragment thereof comprises an HCDR1 of SEQ ID NO: 14 (HCDR1N), an HCDR2 of SEQ ID NO: 30 (HCDR2P), and an HCDR3 of SEQ ID NO: 29 (HCDR2O). In some embodiments, the antibody or antigen-binding fragment thereof comprises an LCDR1 of SEQ ID NO: 57 (LCDR1L), an LCDR2 of SEQ ID NO: 68 (LCDR2K), and an LCDR3 of SEQ ID NO: 82 (LCDR3N).

In some embodiments, the antibody or antigen-binding fragment thereof competes with a reference antibody, wherein the reference antibody comprises an HCDR1 of SEQ ID NO: 14 (HCDR1N), an HCDR2 of SEQ ID NO: 30 (HCDR2P), an HCDR3 of SEQ ID NO: 29 (HCDR2O), an LCDR1 of SEQ ID NO: 57 (LCDR1L), an LCDR2 of SEQ ID NO: 68 (LCDR2K), and an LCDR3 of SEQ ID NO: 82 (LCDR3N).In some embodiments, the integrin α7β1 antibody or antigen binding fragment thereof comprises CDRs with one or more amino acid different from CDRs shown in Table C. In some embodiments, the integrin α7β1 antibody or antigen binding fragment thereof comprises a CDR with no more than 5 amino acids different from any one of the CDRs shown in Table C. In some embodiments, the integrin α7β1 antibody or antigen binding fragment thereof comprises a CDR with no more than 4 amino acids different from any one of the CDRs shown in Table C. In some embodiments, the integrin α7β1 antibody or antigen binding fragment thereof comprises a CDR with no more than 3 amino acids different from any one of the CDRs shown in Table C. In some embodiments, the integrin α7β1 antibody or antigen binding fragment thereof comprises a CDR with no more than 2 amino acids different from any one of the CDRs shown in Table C. In some embodiments, the integrin α7β1 antibody or antigen binding fragment thereof comprises a CDR with no more than 1 amino acid different from any one of the CDRs shown in Table C.

In some embodiments, the integrin α7β1 antibody or antigen binding fragment thereof comprises a HCDR1 comprising an amino acid sequence with no greater than 2 amino acids different from GYSFTRYW (SEQ ID NO: 7, HCDR1G), a HCDR2 comprising an amino acid sequence with no greater than 2 amino acids different from IDPSDSET (SEQ ID NO: 22, HCDR2H), a HCDR3 comprising an amino acid sequence with no greater than 4 amino acids different from ARGGYFGEGWYFDV (SEQ ID NO: 38, HCDR3H), a LCDR1 comprising an amino acid sequence with no greater than 2 amino acids different from QSLVHRNGNTY (SEQ ID NO: 51, LCDR1F), a LCDR2 comprising an amino acid sequence with no greater than 1 amino acid different from KVS (SEQ ID NO: 63, LCDR2F), and a LCDR3 comprising an amino acid sequence with no greater than 4 amino acids different from SQSTHVPWT (SEQ ID NO: 75, LCDR3G).

In some embodiments, the integrin α7β1 antibody or antigen binding fragment thereof comprises a HCDR1 comprising an amino acid sequence with no greater than 2 amino acids different from GYTFTSYW (SEQ ID NO: 8, HCDR1H), a HCDR2 comprising an amino acid sequence with no greater than 2 amino acids different from IYPSDSYT (SEQ ID NO: 23, HCDR2I), a HCDR3 comprising an amino acid sequence with no greater than 4 amino acids different from TRRANYVGAMDY (SEQ ID NO: 39, HCDR3I), a LCDR1 comprising an amino acid sequence with no greater than 2 amino acids different from SSVSY (SEQ ID NO: 52, LCDR1G), a LCDR2 comprising an amino acid sequence with no greater than 1 amino acid different from ATS (SEQ ID NO: 64, LCDR2G), and a LCDR3 comprising an amino acid sequence with no greater than 4 amino acids different from QQWSSNPLT (SEQ ID NO: 76, LCDR3H).

In some embodiments, the integrin α7β1 antibody or antigen binding fragment thereof comprises a HCDR1 comprising an amino acid sequence with no greater than 2 amino acids different from GFSLTSYG (SEQ ID NO: 9, HCDR1I), a HCDR2 comprising an amino acid sequence with no greater than 2 amino acids different from IWVGGIT (SEQ ID NO: 24, HCDR2J), a HCDR3 comprising an amino acid sequence with no greater than 4 amino acids different from ARDGYYGDFDV (SEQ ID NO: 40, HCDR3J), a LCDR1 comprising an amino acid sequence with no greater than 2 amino acids different from QDINKF (SEQ ID NO: 53, LCDR1H), a LCDR2 comprising an amino acid sequence with no greater than 1 amino acid different from YTS (SEQ ID NO: 65, LCDR2H), and a LCDR3 comprising an amino acid sequence with no greater than 4 amino acids different from LQYDNLRT (SEQ ID NO: 77, LCDR3I).

In some embodiments, the integrin α7β1 antibody or antigen binding fragment thereof comprises a HCDR1 comprising an amino acid sequence with no greater than 2 amino acids different from GFSLTSYG (SEQ ID NO: 9, HCDR1I), a HCDR2 comprising an amino acid sequence with no greater than 2 amino acids different from IWAGGIT (SEQ ID NO: 25, HCDR2K), a HCDR3 comprising an amino acid sequence with no greater than 4 amino acids different from ARDGYYGDFDV (SEQ ID NO: 40, HCDR3J), a LCDR1 comprising an amino acid sequence with no greater than 2 amino acids different from QDINKY (SEQ ID NO: 54, LCDR1I), a LCDR2 comprising an amino acid sequence with no greater than 1 amino acid different from YTS (SEQ ID NO: 65, LCDR2H), and a LCDR3 comprising an amino acid sequence with no greater than 4 amino acids different from LQYDNLRT (SEQ ID NO: 77, LCDR3I).

In some embodiments, the integrin α7β1 antibody or antigen binding fragment thereof comprises a HCDR1 comprising an amino acid sequence with no greater than 2 amino acids different from GFSLTSYG (SEQ ID NO: 9, HCDR1I), a HCDR2 comprising an amino acid sequence with no greater than 2 amino acids different from IWAGGIT (SEQ ID NO: 25, HCDR2K), a HCDR3 comprising an amino acid sequence with no greater than 4 amino acids different from ARDGYYGHFDV (SEQ ID NO: 41, HCDR3K), a LCDR1 comprising an amino acid sequence with no greater than 2 amino acids different from QDINKY (SEQ ID NO: 54, LCDR1I), a LCDR2 comprising an amino acid sequence with no greater than 1 amino acid different from YTS (SEQ ID NO: 65, LCDR2H), and a LCDR3 comprising an amino acid sequence with no greater than 4 amino acids different from LQYDNLRA (SEQ ID NO: 78, LCDR3J).

In some embodiments, the integrin α7β1 antibody or antigen binding fragment thereof comprises a HCDR1 comprising an amino acid sequence with no greater than 2 amino acids different from GFTFSSDA (SEQ ID NO: 10, HCDR1J), a HCDR2 comprising an amino acid sequence with no greater than 2 amino acids different from ISIGGSA (SEQ ID NO: 26, HCDR2L), a HCDR3 comprising an amino acid sequence with no greater than 4 amino acids different from ARGGDYDAMDY (SEQ ID NO: 42, HCDR3L), a LCDR1 comprising an amino acid sequence with no greater than 2 amino acids different from QSISDL (SEQ ID NO: 55, LCDR1J), a LCDR2 comprising an amino acid sequence with no greater than 1 amino acid different from YAS (SEQ ID NO: 66, LCDR2I), and a LCDR3 comprising an amino acid sequence with no greater than 4 amino acids different from QNGHNFPFT (SEQ ID NO: 79, LCDR3K).

In some embodiments, the integrin α7β1 antibody or antigen binding fragment thereof comprises a HCDR1 comprising an amino acid sequence with no greater than 2 amino acids different from GFTFSSSA (SEQ ID NO: 11, HCDR1K), a HCDR2 comprising an amino acid sequence with no greater than 2 amino acids different from ISVGGST (SEQ ID NO: 27, HCDR2M), a HCDR3 comprising an amino acid sequence with no greater than 4 amino acids different from ARGGDYDAMDY (SEQ ID NO: 42, HCDR3L), a LCDR1 comprising an amino acid sequence with no greater than 2 amino acids different from QSISDY (SEQ ID NO: 56, LCDR1K), a LCDR2 comprising an amino acid sequence with no greater than 1 amino acid different from YAS (SEQ ID NO: 66, LCDR2I), and a LCDR3 comprising an amino acid sequence with no greater than 4 amino acids different from QNGHSFPFT (SEQ ID NO: 80, LCDR3L).

In some embodiments, the integrin α7β1 antibody or antigen binding fragment thereof comprises a HCDR1 comprising an amino acid sequence with no greater than 2 amino acids different from GFTFSSSA (SEQ ID NO: 11, HCDR1K), a HCDR2 comprising an amino acid sequence with no greater than 2 amino acids different from ISVGGST (SEQ ID NO: 27, HCDR2M), a HCDR3 comprising an amino acid sequence with no greater than 4 amino acids different from ARGGDYDAMDY (SEQ ID NO: 42, HCDR3L), a LCDR1 comprising an amino acid sequence with no greater than 2 amino acids different from QSISDY (SEQ ID NO: 56, LCDR1K), a LCDR2 comprising an amino acid sequence with no greater than 1 amino acid different from YSS (SEQ ID NO: 67, LCDR2J), and a LCDR3 comprising an amino acid sequence with no greater than 4 amino acids different from QNGHSFPFT (SEQ ID NO: 80, LCDR3L).

In some embodiments, the integrin α7β1 antibody or antigen binding fragment thereof comprises a HCDR1 comprising an amino acid sequence with no greater than 2 amino acids different from GFTFSSSA (SEQ ID NO: 11, HCDR1K), a HCDR2 comprising an amino acid sequence with no greater than 2 amino acids different from ISVGGST (SEQ ID NO: 27, HCDR2M), a HCDR3 comprising an amino acid sequence with no greater than 4 amino acids different from ARGGDYDPMDY (SEQ ID NO: 43, HCDR3M), a LCDR1 comprising an amino acid sequence with no greater than 2 amino acids different from QSISDY (SEQ ID NO: 56, LCDR1K), a LCDR2 comprising an amino acid sequence with no greater than 1 amino acid different from YAS (SEQ ID NO: 66, LCDR2I), and a LCDR3 comprising an amino acid sequence with no greater than 4 amino acids different from QNGHSFPFT (SEQ ID NO: 80, LCDR3L).

In some embodiments, the integrin α7β1 antibody or antigen binding fragment thereof comprises a HCDR1 comprising an amino acid sequence with no greater than 2 amino acids different from GFTFSSDA (SEQ ID NO: 10, HCDR1J), a HCDR2 comprising an amino acid sequence with no greater than 2 amino acids different from ISIGGST (SEQ ID NO: 28, HCDR2N), a HCDR3 comprising an amino acid sequence with no greater than 4 amino acids different from ARGGDYDAMDY (SEQ ID NO: 42, HCDR3L), a LCDR1 comprising an amino acid sequence with no greater than 2 amino acids different from QSISDL (SEQ ID NO: 55, LCDR1J), a LCDR2 comprising an amino acid sequence with no greater than 1 amino acid different from YAS (SEQ ID NO: 66, LCDR2I), and a LCDR3 comprising an amino acid sequence with no greater than 4 amino acids different from QNGHNFPFT (SEQ ID NO: 79, LCDR3K).

In some embodiments, the integrin α7β1 antibody or antigen binding fragment thereof comprises a HCDR1 comprising an amino acid sequence with no greater than 2 amino acids different from GFTFSSSA (SEQ ID NO: 11, HCDR1K), a HCDR2 comprising an amino acid sequence with no greater than 2 amino acids different from ISVGGSI (SEQ ID NO: 29, HCDR2O), a HCDR3 comprising an amino acid sequence with no greater than 4 amino acids different from ARGGDYDAMDY (SEQ ID NO: 42, HCDR3L), a LCDR1 comprising an amino acid sequence with no greater than 2 amino acids different from QSISDY (SEQ ID NO: 56, LCDR1K), a LCDR2 comprising an amino acid sequence with no greater than 1 amino acid different from YAS (SEQ ID NO: 66, LCDR2I), and a LCDR3 comprising an amino acid sequence with no greater than 4 amino acids different from QNGHSFPFT (SEQ ID NO: 80, LCDR3L).

In some embodiments, the integrin α7β1 antibody or antigen binding fragment thereof comprises a HCDR1 comprising an amino acid sequence with no greater than 2 amino acids different from GYSFTRYW (SEQ ID NO: 7, HCDR1G), a HCDR2 comprising an amino acid sequence with no greater than 2 amino acids different from IDPSDSET (SEQ ID NO: 22, HCDR2H), a HCDR3 comprising an amino acid sequence with no greater than 4 amino acids different from ARGGYFGEGWYFDV (SEQ ID NO: 38, HCDR3H), a LCDR1 comprising an amino acid sequence with no greater than 2 amino acids different from QSLVHRNGNTY (SEQ ID NO: 51, LCDR1F), a LCDR2 comprising an amino acid sequence with no greater than 1 amino acid different from KVS (SEQ ID NO: 63, LCDR2F), and a LCDR3 comprising an amino acid sequence with no greater than 4 amino acids different from SQSTHVPWT (SEQ ID NO: 75, LCDR3G).

In some embodiments, the integrin α7β1 antibody or antigen binding fragment thereof comprises a HCDR1 comprising an amino acid sequence with no greater than 2 amino acids different from GFTFSSDA (SEQ ID NO: 10, HCDR1J), a HCDR2 comprising an amino acid sequence with no greater than 2 amino acids different from ISIGGST (SEQ ID NO: 28, HCDR2N), a HCDR3 comprising an amino acid sequence with no greater than 4 amino acids different from ARGGDYDAMDY (SEQ ID NO: 42, HCDR3L), a LCDR1 comprising an amino acid sequence with no greater than 2 amino acids different from QSISDL (SEQ ID NO: 55, LCDR1J), a LCDR2 comprising an amino acid sequence with no greater than 1 amino acid different from YAS (SEQ ID NO: 66, LCDR2I), and a LCDR3 comprising an amino acid sequence with no greater than 4 amino acids different from QNGHNFPFT (SEQ ID NO: 79, LCDR3K).

In some embodiments, the integrin α7β1 antibody or antigen binding fragment thereof comprises a HCDR1 comprising an amino acid sequence with no greater than 2 amino acids different from GFTFSNCA (SEQ ID NO: 12, HCDR1L), a HCDR2 comprising an amino acid sequence with no greater than 2 amino acids different from ISIGGST (SEQ ID NO: 28, HCDR2N), a HCDR3 comprising an amino acid sequence with no greater than 4 amino acids different from ARGGDYDAMDY (SEQ ID NO: 42, HCDR3L), a LCDR1 comprising an amino acid sequence with no greater than 2 amino acids different from QSISDL (SEQ ID NO: 55, LCDR1J), a LCDR2 comprising an amino acid sequence with no greater than 1 amino acid different from YAS (SEQ ID NO: 66, LCDR2I), and a LCDR3 comprising an amino acid sequence with no greater than 4 amino acids different from QNGHSFPFT (SEQ ID NO: 80, LCDR3L).

In some embodiments, the integrin α7β1 antibody or antigen binding fragment thereof comprises a HCDR1 comprising an amino acid sequence with no greater than 2 amino acids different from GFTFSSDA (SEQ ID NO: 10, HCDR1J), a HCDR2 comprising an amino acid sequence with no greater than 2 amino acids different from ISIGGST (SEQ ID NO: 28, HCDR2N), a HCDR3 comprising an amino acid sequence with no greater than 4 amino acids different from TRGGDYDAMDY (SEQ ID NO: 44, HCDR3N), a LCDR1 comprising an amino acid sequence with no greater than 2 amino acids different from QSISDL (SEQ ID NO: 55, LCDR1J), a LCDR2 comprising an amino acid sequence with no greater than 1 amino acid different from YAS (SEQ ID NO: 66, LCDR2I), and a LCDR3 comprising an amino acid sequence with no greater than 4 amino acids different from QNGHNFPFT (SEQ ID NO: 79, LCDR3K).

In some embodiments, the integrin α7β1 antibody or antigen binding fragment thereof comprises a HCDR1 comprising an amino acid sequence with no greater than 2 amino acids different from GFSLSTSDMG (SEQ ID NO: 13, HCDR1M), a HCDR2 comprising an amino acid sequence with no greater than 2 amino acids different from IYWDDDK (SEQ ID NO: 30, HCDR2P), a HCDR3 comprising an amino acid sequence with no greater than 4 amino acids different from VRGTAVVAFYWYFDV (SEQ ID NO: 45, HCDR30), a LCDR1 comprising an amino acid sequence with no greater than 2 amino acids different from QDINNY (SEQ ID NO: 57, LCDR1L), a LCDR2 comprising an amino acid sequence with no greater than 1 amino acid different from RAN (SEQ ID NO: 68, LCDR2K), and a LCDR3 comprising an amino acid sequence with no greater than 4 amino acids different from LHYDDFPLT (SEQ ID NO: 81, LCDR3M).

In some embodiments, the integrin α7β1 antibody or antigen binding fragment thereof comprises a HCDR1 comprising an amino acid sequence with no greater than 2 amino acids different from GFSLSTSDLG (SEQ ID NO: 14, HCDR1N), a HCDR2 comprising an amino acid sequence with no greater than 2 amino acids different from IYWDDDK (SEQ ID NO: 30, HCDR2P), a HCDR3 comprising an amino acid sequence with no greater than 4 amino acids different from VRGTAVVAFYWYFDV (SEQ ID NO: 45, HCDR30), a LCDR1 comprising an amino acid sequence with no greater than 2 amino acids different from QDINNY (SEQ ID NO: 57, LCDR1L), a LCDR2 comprising an amino acid sequence with no greater than 1 amino acid different from RAN (SEQ ID NO: 68, LCDR2K), and a LCDR3 comprising an amino acid sequence with no greater than 4 amino acids different from LQYDDFPLT (SEQ ID NO: 82, LCDR3N).

TABLE D
Exemplary VH and VL Sequences of Mouse anti-α7β1 Integrin Antibodies.

| Ab | VH Sequence | VL Sequence |
|---|---|---|
| Ab8 | QVPLQQSGPQLVRPGASVKISCKASGYSF | DVVMTQIPLSLPVSLGDQASISCRSSQSL |
| | TRYWMHWVKQRPGQGLDWIGKIDPSDSET | VHRNGNTYLHWYLQKPGQSPKLLIYKVSN |
| | RLNQEFRDKATLTVDKSSSTAYLQLSSPT | RFSGVPDRFSGSGSGTDFTLKISRVEAED |
| | SEDSAVYYCARGGYFGEGWYFDVWGAGTT | LGVYFCSQSTHVPWTFGGGTKLEIK |
| | VTVSS (SEQ ID NO: 142, VH8) | (SEQ ID NO: 143, VL8) |
| Ab22 | QVTLKESGPGILQPSQTLSLTCSFSGFSL | DIKMTQSPSSMYASLGERVTITCKASQDI |
| | STSDMGVSWIRQPSGKGLEWLAHIYWDDD | NNYLNWFQQKPGKSPKTLIYRANRLVDGV |
| | KRYSPSLKSRLTISKDTSRNQVFLKITSV | PSRFSGSGSGQDYSLTISSLEYEDMGIYY |
| | DTADTATYYCVRGTAVVAFYWYFDVWGAG | CLHYDDFPLTFGAGTKVELK (SEQ ID |
| | TTVTVSS (SEQ ID NO: 144, | NO: 145, VL22) |
| | VH22) | |
| Ab23 | QVTLKESGPGILQPSQTLSLTCSCSGFSL | DIKMTQSPSSMYASLGERVTITCKASQDI |
| | STSDLGVSWIRQPSGKGLEWLAHIYWDDD | NNYLNWFQQKPGKSPKTLIYRANRLVDGV |
| | KRYSPSLKSRLTISKDTSRNQVFLKITSV | PSRFSGSGSGQDYSLTISSLEYEDMGIYY |
| | DTADTATYYCVRGTAVVAFYWYFDVWGAG | CLQYDDFPLTFGAGTKVELK (SEQ ID |
| | TTVTVSS (SEQ ID NO: 146, | NO: 147, VL23) |
| | VH23) | |
| Ab10 | QVQLKESGPGLVAPSQSLSITCTVSGFSL | DIQMTQSPSSLSASLGGKVTITCTASQDI |
| | TSYGVHWVRQPPGKGLEWLGVIWVGGITH | NKFIAWYQHKPGKGPRLLIHYTSTLQPGI |
| | YNSALMSRLSISKDNSKSQVFLKMNSLQT | PSRFSGSGSGRDYSFSISNLEPEDIATYY |
| | DDIAMYYCARDGYYGDFDVWGAGTTVTVS | CLQYDNLRTFGGGTKLEIK (SEQ ID |
| | S (SEQ ID NO: 148, VH10) | NO: 149, VL10) |
| Ab11 | QVQLKESGPGLVAPSQSLSITCTVSGFSL | DIQMTQSPSSLSASLGGKVTITCKASQDI |
| | TSYGVHWVRQPPGKGLEWLGVIWAGGITH | NKYIAWYQHKPGKGPRLLIHYTSTLQPGI |
| | YNSALMSRLSISKDNSKSQVFLKMNSPQT | PSRFSGSGSGRDYSFSISNLVPEDIATYY |
| | DD IAMYYCARDGYYGDFDVWGAGTTVTVS | CLQYDNLRTFGGGTKLEIK (SEQ ID |
| | S (SEQ ID NO: 150, VH11) | NO: 151, VL11) |
| Ab12 | QVQLKESGPGLVAPSQSLSITCTVSGFSL | DIQMTQSPSSLSASLGGKVTITCKASQDI |
| | TSYGVHWVRQPPGKGLEWLGVIWAGGITH | NKYIAWYQHKPGQGPRLLIDYTSTLQPGI |
| | YNSALMSRLSISKDNSKSQVFLKMNSLQT | PSRFSGSGSGRDYSFTISNLEPEDIATYF |
| | DDTAMFYCARDGYYGHFDVWGAGTTVTVS | CLQYDNLRAFGGGTKLEIK (SEQ ID |
| | S (SEQ ID NO: 152, VH12) | NO: 153, VL12) |
| Ab9 | QVQLQQPGAELVRPGASVKLSCKASGYTF | QIVLSQSPAILSASPGEKVTMTCRASSSV |
| | TSYWINWVKQRPGQGLEWIGNIYPSDSYT | SYMYWYQQKPGSSPKPWIYATSNLASGVP |
| | NYNQKFKDKATLTVDKSSSTAYMQLSSPT | ARFSGSGSGTSYSLTISRVEAEDAATYYC |
| | SEDSAVYYCTRRANYVGAMDYWGQGTSVT | QQWSSNPLTFGAGTKLELK (SEQ ID |
| | VSS (SEQ ID NO: 154, VH9) | NO: 155, VL9) |
| Ab13 | EVKLVESGGGLVKPGGSLKLSCAASGFTF | DIVMTQSPATLSVTPGDRVSLSCRASQSI |
| | SSDAMSWVRQTPEKSLEWVASISIGGSAF | SDLLHWYQQKSHESPRLLIKYASQSISGI |
| | YPESVRGRFTISRDNARNILYLQMSSLRS | PSRFSGSGSGSDFTLSINSVEPEDVGVYF |
| | EDTAMYYCARGGDYDAMDYWGQGTSVTVS | CQNGHNFPFTFGSGTKLEIK (SEQ ID |
| | S (SEQ ID NO: 156, VH13) | NO: 157, VL13) |
| Ab14 | EVKLVESGGGLVKPGGSLKLSCAASGFTF | DIVMTQSPATLSVTPGDRVSLSCRASQSI |
| | SSSAMSWVRQTPEKRLEWVASISVGGSTF | SDYLHWFQQKSHESPRLLIKYASQSISGI |
| | YPDSVKGRFTISRDNARKILYLQMSSLRS | PSRFSGSGSGSDFTLSINSVEPEDVGVYY |
| | EDTAMYYCARGGDYDAMDYWGQGTSVTVS | CQNGHSFPFTFGSGTKLEIK (SEQ ID |
| | S (SEQ ID NO: 158, VH14) | NO: 159, VL14) |
| Ab15 | EVKLVESGGGLVKPGGSLKLSCAASGFTF | DIVMTQSPATLSVTPGDRVSLSCRASQSI |
| | SSSAMSWVRQTPEKRLEWVASISVGGSTF | SDYLHWFQQKSHESPRLLIKYSSQSISGI |
| | YPDSVKGRFTISRDNARNILYLQMSSLRS | PSRFSGSGSGSDFTLSINSVEPEDVGVYY |
| | EDTAMYYCARGGDYDAMDYWGQGTSVTVS | CQNGHSFPFTFGSGTKLEIK (SEQ ID |
| | S (SEQ ID NO: 160, VH15) | NO: 161, VL15) |
| Ab16 | EVKLVESGGGLVKPGGSLKLSCAASGFTF | DIVMTQSPATLSVTPGDRVSLSCRASQSI |
| | SSSAMSWVRQTPEKRLEWVASISVGGSTF | SDYLHWFQQKSHESPRLLIKYASQSISGI |
| | YADSVKGRFTISRDNARNILYLQMSSLRS | PSRFSGSGSGSDFTLSINSVEPEDVGVYY |
| | EDTAMYYCARGGDYDPMDYWGQGTSVTVS | CQNGHSFPFTFGSGTKLEIK (SEQ ID |
| | S (SEQ ID NO: 162, VH16) | NO: 163, VL16) |
| Ab17 | EVKLVESGGGLVKPGGSLKLSCAASGFTF | DIVMTQSPATLSVTPGDRVSLSCRASQSI |
| | SSDAMSWVRQTPEKRLEWVASISIGGSTF | SDLLHWYQQKSHESPRLLIKYASQSISGI |
| | YPESVKGRFTISRDNARNILYLQMSSLRS | PSRFSGSGSGSDFTLSINSVEPEDVGVYF |
| | EDTAMYYCARGGDYDAMDYWGQGTSVTVS | CQNGHNFPFTFGSGTKLEIK (SEQ ID |
| | S (SEQ ID NO: 164, VH17) | NO: 165, VL17) |
| Ab18 | EVKVVESGGGLVKPGGSLKLSCATSGFTF | DIVMTQSPATLSVTPGDRVSLSCRASQSI |
| | SSSAMSWVRQTPERRLEWVASISVGGSIF | SDYLHWFQQKSHESPRLLIKYASQSISGI |
| | YPDSVKGRFTISRENARNILYLQMSSLRS | PSRFSGSGSGSDFTLSINSVEPEDVGVYY |
| | EDTAMYYCARGGDYDAMDYWGQGTSVTVS | CQNGHSFPFTFGSGTKLEIK (SEQ ID |
| | S (SEQ ID NO: 166, VH18) | NO: 167, VL18) |
| Ab20 | EVKLVESGGGLVKPGGSLKLSCAASGFTF | DIVMTQFPDTLSVTPGDRVSLSCRASQSI |
| | SNCAMSWVRQTPEKRLEWVASISIGGSTF | SDLLHWYQQKSHESPRLLIKYASQSISEI |
| | YPDSVKGRFTISRDNGRNILYLQMSSLRS | PSRFSGSGSGSDFTLSINSVEPEDVGVYY |
| | EDTALYYCARGGDYDAMDYWGQGTSVTVS | CQNGHSFPFTFGSGTKLEIK (SEQ ID |
| | S (SEQ ID NO: 170, VH20) | NO: 171, VL20) |
| Ab21 | EVKLVESGGGLVKPGGSLKLSCEASGFTF | DIVMTQSPATLSVTPGDRVSLSCRASQSI |
| | SSDAMSWVRQTPEKRLEWVASISIGGSTF | SDLLHWYQQKSHESPRLLIKYASQSISGI |
| | YPESVKGRFTISRDNARNILYLQMSSLRS | PSRFSGSGSGSDFTLSINSVEPEDVGVYF |
| | EDTAMYYCTRGGDYDAMDYWGQGTSVTVS | CQNGHNFPFTFGSGTKLEIK (SEQ ID |
| | S (SEQ ID NO: 172, VH21) | NO: 173, VL21) |

In some embodiments, the integrin α7β1 antibody or antigen binding fragment thereof comprises a variable heavy region comprising an amino acid sequence at least 80% identical to SEQ ID NO: 142 (VH8). In some embodiments, the integrin α7β1 antibody or antigen binding fragment thereof comprises a variable heavy region comprising an amino acid sequence at least 83% identical to SEQ ID NO: 142 (VH8). In some embodiments, the integrin α7β1 antibody or antigen binding fragment thereof comprises a variable heavy region comprising an amino acid sequence at least 85% identical to SEQ ID NO: 142 (VH8). In some embodiments, the integrin α7β1 antibody or antigen binding fragment thereof comprises a variable heavy region comprising an amino acid sequence at least 87% identical to SEQ ID NO: 142 (VH8). In some embodiments, the integrin α7β1 antibody or antigen binding fragment thereof comprises a variable heavy region comprising an amino acid sequence at least 90% identical to SEQ ID NO: 142 (VH8). In some embodiments, the integrin α7β1 antibody or antigen binding fragment thereof comprises a variable heavy region comprising an amino acid sequence at least 95% identical to SEQ ID NO: 142 (VH8). In some embodiments, the integrin α7β1 antibody or antigen binding fragment thereof comprises a variable heavy region comprising an amino acid sequence at least 98% identical to SEQ ID NO: 142 (VH8). In some embodiments, the integrin α7β1 antibody or antigen binding fragment thereof comprises a variable heavy region comprising an amino acid sequence at least 99% identical to SEQ ID NO: 142 (VH8). In some embodiments, the integrin α7β1 antibody or antigen binding fragment thereof comprises a variable heavy region comprising an amino acid sequence identical to SEQ ID NO: 142 (VH8).

In some embodiments, the integrin α7β1 antibody or antigen binding fragment thereof comprises a variable heavy region comprising an amino acid sequence at least 80% identical to SEQ ID NO: 154 (VH9). In some embodiments, the integrin α7β1 antibody or antigen binding fragment thereof comprises a variable heavy region comprising an amino acid sequence at least 83% identical to SEQ ID NO: 154 (VH9). In some embodiments, the integrin α7β1 antibody or antigen binding fragment thereof comprises a variable heavy region comprising an amino acid sequence at least 85% identical to SEQ ID NO: 154 (VH9). In some embodiments, the integrin α7β1 antibody or antigen binding fragment thereof comprises a variable heavy region comprising an amino acid sequence at least 87% identical to SEQ ID NO: 154 (VH9). In some embodiments, the integrin α7β1 antibody or antigen binding fragment thereof comprises a variable heavy region comprising an amino acid sequence at least 90% identical to SEQ ID NO: 154 (VH9). In some embodiments, the integrin α7β1 antibody or antigen binding fragment thereof comprises a variable heavy region comprising an amino acid sequence at least 95% identical to SEQ ID NO: 154 (VH9). In some embodiments, the integrin α7β1 antibody or antigen binding fragment thereof comprises a variable heavy region comprising an amino acid sequence at least 98% identical to SEQ ID NO: 154 (VH9). In some embodiments, the integrin α7β1 antibody or antigen binding fragment thereof comprises a variable heavy region comprising an amino acid sequence at least 99% identical to SEQ ID NO: 154 (VH9). In some embodiments, the integrin α7β1 antibody or antigen binding fragment thereof comprises a variable heavy region comprising an amino acid sequence identical to SEQ ID NO: 154 (VH9).

In some embodiments, the integrin α7β1 antibody or antigen binding fragment thereof comprises a variable heavy region comprising an amino acid sequence at least 80% identical to SEQ ID NO: 148 (VH10). In some embodiments, the integrin α7β1 antibody or antigen binding fragment thereof comprises a variable heavy region comprising an amino acid sequence at least 83% identical to SEQ ID NO: 148 (VH10). In some embodiments, the integrin α7β1 antibody or antigen binding fragment thereof comprises a variable heavy region comprising an amino acid sequence at least 85% identical to SEQ ID NO: 148 (VH10). In some embodiments, the integrin α7β1 antibody or antigen binding fragment thereof comprises a variable heavy region comprising an amino acid sequence at least 87% identical to SEQ ID NO: 148 (VH10). In some embodiments, the integrin α7β1 antibody or antigen binding fragment thereof comprises a variable heavy region comprising an amino acid sequence at least 90% identical to SEQ ID NO: 148 (VH10). In some embodiments, the integrin α7β1 antibody or antigen binding fragment thereof comprises a variable heavy region comprising an amino acid sequence at least 95% identical to SEQ ID NO: 148 (VH10). In some embodiments, the integrin α7β1 antibody or antigen binding fragment thereof comprises a variable heavy region comprising an amino acid sequence at least 98% identical to SEQ ID NO: 148 (VH10). In some embodiments, the integrin α7β1 antibody or antigen binding fragment thereof comprises a variable heavy region comprising an amino acid sequence at least 99% identical to SEQ ID NO: 148 (VH10). In some embodiments, the integrin α7β1 antibody or antigen binding fragment thereof comprises a variable heavy region comprising an amino acid sequence identical to SEQ ID NO: 148 (VH10).

In some embodiments, the integrin α7β1 antibody or antigen binding fragment thereof comprises a variable heavy region comprising an amino acid sequence at least 80% identical to SEQ ID NO: 150 (VH11). In some embodiments, the integrin α7β1 antibody or antigen binding fragment thereof comprises a variable heavy region comprising an amino acid sequence at least 83% identical to SEQ ID NO: 150 (VH11). In some embodiments, the integrin α7β1 antibody or antigen binding fragment thereof comprises a variable heavy region comprising an amino acid sequence at least 85% identical to SEQ ID NO: 150 (VH11). In some embodiments, the integrin α7β1 antibody or antigen binding fragment thereof comprises a variable heavy region comprising an amino acid sequence at least 87% identical to SEQ ID NO: 150 (VH11). In some embodiments, the integrin α7β1 antibody or antigen binding fragment thereof comprises a variable heavy region comprising an amino acid sequence at least 90% identical to SEQ ID NO: 150 (VH11). In some embodiments, the integrin α7β1 antibody or antigen binding fragment thereof comprises a variable heavy region comprising an amino acid sequence at least 95% identical to SEQ ID NO: 150 (VH11). In some embodiments, the integrin α7β1 antibody or antigen binding fragment thereof comprises a variable heavy region comprising an amino acid sequence at least 98% identical to SEQ ID NO: 150 (VH11). In some embodiments, the integrin α7β1 antibody or antigen binding fragment thereof comprises a variable heavy region comprising an amino acid sequence at least 99% identical to SEQ ID NO: 150 (VH11). In some embodiments, the integrin α7β1 antibody or antigen binding fragment thereof comprises a variable heavy region comprising an amino acid sequence identical to SEQ ID NO: 150 (VH11).

In some embodiments, the integrin α7β1 antibody or antigen binding fragment thereof comprises a variable heavy region comprising an amino acid sequence at least 80% identical to SEQ ID NO: 152 (VH12). In some embodiments, the integrin α7β1 antibody or antigen binding fragment thereof comprises a variable heavy region comprising an amino acid sequence at least 83% identical to SEQ ID NO: 152 (VH12). In some embodiments, the integrin α7β1 antibody or antigen binding fragment thereof comprises a variable heavy region comprising an amino acid sequence at least 85% identical to SEQ ID NO: 152 (VH12). In some embodiments, the integrin α7β1 antibody or antigen binding fragment thereof comprises a variable heavy region comprising an amino acid sequence at least 87% identical to SEQ ID NO: 152 (VH12). In some embodiments, the integrin α7β1 antibody or antigen binding fragment thereof comprises a variable heavy region comprising an amino acid sequence at least 90% identical to SEQ ID NO: 152 (VH12). In some embodiments, the integrin α7β1 antibody or antigen binding fragment thereof comprises a variable heavy region comprising an amino acid sequence at least 95% identical to SEQ ID NO: 152 (VH12). In some embodiments, the integrin α7β1 antibody or antigen binding fragment thereof comprises a variable heavy region comprising an amino acid sequence at least 98% identical to SEQ ID NO: 152 (VH12). In some embodiments, the integrin α7β1 antibody or antigen binding fragment thereof comprises a variable heavy region comprising an amino acid sequence at least 99% identical to SEQ ID NO: 152 (VH12). In some embodiments, the integrin α7β1 antibody or antigen binding fragment thereof comprises a variable heavy region comprising an amino acid sequence identical to SEQ ID NO: 152 (VH12).

In some embodiments, the integrin α7β1 antibody or antigen binding fragment thereof comprises a variable heavy region comprising an amino acid sequence at least 80% identical to SEQ ID NO: 156 (VH13). In some embodiments, the integrin α7β1 antibody or antigen binding fragment thereof comprises a variable heavy region comprising an amino acid sequence at least 83% identical to SEQ ID NO: 156 (VH13). In some embodiments, the integrin α7β1 antibody or antigen binding fragment thereof comprises a variable heavy region comprising an amino acid sequence at least 85% identical to SEQ ID NO: 156 (VH13). In some embodiments, the integrin α7β1 antibody or antigen binding fragment thereof comprises a variable heavy region comprising an amino acid sequence at least 87% identical to SEQ ID NO: 156 (VH13). In some embodiments, the integrin α7β1 antibody or antigen binding fragment thereof comprises a variable heavy region comprising an amino acid sequence at least 90% identical to SEQ ID NO: 156 (VH13). In some embodiments, the integrin α7β1 antibody or antigen binding fragment thereof comprises a variable heavy region comprising an amino acid sequence at least 95% identical to SEQ ID NO: 156 (VH13). In some embodiments, the integrin α7β1 antibody or antigen binding fragment thereof comprises a variable heavy region comprising an amino acid sequence at least 98% identical to SEQ ID NO: 156 (VH13). In some embodiments, the integrin α7β1 antibody or antigen binding fragment thereof comprises a variable heavy region comprising an amino acid sequence at least 99% identical to SEQ ID NO: 156 (VH13). In some embodiments, the integrin α7β1 antibody or antigen binding fragment thereof comprises a variable heavy region comprising an amino acid sequence identical to SEQ ID NO: 156 (VH13).

In some embodiments, the integrin α7β1 antibody or antigen binding fragment thereof comprises a variable heavy region comprising an amino acid sequence at least 80% identical to SEQ ID NO: 158 (VH14). In some embodiments, the integrin α7β1 antibody or antigen binding fragment thereof comprises a variable heavy region comprising an amino acid sequence at least 83% identical to SEQ ID NO: 158 (VH14). In some embodiments, the integrin α7β1 antibody or antigen binding fragment thereof comprises a variable heavy region comprising an amino acid sequence at least 85% identical to SEQ ID NO: 158 (VH14). In some embodiments, the integrin α7β1 antibody or antigen binding fragment thereof comprises a variable heavy region comprising an amino acid sequence at least 87% identical to SEQ ID NO: 158 (VH14). In some embodiments, the integrin α7β1 antibody or antigen binding fragment thereof comprises a variable heavy region comprising an amino acid sequence at least 90% identical to SEQ ID NO: 158 (VH14). In some embodiments, the integrin α7β1 antibody or antigen binding fragment thereof comprises a variable heavy region comprising an amino acid sequence at least 95% identical to SEQ ID NO: 158 (VH14). In some embodiments, the integrin α7β1 antibody or antigen binding fragment thereof comprises a variable heavy region comprising an amino acid sequence at least 98% identical to SEQ ID NO: 158 (VH14). In some embodiments, the integrin α7β1 antibody or antigen binding fragment thereof comprises a variable heavy region comprising an amino acid sequence at least 99% identical to SEQ ID NO: 158 (VH14). In some embodiments, the integrin α7β1 antibody or antigen binding fragment thereof comprises a variable heavy region comprising an amino acid sequence identical to SEQ ID NO: 158 (VH14).

In some embodiments, the integrin α7β1 antibody or antigen binding fragment thereof comprises a variable heavy region comprising an amino acid sequence at least 80% identical to SEQ ID NO: 160 (VH15). In some embodiments, the integrin α7β1 antibody or antigen binding fragment thereof comprises a variable heavy region comprising an amino acid sequence at least 83% identical to SEQ ID NO: 160 (VH15). In some embodiments, the integrin α7β1 antibody or antigen binding fragment thereof comprises a variable heavy region comprising an amino acid sequence at least 85% identical to SEQ ID NO: 160 (VH15). In some embodiments, the integrin α7β1 antibody or antigen binding fragment thereof comprises a variable heavy region comprising an amino acid sequence at least 87% identical to SEQ ID NO: 160 (VH15). In some embodiments, the integrin α7β1 antibody or antigen binding fragment thereof comprises a variable heavy region comprising an amino acid sequence at least 90% identical to SEQ ID NO: 160 (VH15). In some embodiments, the integrin α7β1 antibody or antigen binding fragment thereof comprises a variable heavy region comprising an amino acid sequence at least 95% identical to SEQ ID NO: 160 (VH15). In some embodiments, the integrin α7β1 antibody or antigen binding fragment thereof comprises a variable heavy region comprising an amino acid sequence at least 98% identical to SEQ ID NO: 160 (VH15). In some embodiments, the integrin α7β1 antibody or antigen binding fragment thereof comprises a variable heavy region comprising an amino acid sequence at least 99% identical to SEQ ID NO: 160 (VH15). In some embodiments, the integrin α7β1 antibody or antigen binding fragment thereof comprises a variable heavy region comprising an amino acid sequence identical to SEQ ID NO: 160 (VH15).

In some embodiments, the integrin α7β1 antibody or antigen binding fragment thereof comprises a variable heavy region comprising an amino acid sequence at least 80% identical to SEQ ID NO: 162 (VH16). In some embodiments, the integrin α7β1 antibody or antigen binding fragment thereof comprises a variable heavy region comprising an amino acid sequence at least 83% identical to SEQ ID NO: 162 (VH16). In some embodiments, the integrin α7β1 antibody or antigen binding fragment thereof comprises a variable heavy region comprising an amino acid sequence at least 85% identical to SEQ ID NO: 162 (VH16). In some embodiments, the integrin α7β1 antibody or antigen binding fragment thereof comprises a variable heavy region comprising an amino acid sequence at least 87% identical to SEQ ID NO: 162 (VH16). In some embodiments, the integrin α7β1 antibody or antigen binding fragment thereof comprises a variable heavy region comprising an amino acid sequence at least 90% identical to SEQ ID NO: 162 (VH16). In some embodiments, the integrin α7β1 antibody or antigen binding fragment thereof comprises a variable heavy region comprising an amino acid sequence at least 95% identical to SEQ ID NO: 162 (VH16). In some embodiments, the integrin α7β1 antibody or antigen binding fragment thereof comprises a variable heavy region comprising an amino acid sequence at least 98% identical to SEQ ID NO: 162 (VH16). In some embodiments, the integrin α7β1 antibody or antigen binding fragment thereof comprises a variable heavy region comprising an amino acid sequence at least 99% identical to SEQ ID NO: 162 (VH16). In some embodiments, the integrin α7β1 antibody or antigen binding fragment thereof comprises a variable heavy region comprising an amino acid sequence identical to SEQ ID NO: 162 (VH16).

In some embodiments, the integrin α7β1 antibody or antigen binding fragment thereof comprises a variable heavy region comprising an amino acid sequence at least 80% identical to SEQ ID NO: 164 (VH17). In some embodiments, the integrin α7β1 antibody or antigen binding fragment thereof comprises a variable heavy region comprising an amino acid sequence at least 83% identical to SEQ ID NO: 164 (VH17). In some embodiments, the integrin α7β1 antibody or antigen binding fragment thereof comprises a variable heavy region comprising an amino acid sequence at least 85% identical to SEQ ID NO: 164 (VH17). In some embodiments, the integrin α7β1 antibody or antigen binding fragment thereof comprises a variable heavy region comprising an amino acid sequence at least 87% identical to SEQ ID NO: 164 (VH17). In some embodiments, the integrin α7β1 antibody or antigen binding fragment thereof comprises a variable heavy region comprising an amino acid sequence at least 90% identical to SEQ ID NO: 164 (VH17). In some embodiments, the integrin α7β1 antibody or antigen binding fragment thereof comprises a variable heavy region comprising an amino acid sequence at least 95% identical to SEQ ID NO: 164 (VH17). In some embodiments, the integrin α7β1 antibody or antigen binding fragment thereof comprises a variable heavy region comprising an amino acid sequence at least 98% identical to SEQ ID NO: 164 (VH17). In some embodiments, the integrin α7β1 antibody or antigen binding fragment thereof comprises a variable heavy region comprising an amino acid sequence at least 99% identical to SEQ ID NO: 164 (VH17). In some embodiments, the integrin α7β1 antibody or antigen binding fragment thereof comprises a variable heavy region comprising an amino acid sequence identical to SEQ ID NO: 164 (VH17).

In some embodiments, the integrin α7β1 antibody or antigen binding fragment thereof comprises a variable heavy region comprising an amino acid sequence at least 80% identical to SEQ ID NO: 166 (VH18). In some embodiments, the integrin α7β1 antibody or antigen binding fragment thereof comprises a variable heavy region comprising an amino acid sequence at least 83% identical to SEQ ID NO: 166 (VH18). In some embodiments, the integrin α7β1 antibody or antigen binding fragment thereof comprises a variable heavy region comprising an amino acid sequence at least 85% identical to SEQ ID NO: 166 (VH18). In some embodiments, the integrin α7β1 antibody or antigen binding fragment thereof comprises a variable heavy region comprising an amino acid sequence at least 87% identical to SEQ ID NO: 166 (VH18). In some embodiments, the integrin α7β1 antibody or antigen binding fragment thereof comprises a variable heavy region comprising an amino acid sequence at least 90% identical to SEQ ID NO: 166 (VH18). In some embodiments, the integrin α7β1 antibody or antigen binding fragment thereof comprises a variable heavy region comprising an amino acid sequence at least 95% identical to SEQ ID NO: 166 (VH18). In some embodiments, the integrin α7β1 antibody or antigen binding fragment thereof comprises a variable heavy region comprising an amino acid sequence at least 98% identical to SEQ ID NO: 166 (VH18). In some embodiments, the integrin α7β1 antibody or antigen binding fragment thereof comprises a variable heavy region comprising an amino acid sequence at least 99% identical to SEQ ID NO: 166 (VH18). In some embodiments, the integrin α7β1 antibody or antigen binding fragment thereof comprises a variable heavy region comprising an amino acid sequence identical to SEQ ID NO: 166 (VH18).

In some embodiments, the integrin α7β1 antibody or antigen binding fragment thereof comprises a variable heavy region comprising an amino acid sequence at least 80% identical to SEQ ID NO: 170 (VH20). In some embodiments, the integrin α7β1 antibody or antigen binding fragment thereof comprises a variable heavy region comprising an amino acid sequence at least 83% identical to SEQ ID NO: 170 (VH20). In some embodiments, the integrin α7β1 antibody or antigen binding fragment thereof comprises a variable heavy region comprising an amino acid sequence at least 85% identical to SEQ ID NO: 170 (VH20). In some embodiments, the integrin α7β1 antibody or antigen binding fragment thereof comprises a variable heavy region comprising an amino acid sequence at least 87% identical to SEQ ID NO: 170 (VH20). In some embodiments, the integrin α7β1 antibody or antigen binding fragment thereof comprises a variable heavy region comprising an amino acid sequence at least 90% identical to SEQ ID NO: 170 (VH20). In some embodiments, the integrin α7β1 antibody or antigen binding fragment thereof comprises a variable heavy region comprising an amino acid sequence at least 95% identical to SEQ ID NO: 170 (VH20). In some embodiments, the integrin α7β1 antibody or antigen binding fragment thereof comprises a variable heavy region comprising an amino acid sequence at least 98% identical to SEQ ID NO: 170 (VH20). In some embodiments, the integrin α7β1 antibody or antigen binding fragment thereof comprises a variable heavy region comprising an amino acid sequence at least 99% identical to SEQ ID NO: 170 (VH20). In some embodiments, the integrin α7β1 antibody or antigen binding fragment thereof comprises a variable heavy region comprising an amino acid sequence identical to SEQ ID NO: 170 (VH20).

In some embodiments, the integrin α7β1 antibody or antigen binding fragment thereof comprises a variable heavy region comprising an amino acid sequence at least 80% identical to SEQ ID NO: 172 (VH21). In some embodiments, the integrin α7β1 antibody or antigen binding fragment thereof comprises a variable heavy region comprising an amino acid sequence at least 83% identical to SEQ ID NO: 172 (VH21). In some embodiments, the integrin α7β1 antibody or antigen binding fragment thereof comprises a variable heavy region comprising an amino acid sequence at least 85% identical to SEQ ID NO: 172 (VH21). In some embodiments, the integrin α7β1 antibody or antigen binding fragment thereof comprises a variable heavy region comprising an amino acid sequence at least 87% identical to SEQ ID NO: 172 (VH21). In some embodiments, the integrin α7β1 antibody or antigen binding fragment thereof comprises a variable heavy region comprising an amino acid sequence at least 90% identical to SEQ ID NO: 172 (VH21). In some embodiments, the integrin α7β1 antibody or antigen binding fragment thereof comprises a variable heavy region comprising an amino acid sequence at least 95% identical to SEQ ID NO: 172 (VH21). In some embodiments, the integrin α7β1 antibody or antigen binding fragment thereof comprises a variable heavy region comprising an amino acid sequence at least 98% identical to SEQ ID NO: 172 (VH21). In some embodiments, the integrin α7β1 antibody or antigen binding fragment thereof comprises a variable heavy region comprising an amino acid sequence at least 99% identical to SEQ ID NO: 172 (VH21). In some embodiments, the integrin α7β1 antibody or antigen binding fragment thereof comprises a variable heavy region comprising an amino acid sequence identical to SEQ ID NO: 172 (VH21).

In some embodiments, the integrin α7β1 antibody or antigen binding fragment thereof comprises a variable heavy region comprising an amino acid sequence at least 80% identical to SEQ ID NO: 144 (VH22). In some embodiments, the integrin α7β1 antibody or antigen binding fragment thereof comprises a variable heavy region comprising an amino acid sequence at least 83% identical to SEQ ID NO: 144 (VH22). In some embodiments, the integrin α7β1 antibody or antigen binding fragment thereof comprises a variable heavy region comprising an amino acid sequence at least 85% identical to SEQ ID NO: 144 (VH22). In some embodiments, the integrin α7β1 antibody or antigen binding fragment thereof comprises a variable heavy region comprising an amino acid sequence at least 87% identical to SEQ ID NO: 144 (VH22). In some embodiments, the integrin α7β1 antibody or antigen binding fragment thereof comprises a variable heavy region comprising an amino acid sequence at least 90% identical to SEQ ID NO: 144 (VH22). In some embodiments, the integrin α7β1 antibody or antigen binding fragment thereof comprises a variable heavy region comprising an amino acid sequence at least 95% identical to SEQ ID NO: 144 (VH22). In some embodiments, the integrin α7β1 antibody or antigen binding fragment thereof comprises a variable heavy region comprising an amino acid sequence at least 98% identical to SEQ ID NO: 144 (VH22). In some embodiments, the integrin α7β1 antibody or antigen binding fragment thereof comprises a variable heavy region comprising an amino acid sequence at least 99% identical to SEQ ID NO: 144 (VH22). In some embodiments, the integrin α7β1 antibody or antigen binding fragment thereof comprises a variable heavy region comprising an amino acid sequence identical to SEQ ID NO: 144 (VH22).

In some embodiments, the integrin α7β1 antibody or antigen binding fragment thereof comprises a variable heavy region comprising an amino acid sequence at least 80% identical to SEQ ID NO: 146 (VH23). In some embodiments, the integrin α7β1 antibody or antigen binding fragment thereof comprises a variable heavy region comprising an amino acid sequence at least 83% identical to SEQ ID NO: 146 (VH23). In some embodiments, the integrin α7β1 antibody or antigen binding fragment thereof comprises a variable heavy region comprising an amino acid sequence at least 85% identical to SEQ ID NO: 146 (VH23). In some embodiments, the integrin α7β1 antibody or antigen binding fragment thereof comprises a variable heavy region comprising an amino acid sequence at least 87% identical to SEQ ID NO: 146 (VH23). In some embodiments, the integrin α7β1 antibody or antigen binding fragment thereof comprises a variable heavy region comprising an amino acid sequence at least 90% identical to SEQ ID NO: 146 (VH23). In some embodiments, the integrin α7β1 antibody or antigen binding fragment thereof comprises a variable heavy region comprising an amino acid sequence at least 95% identical to SEQ ID NO: 146 (VH23). In some embodiments, the integrin α7β1 antibody or antigen binding fragment thereof comprises a variable heavy region comprising an amino acid sequence at least 98% identical to SEQ ID NO: 146 (VH23). In some embodiments, the integrin α7β1 antibody or antigen binding fragment thereof comprises a variable heavy region comprising an amino acid sequence at least 99% identical to SEQ ID NO: 146 (VH23). In some embodiments, the integrin α7β1 antibody or antigen binding fragment thereof comprises a variable heavy region comprising an amino acid sequence identical to SEQ ID NO: 146 (VH23).

In some embodiments, the integrin α7β1 antibody or antigen binding fragment thereof comprises a variable light region comprising an amino acid sequence at least 80% identical to SEQ ID NO: 143 (VL8). In some embodiments, the integrin α7β1 antibody or antigen binding fragment thereof comprises a variable light region comprising an amino acid sequence at least 83% identical to SEQ ID NO: 134 (VL8). In some embodiments, the integrin α7β1 antibody or antigen binding fragment thereof comprises a variable light region comprising an amino acid sequence at least 85% identical to SEQ ID NO: 143 (VL8). In some embodiments, the integrin α7β1 antibody or antigen binding fragment thereof comprises a variable light region comprising an amino acid sequence at least 87% identical to SEQ ID NO: 143 (VL8). In some embodiments, the integrin α7β1 antibody or antigen binding fragment thereof comprises a variable light region comprising an amino acid sequence at least 90% identical to SEQ ID NO: 143 (VL8). In some embodiments, the integrin α7β1 antibody or antigen binding fragment thereof comprises a variable light region comprising an amino acid sequence at least 95% identical to SEQ ID NO: 143 (VL8). In some embodiments, the integrin α7β1 antibody or antigen binding fragment thereof comprises a variable light region comprising an amino acid sequence at least 98% identical to SEQ ID NO: 143 (VL8). In some embodiments, the integrin α7β1 antibody or antigen binding fragment thereof comprises a variable light region comprising an amino acid sequence at least 99% identical to SEQ ID NO: 143 (VL8). In some embodiments, the integrin α7β1 antibody or antigen binding fragment thereof comprises a variable light region comprising an amino acid sequence identical to SEQ ID NO: 143 (VL8).

In some embodiments, the integrin α7β1 antibody or antigen binding fragment thereof comprises a variable light region comprising an amino acid sequence at least 80% identical to SEQ ID NO: 155 (VL9). In some embodiments, the integrin α7β1 antibody or antigen binding fragment thereof comprises a variable light region comprising an amino acid sequence at least 83% identical to SEQ ID NO: 155 (VL9). In some embodiments, the integrin α7β1 antibody or antigen binding fragment thereof comprises a variable light region comprising an amino acid sequence at least 85% identical to SEQ ID NO: 155 (VL9). In some embodiments, the integrin α7β1 antibody or antigen binding fragment thereof comprises a variable light region comprising an amino acid sequence at least 87% identical to SEQ ID NO: 155 (VL9). In some embodiments, the integrin α7β1 antibody or antigen binding fragment thereof comprises a variable light region comprising an amino acid sequence at least 90% identical to SEQ ID NO: 155 (VL9). In some embodiments, the integrin α7β1 antibody or antigen binding fragment thereof comprises a variable light region comprising an amino acid sequence at least 95% identical to SEQ ID NO: 155 (VL9). In some embodiments, the integrin α7β1 antibody or antigen binding fragment thereof comprises a variable light region comprising an amino acid sequence at least 98% identical to SEQ ID NO: 155 (VL9). In some embodiments, the integrin α7β1 antibody or antigen binding fragment thereof comprises a variable light region comprising an amino acid sequence at least 99% identical to SEQ ID NO: 155 (VL9). In some embodiments, the integrin α7β1 antibody or antigen binding fragment thereof comprises a variable light region comprising an amino acid sequence identical to SEQ ID NO: 155 (VL9).

In some embodiments, the integrin α7β1 antibody or antigen binding fragment thereof comprises a variable light region comprising an amino acid sequence at least 80% identical to SEQ ID NO: 149 (VL10). In some embodiments, the integrin α7β1 antibody or antigen binding fragment thereof comprises a variable light region comprising an amino acid sequence at least 83% identical to SEQ ID NO: 149 (VL10). In some embodiments, the integrin α7β1 antibody or antigen binding fragment thereof comprises a variable light region comprising an amino acid sequence at least 85% identical to SEQ ID NO: 149 (VL10). In some embodiments, the integrin α7β1 antibody or antigen binding fragment thereof comprises a variable light region comprising an amino acid sequence at least 87% identical to SEQ ID NO: 149 (VL10). In some embodiments, the integrin α7β1 antibody or antigen binding fragment thereof comprises a variable light region comprising an amino acid sequence at least 90% identical to SEQ ID NO: 149 (VL10). In some embodiments, the integrin α7β1 antibody or antigen binding fragment thereof comprises a variable light region comprising an amino acid sequence at least 95% identical to SEQ ID NO: 149 (VL10). In some embodiments, the integrin α7β1 antibody or antigen binding fragment thereof comprises a variable light region comprising an amino acid sequence at least 98% identical to SEQ ID NO: 149 (VL10). In some embodiments, the integrin α7β1 antibody or antigen binding fragment thereof comprises a variable light region comprising an amino acid sequence at least 99% identical to SEQ ID NO: 149 (VL10). In some embodiments, the integrin α7β1 antibody or antigen binding fragment thereof comprises a variable light region comprising an amino acid sequence identical to SEQ ID NO: 149 (VL10).

In some embodiments, the integrin α7β1 antibody or antigen binding fragment thereof comprises a variable light region comprising an amino acid sequence at least 80% identical to SEQ ID NO: 151 (VL11). In some embodiments, the integrin α7β1 antibody or antigen binding fragment thereof comprises a variable light region comprising an amino acid sequence at least 83% identical to SEQ ID NO: 151 (VL11). In some embodiments, the integrin α7β1 antibody or antigen binding fragment thereof comprises a variable light region comprising an amino acid sequence at least 85% identical to SEQ ID NO: 151 (VL11). In some embodiments, the integrin α7β1 antibody or antigen binding fragment thereof comprises a variable light region comprising an amino acid sequence at least 87% identical to SEQ ID NO: 151 (VL11). In some embodiments, the integrin α7β1 antibody or antigen binding fragment thereof comprises a variable light region comprising an amino acid sequence at least 90% identical to SEQ ID NO: 151 (VL11). In some embodiments, the integrin α7β1 antibody or antigen binding fragment thereof comprises a variable light region comprising an amino acid sequence at least 95% identical to SEQ ID NO: 151 (VL11). In some embodiments, the integrin α7β1 antibody or antigen binding fragment thereof comprises a variable light region comprising an amino acid sequence at least 98% identical to SEQ ID NO: 151 (VL11). In some embodiments, the integrin α7β1 antibody or antigen binding fragment thereof comprises a variable light region comprising an amino acid sequence at least 99% identical to SEQ ID NO: 151 (VL11). In some embodiments, the integrin α7β1 antibody or antigen binding fragment thereof comprises a variable light region comprising an amino acid sequence identical to SEQ ID NO: 151 (VL11).

In some embodiments, the integrin α7β1 antibody or antigen binding fragment thereof comprises a variable light region comprising an amino acid sequence at least 80% identical to SEQ ID NO: 153 (VL12). In some embodiments, the integrin α7β1 antibody or antigen binding fragment thereof comprises a variable light region comprising an amino acid sequence at least 83% identical to SEQ ID NO: 153 (VL12). In some embodiments, the integrin α7β1 antibody or antigen binding fragment thereof comprises a variable light region comprising an amino acid sequence at least 85% identical to SEQ ID NO: 153 (VL12). In some embodiments, the integrin α7β1 antibody or antigen binding fragment thereof comprises a variable light region comprising an amino acid sequence at least 87% identical to SEQ ID NO: 153 (VL12). In some embodiments, the integrin α7β1 antibody or antigen binding fragment thereof comprises a variable light region comprising an amino acid sequence at least 90% identical to SEQ ID NO: 153 (VL12). In some embodiments, the integrin α7β1 antibody or antigen binding fragment thereof comprises a variable light region comprising an amino acid sequence at least 95% identical to SEQ ID NO: 153 (VL12). In some embodiments, the integrin α7β1 antibody or antigen binding fragment thereof comprises a variable light region comprising an amino acid sequence at least 98% identical to SEQ ID NO: 153 (VL12). In some embodiments, the integrin α7β1 antibody or antigen binding fragment thereof comprises a variable light region comprising an amino acid sequence at least 99% identical to SEQ ID NO: 153 (VL12). In some embodiments, the integrin α7β1 antibody or antigen binding fragment thereof comprises a variable light region comprising an amino acid sequence identical to SEQ ID NO: 153 (VL12).

In some embodiments, the integrin α7β1 antibody or antigen binding fragment thereof comprises a variable light region comprising an amino acid sequence at least 80% identical to SEQ ID NO: 157 (VL13). In some embodiments, the integrin α7β1 antibody or antigen binding fragment thereof comprises a variable light region comprising an amino acid sequence at least 83% identical to SEQ ID NO: 157 (VL13). In some embodiments, the integrin α7β1 antibody or antigen binding fragment thereof comprises a variable light region comprising an amino acid sequence at least 85% identical to SEQ ID NO: 157 (VL13). In some embodiments, the integrin α7β1 antibody or antigen binding fragment thereof comprises a variable light region comprising an amino acid sequence at least 87% identical to SEQ ID NO: 157 (VL13). In some embodiments, the integrin α7β1 antibody or antigen binding fragment thereof comprises a variable light region comprising an amino acid sequence at least 90% identical to SEQ ID NO: 157 (VL13). In some embodiments, the integrin α7β1 antibody or antigen binding fragment thereof comprises a variable light region comprising an amino acid sequence at least 95% identical to SEQ ID NO: 157 (VL13). In some embodiments, the integrin α7β1 antibody or antigen binding fragment thereof comprises a variable light region comprising an amino acid sequence at least 98% identical to SEQ ID NO: 157 (VL13). In some embodiments, the integrin α7β1 antibody or antigen binding fragment thereof comprises a variable light region comprising an amino acid sequence at least 99% identical to SEQ ID NO: 157 (VL13). In some embodiments, the integrin α7β1 antibody or antigen binding fragment thereof comprises a variable light region comprising an amino acid sequence identical to SEQ ID NO: 157 (VL13).

In some embodiments, the integrin α7β1 antibody or antigen binding fragment thereof comprises a variable light region comprising an amino acid sequence at least 80% identical to SEQ ID NO: 159 (VL14). In some embodiments, the integrin α7β1 antibody or antigen binding fragment thereof comprises a variable light region comprising an amino acid sequence at least 83% identical to SEQ ID NO: 159 (VL14). In some embodiments, the integrin α7β1 antibody or antigen binding fragment thereof comprises a variable light region comprising an amino acid sequence at least 85% identical to SEQ ID NO: 159 (VL14). In some embodiments, the integrin α7β1 antibody or antigen binding fragment thereof comprises a variable light region comprising an amino acid sequence at least 87% identical to SEQ ID NO: 159 (VL14). In some embodiments, the integrin α7β1 antibody or antigen binding fragment thereof comprises a variable light region comprising an amino acid sequence at least 90% identical to SEQ ID NO: 159 (VL14). In some embodiments, the integrin α7β1 antibody or antigen binding fragment thereof comprises a variable light region comprising an amino acid sequence at least 95% identical to SEQ ID NO: 159 (VL14). In some embodiments, the integrin α7β1 antibody or antigen binding fragment thereof comprises a variable light region comprising an amino acid sequence at least 98% identical to SEQ ID NO: 159 (VL14). In some embodiments, the integrin α7β1 antibody or antigen binding fragment thereof comprises a variable light region comprising an amino acid sequence at least 99% identical to SEQ ID NO: 159 (VL14). In some embodiments, the integrin α7β1 antibody or antigen binding fragment thereof comprises a variable light region comprising an amino acid sequence identical to SEQ ID NO: 159 (VL14).

In some embodiments, the integrin α7β1 antibody or antigen binding fragment thereof comprises a variable light region comprising an amino acid sequence at least 80% identical to SEQ ID NO: 161 (VL15). In some embodiments, the integrin α7β1 antibody or antigen binding fragment thereof comprises a variable light region comprising an amino acid sequence at least 83% identical to SEQ ID NO: 161 (VL15). In some embodiments, the integrin α7β1 antibody or antigen binding fragment thereof comprises a variable light region comprising an amino acid sequence at least 85% identical to SEQ ID NO: 161 (VL15). In some embodiments, the integrin α7β1 antibody or antigen binding fragment thereof comprises a variable light region comprising an amino acid sequence at least 87% identical to SEQ ID NO: 161 (VL15). In some embodiments, the integrin α7β1 antibody or antigen binding fragment thereof comprises a variable light region comprising an amino acid sequence at least 90% identical to SEQ ID NO: 161 (VL15). In some embodiments, the integrin α7β1 antibody or antigen binding fragment thereof comprises a variable light region comprising an amino acid sequence at least 95% identical to SEQ ID NO: 161 (VL15). In some embodiments, the integrin α7β1 antibody or antigen binding fragment thereof comprises a variable light region comprising an amino acid sequence at least 98% identical to SEQ ID NO: 161 (VL15). In some embodiments, the integrin α7β1 antibody or antigen binding fragment thereof comprises a variable light region comprising an amino acid sequence at least 99% identical to SEQ ID NO: 161 (VL15). In some embodiments, the integrin α7β1 antibody or antigen binding fragment thereof comprises a variable light region comprising an amino acid sequence identical to SEQ ID NO: 161 (VL15).

In some embodiments, the integrin α7β1 antibody or antigen binding fragment thereof comprises a variable light region comprising an amino acid sequence at least 80% identical to SEQ ID NO: 163 (VL16). In some embodiments, the integrin α7β1 antibody or antigen binding fragment thereof comprises a variable light region comprising an amino acid sequence at least 83% identical to SEQ ID NO: 163 (VL16). In some embodiments, the integrin α7β1 antibody or antigen binding fragment thereof comprises a variable light region comprising an amino acid sequence at least 85% identical to SEQ ID NO: 163 (VL16). In some embodiments, the integrin α7β1 antibody or antigen binding fragment thereof comprises a variable light region comprising an amino acid sequence at least 87% identical to SEQ ID NO: 163 (VL16). In some embodiments, the integrin α7β1 antibody or antigen binding fragment thereof comprises a variable light region comprising an amino acid sequence at least 90% identical to SEQ ID NO: 163 (VL16). In some embodiments, the integrin α7β1 antibody or antigen binding fragment thereof comprises a variable light region comprising an amino acid sequence at least 95% identical to SEQ ID NO: 163 (VL16). In some embodiments, the integrin α7β1 antibody or antigen binding fragment thereof comprises a variable light region comprising an amino acid sequence at least 98% identical to SEQ ID NO: 163 (VL16). In some embodiments, the integrin α7β1 antibody or antigen binding fragment thereof comprises a variable light region comprising an amino acid sequence at least 99% identical to SEQ ID NO: 163 (VL16). In some embodiments, the integrin α7β1 antibody or antigen binding fragment thereof comprises a variable light region comprising an amino acid sequence identical to SEQ ID NO: 163 (VL16).

In some embodiments, the integrin α7β1 antibody or antigen binding fragment thereof comprises a variable light region comprising an amino acid sequence at least 80% identical to SEQ ID NO: 165 (VL17). In some embodiments, the integrin α7β1 antibody or antigen binding fragment thereof comprises a variable light region comprising an amino acid sequence at least 83% identical to SEQ ID NO: 165 (VL17). In some embodiments, the integrin α7β1 antibody or antigen binding fragment thereof comprises a variable light region comprising an amino acid sequence at least 85% identical to SEQ ID NO: 165 (VL17). In some embodiments, the integrin α7β1 antibody or antigen binding fragment thereof comprises a variable light region comprising an amino acid sequence at least 87% identical to SEQ ID NO: 165 (VL17). In some embodiments, the integrin α7β1 antibody or antigen binding fragment thereof comprises a variable light region comprising an amino acid sequence at least 90% identical to SEQ ID NO: 165 (VL17). In some embodiments, the integrin α7β1 antibody or antigen binding fragment thereof comprises a variable light region comprising an amino acid sequence at least 95% identical to SEQ ID NO: 165 (VL17). In some embodiments, the integrin α7β1 antibody or antigen binding fragment thereof comprises a variable light region comprising an amino acid sequence at least 98% identical to SEQ ID NO: 165 (VL17). In some embodiments, the integrin α7β1 antibody or antigen binding fragment thereof comprises a variable light region comprising an amino acid sequence at least 99% identical to SEQ ID NO: 165 (VL17). In some embodiments, the integrin α7β1 antibody or antigen binding fragment thereof comprises a variable light region comprising an amino acid sequence identical to SEQ ID NO: 165 (VL17).

In some embodiments, the integrin α7β1 antibody or antigen binding fragment thereof comprises a variable light region comprising an amino acid sequence at least 80% identical to SEQ ID NO: 167 (VL18). In some embodiments, the integrin α7β1 antibody or antigen binding fragment thereof comprises a variable light region comprising an amino acid sequence at least 83% identical to SEQ ID NO: 167 (VL18). In some embodiments, the integrin α7β1 antibody or antigen binding fragment thereof comprises a variable light region comprising an amino acid sequence at least 85% identical to SEQ ID NO: 167 (VL18). In some embodiments, the integrin α7β1 antibody or antigen binding fragment thereof comprises a variable light region comprising an amino acid sequence at least 87% identical to SEQ ID NO: 167 (VL18). In some embodiments, the integrin α7β1 antibody or antigen binding fragment thereof comprises a variable light region comprising an amino acid sequence at least 90% identical to SEQ ID NO: 167 (VL18). In some embodiments, the integrin α7β1 antibody or antigen binding fragment thereof comprises a variable light region comprising an amino acid sequence at least 95% identical to SEQ ID NO: 167 (VL18). In some embodiments, the integrin α7β1 antibody or antigen binding fragment thereof comprises a variable light region comprising an amino acid sequence at least 98% identical to SEQ ID NO: 167 (VL18). In some embodiments, the integrin α7β1 antibody or antigen binding fragment thereof comprises a variable light region comprising an amino acid sequence at least 99% identical to SEQ ID NO: 167 (VL18). In some embodiments, the integrin α7β1 antibody or antigen binding fragment thereof comprises a variable light region comprising an amino acid sequence identical to SEQ ID NO: 167 (VL18).

In some embodiments, the integrin α7β1 antibody or antigen binding fragment thereof comprises a variable light region comprising an amino acid sequence at least 80% identical to SEQ ID NO: 167 (VL18). In some embodiments, the integrin α7β1 antibody or antigen binding fragment thereof comprises a variable light region comprising an amino acid sequence at least 83% identical to SEQ ID NO: 167 (VL18). In some embodiments, the integrin α7β1 antibody or antigen binding fragment thereof comprises a variable light region comprising an amino acid sequence at least 85% identical to SEQ ID NO: 167 (VL18). In some embodiments, the integrin α7β1 antibody or antigen binding fragment thereof comprises a variable light region comprising an amino acid sequence at least 87% identical to SEQ ID NO: 167 (VL18). In some embodiments, the integrin α7β1 antibody or antigen binding fragment thereof comprises a variable light region comprising an amino acid sequence at least 90% identical to SEQ ID NO: 167 (VL18). In some embodiments, the integrin α7β1 antibody or antigen binding fragment thereof comprises a variable light region comprising an amino acid sequence at least 95% identical to SEQ ID NO: 167 (VL18). In some embodiments, the integrin α7β1 antibody or antigen binding fragment thereof comprises a variable light region comprising an amino acid sequence at least 98% identical to SEQ ID NO: 167 (VL18). In some embodiments, the integrin α7β1 antibody or antigen binding fragment thereof comprises a variable light region comprising an amino acid sequence at least 99% identical to SEQ ID NO: 167 (VL18). In some embodiments, the integrin α7β1 antibody or antigen binding fragment thereof comprises a variable light region comprising an amino acid sequence identical to SEQ ID NO: 167 (VL18).

In some embodiments, the integrin α7β1 antibody or antigen binding fragment thereof comprises a variable light region comprising an amino acid sequence at least 80% identical to SEQ ID NO: 169 (VL19). In some embodiments, the integrin α7β1 antibody or antigen binding fragment thereof comprises a variable light region comprising an amino acid sequence at least 83% identical to SEQ ID NO: 169 (VL19). In some embodiments, the integrin α7β1 antibody or antigen binding fragment thereof comprises a variable light region comprising an amino acid sequence at least 85% identical to SEQ ID NO: 169 (VL19). In some embodiments, the integrin α7β1 antibody or antigen binding fragment thereof comprises a variable light region comprising an amino acid sequence at least 87% identical to SEQ ID NO: 169 (VL19). In some embodiments, the integrin α7β1 antibody or antigen binding fragment thereof comprises a variable light region comprising an amino acid sequence at least 90% identical to SEQ ID NO: 169 (VL19). In some embodiments, the integrin α7β1 antibody or antigen binding fragment thereof comprises a variable light region comprising an amino acid sequence at least 95% identical to SEQ ID NO: 169 (VL19). In some embodiments, the integrin α7β1 antibody or antigen binding fragment thereof comprises a variable light region comprising an amino acid sequence at least 98% identical to SEQ ID NO: 169 (VL19). In some embodiments, the integrin α7β1 antibody or antigen binding fragment thereof comprises a variable light region comprising an amino acid sequence at least 99% identical to SEQ ID NO: 169 (VL19). In some embodiments, the integrin α7β1 antibody or antigen binding fragment thereof comprises a variable light region comprising an amino acid sequence identical to SEQ ID NO: 169 (VL19).

In some embodiments, the integrin α7β1 antibody or antigen binding fragment thereof comprises a variable light region comprising an amino acid sequence at least 80% identical to SEQ ID NO: 171 (VL20). In some embodiments, the integrin α7β1 antibody or antigen binding fragment thereof comprises a variable light region comprising an amino acid sequence at least 83% identical to SEQ ID NO: 171 (VL20). In some embodiments, the integrin α7β1 antibody or antigen binding fragment thereof comprises a variable light region comprising an amino acid sequence at least 85% identical to SEQ ID NO: 171 (VL20). In some embodiments, the integrin α7β1 antibody or antigen binding fragment thereof comprises a variable light region comprising an amino acid sequence at least 87% identical to SEQ ID NO: 171 (VL20). In some embodiments, the integrin α7β1 antibody or antigen binding fragment thereof comprises a variable light region comprising an amino acid sequence at least 90% identical to SEQ ID NO: 171 (VL20). In some embodiments, the integrin α7β1 antibody or antigen binding fragment thereof comprises a variable light region comprising an amino acid sequence at least 95% identical to SEQ ID NO: 171 (VL20). In some embodiments, the integrin α7β1 antibody or antigen binding fragment thereof comprises a variable light region comprising an amino acid sequence at least 98% identical to SEQ ID NO: 171 (VL20). In some embodiments, the integrin α7β1 antibody or antigen binding fragment thereof comprises a variable light region comprising an amino acid sequence at least 99% identical to SEQ ID NO: 171 (VL20). In some embodiments, the integrin α7β1 antibody or antigen binding fragment thereof comprises a variable light region comprising an amino acid sequence identical to SEQ ID NO: 171 (VL20).

In some embodiments, the integrin α7β1 antibody or antigen binding fragment thereof comprises a variable light region comprising an amino acid sequence at least 80% identical to SEQ ID NO: 173 (VL21). In some embodiments, the integrin α7β1 antibody or antigen binding fragment thereof comprises a variable light region comprising an amino acid sequence at least 83% identical to SEQ ID NO: 173 (VL21). In some embodiments, the integrin α7β1 antibody or antigen binding fragment thereof comprises a variable light region comprising an amino acid sequence at least 85% identical to SEQ ID NO: 173 (VL21). In some embodiments, the integrin α7β1 antibody or antigen binding fragment thereof comprises a variable light region comprising an amino acid sequence at least 87% identical to SEQ ID NO: 173 (VL21). In some embodiments, the integrin α7β1 antibody or antigen binding fragment thereof comprises a variable light region comprising an amino acid sequence at least 90% identical to SEQ ID NO: 173 (VL21). In some embodiments, the integrin α7β1 antibody or antigen binding fragment thereof comprises a variable light region comprising an amino acid sequence at least 95% identical to SEQ ID NO: 173 (VL21). In some embodiments, the integrin α7β1 antibody or antigen binding fragment thereof comprises a variable light region comprising an amino acid sequence at least 98% identical to SEQ ID NO: 173 (VL21). In some embodiments, the integrin α7β1 antibody or antigen binding fragment thereof comprises a variable light region comprising an amino acid sequence at least 99% identical to SEQ ID NO: 73 (VL21). In some embodiments, the integrin α7β1 antibody or antigen binding fragment thereof comprises a variable light region comprising an amino acid sequence identical to SEQ ID NO: 173 (VL21).

In some embodiments, the integrin α7β1 antibody or antigen binding fragment thereof comprises a variable light region comprising an amino acid sequence at least 80% identical to SEQ ID NO: 145 (VL22). In some embodiments, the integrin α7β1 antibody or antigen binding fragment thereof comprises a variable light region comprising an amino acid sequence at least 83% identical to SEQ ID NO: 145 (VL22). In some embodiments, the integrin α7β1 antibody or antigen binding fragment thereof comprises a variable light region comprising an amino acid sequence at least 85% identical to SEQ ID NO: 145 (VL22). In some embodiments, the integrin α7β1 antibody or antigen binding fragment thereof comprises a variable light region comprising an amino acid sequence at least 87% identical to SEQ ID NO: 145 (VL22). In some embodiments, the integrin α7β1 antibody or antigen binding fragment thereof comprises a variable light region comprising an amino acid sequence at least 90% identical to SEQ ID NO: 145 (VL22). In some embodiments, the integrin α7β1 antibody or antigen binding fragment thereof comprises a variable light region comprising an amino acid sequence at least 95% identical to SEQ ID NO: 145 (VL22). In some embodiments, the integrin α7β1 antibody or antigen binding fragment thereof comprises a variable light region comprising an amino acid sequence at least 98% identical to SEQ ID NO: 145 (VL22). In some embodiments, the integrin α7β1 antibody or antigen binding fragment thereof comprises a variable light region comprising an amino acid sequence at least 99% identical to SEQ ID NO: 145 (VL22). In some embodiments, the integrin α7β1 antibody or antigen binding fragment thereof comprises a variable light region comprising an amino acid sequence identical to SEQ ID NO: 145 (VL22).

In some embodiments, the integrin α7β1 antibody or antigen binding fragment thereof comprises a variable light region comprising an amino acid sequence at least 80% identical to SEQ ID NO: 147 (VL23). In some embodiments, the integrin α7β1 antibody or antigen binding fragment thereof comprises a variable light region comprising an amino acid sequence at least 83% identical to SEQ ID NO: 147 (VL23). In some embodiments, the integrin α7β1 antibody or antigen binding fragment thereof comprises a variable light region comprising an amino acid sequence at least 85% identical to SEQ ID NO: 147 (VL23). In some embodiments, the integrin α7β1 antibody or antigen binding fragment thereof comprises a variable light region comprising an amino acid sequence at least 87% identical to SEQ ID NO: 147 (VL23). In some embodiments, the integrin α7β1 antibody or antigen binding fragment thereof comprises a variable light region comprising an amino acid sequence at least 90% identical to SEQ ID NO: 147 (VL23). In some embodiments, the integrin α7β1 antibody or antigen binding fragment thereof comprises a variable light region comprising an amino acid sequence at least 95% identical to SEQ ID NO: 147 (VL23). In some embodiments, the integrin α7β1 antibody or antigen binding fragment thereof comprises a variable light region comprising an amino acid sequence at least 98% identical to SEQ ID NO: 147 (VL23). In some embodiments, the integrin α7β1 antibody or antigen binding fragment thereof comprises a variable light region comprising an amino acid sequence at least 99% identical to SEQ ID NO: 147 (VL23). In some embodiments, the integrin α7β1 antibody or antigen binding fragment thereof comprises a variable light region comprising an amino acid sequence identical to SEQ ID NO: 147 (VL23).

In some embodiments, the integrin α7β1 antibody or antigen binding fragment thereof comprises a heavy variable region comprising an amino acid sequence of SEQ ID NO: 142 (VH8) and a light variable region comprising an amino acid sequence of SEQ ID NO: 143 (VL8).

In some embodiments, the integrin α7β1 antibody or antigen binding fragment thereof comprises a heavy variable region comprising an amino acid sequence of SEQ ID NO: 154 (VH9) and a light variable region comprising an amino acid sequence of SEQ ID NO: 155 (VL9).

In some embodiments, the integrin α7β1 antibody or antigen binding fragment thereof comprises a heavy variable region comprising an amino acid sequence of SEQ ID NO: 149 (VH10) and a light variable region comprising an amino acid sequence of SEQ ID NO: 149 (VL10).

In some embodiments, the integrin α7β1 antibody or antigen binding fragment thereof comprises a heavy variable region comprising an amino acid sequence of SEQ ID NO: 150 (VH11) and a light variable region comprising an amino acid sequence of SEQ ID NO: 151 (VL11).

In some embodiments, the integrin α7β1 antibody or antigen binding fragment thereof comprises a heavy variable region comprising an amino acid sequence of SEQ ID NO: 152 (VH12) and a light variable region comprising an amino acid sequence of SEQ ID NO: 153 (VL12).

In some embodiments, the integrin α7β1 antibody or antigen binding fragment thereof comprises a heavy variable region comprising an amino acid sequence of SEQ ID NO: 156 (VH13) and a light variable region comprising an amino acid sequence of SEQ ID NO: 157 (VL13).

In some embodiments, the integrin α7β1 antibody or antigen binding fragment thereof comprises a heavy variable region comprising an amino acid sequence of SEQ ID NO: 158 (VH14) and a light variable region comprising an amino acid sequence of SEQ ID NO: 159 (VL14).

In some embodiments, the integrin α7β1 antibody or antigen binding fragment thereof comprises a heavy variable region comprising an amino acid sequence of SEQ ID NO: 160 (VH15) and a light variable region comprising an amino acid sequence of SEQ ID NO: 161 (VL15).

In some embodiments, the integrin α7β1 antibody or antigen binding fragment thereof comprises a heavy variable region comprising an amino acid sequence of SEQ ID NO: 162 (VH16) and a light variable region comprising an amino acid sequence of SEQ ID NO: 163 (VL16).

In some embodiments, the integrin α7β1 antibody or antigen binding fragment thereof comprises a heavy variable region comprising an amino acid sequence of SEQ ID NO: 164 (VH17) and a light variable region comprising an amino acid sequence of SEQ ID NO: 165 (VL17).

In some embodiments, the integrin α7β1 antibody or antigen binding fragment thereof comprises a heavy variable region comprising an amino acid sequence of SEQ ID NO: 166 (VH18) and a light variable region comprising an amino acid sequence of SEQ ID NO: 167 (VL18).

In some embodiments, the integrin α7β1 antibody or antigen binding fragment thereof comprises a heavy variable region comprising an amino acid sequence of SEQ ID NO: 170 (VH20) and a light variable region comprising an amino acid sequence of SEQ ID NO: 171 (VL20).

In some embodiments, the integrin α7β1 antibody or antigen binding fragment thereof comprises a heavy variable region comprising an amino acid sequence of SEQ ID NO: 172 (VH21) and a light variable region comprising an amino acid sequence of SEQ ID NO: 173 (VL21).

In some embodiments, the integrin α7β1 antibody or antigen binding fragment thereof comprises a heavy variable region comprising an amino acid sequence of SEQ ID NO: 144 (VH22) and a light variable region comprising an amino acid sequence of SEQ ID NO: 145 (VL22).

In some embodiments, the integrin α7β1 antibody or antigen binding fragment thereof comprises a heavy variable region comprising an amino acid sequence of SEQ ID NO: 146 (VH23) and a light variable region comprising an amino acid sequence of SEQ ID NO: 147 (VL23).

Humanized, Affinity-Optimized Anti-α7β1 Integrin Antibodies

In some embodiments, the integrin α7β1 antibody or antigen binding fragment comprises at least one, two, or three complementary determining regions (CDRs) from a heavy chain variable region having an amino acid sequence as set forth in Table E, or a sequence substantially homologous thereto (e.g., a sequence at least about 85%, 90%, 95%, 99% or more identical thereto, and/or having one or more substitutions, e.g., conserved substitutions). In some embodiments, integrin α7β1 antibody or antigen binding fragment thereof comprises at least one, two, or three CDRs from a light chain variable region having an amino acid sequence as set forth in Table E, or a sequence substantially homologous thereto (e.g., a sequence at least about 85%, 90%, 95%, 99% or more identical thereto, and/or having one or more substitutions, e.g., conserved substitutions). In an embodiment, the integrin α7β1 antibody or antigen binding fragment thereof comprises at least one, two, three, four, five, or six CDRs from heavy and light chain variable regions having an amino acid sequence as set forth in Table E, or a sequence substantially homologous thereto (e.g., a sequence at least about 85%, 90%, 95%, 99% or more identical thereto, and/or having one or more substitutions, e.g., conserved substitutions).

In some embodiments, the antibody or antigen-binding fragment thereof is a humanized α7β1 antibody. In some embodiments, the antibody or antigen-binding fragment thereof comprises CDRs selected from Table E. Unless indicated otherwise, the CDR sequences shown in the present application are in the IMGT numbering scheme.

TABLE E
Exemplary CDRs of humanized, affinity optimized anti-α7β1 antibodies.

| Ab | HCDR1 | HCDR2 | HCDR3 | LCDR1 | LCDR2 | LCDR3 |
|---|---|---|---|---|---|---|
| Ab-P | GYSFTRYW | IDPSDSET | ARGGYFGEGWYF | QSLVHRNG | KVS (SEQ | SQSTHVPWT |
| | (SEQ ID | (SEQ ID | DV (SEQ ID | NTY (SEQ | ID NO: | (SEQ ID NO: |
| | NO: 101) | NO: 27) | NO: 38) | ID NO: | 63) | 75) |
| | | | | 105) | | |
| Ab24 | GYSFTRYW | IDPSDSET | ARGGYFGREWYY | QSLVHRNG | EVS (SEQ | SQSTHVPWT |
| | (SEQ ID | (SEQ ID | DV (SEQ ID | NTY (SEQ | ID NO: | (SEQ ID |
| | NO: 101) | NO: 102) | NO: 103) | ID NO: | 106) | NO: 75, |
| | | | | 105) | | LCDR3G) |
| Ab25 | GYSFTRYW | IDPSDSET | ARGGYFGREWYY | QSLVHRNG | EVS (SEQ | SQSTHVPWT |
| | (SEQ ID | (SEQ ID | DV (SEQ ID | NTY (SEQ | ID NO: | (SEQ ID NO: |
| | NO: 101) | NO: 102) | NO: 103) | ID NO: | 106) | 75, LCDR3G) |
| | | | | 105) | | |
| Ab26 | GYSFTRYW | IDPSDSET | ARGGYFGEEWYH | QSLVHRNG | EVS (SEQ | AQSTQLPYT |
| | (SEQ ID | (SEQ ID | DV (SEQ ID | NTY (SEQ | ID No: | (SEQ ID NO: |
| | NO: 101) | NO: 102) | NO: 104) | ID NO: | 106) | 107) |
| | | | | 105) | | |
| Ab27 | GYSFTRYW | IDPSDSET | ARGGYFGEEWYH | QSLVHRNG | EVS (SEQ | AQSTOLPYT |
| | (SEQ ID | (SEQ ID | DV (SEQ ID | NTY (SEQ | ID NO: | (SEQ ID NO: |
| | NO: 101) | NO: 102) | NO: 104) | ID NO: | 106) | 107) |
| | | | | 105) | | |
| Ab30 | GYSFTRYW | IDPSDSET | ARGGYFGEGWYH | QSLVHRNG | EVS (SEQ | AQSIQVPWT |
| | (SEQ ID | (SEQ ID | DV (SEQ ID | NTY (SEQ | ID NO: | (SEQ ID NO: |
| | NO: 101) | NO: 102) | NO: 188) | ID NO: | 106) | 198) |
| | | | | 105) | | |
| Ab31 | GYSFTRYW | IDPSDSET | ARGGYFGRQWYF | QSLVHRNG | EVS (SEQ | SQSTHVPWT |
| | (SEQ ID | (SEQ ID | DV (SEQ ID | NTY (SEQ | ID NO: | (SEQ ID NO: |
| | NO: 101) | NO: 102) | NO: 189) | ID NO: | 106) | 75) |
| | | | | 105) | | |
| Ab32 | GYSFTRYW | IDPSDSET | ARGGYFGEQWYH | QSLVHRNG | EVS (SEQ | SQSTHVPWT |
| | (SEQ ID | (SEQ ID | DV (SEQ ID | NTY (SEQ | ID NO: | (SEQ ID NO: |
| | NO: 101) | NO: 102) | NO: 190) | ID NO: | 106) | 75) |
| | | | | 105) | | |
| Ab33 | GYSFTRYW | IDPSDSET | ARGGYFGVGWYN | QSLVHRNG | EVS (SEQ | SQSTHVPWT |
| | (SEQ ID | (SEQ ID | DV (SEQ ID | NTY (SEQ | ID NO: | (SEQ ID NO: |
| | NO: 101) | NO: 102) | NO: 191) | ID NO: | 106) | 75) |
| | | | | 105) | | |
| Ab34 | GYSFTRYW | IDPSDSET | ARGGYFGETWYH | QSLVHRNG | EVS (SEQ | SQSTHVPWT |
| | (SEQ ID | (SEQ ID | DV (SEQ ID | NTY (SEQ | ID NO: | (SEQ ID NO: |
| | NO: 101) | NO: 102) | NO: 192) | ID NO: | 106) | 75) |
| | | | | 105) | | |
| Ab35 | GYSFTRYW | IDPSDSET | ARGGYFGENWYH | QSLVHRNG | EVS (SEQ | AQSIQVPWT |
| | (SEQ ID | (SEQ ID | DV (SEQ ID | NTY (SEQ | ID NO: | (SEQ ID NO: |
| | NO: 101) | NO: 102) | NO: 193) | ID NO: | 106) | 198) |
| | | | | 105) | | |
| Ab36 | GYSFTRYW | IDPSDSET | ARGGYFGEKWYH | QSLVHRNG | EVS (SEQ | AQSTOLPYT |
| | (SEQ ID | (SEQ ID | DV (SEQ ID | NTY (SEQ | ID NO: | (SEQ ID NO: |
| | NO: 101) | NO: 102) | NO: 194) | ID NO: | 106) | 107) |
| | | | | 105) | | |
| Ab37 | GYSFTRYW | IDPSDSET | ARGGYFGEKWYH | QSLVHRNG | GVS (SEQ | AQSTQLPYT |
| | (SEQ ID | (SEQ ID | DV (SEQ ID | NTY (SEQ | ID NO: | (SEQ ID NO: |
| | NO: 101) | NO: 102) | NO: 194) | ID NO: | 196) | 107) |
| | | | | 105) | | |
| Ab38 | GYSFTRYW | IDPSDSET | ARGGYFGEEWYN | QSLVHRNG | KVS (SEQ | SQSTHVPWT |
| | (SEQ ID | (SEQ ID | DV (SEQ ID | NTY (SEQ | ID NO: | (SEQ ID NO: |
| | NO: 101) | NO: 102) | NO: 195) | ID NO: | 63) | 75) |
| | | | | 105) | | |
| Ab39 | GYSFTRYW | IDPSDSET | ARGGYFGEEWYH | QSLVHRNG | KVS (SEQ | SQSTHVPWT |
| | (SEQ ID | (SEQ ID | DV (SEQ ID | NTY (SEQ | ID NO: | (SEQ ID NO: |
| | NO: 101) | NO: 102) | NO: 104) | ID NO: | 63) | 75) |
| | | | | 105) | | |
| Ab40 | GYSFTRYW | IDPSDSET | ARGGYFGEEWYH | QSLVHRNG | EGS (SEQ | SQSTHVPWT |
| | (SEQ ID | (SEQ ID | DV (SEQ ID | NTY (SEQ | ID NO: | (SEQ ID NO: |
| | NO: 101) | NO: 102) | NO: 104) | ID NO: | 197) | 75) |
| | | | | 105) | | |
| Ab41 | GYSFTRYW | IDPSDSET | ARGGYFGEQWYH | QSLVHRNG | EVS (SEQ | AQSIQVPWT |
| | (SEQ ID | (SEQ ID | DV (SEQ ID | NTY (SEQ | ID NO: | (SEQ ID NO: |
| | NO: 101) | NO: 102) | NO: 190) | ID NO: | 106) | 198) |
| | | | | 105) | | |
| Ab42 | GYSFTRYW | IDPSDSET | ARGGYFGRQWYF | QSLVHRNG | EGS (SEQ | (SEQ ID NO: |
| | (SEQ ID | (SEQ ID | DV (SEQ ID | NTY (SEQ | ID NO: | AQSTQLPYT |
| | NO: 101) | NO: 102) | NO: 189) | ID NO: | 197) | 107) |
| | | | | 105) | | |

In some embodiments, the antibody or antigen-binding fragment thereof comprises an HCDR1 of SEQ ID NO: 101, an HCDR2 of SEQ ID NO: 27, and an HCDR3 of SEQ ID NO: 38. In some embodiments, the antibody or antigen-binding fragment thereof comprises an LCDR1 of SEQ ID NO: 105, an LCDR2 of SEQ ID NO: 63, and an LCDR3 of SEQ ID NO: 75. In some embodiments, the antibody or antigen-binding fragment thereof comprises an HCDR1 of SEQ ID NO: 101, an HCDR2 of SEQ ID NO: 27, an HCDR3 of SEQ ID NO: 38, an LCDR1 of SEQ ID NO: 105, an LCDR2 of SEQ ID NO: 63, and an LCDR3 of SEQ ID NO: 75.

In some embodiments, the antibody or antigen-binding fragment thereof competes with a reference antibody, wherein the reference antibody comprises an HCDR1 of SEQ ID NO: 101, an HCDR2 of SEQ ID NO: 27, an HCDR3 of SEQ ID NO: 38, an LCDR1 of SEQ ID NO: 105, an LCDR2 of SEQ ID NO: 63, and an LCDR3 of SEQ ID NO: 75.

In some embodiments, the antibody or antigen-binding fragment thereof comprises an HCDR1 of SEQ ID NO: 101, an HCDR2 of SEQ ID NO: 102, and an HCDR3 of SEQ ID NO: 103. In some embodiments, the antibody or antigen-binding fragment thereof comprises an LCDR1 of SEQ ID NO: 105, an LCDR2 of SEQ ID NO: 106, and an LCDR3 of SEQ ID NO: 75. In some embodiments, the antibody or antigen-binding fragment thereof comprises an HCDR1 of SEQ ID NO: 101, an HCDR2 of SEQ ID NO: 102, an HCDR3 of SEQ ID NO: 103, an LCDR1 of SEQ ID NO: 105, an LCDR2 of SEQ ID NO: 106, and an LCDR3 of SEQ ID NO: 75.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 103, an LCDR1 of SEQ ID NO: 105, an LCDR2 of SEQ ID NO: 106, and an LCDR3 of SEQ ID NO: 75.

In some embodiments, the antibody or antigen-binding fragment thereof comprises an HCDR1 of SEQ ID NO: 101, an HCDR2 of SEQ ID NO: 102, and an HCDR3 of SEQ ID NO: 104. In some embodiments, the antibody or antigen-binding fragment thereof comprises an LCDR1 of SEQ ID NO: 105, an LCDR2 of SEQ ID NO: 106, and an LCDR3 of SEQ ID NO: 107. In some embodiments, the antibody or antigen-binding fragment thereof comprises an HCDR1 of SEQ ID NO: 101, an HCDR2 of SEQ ID NO: 102, an HCDR3 of SEQ ID NO: 104, an LCDR1 of SEQ ID NO: 105, an LCDR2 of SEQ ID NO: 106, and an LCDR3 of SEQ ID NO: 107.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 104, an LCDR1 of SEQ ID NO: 105, an LCDR2 of SEQ ID NO: 106, and an LCDR3 of SEQ ID NO: 107.

In some embodiments, the antibody or antigen-binding fragment thereof comprises an HCDR1 of SEQ ID NO: 101, an HCDR2 of SEQ ID NO: 102, and an HCDR3 of SEQ ID NO: 188. In some embodiments, the antibody or antigen-binding fragment thereof comprises an LCDR1 of SEQ ID NO: 105, an LCDR2 of SEQ ID NO: 106, and an LCDR3 of SEQ ID NO: 198. In some embodiments, the antibody or antigen-binding fragment thereof comprises an HCDR1 of SEQ ID NO: 101, an HCDR2 of SEQ ID NO: 102, an HCDR3 of SEQ ID NO: 188, an LCDR1 of SEQ ID NO: 105, an LCDR2 of SEQ ID NO: 106, and an LCDR3 of SEQ ID NO: 198.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 188, an LCDR1 of SEQ ID NO: 105, an LCDR2 of SEQ ID NO: 106, and an LCDR3 of SEQ ID NO: 198.

In some embodiments, the antibody or antigen-binding fragment thereof comprises an HCDR1 of SEQ ID NO: 101, an HCDR2 of SEQ ID NO: 102, and an HCDR3 of SEQ ID NO: 189. In some embodiments, the antibody or antigen-binding fragment thereof comprises an LCDR1 of SEQ ID NO: 105, an LCDR2 of SEQ ID NO: 106, and an LCDR3 of SEQ ID NO: 75. In some embodiments, the antibody or antigen-binding fragment thereof comprises an HCDR1 of SEQ ID NO: 101, an HCDR2 of SEQ ID NO: 102, an HCDR3 of SEQ ID NO: 189, an LCDR1 of SEQ ID NO: 105, an LCDR2 of SEQ ID NO: 106, and an LCDR3 of SEQ ID NO: 75.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 189, an LCDR1 of SEQ ID NO: 105, an LCDR2 of SEQ ID NO: 106, and an LCDR3 of SEQ ID NO: 75.

In some embodiments, the antibody or antigen-binding fragment thereof comprises an HCDR1 of SEQ ID NO: 101, an HCDR2 of SEQ ID NO: 102, and an HCDR3 of SEQ ID NO: 190. In some embodiments, the antibody or antigen-binding fragment thereof comprises an LCDR1 of SEQ ID NO: 105, an LCDR2 of SEQ ID NO: 106, and an LCDR3 of SEQ ID NO: 75. In some embodiments, the antibody or antigen-binding fragment thereof comprises an HCDR1 of SEQ ID NO: 101, an HCDR2 of SEQ ID NO: 102, an HCDR3 of SEQ ID NO: 190, an LCDR1 of SEQ ID NO: 105, an LCDR2 of SEQ ID NO: 106, and an LCDR3 of SEQ ID NO: 75.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 190, an LCDR1 of SEQ ID NO: 105, an LCDR2 of SEQ ID NO: 106, and an LCDR3 of SEQ ID NO: 75.

In some embodiments, the antibody or antigen-binding fragment thereof comprises an HCDR1 of SEQ ID NO: 101, an HCDR2 of SEQ ID NO: 102, and an HCDR3 of SEQ ID NO: 191. In some embodiments, the antibody or antigen-binding fragment thereof comprises an LCDR1 of SEQ ID NO: 105, an LCDR2 of SEQ ID NO: 106, and an LCDR3 of SEQ ID NO: 75. In some embodiments, the antibody or antigen-binding fragment thereof comprises an HCDR1 of SEQ ID NO: 101, an HCDR2 of SEQ ID NO: 102, an HCDR3 of SEQ ID NO: 191, an LCDR1 of SEQ ID NO: 105, an LCDR2 of SEQ ID NO: 106, and an LCDR3 of SEQ ID NO: 75.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 191, an LCDR1 of SEQ ID NO: 105, an LCDR2 of SEQ ID NO: 106, and an LCDR3 of SEQ ID NO: 75.

In some embodiments, the antibody or antigen-binding fragment thereof comprises an HCDR1 of SEQ ID NO: 101, an HCDR2 of SEQ ID NO: 102, and an HCDR3 of SEQ ID NO: 192. In some embodiments, the antibody or antigen-binding fragment thereof comprises an LCDR1 of SEQ ID NO: 105, an LCDR2 of SEQ ID NO: 106, and an LCDR3 of SEQ ID NO: 75. In some embodiments, the antibody or antigen-binding fragment thereof comprises an HCDR1 of SEQ ID NO: 101, an HCDR2 of SEQ ID NO: 102, an HCDR3 of SEQ ID NO: 192, an LCDR1 of SEQ ID NO: 105, an LCDR2 of SEQ ID NO: 106, and an LCDR3 of SEQ ID NO: 75.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 192, an LCDR1 of SEQ ID NO: 105, an LCDR2 of SEQ ID NO: 106, and an LCDR3 of SEQ ID NO: 75

In some embodiments, the antibody or antigen-binding fragment thereof comprises an HCDR1 of SEQ ID NO: 101, an HCDR2 of SEQ ID NO: 102, and an HCDR3 of SEQ ID NO: 193. In some embodiments, the antibody or antigen-binding fragment thereof comprises an LCDR1 of SEQ ID NO: 105, an LCDR2 of SEQ ID NO: 106, and an LCDR3 of SEQ ID NO: 198. In some embodiments, the antibody or antigen-binding fragment thereof comprises an HCDR1 of SEQ ID NO: 101, an HCDR2 of SEQ ID NO: 102, an HCDR3 of SEQ ID NO: 193, an LCDR1 of SEQ ID NO: 105, an LCDR2 of SEQ ID NO: 106, and an LCDR3 of SEQ ID NO: 198.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 193, an LCDR1 of SEQ ID NO: 105, an LCDR2 of SEQ ID NO: 106, and an LCDR3 of SEQ ID NO: 198.

In some embodiments, the antibody or antigen-binding fragment thereof comprises an HCDR1 of SEQ ID NO: 101, an HCDR2 of SEQ ID NO: 102, and an HCDR3 of SEQ ID NO: 194. In some embodiments, the antibody or antigen-binding fragment thereof comprises an LCDR1 of SEQ ID NO: 105, an LCDR2 of SEQ ID NO: 106, and an LCDR3 of SEQ ID NO: 107. In some embodiments, the antibody or antigen-binding fragment thereof comprises an HCDR1 of SEQ ID NO: 101, an HCDR2 of SEQ ID NO: 102, an HCDR3 of SEQ ID NO: 194, an LCDR1 of SEQ ID NO: 105, an LCDR2 of SEQ ID NO: 106, and an LCDR3 of SEQ ID NO: 107.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 194, an LCDR1 of SEQ ID NO: 105, an LCDR2 of SEQ ID NO: 106, and an LCDR3 of SEQ ID NO: 107.

In some embodiments, the antibody or antigen-binding fragment thereof comprises an HCDR1 of SEQ ID NO: 101, an HCDR2 of SEQ ID NO: 102, and an HCDR3 of SEQ ID NO: 194. In some embodiments, the antibody or antigen-binding fragment thereof comprises an LCDR1 of SEQ ID NO: 105, an LCDR2 of SEQ ID NO: 196, and an LCDR3 of SEQ ID NO: 107. In some embodiments, the antibody or antigen-binding fragment thereof comprises an HCDR1 of SEQ ID NO: 101, an HCDR2 of SEQ ID NO: 102, an HCDR3 of SEQ ID NO: 194, an LCDR1 of SEQ ID NO: 105, an LCDR2 of SEQ ID NO: 196, and an LCDR3 of SEQ ID NO: 107.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 194, an LCDR1 of SEQ ID NO: 105, an LCDR2 of SEQ ID NO: 196, and an LCDR3 of SEQ ID NO: 107.

In some embodiments, the antibody or antigen-binding fragment thereof comprises an HCDR1 of SEQ ID NO: 101, an HCDR2 of SEQ ID NO: 102, and an HCDR3 of SEQ ID NO: 195. In some embodiments, the antibody or antigen-binding fragment thereof comprises an LCDR1 of SEQ ID NO: 105, an LCDR2 of SEQ ID NO: 63, and an LCDR3 of SEQ ID NO: 75. In some embodiments, the antibody or antigen-binding fragment thereof comprises an HCDR1 of SEQ ID NO: 101, an HCDR2 of SEQ ID NO: 102, an HCDR3 of SEQ ID NO: 195, an LCDR1 of SEQ ID NO: 105, an LCDR2 of SEQ ID NO: 63, and an LCDR3 of SEQ ID NO: 75.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 195, an LCDR1 of SEQ ID NO: 105, an LCDR2 of SEQ ID NO: 63, and an LCDR3 of SEQ ID NO: 75.

In some embodiments, the antibody or antigen-binding fragment thereof comprises an HCDR1 of SEQ ID NO: 101, an HCDR2 of SEQ ID NO: 102, and an HCDR3 of SEQ ID NO: 104. In some embodiments, the antibody or antigen-binding fragment thereof comprises an LCDR1 of SEQ ID NO: 105, an LCDR2 of SEQ ID NO: 63, and an LCDR3 of SEQ ID NO: 75. In some embodiments, the antibody or antigen-binding fragment thereof comprises an HCDR1 of SEQ ID NO: 101, an HCDR2 of SEQ ID NO: 102, an HCDR3 of SEQ ID NO: 104, an LCDR1 of SEQ ID NO: 105, an LCDR2 of SEQ ID NO: 63, and an LCDR3 of SEQ ID NO: 75.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 104, an LCDR1 of SEQ ID NO: 105, an LCDR2 of SEQ ID NO: 63, and an LCDR3 of SEQ ID NO: 75.

In some embodiments, the antibody or antigen-binding fragment thereof comprises an HCDR1 of SEQ ID NO: 101, an HCDR2 of SEQ ID NO: 102, and an HCDR3 of SEQ ID NO: 104. In some embodiments, the antibody or antigen-binding fragment thereof comprises an LCDR1 of SEQ ID NO: 105, an LCDR2 of SEQ ID NO: 197, and an LCDR3 of SEQ ID NO: 75. In some embodiments, the antibody or antigen-binding fragment thereof comprises an HCDR1 of SEQ ID NO: 101, an HCDR2 of SEQ ID NO: 102, an HCDR3 of SEQ ID NO: 104, an LCDR1 of SEQ ID NO: 105, an LCDR2 of SEQ ID NO: 197, and an LCDR3 of SEQ ID NO: 75.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 104, an LCDR1 of SEQ ID NO: 105, an LCDR2 of SEQ ID NO: 197, and an LCDR3 of SEQ ID NO: 75.

In some embodiments, the antibody or antigen-binding fragment thereof comprises an HCDR1 of SEQ ID NO: 101, an HCDR2 of SEQ ID NO: 102, and an HCDR3 of SEQ ID NO: 190. In some embodiments, the antibody or antigen-binding fragment thereof comprises an LCDR1 of SEQ ID NO: 105, an LCDR2 of SEQ ID NO: 106, and an LCDR3 of SEQ ID NO: 198. In some embodiments, the antibody or antigen-binding fragment thereof comprises an HCDR1 of SEQ ID NO: 101, an HCDR2 of SEQ ID NO: 102, an HCDR3 of SEQ ID NO: 190, an LCDR1 of SEQ ID NO: 105, an LCDR2 of SEQ ID NO: 106, and an LCDR3 of SEQ ID NO: 198.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 190, an LCDR1 of SEQ ID NO: 105, an LCDR2 of SEQ ID NO: 106, and an LCDR3 of SEQ ID NO: 198.

In some embodiments, the antibody or antigen-binding fragment thereof comprises an HCDR1 of SEQ ID NO: 101, an HCDR2 of SEQ ID NO: 102, and an HCDR3 of SEQ ID NO: 189. In some embodiments, the antibody or antigen-binding fragment thereof comprises an LCDR1 of SEQ ID NO: 105, an LCDR2 of SEQ ID NO: 197, and an LCDR3 of SEQ ID NO: 107. In some embodiments, the antibody or antigen-binding fragment thereof comprises an HCDR1 of SEQ ID NO: 101, an HCDR2 of SEQ ID NO: 102, an HCDR3 of SEQ ID NO: 189, an LCDR1 of SEQ ID NO: 105, an LCDR2 of SEQ ID NO: 197, and an LCDR3 of SEQ ID NO: 107.

In some embodiments, the antibody or antigen-binding fragment thereof competes with a reference antibody, wherein the reference antibody comprises an HCDR1 of SEQ ID NO: 101, an HCDR2 of SEQ ID NO: 102, an HCDR3 of SEQ ID NO: 189, an LCDR1 of SEQ ID NO: 105, an LCDR2 of SEQ ID NO: 197, and an LCDR3 of SEQ ID NO: 107.

In some embodiments, the integrin α7β1 antibody or antigen binding fragment thereof comprises CDRs with one or more amino acid different from CDRs shown in Table E. In some embodiments, the integrin α7β1 antibody or antigen binding fragment thereof comprises a CDR with no more than 5 amino acids different from any one of the CDRs shown in Table E. In some embodiments, the integrin α7β1 antibody or antigen binding fragment thereof comprises a CDR with no more than 4 amino acids different from any one of the CDRs shown in Table E. In some embodiments, the integrin α7β1 antibody or antigen binding fragment thereof comprises a CDR with no more than 3 amino acids different from any one of the CDRs shown in Table E. In some embodiments, the integrin α7β1 antibody or antigen binding fragment thereof comprises a CDR with no more than 2 amino acids different from any one of the CDRs shown in Table E. In some embodiments, the integrin α7β1 antibody or antigen binding fragment thereof comprises a CDR with no more than 1 amino acid different from any one of the CDRs shown in Table E.

In some embodiments, the integrin α7β1 antibody or antigen binding fragment thereof comprises a HCDR1 comprising an amino acid sequence with no greater than 2 amino acids different from GYSFTRYW (SEQ ID NO: 101), a HCDR2 comprising an amino acid sequence with no greater than 1 amino acid different from IDPSDSET (SEQ ID 0: 102), a HCDR3 comprising an amino acid sequence with no greater than 4 amino acids different from ARGGYFGREWYYDV (SEQ ID NO: 103), a LCDR1 comprising an amino acid sequence with no greater than 2 amino acids different from QSLVHRNGNTY (SEQ ID NO: 105), a LCDR2 comprising an amino acid sequence with no greater than 1 amino acid different from EVS (SEQ ID NO: 106), and a LCDR3 comprising an amino acid sequence with no greater than 4 amino acids different from SQSTHVPWT (SEQ ID NO:75, LCDR3G).

In some embodiments, the integrin α7β1 antibody or antigen binding fragment thereof comprises a HCDR1 comprising an amino acid sequence with no greater than 2 amino acids different from GYSFTRYW (SEQ ID NO: 101), a HCDR2 comprising an amino acid sequence with no greater than 1 amino acid different from IDPSDSET (SEQ ID NO: 102), a HCDR3 comprising an amino acid sequence with no greater than 4 amino acids different from ARGGYFGEEWYHDV (SEQ ID NO: 104), a LCDR1 comprising an amino acid sequence with no greater than 2 amino acids different from QSLVHRNGNTY (SEQ ID NO: 105), a LCDR2 comprising an amino acid sequence with no greater than 1 amino acid different from EVS (SEQ ID No: 106), and a LCDR3 comprising an amino acid sequence with no greater than 4 amino acids different from AQSTQLPYT (SEQ ID NO: 107).

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 188, a LCDR1 comprising an amino acid sequence with no greater than 2 amino acids different from SEQ ID NO: 105, a LCDR2 comprising an amino acid sequence with no greater than 1 amino acid different from SEQ ID NO: 106, and a LCDR3 comprising an amino acid sequence with no greater than 4 amino acids different from SEQ ID NO: 198.

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 189, a LCDR1 comprising an amino acid sequence with no greater than 2 amino acids different from SEQ ID NO: 105, a LCDR2 comprising an amino acid sequence with no greater than 1 amino acid different from SEQ ID NO: 106, and a LCDR3 comprising an amino acid sequence with no greater than 4 amino acids different from SEQ ID NO: 75.

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 190, a LCDR1 comprising an amino acid sequence with no greater than 2 amino acids different from SEQ ID NO: 105, a LCDR2 comprising an amino acid sequence with no greater than 1 amino acid different from SEQ ID NO: 106, and a LCDR3 comprising an amino acid sequence with no greater than 4 amino acids different from SEQ ID NO: 75.

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 191, a LCDR1 comprising an amino acid sequence with no greater than 2 amino acids different from SEQ ID NO: 105, a LCDR2 comprising an amino acid sequence with no greater than 1 amino acid different from SEQ ID NO: 106, and a LCDR3 comprising an amino acid sequence with no greater than 4 amino acids different from SEQ ID NO: 75.

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 192, a LCDR1 comprising an amino acid sequence with no greater than 2 amino acids different from SEQ ID NO: 105, a LCDR2 comprising an amino acid sequence with no greater than 1 amino acid different from SEQ ID NO: 106, and a LCDR3 comprising an amino acid sequence with no greater than 4 amino acids different from SEQ ID NO: 75.

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 193, a LCDR1 comprising an amino acid sequence with no greater than 2 amino acids different from SEQ ID NO: 105, a LCDR2 comprising an amino acid sequence with no greater than 1 amino acid different from SEQ ID NO: 106, and a LCDR3 comprising an amino acid sequence with no greater than 4 amino acids different from SEQ ID NO: 198.

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 194, a LCDR1 comprising an amino acid sequence with no greater than 2 amino acids different from SEQ ID NO: 105, a LCDR2 comprising an amino acid sequence with no greater than 1 amino acid different from SEQ ID NO: 106, and a LCDR3 comprising an amino acid sequence with no greater than 4 amino acids different from SEQ ID NO: 107.

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 194, a LCDR1 comprising an amino acid sequence with no greater than 2 amino acids different from SEQ ID NO: 105, a LCDR2 comprising an amino acid sequence with no greater than 1 amino acid different from SEQ ID NO: 196, and a LCDR3 comprising an amino acid sequence with no greater than 4 amino acids different from SEQ ID NO: 107.

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 195, a LCDR1 comprising an amino acid sequence with no greater than 2 amino acids different from SEQ ID NO: 105, a LCDR2 comprising an amino acid sequence with no greater than 1 amino acid different from SEQ ID NO: 63, and a LCDR3 comprising an amino acid sequence with no greater than 4 amino acids different from SEQ ID NO: 75.

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 104, a LCDR1 comprising an amino acid sequence with no greater than 2 amino acids different from SEQ ID NO: 105, a LCDR2 comprising an amino acid sequence with no greater than 1 amino acid different from SEQ ID NO: 63, and a LCDR3 comprising an amino acid sequence with no greater than 4 amino acids different from SEQ ID NO: 75.

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 104, a LCDR1 comprising an amino acid sequence with no greater than 2 amino acids different from SEQ ID NO: 105, a LCDR2 comprising an amino acid sequence with no greater than 1 amino acid different from SEQ ID NO: 197, and a LCDR3 comprising an amino acid sequence with no greater than 4 amino acids different from SEQ ID NO: 75.

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 190, a LCDR1 comprising an amino acid sequence with no greater than 2 amino acids different from SEQ ID NO: 105, a LCDR2 comprising an amino acid sequence with no greater than 1 amino acid different from SEQ ID NO: 106, and a LCDR3 comprising an amino acid sequence with no greater than 4 amino acids different from SEQ ID NO: 198.

In some embodiments, the integrin α7β1 antibody or antigen binding fragment thereof comprises a HCDR1 comprising an amino acid sequence with no greater than 2 amino acids different from SEQ ID NO: 101, a HCDR2 comprising an amino acid sequence with no greater than 1 amino acid different from SEQ ID NO: 102, a HCDR3 comprising an amino acid sequence with no greater than 4 amino acids different from SEQ ID NO: 189, a LCDR1 comprising an amino acid sequence with no greater than 2 amino acids different from SEQ ID NO: 105, a LCDR2 comprising an amino acid sequence with no greater than 1 amino acid different from SEQ ID NO: 197, and a LCDR3 comprising an amino acid sequence with no greater than 4 amino acids different from SEQ ID NO: 107.

TABLE F
Exemplary VH and VL Sequences Qf Humanized anti-α7β1 Integrin AntibQdies.

| Ab | VH Sequence | VL Sequence |
|---|---|---|
| AB-P | QVQLVQSGAEVKKPGSSVKVSCKASGYSFTR | DVVMTQSPLSLPVTPGEPASISCRSSQSLVH |
| | YWMHWVRQAPGQGLEWIGKIDPSDSETRLNQ | RNGNTYLHWYLQKPGQSPQLLIYKVSNRFSG |
| | EFRDRATLTVDKSTSTAYMELSSLRSEDTAV | VPDRFSGSGSGTDFTLKISRVEAEDVGVYFC |
| | YYCARGGYFGEGWYFDVWGQGTLVTVSS | SQSTHVPWTFGGGTKLEIK (SEQ ID NO: |
| | (SEQ ID NO: 186) | 187) |
| Ab24 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSNRAAG |
| | EFRDRATLTVDKSTSTAYMELSSLRSEDTAV | VPDRFSGSGSGTDFTLKISRVEAEDVGVYFC |
| | YYCARGGYFGREWYYDVWGQGTLVTVSS | SQSTHVPWTFGGGTKLEIK (SEQ ID NO: |
| | (SEQ ID NO: 108) | 109) |
| Ab25 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSNRAAG |
| | EFRDRATLTVDKSTSTAYMELSSLRSEDTAV | VPDRFSGSGSGTDFTLKISRVEAEDVGVYFC |
| | YYCARGGYFGREWYYDVWGQGTLVTVSS | SQSTHVPWTFGGGTKLEIK (SEQ ID NO: |
| | (SEQ ID NO: 108) | 109) |
| Ab26 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSNRFTG |
| | EFRDRATLTVDKSTSTAYMELSSLRSEDTAV | VPDRFSGSGSGTDFTLKISRVEAEDVGVYFC |
| | YYCARGGYFGEEWYHDVWGQGTLVTVSS | AQSTQLPYTFGGGTKLEIK (SEQ ID NO: |
| | (SEQ ID NO: 110) | 111) |
| Ab27 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSNRFTG |
| | EFRDRATLTVDKSTSTAYMELSSLRSEDTAV | VPDRFSGSGSGTDFTLKISRVEAEDVGVYFC |
| | YYCARGGYFGEEWYHDVWGQGTLVTVSS | AQSTQLPYTFGGGTKLEIK (SEQ ID NO: |
| | (SEQ ID NO: 110) | 111) |
| Ab30 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSKRASG |
| | EFRDRATLTVDKSTSTAYMELSSLRSEDTAV | VPDRFSGSGSGTDFTLKISRVEAEDVGVYFC |
| | YYCARGGYFGEGWYHDVWGQGTLVTVSS | AQSIQVPWTFGGGTKLEIK (SEQ ID NO: |
| | (SEQ ID NO: 199) | 200) |
| Ab31 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSNRASG |
| | EFRDRATLTVDKSTSTAYMELSSLRSEDTAV | VPDRFSGSGSGTDFTLKISRVEAEDVGVYFC |
| | YYCARGGYFGRQWYFDVWGQGTLVTVSS | SQSTHVPWTFGGGTKLEIK (SEQ ID NO: |
| | (SEQ ID NO: 201) | 202) |
| Ab32 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSNRASG |
| | EFRDRATLTVDKSTSTAYMELSSLRSEDTAV | VPDRFSGSGSGTDFTLKISRVEAEDVGVYFC |
| | YYCARGGYFGEQWYHDVWGQGTLVTVSS | SQSTHVPWTFGGGTKLEIK (SEQ ID NO: |
| | (SEQ ID NO: 203) | 204) |
| Ab33 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSNRVPG |
| | EFRDRATLTVDKSTSTAYMELSSLRSEDTAV | VPDRFSGSGSGTDFTLKISRVEAEDVGVYFC |
| | YYCARGGYFGVGWYNDVWGQGTLVTVSS | SQSTHVPWTFGGGTKLEIK (SEQ ID NO: |
| | (SEQ ID NO: 205) | 206) |
| Ab34 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSHRASG |
| | EFRDRATLTVDKSTSTAYMELSSLRSEDTAV | VPDRFSGSGSGTDFTLKISRVEAEDVGVYFC |
| | YYCARGGYFGETWYHDVWGQGTLVTVSS | SQSTHVPWTFGGGTKLEIK (SEQ ID NO: |
| | (SEQ ID NO: 207) | 208) |
| Ab35 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSNRYSG |
| | EFRDRATLTVDKSTSTAYMELSSLRSEDTAV | VPDRFSGSGSGTDFTLKISRVEAEDVGVYFC |
| | YYCARGGYFGENWYHDVWGQGTLVTVSS | AQSIQVPWTFGGGTKLEIK (SEQ ID NO: |
| | (SEQ ID NO: 209) | 210) |
| Ab36 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSHRFPG |
| | EFRDRATLTVDKSTSTAYMELSSLRSEDTAV | VPDRFSGSGSGTDFTLKISRVEAEDVGVYFC |
| | YYCARGGYFGEKWYHDVWGQGTLVTVSS | AQSTQLPYTFGGGTKLEIK (SEQ ID NO: |
| | (SEQ ID NO: 211) | 212) |
| Ab37 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYGVSNRFAG |
| | EFRDRATLTVDKSTSTAYMELSSLRSEDTAV | VPDRFSGSGSGTDFTLKISRVEAEDVGVYFC |
| | YYCARGGYFGEKWYHDVWGQGTLVTVSS | AQSTQLPYTFGGGTKLEIK (SEQ ID NO: |
| | (SEQ ID NO: 213) | 214) |
| Ab38 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYKVSNRASG |
| | EFRDRATLTVDKSTSTAYMELSSLRSEDTAV | VPDRFSGSGSGTDFTLKISRVEAEDVGVYFC |
| | YYCARGGYFGEEWYNDVWGQGTLVTVSS | SQSTHVPWTFGGGTKLEIK (SEQ ID NO: |
| | (SEQ ID NO: 215) | 216) |
| Ab39 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYKVSNRASG |
| | EFRDRATLTVDKSTSTAYMELSSLRSEDTAV | VPDRFSGSGSGTDFTLKISRVEAEDVGVYFC |
| | YYCARGGYFGEEWYHDVWGQGTLVTVSS | SQSTHVPWTFGGGTKLEIK (SEQ ID NO: |
| | (SEQ ID NO: 217) | 218) |
| Ab40 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEGSKRFSG |
| | EFRDRATLTVDKSTSTAYMELSSLRSEDTAV | VPDRFSGSGSGTDFTLKISRVEAEDVGVYFC |
| | YYCARGGYFGEEWYHDVWGQGTLVTVSS | SQSTHVPWTFGGGTKLEIK (SEQ ID NO: |
| | (SEQ ID NO: 219) | 220) |
| Ab41 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSKRFSG |
| | EFRDRATLTVDKSTSTAYMELSSLRSEDTAV | VPDRFSGSGSGTDFTLKISRVEAEDVGVYFC |
| | YYCARGGYF GEQWYHDVWGQGTLVTVSS | AQSIQVPWTFGGGTKLEIK (SEQ ID NO: |
| | (SEQ ID NO: 221) | 222) |
| Ab42 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEGSKRFSG |
| | EFRDRATLTVDKSTSTAYMELSSLRSEDTAV | VPDRFSGSGSGTDFTLKISRVEAEDVGVYFC |
| | YYCARGGYFGRQWYFDVWGQGTLVTVSS | AQSTQLPYTFGGGTKLEIK (SEQ ID NO: |
| | (SEQ ID NO: 223) | 224) |

In some embodiments, the integrin α7β1 antibody or antigen binding fragment thereof comprises a variable heavy region comprising an amino acid sequence at least 80% identical to SEQ ID NO: 186. In some embodiments, the integrin α7β1 antibody or antigen binding fragment thereof comprises a variable heavy region comprising an amino acid sequence at least 83% identical to SEQ ID NO: 186. In some embodiments, the integrin α7β1 antibody or antigen binding fragment thereof comprises a variable heavy region comprising an amino acid sequence at least 85% identical to SEQ ID NO: 186. In some embodiments, the integrin α7β1 antibody or antigen binding fragment thereof comprises a variable heavy region comprising an amino acid sequence at least 87% identical to SEQ ID NO: 186. In some embodiments, the integrin α7β1 antibody or antigen binding fragment thereof comprises a variable heavy region comprising an amino acid sequence at least 90% identical to SEQ ID NO: 186. In some embodiments, the integrin α7β1 antibody or antigen binding fragment thereof comprises a variable heavy region comprising an amino acid sequence at least 92% identical to SEQ ID NO: 186. In some embodiments, the integrin α7β1 antibody or antigen binding fragment thereof comprises a variable heavy region comprising an amino acid sequence at least 93% identical to SEQ ID NO: 186. In some embodiments, the integrin α7β1 antibody or antigen binding fragment thereof comprises a variable heavy region comprising an amino acid sequence at least 94% identical to SEQ ID NO: 186. In some embodiments, the integrin α7β1 antibody or antigen binding fragment thereof comprises a variable heavy region comprising an amino acid sequence at least 95% identical to SEQ ID NO: 186. In some embodiments, the integrin α7β1 antibody or antigen binding fragment thereof comprises a variable heavy region comprising an amino acid sequence at least 97% identical to SEQ ID NO: 186. In some embodiments, the integrin α7β1 antibody or antigen binding fragment thereof comprises a variable heavy region comprising an amino acid sequence at least 98% identical to SEQ ID NO: 186. In some embodiments, the integrin α7β1 antibody or antigen binding fragment thereof comprises a variable heavy region comprising an amino acid sequence at least 99% identical to SEQ ID NO: 186. In some embodiments, the integrin α7β1 antibody or antigen binding fragment thereof comprises a variable heavy region comprising an amino acid sequence identical to SEQ ID NO: 186.

In some embodiments, the integrin α7β1 antibody or antigen binding fragment thereof comprises a variable heavy region comprising an amino acid sequence at least 80% identical to SEQ ID NO: 108. In some embodiments, the integrin α7β1 antibody or antigen binding fragment thereof comprises a variable heavy region comprising an amino acid sequence at least 83% identical to SEQ ID NO: 108. In some embodiments, the integrin α7β1 antibody or antigen binding fragment thereof comprises a variable heavy region comprising an amino acid sequence at least 85% identical to SEQ ID NO: 108. In some embodiments, the integrin α7β1 antibody or antigen binding fragment thereof comprises a variable heavy region comprising an amino acid sequence at least 87% identical to SEQ ID NO: 108. In some embodiments, the integrin α7β1 antibody or antigen binding fragment thereof comprises a variable heavy region comprising an amino acid sequence at least 90% identical to SEQ ID NO: 108. In some embodiments, the integrin α7β1 antibody or antigen binding fragment thereof comprises a variable heavy region comprising an amino acid sequence at least 92% identical to SEQ ID NO: 108. In some embodiments, the integrin α7β1 antibody or antigen binding fragment thereof comprises a variable heavy region comprising an amino acid sequence at least 93% identical to SEQ ID NO: 108. In some embodiments, the integrin α7β1 antibody or antigen binding fragment thereof comprises a variable heavy region comprising an amino acid sequence at least 94% identical to SEQ ID NO: 108. In some embodiments, the integrin α7β1 antibody or antigen binding fragment thereof comprises a variable heavy region comprising an amino acid sequence at least 95% identical to SEQ ID NO: 108. In some embodiments, the integrin α7β1 antibody or antigen binding fragment thereof comprises a variable heavy region comprising an amino acid sequence at least 97% identical to SEQ ID NO: 108. In some embodiments, the integrin α7β1 antibody or antigen binding fragment thereof comprises a variable heavy region comprising an amino acid sequence at least 98% identical to SEQ ID NO: 108. In some embodiments, the integrin α7β1 antibody or antigen binding fragment thereof comprises a variable heavy region comprising an amino acid sequence at least 99% identical to SEQ ID NO: 108. In some embodiments, the integrin α7β1 antibody or antigen binding fragment thereof comprises a variable heavy region comprising an amino acid sequence identical to SEQ ID NO: 108.

In some embodiments, the integrin α7β1 antibody or antigen binding fragment thereof comprises a variable heavy region comprising an amino acid sequence at least 80% identical to SEQ ID NO: 110. In some embodiments, the integrin α7β1 antibody or antigen binding fragment thereof comprises a variable heavy region comprising an amino acid sequence at least 83% identical to SEQ ID NO: 110. In some embodiments, the integrin α7β1 antibody or antigen binding fragment thereof comprises a variable heavy region comprising an amino acid sequence at least 85% identical to SEQ ID NO: 110. In some embodiments, the integrin α7β1 antibody or antigen binding fragment thereof comprises a variable heavy region comprising an amino acid sequence at least 87% identical to SEQ ID No: 110. In some embodiments, the integrin α7β1 antibody or antigen binding fragment thereof comprises a variable heavy region comprising an amino acid sequence at least 90% identical to SEQ ID NO: 110. In some embodiments, the integrin α7β1 antibody or antigen binding fragment thereof comprises a variable heavy region comprising an amino acid sequence at least 92% identical to SEQ ID NO: 110. In some embodiments, the integrin α7β1 antibody or antigen binding fragment thereof comprises a variable heavy region comprising an amino acid sequence at least 93% identical to SEQ ID NO: 110. In some embodiments, the integrin α7β1 antibody or antigen binding fragment thereof comprises a variable heavy region comprising an amino acid sequence at least 94% identical to SEQ ID NO: 110. In some embodiments, the integrin α7β1 antibody or antigen binding fragment thereof comprises a variable heavy region comprising an amino acid sequence at least 95% identical to SEQ ID NO: 110. In some embodiments, the integrin α7β1 antibody or antigen binding fragment thereof comprises a variable heavy region comprising an amino acid sequence at least 97% identical to SEQ ID NO: 110. In some embodiments, the integrin α7β1 antibody or antigen binding fragment thereof comprises a variable heavy region comprising an amino acid sequence at least 98% identical to SEQ ID NO: 110. In some embodiments, the integrin α7β1 antibody or antigen binding fragment thereof comprises a variable heavy region comprising an amino acid sequence at least 99% identical to SEQ ID NO: 110. In some embodiments, the integrin α7β1 antibody or antigen binding fragment thereof comprises a variable heavy region comprising an amino acid sequence identical to SEQ ID NO: 110.

In some embodiments, the integrin α7β1 antibody or antigen binding fragment thereof comprises a variable heavy region comprising an amino acid sequence at least 80% identical to SEQ ID NO: 199. In some embodiments, the integrin α7β1 antibody or antigen binding fragment thereof comprises a variable heavy region comprising an amino acid sequence at least 83% identical to SEQ ID NO: 199. In some embodiments, the integrin α7β1 antibody or antigen binding fragment thereof comprises a variable heavy region comprising an amino acid sequence at least 85% identical to SEQ ID NO: 199. In some embodiments, the integrin α7β1 antibody or antigen binding fragment thereof comprises a variable heavy region comprising an amino acid sequence at least 87% identical to SEQ ID NO: 199. In some embodiments, the integrin α7β1 antibody or antigen binding fragment thereof comprises a variable heavy region comprising an amino acid sequence at least 90% identical to SEQ ID NO: 199. In some embodiments, the integrin α7β1 antibody or antigen binding fragment thereof comprises a variable heavy region comprising an amino acid sequence at least 92% identical to SEQ ID NO: 199. In some embodiments, the integrin α7β1 antibody or antigen binding fragment thereof comprises a variable heavy region comprising an amino acid sequence at least 93% identical to SEQ ID NO: 199. In some embodiments, the integrin α7β1 antibody or antigen binding fragment thereof comprises a variable heavy region comprising an amino acid sequence at least 94% identical to SEQ ID NO: 199. In some embodiments, the integrin α7β1 antibody or antigen binding fragment thereof comprises a variable heavy region comprising an amino acid sequence at least 95% identical to SEQ ID NO: 199. In some embodiments, the integrin α7β1 antibody or antigen binding fragment thereof comprises a variable heavy region comprising an amino acid sequence at least 97% identical to SEQ ID NO: 199. In some embodiments, the integrin α7β1 antibody or antigen binding fragment thereof comprises a variable heavy region comprising an amino acid sequence at least 98% identical to SEQ ID NO: 199. In some embodiments, the integrin α7β1 antibody or antigen binding fragment thereof comprises a variable heavy region comprising an amino acid sequence at least 99% identical to SEQ ID NO: 199. In some embodiments, the integrin α7β1 antibody or antigen binding fragment thereof comprises a variable heavy region comprising an amino acid sequence identical to SEQ ID NO: 199.

In some embodiments, the integrin α7β1 antibody or antigen binding fragment thereof comprises a variable heavy region comprising an amino acid sequence at least 80% identical to SEQ ID NO: 201. In some embodiments, the integrin α7β1 antibody or antigen binding fragment thereof comprises a variable heavy region comprising an amino acid sequence at least 83% identical to SEQ ID NO: 201. In some embodiments, the integrin α7β1 antibody or antigen binding fragment thereof comprises a variable heavy region comprising an amino acid sequence at least 85% identical to SEQ ID NO: 201. In some embodiments, the integrin α7β1 antibody or antigen binding fragment thereof comprises a variable heavy region comprising an amino acid sequence at least 87% identical to SEQ ID NO: 201. In some embodiments, the integrin α7β1 antibody or antigen binding fragment thereof comprises a variable heavy region comprising an amino acid sequence at least 90% identical to SEQ ID NO: 201. In some embodiments, the integrin α7β1 antibody or antigen binding fragment thereof comprises a variable heavy region comprising an amino acid sequence at least 92% identical to SEQ ID NO: 201. In some embodiments, the integrin α7β1 antibody or antigen binding fragment thereof comprises a variable heavy region comprising an amino acid sequence at least 93% identical to SEQ ID NO: 201. In some embodiments, the integrin α7β1 antibody or antigen binding fragment thereof comprises a variable heavy region comprising an amino acid sequence at least 94% identical to SEQ ID NO: 201. In some embodiments, the integrin α7β1 antibody or antigen binding fragment thereof comprises a variable heavy region comprising an amino acid sequence at least 95% identical to SEQ ID NO: 201. In some embodiments, the integrin α7β1 antibody or antigen binding fragment thereof comprises a variable heavy region comprising an amino acid sequence at least 97% identical to SEQ ID NO: 201. In some embodiments, the integrin α7β1 antibody or antigen binding fragment thereof comprises a variable heavy region comprising an amino acid sequence at least 98% identical to SEQ ID NO: 201. In some embodiments, the integrin α7β1 antibody or antigen binding fragment thereof comprises a variable heavy region comprising an amino acid sequence at least 99% identical to SEQ ID NO: 201. In some embodiments, the integrin α7β1 antibody or antigen binding fragment thereof comprises a variable heavy region comprising an amino acid sequence identical to SEQ ID NO: 201.

In some embodiments, the integrin α7β1 antibody or antigen binding fragment thereof comprises a variable heavy region comprising an amino acid sequence at least 80% identical to SEQ ID NO: 203. In some embodiments, the integrin α7β1 antibody or antigen binding fragment thereof comprises a variable heavy region comprising an amino acid sequence at least 83% identical to SEQ ID NO: 203. In some embodiments, the integrin α7β1 antibody or antigen binding fragment thereof comprises a variable heavy region comprising an amino acid sequence at least 85% identical to SEQ ID NO: 203. In some embodiments, the integrin α7β1 antibody or antigen binding fragment thereof comprises a variable heavy region comprising an amino acid sequence at least 87% identical to SEQ ID NO: 203. In some embodiments, the integrin α7β1 antibody or antigen binding fragment thereof comprises a variable heavy region comprising an amino acid sequence at least 90% identical to SEQ ID NO: 203. In some embodiments, the integrin α7β1 antibody or antigen binding fragment thereof comprises a variable heavy region comprising an amino acid sequence at least 92% identical to SEQ ID NO: 203. In some embodiments, the integrin α7β1 antibody or antigen binding fragment thereof comprises a variable heavy region comprising an amino acid sequence at least 93% identical to SEQ ID NO: 203. In some embodiments, the integrin α7β1 antibody or antigen binding fragment thereof comprises a variable heavy region comprising an amino acid sequence at least 94% identical to SEQ ID NO: 203. In some embodiments, the integrin α7β1 antibody or antigen binding fragment thereof comprises a variable heavy region comprising an amino acid sequence at least 95% identical to SEQ ID NO: 203. In some embodiments, the integrin α7β1 antibody or antigen binding fragment thereof comprises a variable heavy region comprising an amino acid sequence at least 97% identical to SEQ ID NO: 203. In some embodiments, the integrin α7β1 antibody or antigen binding fragment thereof comprises a variable heavy region comprising an amino acid sequence at least 98% identical to SEQ ID NO: 203. In some embodiments, the integrin α7β1 antibody or antigen binding fragment thereof comprises a variable heavy region comprising an amino acid sequence at least 99% identical to SEQ ID NO: 203. In some embodiments, the integrin α7β1 antibody or antigen binding fragment thereof comprises a variable heavy region comprising an amino acid sequence identical to SEQ ID NO: 203.

In some embodiments, the integrin α7β1 antibody or antigen binding fragment thereof comprises a variable heavy region comprising an amino acid sequence at least 80% identical to SEQ ID NO: 205. In some embodiments, the integrin α7β1 antibody or antigen binding fragment thereof comprises a variable heavy region comprising an amino acid sequence at least 83% identical to SEQ ID NO: 205. In some embodiments, the integrin α7β1 antibody or antigen binding fragment thereof comprises a variable heavy region comprising an amino acid sequence at least 85% identical to SEQ ID NO: 205. In some embodiments, the integrin α7β1 antibody or antigen binding fragment thereof comprises a variable heavy region comprising an amino acid sequence at least 87% identical to SEQ ID NO: 205. In some embodiments, the integrin α7β1 antibody or antigen binding fragment thereof comprises a variable heavy region comprising an amino acid sequence at least 90% identical to SEQ ID NO: 205. In some embodiments, the integrin α7β1 antibody or antigen binding fragment thereof comprises a variable heavy region comprising an amino acid sequence at least 92% identical to SEQ ID NO: 205. In some embodiments, the integrin α7β1 antibody or antigen binding fragment thereof comprises a variable heavy region comprising an amino acid sequence at least 93% identical to SEQ ID NO: 205. In some embodiments, the integrin α7β1 antibody or antigen binding fragment thereof comprises a variable heavy region comprising an amino acid sequence at least 94% identical to SEQ ID NO: 205. In some embodiments, the integrin α7β1 antibody or antigen binding fragment thereof comprises a variable heavy region comprising an amino acid sequence at least 95% identical to SEQ ID NO: 205. In some embodiments, the integrin α7β1 antibody or antigen binding fragment thereof comprises a variable heavy region comprising an amino acid sequence at least 97% identical to SEQ ID NO: 205. In some embodiments, the integrin α7β1 antibody or antigen binding fragment thereof comprises a variable heavy region comprising an amino acid sequence at least 98% identical to SEQ ID NO: 205. In some embodiments, the integrin α7β1 antibody or antigen binding fragment thereof comprises a variable heavy region comprising an amino acid sequence at least 99% identical to SEQ ID NO: 205. In some embodiments, the integrin α7β1 antibody or antigen binding fragment thereof comprises a variable heavy region comprising an amino acid sequence identical to SEQ ID NO: 205.

In some embodiments, the integrin α7β1 antibody or antigen binding fragment thereof comprises a variable heavy region comprising an amino acid sequence at least 80% identical to SEQ ID NO: 207. In some embodiments, the integrin α7β1 antibody or antigen binding fragment thereof comprises a variable heavy region comprising an amino acid sequence at least 83% identical to SEQ ID NO: 207. In some embodiments, the integrin α7β1 antibody or antigen binding fragment thereof comprises a variable heavy region comprising an amino acid sequence at least 85% identical to SEQ ID NO: 207. In some embodiments, the integrin α7β1 antibody or antigen binding fragment thereof comprises a variable heavy region comprising an amino acid sequence at least 87% identical to SEQ ID NO: 207. In some embodiments, the integrin α7β1 antibody or antigen binding fragment thereof comprises a variable heavy region comprising an amino acid sequence at least 90% identical to SEQ ID NO: 207. In some embodiments, the integrin α7β1 antibody or antigen binding fragment thereof comprises a variable heavy region comprising an amino acid sequence at least 92% identical to SEQ ID NO: 207. In some embodiments, the integrin α7β1 antibody or antigen binding fragment thereof comprises a variable heavy region comprising an amino acid sequence at least 93% identical to SEQ ID NO: 207. In some embodiments, the integrin α7β1 antibody or antigen binding fragment thereof comprises a variable heavy region comprising an amino acid sequence at least 94% identical to SEQ ID NO: 207. In some embodiments, the integrin α7β1 antibody or antigen binding fragment thereof comprises a variable heavy region comprising an amino acid sequence at least 95% identical to SEQ ID NO: 207. In some embodiments, the integrin α7β1 antibody or antigen binding fragment thereof comprises a variable heavy region comprising an amino acid sequence at least 97% identical to SEQ ID NO: 207. In some embodiments, the integrin α7β1 antibody or antigen binding fragment thereof comprises a variable heavy region comprising an amino acid sequence at least 98% identical to SEQ ID NO: 207. In some embodiments, the integrin α7β1 antibody or antigen binding fragment thereof comprises a variable heavy region comprising an amino acid sequence at least 99% identical to SEQ ID NO: 207. In some embodiments, the integrin α7β1 antibody or antigen binding fragment thereof comprises a variable heavy region comprising an amino acid sequence identical to SEQ ID NO: 207.

In some embodiments, the integrin α7β1 antibody or antigen binding fragment thereof comprises a variable heavy region comprising an amino acid sequence at least 80% identical to SEQ ID NO: 209. In some embodiments, the integrin α7β1 antibody or antigen binding fragment thereof comprises a variable heavy region comprising an amino acid sequence at least 83% identical to SEQ ID NO: 209. In some embodiments, the integrin α7β1 antibody or antigen binding fragment thereof comprises a variable heavy region comprising an amino acid sequence at least 85% identical to SEQ ID NO: 209. In some embodiments, the integrin α7β1 antibody or antigen binding fragment thereof comprises a variable heavy region comprising an amino acid sequence at least 87% identical to SEQ ID NO: 209. In some embodiments, the integrin α7β1 antibody or antigen binding fragment thereof comprises a variable heavy region comprising an amino acid sequence at least 90% identical to SEQ ID NO: 209. In some embodiments, the integrin α7β1 antibody or antigen binding fragment thereof comprises a variable heavy region comprising an amino acid sequence at least 92% identical to SEQ ID NO: 209. In some embodiments, the integrin α7β1 antibody or antigen binding fragment thereof comprises a variable heavy region comprising an amino acid sequence at least 93% identical to SEQ ID NO: 9. In some embodiments, the integrin α7β1 antibody or antigen binding fragment thereof comprises a variable heavy region comprising an amino acid sequence at least 94% identical to SEQ ID NO: 209. In some embodiments, the integrin α7β1 antibody or antigen binding fragment thereof comprises a variable heavy region comprising an amino acid sequence at least 95% identical to SEQ ID NO: 209. In some embodiments, the integrin α7β1 antibody or antigen binding fragment thereof comprises a variable heavy region comprising an amino acid sequence at least 97% identical to SEQ ID NO: 209. In some embodiments, the integrin α7β1 antibody or antigen binding fragment thereof comprises a variable heavy region comprising an amino acid sequence at least 98% identical to SEQ ID NO: 209. In some embodiments, the integrin α7β1 antibody or antigen binding fragment thereof comprises a variable heavy region comprising an amino acid sequence at least 99% identical to SEQ ID NO: 209. In some embodiments, the integrin α7β1 antibody or antigen binding fragment thereof comprises a variable heavy region comprising an amino acid sequence identical to SEQ ID NO: 209.

In some embodiments, the integrin α7β1 antibody or antigen binding fragment thereof comprises a variable heavy region comprising an amino acid sequence at least 80% identical to SEQ ID NO: 211. In some embodiments, the integrin α7β1 antibody or antigen binding fragment thereof comprises a variable heavy region comprising an amino acid sequence at least 83% identical to SEQ ID NO: 211. In some embodiments, the integrin α7β1 antibody or antigen binding fragment thereof comprises a variable heavy region comprising an amino acid sequence at least 85% identical to SEQ ID NO: 211. In some embodiments, the integrin α7β1 antibody or antigen binding fragment thereof comprises a variable heavy region comprising an amino acid sequence at least 87% identical to SEQ ID NO: 211. In some embodiments, the integrin α7β1 antibody or antigen binding fragment thereof comprises a variable heavy region comprising an amino acid sequence at least 90% identical to SEQ ID NO: 211. In some embodiments, the integrin α7β1 antibody or antigen binding fragment thereof comprises a variable heavy region comprising an amino acid sequence at least 92% identical to SEQ ID NO: 211. In some embodiments, the integrin α7β1 antibody or antigen binding fragment thereof comprises a variable heavy region comprising an amino acid sequence at least 93% identical to SEQ ID NO: 211. In some embodiments, the integrin α7β1 antibody or antigen binding fragment thereof comprises a variable heavy region comprising an amino acid sequence at least 94% identical to SEQ ID NO: 211. In some embodiments, the integrin α7β1 antibody or antigen binding fragment thereof comprises a variable heavy region comprising an amino acid sequence at least 95% identical to SEQ ID NO: 211. In some embodiments, the integrin α7β1 antibody or antigen binding fragment thereof comprises a variable heavy region comprising an amino acid sequence at least 97% identical to SEQ ID NO: 211. In some embodiments, the integrin α7β1 antibody or antigen binding fragment thereof comprises a variable heavy region comprising an amino acid sequence at least 98% identical to SEQ ID NO: 211. In some embodiments, the integrin α7β1 antibody or antigen binding fragment thereof comprises a variable heavy region comprising an amino acid sequence at least 99% identical to SEQ ID NO: 211. In some embodiments, the integrin α7β1 antibody or antigen binding fragment thereof comprises a variable heavy region comprising an amino acid sequence identical to SEQ ID NO: 211.

In some embodiments, the integrin α7β1 antibody or antigen binding fragment thereof comprises a variable heavy region comprising an amino acid sequence at least 80% identical to SEQ ID NO: 213. In some embodiments, the integrin α7β1 antibody or antigen binding fragment thereof comprises a variable heavy region comprising an amino acid sequence at least 83% identical to SEQ ID NO: 213. In some embodiments, the integrin α7β1 antibody or antigen binding fragment thereof comprises a variable heavy region comprising an amino acid sequence at least 85% identical to SEQ ID NO: 213. In some embodiments, the integrin α7β1 antibody or antigen binding fragment thereof comprises a variable heavy region comprising an amino acid sequence at least 87% identical to SEQ ID NO: 213. In some embodiments, the integrin α7β1 antibody or antigen binding fragment thereof comprises a variable heavy region comprising an amino acid sequence at least 90% identical to SEQ ID NO: 213. In some embodiments, the integrin α7β1 antibody or antigen binding fragment thereof comprises a variable heavy region comprising an amino acid sequence at least 92% identical to SEQ ID NO: 213. In some embodiments, the integrin α7β1 antibody or antigen binding fragment thereof comprises a variable heavy region comprising an amino acid sequence at least 93% identical to SEQ ID NO: 213. In some embodiments, the integrin α7β1 antibody or antigen binding fragment thereof comprises a variable heavy region comprising an amino acid sequence at least 94% identical to SEQ ID NO: 213. In some embodiments, the integrin α7β1 antibody or antigen binding fragment thereof comprises a variable heavy region comprising an amino acid sequence at least 95% identical to SEQ ID NO: 213. In some embodiments, the integrin α7β1 antibody or antigen binding fragment thereof comprises a variable heavy region comprising an amino acid sequence at least 97% identical to SEQ ID NO: 213. In some embodiments, the integrin α7β1 antibody or antigen binding fragment thereof comprises a variable heavy region comprising an amino acid sequence at least 98% identical to SEQ ID NO: 213. In some embodiments, the integrin α7β1 antibody or antigen binding fragment thereof comprises a variable heavy region comprising an amino acid sequence at least 99% identical to SEQ ID NO: 213. In some embodiments, the integrin α7β1 antibody or antigen binding fragment thereof comprises a variable heavy region comprising an amino acid sequence identical to SEQ ID NO: 213.

In some embodiments, the integrin α7β1 antibody or antigen binding fragment thereof comprises a variable heavy region comprising an amino acid sequence at least 80% identical to SEQ ID NO: 215. In some embodiments, the integrin α7β1 antibody or antigen binding fragment thereof comprises a variable heavy region comprising an amino acid sequence at least 83% identical to SEQ ID NO: 215. In some embodiments, the integrin α7β1 antibody or antigen binding fragment thereof comprises a variable heavy region comprising an amino acid sequence at least 85% identical to SEQ ID NO: 215. In some embodiments, the integrin α7β1 antibody or antigen binding fragment thereof comprises a variable heavy region comprising an amino acid sequence at least 87% identical to SEQ ID NO: 215. In some embodiments, the integrin α7β1 antibody or antigen binding fragment thereof comprises a variable heavy region comprising an amino acid sequence at least 90% identical to SEQ ID NO: 215. In some embodiments, the integrin α7β1 antibody or antigen binding fragment thereof comprises a variable heavy region comprising an amino acid sequence at least 92% identical to SEQ ID NO: 215. In some embodiments, the integrin α7β1 antibody or antigen binding fragment thereof comprises a variable heavy region comprising an amino acid sequence at least 93% identical to SEQ ID NO: 215. In some embodiments, the integrin α7β1 antibody or antigen binding fragment thereof comprises a variable heavy region comprising an amino acid sequence at least 94% identical to SEQ ID NO: 215. In some embodiments, the integrin α7β1 antibody or antigen binding fragment thereof comprises a variable heavy region comprising an amino acid sequence at least 95% identical to SEQ ID NO: 215. In some embodiments, the integrin α7β1 antibody or antigen binding fragment thereof comprises a variable heavy region comprising an amino acid sequence at least 97% identical to SEQ ID NO: 215. In some embodiments, the integrin α7β1 antibody or antigen binding fragment thereof comprises a variable heavy region comprising an amino acid sequence at least 98% identical to SEQ ID NO: 215. In some embodiments, the integrin α7β1 antibody or antigen binding fragment thereof comprises a variable heavy region comprising an amino acid sequence at least 99% identical to SEQ ID NO: 215. In some embodiments, the integrin α7β1 antibody or antigen binding fragment thereof comprises a variable heavy region comprising an amino acid sequence identical to SEQ ID NO: 215.

In some embodiments, the integrin α7β1 antibody or antigen binding fragment thereof comprises a variable heavy region comprising an amino acid sequence at least 80% identical to SEQ ID NO: 217. In some embodiments, the integrin α7β1 antibody or antigen binding fragment thereof comprises a variable heavy region comprising an amino acid sequence at least 83% identical to SEQ ID NO: 217. In some embodiments, the integrin α7β1 antibody or antigen binding fragment thereof comprises a variable heavy region comprising an amino acid sequence at least 85% identical to SEQ ID NO: 217. In some embodiments, the integrin α7β1 antibody or antigen binding fragment thereof comprises a variable heavy region comprising an amino acid sequence at least 87% identical to SEQ ID NO: 217. In some embodiments, the integrin α7β1 antibody or antigen binding fragment thereof comprises a variable heavy region comprising an amino acid sequence at least 90% identical to SEQ ID NO: 217. In some embodiments, the integrin α7β1 antibody or antigen binding fragment thereof comprises a variable heavy region comprising an amino acid sequence at least 92% identical to SEQ ID NO: 217. In some embodiments, the integrin α7β1 antibody or antigen binding fragment thereof comprises a variable heavy region comprising an amino acid sequence at least 93% identical to SEQ ID NO: 217. In some embodiments, the integrin α7β1 antibody or antigen binding fragment thereof comprises a variable heavy region comprising an amino acid sequence at least 94% identical to SEQ ID NO: 217. In some embodiments, the integrin α7β1 antibody or antigen binding fragment thereof comprises a variable heavy region comprising an amino acid sequence at least 95% identical to SEQ ID NO: 217. In some embodiments, the integrin α7β1 antibody or antigen binding fragment thereof comprises a variable heavy region comprising an amino acid sequence at least 97% identical to SEQ ID NO: 217. In some embodiments, the integrin α7β1 antibody or antigen binding fragment thereof comprises a variable heavy region comprising an amino acid sequence at least 98% identical to SEQ ID NO: 217. In some embodiments, the integrin α7β1 antibody or antigen binding fragment thereof comprises a variable heavy region comprising an amino acid sequence at least 99% identical to SEQ ID NO: 217. In some embodiments, the integrin α7β1 antibody or antigen binding fragment thereof comprises a variable heavy region comprising an amino acid sequence identical to SEQ ID NO: 217.

In some embodiments, the integrin α7β1 antibody or antigen binding fragment thereof comprises a variable heavy region comprising an amino acid sequence at least 80% identical to SEQ ID NO: 219. In some embodiments, the integrin α7β1 antibody or antigen binding fragment thereof comprises a variable heavy region comprising an amino acid sequence at least 83% identical to SEQ ID NO: 219. In some embodiments, the integrin α7β1 antibody or antigen binding fragment thereof comprises a variable heavy region comprising an amino acid sequence at least 85% identical to SEQ ID NO: 219. In some embodiments, the integrin α7β1 antibody or antigen binding fragment thereof comprises a variable heavy region comprising an amino acid sequence at least 87% identical to SEQ ID NO: 219. In some embodiments, the integrin α7β1 antibody or antigen binding fragment thereof comprises a variable heavy region comprising an amino acid sequence at least 90% identical to SEQ ID NO: 219. In some embodiments, the integrin α7β1 antibody or antigen binding fragment thereof comprises a variable heavy region comprising an amino acid sequence at least 92% identical to SEQ ID NO: 219. In some embodiments, the integrin α7β1 antibody or antigen binding fragment thereof comprises a variable heavy region comprising an amino acid sequence at least 93% identical to SEQ ID NO: 219. In some embodiments, the integrin α7β1 antibody or antigen binding fragment thereof comprises a variable heavy region comprising an amino acid sequence at least 94% identical to SEQ ID NO: 219. In some embodiments, the integrin α7β1 antibody or antigen binding fragment thereof comprises a variable heavy region comprising an amino acid sequence at least 95% identical to SEQ ID NO: 219. In some embodiments, the integrin α7β1 antibody or antigen binding fragment thereof comprises a variable heavy region comprising an amino acid sequence at least 97% identical to SEQ ID NO: 219. In some embodiments, the integrin α7β1 antibody or antigen binding fragment thereof comprises a variable heavy region comprising an amino acid sequence at least 98% identical to SEQ ID NO: 219. In some embodiments, the integrin α7β1 antibody or antigen binding fragment thereof comprises a variable heavy region comprising an amino acid sequence at least 99% identical to SEQ ID NO: 219. In some embodiments, the integrin α7β1 antibody or antigen binding fragment thereof comprises a variable heavy region comprising an amino acid sequence identical to SEQ ID NO: 219.

In some embodiments, the integrin α7β1 antibody or antigen binding fragment thereof comprises a variable heavy region comprising an amino acid sequence at least 80% identical to SEQ ID NO: 221. In some embodiments, the integrin α7β1 antibody or antigen binding fragment thereof comprises a variable heavy region comprising an amino acid sequence at least 83% identical to SEQ ID NO: 221. In some embodiments, the integrin α7β1 antibody or antigen binding fragment thereof comprises a variable heavy region comprising an amino acid sequence at least 85% identical to SEQ ID NO: 221. In some embodiments, the integrin α7β1 antibody or antigen binding fragment thereof comprises a variable heavy region comprising an amino acid sequence at least 87% identical to SEQ ID NO: 221. In some embodiments, the integrin α7β1 antibody or antigen binding fragment thereof comprises a variable heavy region comprising an amino acid sequence at least 90% identical to SEQ ID NO: 221. In some embodiments, the integrin α7β1 antibody or antigen binding fragment thereof comprises a variable heavy region comprising an amino acid sequence at least 92% identical to SEQ ID NO: 221. In some embodiments, the integrin α7β1 antibody or antigen binding fragment thereof comprises a variable heavy region comprising an amino acid sequence at least 93% identical to SEQ ID NO: 221. In some embodiments, the integrin α7β1 antibody or antigen binding fragment thereof comprises a variable heavy region comprising an amino acid sequence at least 94% identical to SEQ ID NO: 221. In some embodiments, the integrin α7β1 antibody or antigen binding fragment thereof comprises a variable heavy region comprising an amino acid sequence at least 95% identical to SEQ ID NO: 221. In some embodiments, the integrin α7β1 antibody or antigen binding fragment thereof comprises a variable heavy region comprising an amino acid sequence at least 97% identical to SEQ ID NO: 221. In some embodiments, the integrin α7β1 antibody or antigen binding fragment thereof comprises a variable heavy region comprising an amino acid sequence at least 98% identical to SEQ ID NO: 221. In some embodiments, the integrin α7β1 antibody or antigen binding fragment thereof comprises a variable heavy region comprising an amino acid sequence at least 99% identical to SEQ ID NO: 221. In some embodiments, the integrin α7β1 antibody or antigen binding fragment thereof comprises a variable heavy region comprising an amino acid sequence identical to SEQ ID NO: 221.

In some embodiments, the integrin α7β1 antibody or antigen binding fragment thereof comprises a variable heavy region comprising an amino acid sequence at least 80% identical to SEQ ID NO: 223. In some embodiments, the integrin α7β1 antibody or antigen binding fragment thereof comprises a variable heavy region comprising an amino acid sequence at least 83% identical to SEQ ID NO: 223. In some embodiments, the integrin α7β1 antibody or antigen binding fragment thereof comprises a variable heavy region comprising an amino acid sequence at least 85% identical to SEQ ID NO: 223. In some embodiments, the integrin α7β1 antibody or antigen binding fragment thereof comprises a variable heavy region comprising an amino acid sequence at least 87% identical to SEQ ID NO: 223. In some embodiments, the integrin α7β1 antibody or antigen binding fragment thereof comprises a variable heavy region comprising an amino acid sequence at least 90% identical to SEQ ID NO: 223. In some embodiments, the integrin α7β1 antibody or antigen binding fragment thereof comprises a variable heavy region comprising an amino acid sequence at least 92% identical to SEQ ID NO: 223. In some embodiments, the integrin α7β1 antibody or antigen binding fragment thereof comprises a variable heavy region comprising an amino acid sequence at least 93% identical to SEQ ID NO: 223. In some embodiments, the integrin α7β1 antibody or antigen binding fragment thereof comprises a variable heavy region comprising an amino acid sequence at least 94% identical to SEQ ID NO: 223. In some embodiments, the integrin α7β1 antibody or antigen binding fragment thereof comprises a variable heavy region comprising an amino acid sequence at least 95% identical to SEQ ID NO: 223. In some embodiments, the integrin α7β1 antibody or antigen binding fragment thereof comprises a variable heavy region comprising an amino acid sequence at least 97% identical to SEQ ID NO: 223. In some embodiments, the integrin α7β1 antibody or antigen binding fragment thereof comprises a variable heavy region comprising an amino acid sequence at least 98% identical to SEQ ID NO: 223. In some embodiments, the integrin α7β1 antibody or antigen binding fragment thereof comprises a variable heavy region comprising an amino acid sequence at least 99% identical to SEQ ID NO: 223. In some embodiments, the integrin α7β1 antibody or antigen binding fragment thereof comprises a variable heavy region comprising an amino acid sequence identical to SEQ ID NO: 223.

In some embodiments, the integrin α7β1 antibody or antigen binding fragment thereof comprises a variable light region comprising an amino acid sequence at least 80% identical to SEQ ID NO: 187. In some embodiments, the integrin α7β1 antibody or antigen binding fragment thereof comprises a variable light region comprising an amino acid sequence at least 83% identical to SEQ ID NO: 187. In some embodiments, the integrin α7β1 antibody or antigen binding fragment thereof comprises a variable light region comprising an amino acid sequence at least 85% identical to SEQ ID NO: 187. In some embodiments, the integrin α7β1 antibody or antigen binding fragment thereof comprises a variable light region comprising an amino acid sequence at least 87% identical to SEQ ID NO: 187. In some embodiments, the integrin α7β1 antibody or antigen binding fragment thereof comprises a variable light region comprising an amino acid sequence at least 90% identical to SEQ ID NO: 187. In some embodiments, the integrin α7β1 antibody or antigen binding fragment thereof comprises a variable light region comprising an amino acid sequence at least 92% identical to SEQ ID NO: 187. In some embodiments, the integrin α7β1 antibody or antigen binding fragment thereof comprises a variable light region comprising an amino acid sequence at least 93% identical to SEQ ID NO: 187. In some embodiments, the integrin α7β1 antibody or antigen binding fragment thereof comprises a variable light region comprising an amino acid sequence at least 94% identical to SEQ ID NO: 187. In some embodiments, the integrin α7β1 antibody or antigen binding fragment thereof comprises a variable light region comprising an amino acid sequence at least 95% identical to SEQ ID NO: 187. In some embodiments, the integrin α7β1 antibody or antigen binding fragment thereof comprises a variable light region comprising an amino acid sequence at least 97% identical to SEQ ID NO: 187. In some embodiments, the integrin α7β1 antibody or antigen binding fragment thereof comprises a variable light region comprising an amino acid sequence at least 98% identical to SEQ ID NO: 187. In some embodiments, the integrin α7β1 antibody or antigen binding fragment thereof comprises a variable light region comprising an amino acid sequence at least 99% identical to SEQ ID NO: 187. In some embodiments, the integrin α7β1 antibody or antigen binding fragment thereof comprises a variable light region comprising an amino acid sequence identical to SEQ ID NO: 187.

In some embodiments, the integrin α7β1 antibody or antigen binding fragment thereof comprises a variable light region comprising an amino acid sequence at least 80% identical to SEQ ID NO: 109. In some embodiments, the integrin α7β1 antibody or antigen binding fragment thereof comprises a variable light region comprising an amino acid sequence at least 83% identical to SEQ ID NO: 109. In some embodiments, the integrin α7β1 antibody or antigen binding fragment thereof comprises a variable light region comprising an amino acid sequence at least 85% identical to SEQ ID NO: 109. In some embodiments, the integrin α7β1 antibody or antigen binding fragment thereof comprises a variable light region comprising an amino acid sequence at least 87% identical to SEQ ID NO: 109. In some embodiments, the integrin α7β1 antibody or antigen binding fragment thereof comprises a variable light region comprising an amino acid sequence at least 90% identical to SEQ ID NO: 109. In some embodiments, the integrin α7β1 antibody or antigen binding fragment thereof comprises a variable light region comprising an amino acid sequence at least 92% identical to SEQ ID NO: 109. In some embodiments, the integrin α7β1 antibody or antigen binding fragment thereof comprises a variable light region comprising an amino acid sequence at least 93% identical to SEQ ID NO: 109. In some embodiments, the integrin α7β1 antibody or antigen binding fragment thereof comprises a variable light region comprising an amino acid sequence at least 94% identical to SEQ ID NO: 109. In some embodiments, the integrin α7β1 antibody or antigen binding fragment thereof comprises a variable light region comprising an amino acid sequence at least 95% identical to SEQ ID NO: 109. In some embodiments, the integrin α7β1 antibody or antigen binding fragment thereof comprises a variable light region comprising an amino acid sequence at least 97% identical to SEQ ID NO: 109. In some embodiments, the integrin α7β1 antibody or antigen binding fragment thereof comprises a variable light region comprising an amino acid sequence at least 98% identical to SEQ ID NO: 109. In some embodiments, the integrin α7β1 antibody or antigen binding fragment thereof comprises a variable light region comprising an amino acid sequence at least 99% identical to SEQ ID NO: 109. In some embodiments, the integrin α7β1 antibody or antigen binding fragment thereof comprises a variable light region comprising an amino acid sequence identical to SEQ ID NO: 109.

In some embodiments, the integrin α7β1 antibody or antigen binding fragment thereof comprises a variable light region comprising an amino acid sequence at least 80% identical to SEQ ID NO: 111. In some embodiments, the integrin α7β1 antibody or antigen binding fragment thereof comprises a variable light region comprising an amino acid sequence at least 83% identical to SEQ ID NO: 111. In some embodiments, the integrin α7β1 antibody or antigen binding fragment thereof comprises a variable light region comprising an amino acid sequence at least 85% identical to SEQ ID NO: 111. In some embodiments, the integrin α7β1 antibody or antigen binding fragment thereof comprises a variable light region comprising an amino acid sequence at least 87% identical to SEQ ID NO: 111. In some embodiments, the integrin α7β1 antibody or antigen binding fragment thereof comprises a variable light region comprising an amino acid sequence at least 90% identical to SEQ ID NO: 111. In some embodiments, the integrin α7β1 antibody or antigen binding fragment thereof comprises a variable light region comprising an amino acid sequence at least 92% identical to SEQ ID NO: 111. In some embodiments, the integrin α7β1 antibody or antigen binding fragment thereof comprises a variable light region comprising an amino acid sequence at least 93% identical to SEQ ID NO: 111. In some embodiments, the integrin α7β1 antibody or antigen binding fragment thereof comprises a variable light region comprising an amino acid sequence at least 94% identical to SEQ ID NO: 111. In some embodiments, the integrin α7β1 antibody or antigen binding fragment thereof comprises a variable light region comprising an amino acid sequence at least 95% identical to SEQ ID NO: 111. In some embodiments, the integrin α7β1 antibody or antigen binding fragment thereof comprises a variable light region comprising an amino acid sequence at least 97% identical to SEQ ID NO: 111. In some embodiments, the integrin α7β1 antibody or antigen binding fragment thereof comprises a variable light region comprising an amino acid sequence at least 98% identical to SEQ ID NO: 111. In some embodiments, the integrin α7β1 antibody or antigen binding fragment thereof comprises a variable light region comprising an amino acid sequence at least 99% identical to SEQ ID NO: 111. In some embodiments, the integrin α7β1 antibody or antigen binding fragment thereof comprises a variable light region comprising an amino acid sequence identical to SEQ ID NO: 111.

In some embodiments, the integrin α7β1 antibody or antigen binding fragment thereof comprises a variable light region comprising an amino acid sequence at least 80% identical to SEQ ID NO: 200. In some embodiments, the integrin α7β1 antibody or antigen binding fragment thereof comprises a variable light region comprising an amino acid sequence at least 83% identical to SEQ ID NO: 200. In some embodiments, the integrin α7β1 antibody or antigen binding fragment thereof comprises a variable light region comprising an amino acid sequence at least 85% identical to SEQ ID NO: 200. In some embodiments, the integrin α7β1 antibody or antigen binding fragment thereof comprises a variable light region comprising an amino acid sequence at least 87% identical to SEQ ID NO: 200. In some embodiments, the integrin α7β1 antibody or antigen binding fragment thereof comprises a variable light region comprising an amino acid sequence at least 90% identical to SEQ ID NO: 200. In some embodiments, the integrin α7β1 antibody or antigen binding fragment thereof comprises a variable light region comprising an amino acid sequence at least 92% identical to SEQ ID NO: 200. In some embodiments, the integrin α7β1 antibody or antigen binding fragment thereof comprises a variable light region comprising an amino acid sequence at least 93% identical to SEQ ID NO: 200. In some embodiments, the integrin α7β1 antibody or antigen binding fragment thereof comprises a variable light region comprising an amino acid sequence at least 94% identical to SEQ ID NO: 200. In some embodiments, the integrin α7β1 antibody or antigen binding fragment thereof comprises a variable light region comprising an amino acid sequence at least 95% identical to SEQ ID NO: 200. In some embodiments, the integrin α7β1 antibody or antigen binding fragment thereof comprises a variable light region comprising an amino acid sequence at least 97% identical to SEQ ID NO: 200. In some embodiments, the integrin α7β1 antibody or antigen binding fragment thereof comprises a variable light region comprising an amino acid sequence at least 98% identical to SEQ ID NO: 200. In some embodiments, the integrin α7β1 antibody or antigen binding fragment thereof comprises a variable light region comprising an amino acid sequence at least 99% identical to SEQ ID NO: 200. In some embodiments, the integrin α7β1 antibody or antigen binding fragment thereof comprises a variable light region comprising an amino acid sequence identical to SEQ ID NO: 200.

In some embodiments, the integrin α7β1 antibody or antigen binding fragment thereof comprises a variable light region comprising an amino acid sequence at least 80% identical to SEQ ID NO: 202. In some embodiments, the integrin α7β1 antibody or antigen binding fragment thereof comprises a variable light region comprising an amino acid sequence at least 83% identical to SEQ ID NO: 202. In some embodiments, the integrin α7β1 antibody or antigen binding fragment thereof comprises a variable light region comprising an amino acid sequence at least 85% identical to SEQ ID NO: 202. In some embodiments, the integrin α7β1 antibody or antigen binding fragment thereof comprises a variable light region comprising an amino acid sequence at least 87% identical to SEQ ID NO: 202. In some embodiments, the integrin α7β1 antibody or antigen binding fragment thereof comprises a variable light region comprising an amino acid sequence at least 90% identical to SEQ ID NO: 202. In some embodiments, the integrin α7β1 antibody or antigen binding fragment thereof comprises a variable light region comprising an amino acid sequence at least 92% identical to SEQ ID NO: 202. In some embodiments, the integrin α7β1 antibody or antigen binding fragment thereof comprises a variable light region comprising an amino acid sequence at least 93% identical to SEQ ID NO: 202. In some embodiments, the integrin α7β1 antibody or antigen binding fragment thereof comprises a variable light region comprising an amino acid sequence at least 94% identical to SEQ ID NO: 202. In some embodiments, the integrin α7β1 antibody or antigen binding fragment thereof comprises a variable light region comprising an amino acid sequence at least 95% identical to SEQ ID NO: 202. In some embodiments, the integrin α7β1 antibody or antigen binding fragment thereof comprises a variable light region comprising an amino acid sequence at least 97% identical to SEQ ID NO: 202. In some embodiments, the integrin α7β1 antibody or antigen binding fragment thereof comprises a variable light region comprising an amino acid sequence at least 98% identical to SEQ ID NO: 202. In some embodiments, the integrin α7β1 antibody or antigen binding fragment thereof comprises a variable light region comprising an amino acid sequence at least 99% identical to SEQ ID NO: 202. In some embodiments, the integrin α7β1 antibody or antigen binding fragment thereof comprises a variable light region comprising an amino acid sequence identical to SEQ ID NO: 202.

In some embodiments, the integrin α7β1 antibody or antigen binding fragment thereof comprises a variable light region comprising an amino acid sequence at least 80% identical to SEQ ID NO: 204. In some embodiments, the integrin α7β1 antibody or antigen binding fragment thereof comprises a variable light region comprising an amino acid sequence at least 83% identical to SEQ ID NO: 204. In some embodiments, the integrin α7β1 antibody or antigen binding fragment thereof comprises a variable light region comprising an amino acid sequence at least 85% identical to SEQ ID NO: 204. In some embodiments, the integrin α7β1 antibody or antigen binding fragment thereof comprises a variable light region comprising an amino acid sequence at least 87% identical to SEQ ID NO: 204. In some embodiments, the integrin α7β1 antibody or antigen binding fragment thereof comprises a variable light region comprising an amino acid sequence at least 90% identical to SEQ ID NO: 204. In some embodiments, the integrin α7β1 antibody or antigen binding fragment thereof comprises a variable light region comprising an amino acid sequence at least 92% identical to SEQ ID NO: 204. In some embodiments, the integrin α7β1 antibody or antigen binding fragment thereof comprises a variable light region comprising an amino acid sequence at least 93% identical to SEQ ID NO: 204. In some embodiments, the integrin α7β1 antibody or antigen binding fragment thereof comprises a variable light region comprising an amino acid sequence at least 94% identical to SEQ ID NO: 204. In some embodiments, the integrin α7β1 antibody or antigen binding fragment thereof comprises a variable light region comprising an amino acid sequence at least 95% identical to SEQ ID NO: 204. In some embodiments, the integrin α7β1 antibody or antigen binding fragment thereof comprises a variable light region comprising an amino acid sequence at least 97% identical to SEQ ID NO: 204. In some embodiments, the integrin α7β1 antibody or antigen binding fragment thereof comprises a variable light region comprising an amino acid sequence at least 98% identical to SEQ ID NO: 204. In some embodiments, the integrin α7β1 antibody or antigen binding fragment thereof comprises a variable light region comprising an amino acid sequence at least 99% identical to SEQ ID NO: 204. In some embodiments, the integrin α7β1 antibody or antigen binding fragment thereof comprises a variable light region comprising an amino acid sequence identical to SEQ ID NO: 204.

In some embodiments, the integrin α7β1 antibody or antigen binding fragment thereof comprises a variable light region comprising an amino acid sequence at least 80% identical to SEQ ID NO: 206. In some embodiments, the integrin α7β1 antibody or antigen binding fragment thereof comprises a variable light region comprising an amino acid sequence at least 83% identical to SEQ ID NO: 206. In some embodiments, the integrin α7β1 antibody or antigen binding fragment thereof comprises a variable light region comprising an amino acid sequence at least 85% identical to SEQ ID NO: 206. In some embodiments, the integrin α7β1 antibody or antigen binding fragment thereof comprises a variable light region comprising an amino acid sequence at least 87% identical to SEQ ID NO: 206. In some embodiments, the integrin α7β1 antibody or antigen binding fragment thereof comprises a variable light region comprising an amino acid sequence at least 90% identical to SEQ ID NO: 206. In some embodiments, the integrin α7β1 antibody or antigen binding fragment thereof comprises a variable light region comprising an amino acid sequence at least 92% identical to SEQ ID NO: 206. In some embodiments, the integrin α7β1 antibody or antigen binding fragment thereof comprises a variable light region comprising an amino acid sequence at least 93% identical to SEQ ID NO: 206. In some embodiments, the integrin α7β1 antibody or antigen binding fragment thereof comprises a variable light region comprising an amino acid sequence at least 94% identical to SEQ ID NO: 206. In some embodiments, the integrin α7β1 antibody or antigen binding fragment thereof comprises a variable light region comprising an amino acid sequence at least 95% identical to SEQ ID NO: 206. In some embodiments, the integrin α7β1 antibody or antigen binding fragment thereof comprises a variable light region comprising an amino acid sequence at least 97% identical to SEQ ID NO: 206. In some embodiments, the integrin α7β1 antibody or antigen binding fragment thereof comprises a variable light region comprising an amino acid sequence at least 98% identical to SEQ ID NO: 206. In some embodiments, the integrin α7β1 antibody or antigen binding fragment thereof comprises a variable light region comprising an amino acid sequence at least 99% identical to SEQ ID NO: 206. In some embodiments, the integrin α7β1 antibody or antigen binding fragment thereof comprises a variable light region comprising an amino acid sequence identical to SEQ ID NO: 206.

In some embodiments, the integrin α7β1 antibody or antigen binding fragment thereof comprises a variable light region comprising an amino acid sequence at least 80% identical to SEQ ID NO: 208. In some embodiments, the integrin α7β1 antibody or antigen binding fragment thereof comprises a variable light region comprising an amino acid sequence at least 83% identical to SEQ ID NO: 208. In some embodiments, the integrin α7β1 antibody or antigen binding fragment thereof comprises a variable light region comprising an amino acid sequence at least 85% identical to SEQ ID NO: 208. In some embodiments, the integrin α7β1 antibody or antigen binding fragment thereof comprises a variable light region comprising an amino acid sequence at least 87% identical to SEQ ID NO: 208. In some embodiments, the integrin α7β1 antibody or antigen binding fragment thereof comprises a variable light region comprising an amino acid sequence at least 90% identical to SEQ ID NO: 208. In some embodiments, the integrin α7β1 antibody or antigen binding fragment thereof comprises a variable light region comprising an amino acid sequence at least 92% identical to SEQ ID NO: 208. In some embodiments, the integrin α7β1 antibody or antigen binding fragment thereof comprises a variable light region comprising an amino acid sequence at least 93% identical to SEQ ID NO: 208. In some embodiments, the integrin α7β1 antibody or antigen binding fragment thereof comprises a variable light region comprising an amino acid sequence at least 94% identical to SEQ ID NO: 208. In some embodiments, the integrin α7β1 antibody or antigen binding fragment thereof comprises a variable light region comprising an amino acid sequence at least 95% identical to SEQ ID NO: 208. In some embodiments, the integrin α7β1 antibody or antigen binding fragment thereof comprises a variable light region comprising an amino acid sequence at least 97% identical to SEQ ID NO: 208. In some embodiments, the integrin α7β1 antibody or antigen binding fragment thereof comprises a variable light region comprising an amino acid sequence at least 98% identical to SEQ ID NO: 208. In some embodiments, the integrin α7β1 antibody or antigen binding fragment thereof comprises a variable light region comprising an amino acid sequence at least 99% identical to SEQ ID NO: 208. In some embodiments, the integrin α7β1 antibody or antigen binding fragment thereof comprises a variable light region comprising an amino acid sequence identical to SEQ ID NO: 208.

In some embodiments, the integrin α7β1 antibody or antigen binding fragment thereof comprises a variable light region comprising an amino acid sequence at least 80% identical to SEQ ID NO: 210. In some embodiments, the integrin α7β1 antibody or antigen binding fragment thereof comprises a variable light region comprising an amino acid sequence at least 83% identical to SEQ ID NO: 210. In some embodiments, the integrin α7β1 antibody or antigen binding fragment thereof comprises a variable light region comprising an amino acid sequence at least 85% identical to SEQ ID NO: 210. In some embodiments, the integrin α7β1 antibody or antigen binding fragment thereof comprises a variable light region comprising an amino acid sequence at least 87% identical to SEQ ID NO: 210. In some embodiments, the integrin α7β1 antibody or antigen binding fragment thereof comprises a variable light region comprising an amino acid sequence at least 90% identical to SEQ ID NO: 210. In some embodiments, the integrin α7β1 antibody or antigen binding fragment thereof comprises a variable light region comprising an amino acid sequence at least 92% identical to SEQ ID NO: 210. In some embodiments, the integrin α7β1 antibody or antigen binding fragment thereof comprises a variable light region comprising an amino acid sequence at least 93% identical to SEQ ID NO: 210. In some embodiments, the integrin α7β1 antibody or antigen binding fragment thereof comprises a variable light region comprising an amino acid sequence at least 94% identical to SEQ ID NO: 210. In some embodiments, the integrin α7β1 antibody or antigen binding fragment thereof comprises a variable light region comprising an amino acid sequence at least 95% identical to SEQ ID NO: 210. In some embodiments, the integrin α7β1 antibody or antigen binding fragment thereof comprises a variable light region comprising an amino acid sequence at least 97% identical to SEQ ID NO: 210. In some embodiments, the integrin α7β1 antibody or antigen binding fragment thereof comprises a variable light region comprising an amino acid sequence at least 98% identical to SEQ ID NO: 210. In some embodiments, the integrin α7β1 antibody or antigen binding fragment thereof comprises a variable light region comprising an amino acid sequence at least 99% identical to SEQ ID NO: 210. In some embodiments, the integrin α7β1 antibody or antigen binding fragment thereof comprises a variable light region comprising an amino acid sequence identical to SEQ ID NO: 210.

In some embodiments, the integrin α7β1 antibody or antigen binding fragment thereof comprises a variable light region comprising an amino acid sequence at least 80% identical to SEQ ID NO: 212. In some embodiments, the integrin α7β1 antibody or antigen binding fragment thereof comprises a variable light region comprising an amino acid sequence at least 83% identical to SEQ ID NO: 212. In some embodiments, the integrin α7β1 antibody or antigen binding fragment thereof comprises a variable light region comprising an amino acid sequence at least 85% identical to SEQ ID NO: 212. In some embodiments, the integrin α7β1 antibody or antigen binding fragment thereof comprises a variable light region comprising an amino acid sequence at least 87% identical to SEQ ID NO: 212. In some embodiments, the integrin α7β1 antibody or antigen binding fragment thereof comprises a variable light region comprising an amino acid sequence at least 90% identical to SEQ ID NO: 212. In some embodiments, the integrin α7β1 antibody or antigen binding fragment thereof comprises a variable light region comprising an amino acid sequence at least 92% identical to SEQ ID NO: 212. In some embodiments, the integrin α7β1 antibody or antigen binding fragment thereof comprises a variable light region comprising an amino acid sequence at least 93% identical to SEQ ID NO: 212. In some embodiments, the integrin α7β1 antibody or antigen binding fragment thereof comprises a variable light region comprising an amino acid sequence at least 94% identical to SEQ ID NO: 212. In some embodiments, the integrin α7β1 antibody or antigen binding fragment thereof comprises a variable light region comprising an amino acid sequence at least 95% identical to SEQ ID NO: 212. In some embodiments, the integrin α7β1 antibody or antigen binding fragment thereof comprises a variable light region comprising an amino acid sequence at least 97% identical to SEQ ID NO: 212. In some embodiments, the integrin α7β1 antibody or antigen binding fragment thereof comprises a variable light region comprising an amino acid sequence at least 98% identical to SEQ ID NO: 212. In some embodiments, the integrin α7β1 antibody or antigen binding fragment thereof comprises a variable light region comprising an amino acid sequence at least 99% identical to SEQ ID NO: 212. In some embodiments, the integrin α7β1 antibody or antigen binding fragment thereof comprises a variable light region comprising an amino acid sequence identical to SEQ ID NO: 212.

In some embodiments, the integrin α7β1 antibody or antigen binding fragment thereof comprises a variable light region comprising an amino acid sequence at least 80% identical to SEQ ID NO: 214. In some embodiments, the integrin α7β1 antibody or antigen binding fragment thereof comprises a variable light region comprising an amino acid sequence at least 83% identical to SEQ ID NO: 214. In some embodiments, the integrin α7β1 antibody or antigen binding fragment thereof comprises a variable light region comprising an amino acid sequence at least 85% identical to SEQ ID NO: 214. In some embodiments, the integrin α7β1 antibody or antigen binding fragment thereof comprises a variable light region comprising an amino acid sequence at least 87% identical to SEQ ID NO: 214. In some embodiments, the integrin α7β1 antibody or antigen binding fragment thereof comprises a variable light region comprising an amino acid sequence at least 90% identical to SEQ ID NO: 214. In some embodiments, the integrin α7β1 antibody or antigen binding fragment thereof comprises a variable light region comprising an amino acid sequence at least 92% identical to SEQ ID NO: 214. In some embodiments, the integrin α7β1 antibody or antigen binding fragment thereof comprises a variable light region comprising an amino acid sequence at least 93% identical to SEQ ID NO: 214. In some embodiments, the integrin α7β1 antibody or antigen binding fragment thereof comprises a variable light region comprising an amino acid sequence at least 94% identical to SEQ ID NO: 214. In some embodiments, the integrin α7β1 antibody or antigen binding fragment thereof comprises a variable light region comprising an amino acid sequence at least 95% identical to SEQ ID NO: 214. In some embodiments, the integrin α7β1 antibody or antigen binding fragment thereof comprises a variable light region comprising an amino acid sequence at least 97% identical to SEQ ID NO: 214. In some embodiments, the integrin α7β1 antibody or antigen binding fragment thereof comprises a variable light region comprising an amino acid sequence at least 98% identical to SEQ ID NO: 214. In some embodiments, the integrin α7β1 antibody or antigen binding fragment thereof comprises a variable light region comprising an amino acid sequence at least 99% identical to SEQ ID NO: 214. In some embodiments, the integrin α7β1 antibody or antigen binding fragment thereof comprises a variable light region comprising an amino acid sequence identical to SEQ ID NO: 214.

In some embodiments, the integrin α7β1 antibody or antigen binding fragment thereof comprises a variable light region comprising an amino acid sequence at least 80% identical to SEQ ID NO: 216. In some embodiments, the integrin α7β1 antibody or antigen binding fragment thereof comprises a variable light region comprising an amino acid sequence at least 83% identical to SEQ ID NO: 216. In some embodiments, the integrin α7β1 antibody or antigen binding fragment thereof comprises a variable light region comprising an amino acid sequence at least 85% identical to SEQ ID NO: 216. In some embodiments, the integrin α7β1 antibody or antigen binding fragment thereof comprises a variable light region comprising an amino acid sequence at least 87% identical to SEQ ID NO: 216. In some embodiments, the integrin α7β1 antibody or antigen binding fragment thereof comprises a variable light region comprising an amino acid sequence at least 90% identical to SEQ ID NO: 216. In some embodiments, the integrin α7β1 antibody or antigen binding fragment thereof comprises a variable light region comprising an amino acid sequence at least 92% identical to SEQ ID NO: 216. In some embodiments, the integrin α7β1 antibody or antigen binding fragment thereof comprises a variable light region comprising an amino acid sequence at least 93% identical to SEQ ID NO: 216. In some embodiments, the integrin α7β1 antibody or antigen binding fragment thereof comprises a variable light region comprising an amino acid sequence at least 94% identical to SEQ ID NO: 216. In some embodiments, the integrin α7β1 antibody or antigen binding fragment thereof comprises a variable light region comprising an amino acid sequence at least 95% identical to SEQ ID NO: 216. In some embodiments, the integrin α7β1 antibody or antigen binding fragment thereof comprises a variable light region comprising an amino acid sequence at least 97% identical to SEQ ID NO: 216. In some embodiments, the integrin α7β1 antibody or antigen binding fragment thereof comprises a variable light region comprising an amino acid sequence at least 98% identical to SEQ ID NO: 216. In some embodiments, the integrin α7β1 antibody or antigen binding fragment thereof comprises a variable light region comprising an amino acid sequence at least 99% identical to SEQ ID NO: 216. In some embodiments, the integrin α7β1 antibody or antigen binding fragment thereof comprises a variable light region comprising an amino acid sequence identical to SEQ ID NO: 216.

In some embodiments, the integrin α7β1 antibody or antigen binding fragment thereof comprises a variable light region comprising an amino acid sequence at least 80% identical to SEQ ID NO: 218. In some embodiments, the integrin α7β1 antibody or antigen binding fragment thereof comprises a variable light region comprising an amino acid sequence at least 83% identical to SEQ ID NO: 218. In some embodiments, the integrin α7β1 antibody or antigen binding fragment thereof comprises a variable light region comprising an amino acid sequence at least 85% identical to SEQ ID NO: 218. In some embodiments, the integrin α7β1 antibody or antigen binding fragment thereof comprises a variable light region comprising an amino acid sequence at least 87% identical to SEQ ID NO: 218. In some embodiments, the integrin α7β1 antibody or antigen binding fragment thereof comprises a variable light region comprising an amino acid sequence at least 90% identical to SEQ ID NO: 218. In some embodiments, the integrin α7β1 antibody or antigen binding fragment thereof comprises a variable light region comprising an amino acid sequence at least 92% identical to SEQ ID NO: 218. In some embodiments, the integrin α7β1 antibody or antigen binding fragment thereof comprises a variable light region comprising an amino acid sequence at least 93% identical to SEQ ID NO: 218. In some embodiments, the integrin α7β1 antibody or antigen binding fragment thereof comprises a variable light region comprising an amino acid sequence at least 94% identical to SEQ ID NO: 218. In some embodiments, the integrin α7β1 antibody or antigen binding fragment thereof comprises a variable light region comprising an amino acid sequence at least 95% identical to SEQ ID NO: 218. In some embodiments, the integrin α7β1 antibody or antigen binding fragment thereof comprises a variable light region comprising an amino acid sequence at least 97% identical to SEQ ID NO: 218. In some embodiments, the integrin α7β1 antibody or antigen binding fragment thereof comprises a variable light region comprising an amino acid sequence at least 98% identical to SEQ ID NO: 218. In some embodiments, the integrin α7β1 antibody or antigen binding fragment thereof comprises a variable light region comprising an amino acid sequence at least 99% identical to SEQ ID NO: 218. In some embodiments, the integrin α7β1 antibody or antigen binding fragment thereof comprises a variable light region comprising an amino acid sequence identical to SEQ ID NO: 218.

In some embodiments, the integrin α7β1 antibody or antigen binding fragment thereof comprises a variable light region comprising an amino acid sequence at least 80% identical to SEQ ID NO: 220. In some embodiments, the integrin α7β1 antibody or antigen binding fragment thereof comprises a variable light region comprising an amino acid sequence at least 83% identical to SEQ ID NO: 220. In some embodiments, the integrin α7β1 antibody or antigen binding fragment thereof comprises a variable light region comprising an amino acid sequence at least 85% identical to SEQ ID NO: 220. In some embodiments, the integrin α7β1 antibody or antigen binding fragment thereof comprises a variable light region comprising an amino acid sequence at least 87% identical to SEQ ID NO: 220. In some embodiments, the integrin α7β1 antibody or antigen binding fragment thereof comprises a variable light region comprising an amino acid sequence at least 90% identical to SEQ ID NO: 220. In some embodiments, the integrin α7β1 antibody or antigen binding fragment thereof comprises a variable light region comprising an amino acid sequence at least 92% identical to SEQ ID NO: 220. In some embodiments, the integrin α7β1 antibody or antigen binding fragment thereof comprises a variable light region comprising an amino acid sequence at least 93% identical to SEQ ID NO: 220. In some embodiments, the integrin α7β1 antibody or antigen binding fragment thereof comprises a variable light region comprising an amino acid sequence at least 94% identical to SEQ ID NO: 220. In some embodiments, the integrin α7β1 antibody or antigen binding fragment thereof comprises a variable light region comprising an amino acid sequence at least 95% identical to SEQ ID NO: 220. In some embodiments, the integrin α7β1 antibody or antigen binding fragment thereof comprises a variable light region comprising an amino acid sequence at least 97% identical to SEQ ID NO: 220. In some embodiments, the integrin α7β1 antibody or antigen binding fragment thereof comprises a variable light region comprising an amino acid sequence at least 98% identical to SEQ ID NO: 220. In some embodiments, the integrin α7β1 antibody or antigen binding fragment thereof comprises a variable light region comprising an amino acid sequence at least 99% identical to SEQ ID NO: 220. In some embodiments, the integrin α7β1 antibody or antigen binding fragment thereof comprises a variable light region comprising an amino acid sequence identical to SEQ ID NO: 220.

In some embodiments, the integrin α7β1 antibody or antigen binding fragment thereof comprises a variable light region comprising an amino acid sequence at least 80% identical to SEQ ID NO: 222. In some embodiments, the integrin α7β1 antibody or antigen binding fragment thereof comprises a variable light region comprising an amino acid sequence at least 83% identical to SEQ ID NO: 222. In some embodiments, the integrin α7β1 antibody or antigen binding fragment thereof comprises a variable light region comprising an amino acid sequence at least 85% identical to SEQ ID NO: 222. In some embodiments, the integrin α7β1 antibody or antigen binding fragment thereof comprises a variable light region comprising an amino acid sequence at least 87% identical to SEQ ID NO: 222. In some embodiments, the integrin α7β1 antibody or antigen binding fragment thereof comprises a variable light region comprising an amino acid sequence at least 90% identical to SEQ ID NO: 222. In some embodiments, the integrin α7β1 antibody or antigen binding fragment thereof comprises a variable light region comprising an amino acid sequence at least 92% identical to SEQ ID NO: 222. In some embodiments, the integrin α7β1 antibody or antigen binding fragment thereof comprises a variable light region comprising an amino acid sequence at least 93% identical to SEQ ID NO: 222. In some embodiments, the integrin α7β1 antibody or antigen binding fragment thereof comprises a variable light region comprising an amino acid sequence at least 94% identical to SEQ ID NO: 222. In some embodiments, the integrin α7β1 antibody or antigen binding fragment thereof comprises a variable light region comprising an amino acid sequence at least 95% identical to SEQ ID NO: 222. In some embodiments, the integrin α7β1 antibody or antigen binding fragment thereof comprises a variable light region comprising an amino acid sequence at least 97% identical to SEQ ID NO: 222. In some embodiments, the integrin α7β1 antibody or antigen binding fragment thereof comprises a variable light region comprising an amino acid sequence at least 98% identical to SEQ ID NO: 222. In some embodiments, the integrin α7β1 antibody or antigen binding fragment thereof comprises a variable light region comprising an amino acid sequence at least 99% identical to SEQ ID NO: 222. In some embodiments, the integrin α7β1 antibody or antigen binding fragment thereof comprises a variable light region comprising an amino acid sequence identical to SEQ ID NO: 222.

In some embodiments, the integrin α7β1 antibody or antigen binding fragment thereof comprises a variable light region comprising an amino acid sequence at least 80% identical to SEQ ID NO: 224. In some embodiments, the integrin α7β1 antibody or antigen binding fragment thereof comprises a variable light region comprising an amino acid sequence at least 83% identical to SEQ ID NO: 224. In some embodiments, the integrin α7β1 antibody or antigen binding fragment thereof comprises a variable light region comprising an amino acid sequence at least 85% identical to SEQ ID NO: 224. In some embodiments, the integrin α7β1 antibody or antigen binding fragment thereof comprises a variable light region comprising an amino acid sequence at least 87% identical to SEQ ID NO: 224. In some embodiments, the integrin α7β1 antibody or antigen binding fragment thereof comprises a variable light region comprising an amino acid sequence at least 90% identical to SEQ ID NO: 224. In some embodiments, the integrin α7β1 antibody or antigen binding fragment thereof comprises a variable light region comprising an amino acid sequence at least 92% identical to SEQ ID NO: 224. In some embodiments, the integrin α7β1 antibody or antigen binding fragment thereof comprises a variable light region comprising an amino acid sequence at least 93% identical to SEQ ID NO: 224. In some embodiments, the integrin α7β1 antibody or antigen binding fragment thereof comprises a variable light region comprising an amino acid sequence at least 94% identical to SEQ ID NO: 224. In some embodiments, the integrin α7β1 antibody or antigen binding fragment thereof comprises a variable light region comprising an amino acid sequence at least 95% identical to SEQ ID NO: 224. In some embodiments, the integrin α7β1 antibody or antigen binding fragment thereof comprises a variable light region comprising an amino acid sequence at least 97% identical to SEQ ID NO: 224. In some embodiments, the integrin α7β1 antibody or antigen binding fragment thereof comprises a variable light region comprising an amino acid sequence at least 98% identical to SEQ ID NO: 224. In some embodiments, the integrin α7β1 antibody or antigen binding fragment thereof comprises a variable light region comprising an amino acid sequence at least 99% identical to SEQ ID NO: 224. In some embodiments, the integrin α7β1 antibody or antigen binding fragment thereof comprises a variable light region comprising an amino acid sequence identical to SEQ ID NO: 224.

In some embodiments, the integrin α7β1 antibody or antigen binding fragment thereof comprises a heavy variable region comprising an amino acid sequence of SEQ ID NO: 108 and a light variable region comprising an amino acid sequence of SEQ ID NO: 109.

In some embodiments, the integrin α7β1 antibody or antigen binding fragment thereof comprises a heavy variable region comprising an amino acid sequence of SEQ ID NO: 110 and a light variable region comprising an amino acid sequence of SEQ ID NO: 111.

In some embodiments, the integrin α7β1 antibody or antigen binding fragment thereof comprises a heavy variable region comprising an amino acid sequence of SEQ ID NO: 199 and a light variable region comprising an amino acid sequence of SEQ ID NO: 200.

In some embodiments, the integrin α7β1 antibody or antigen binding fragment thereof comprises a heavy variable region comprising an amino acid sequence of SEQ ID NO: 201 and a light variable region comprising an amino acid sequence of SEQ ID NO: 202.

In some embodiments, the integrin α7β1 antibody or antigen binding fragment thereof comprises a heavy variable region comprising an amino acid sequence of SEQ ID NO: 203 and a light variable region comprising an amino acid sequence of SEQ ID NO: 204.

In some embodiments, the integrin α7β1 antibody or antigen binding fragment thereof comprises a heavy variable region comprising an amino acid sequence of SEQ ID NO: 205 and a light variable region comprising an amino acid sequence of SEQ ID NO: 206.

In some embodiments, the integrin α7β1 antibody or antigen binding fragment thereof comprises a heavy variable region comprising an amino acid sequence of SEQ ID NO: 207 and a light variable region comprising an amino acid sequence of SEQ ID NO: 208.

In some embodiments, the integrin α7β1 antibody or antigen binding fragment thereof comprises a heavy variable region comprising an amino acid sequence of SEQ ID NO: 209 and a light variable region comprising an amino acid sequence of SEQ ID NO: 210.

In some embodiments, the integrin α7β1 antibody or antigen binding fragment thereof comprises a heavy variable region comprising an amino acid sequence of SEQ ID NO: 211 and a light variable region comprising an amino acid sequence of SEQ ID NO: 212.

In some embodiments, the integrin α7β1 antibody or antigen binding fragment thereof comprises a heavy variable region comprising an amino acid sequence of SEQ ID NO: 213 and a light variable region comprising an amino acid sequence of SEQ ID NO: 214.

In some embodiments, the integrin α7β1 antibody or antigen binding fragment thereof comprises a heavy variable region comprising an amino acid sequence of SEQ ID NO: 215 and a light variable region comprising an amino acid sequence of SEQ ID NO: 216.

In some embodiments, the integrin Ca1 antibody or antigen binding fragment thereof comprises a heavy variable region comprising an amino acid sequence of SEQ ID NO: 217 and a light variable region comprising an amino acid sequence of SEQ ID NO: 218.

In some embodiments, the integrin α7β1 antibody or antigen binding fragment thereof comprises a heavy variable region comprising an amino acid sequence of SEQ ID NO: 219 and a light variable region comprising an amino acid sequence of SEQ ID NO: 220.

In some embodiments, the integrin α7β1 antibody or antigen binding fragment thereof comprises a heavy variable region comprising an amino acid sequence of SEQ ID NO: 221 and a light variable region comprising an amino acid sequence of SEQ ID NO: 222.

In some embodiments, the integrin α7β1 antibody or antigen binding fragment thereof comprises a heavy variable region comprising an amino acid sequence of SEQ ID NO: 223 and a light variable region comprising an amino acid sequence of SEQ ID NO: 224.

TABLE G
Exemplary Heavy Chain and Light Chain Sequences of Humanized
anti-α7β1 Integrin Antibodies.

| Ab | Heavy Chain Sequence | Light Chain Sequence |
|---|---|---|
| Ab24 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSNRAAG |
| | EFRDRATLTVDKSTSTAYMELSSLRSEDTAV | VPDRFSGSGSGTDFTLKISRVEAEDVGVYFC |
| | YYCARGGYFGREWYYDVWGQGTLVTVSSAST | SQSTHVPWTFGGGTKLEIKRTVAAPSVFIFP |
| | KGPSVFPLAPSSKSTSGGTAALGCLVKDYFP | PSDEQLKSGTASVVCLLNNFYPREAKVQWKV |
| | EPVTVSWNSGALTSGVHTFPAVLQSSGLYSL | DNALQSGNSQESVTEQDSKDSTYSLSSTLTL |
| | SSVVTVPSSSLGTQTYICNVNHKPSNTKVDK | SKADYEKHKVYACEVTHQGLSSPVTKSENRG |
| | KVEPKSCDKTHTCPPCPAPEAAGGPSVFLFP | EC (SEQ ID NO: 113) |
| | PKPKDTLMISRTPEVTCVVVDVSHEDPEVKF | |
| | NWYVDGVEVHNAKTKPREEQYNSTYRVVSVL | |
| | TVLHQDWLNGKEYKCKVSNKALAAPIEKTIS | |
| | KAKGQPREPQVYTLPPSRDELTKNQVSLTCL | |
| | VKGFYPSDIAVEWESNGQPENNYKTTPPVLD | |
| | SDGSFFLYSKLTVDKSRWQQGNVFSCSVLHE | |
| | ALHSHYTQKSLSLSPG | |
| | (SEQ ID NO: 112) | |
| Ab25 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSNRAAG |
| | EFRDRATLTVDKSTSTAYMELSSLRSEDTAV | VPDRFSGSGSGTDFTLKISRVEAEDVGVYFC |
| | YYCARGGYFGREWYYDVWGQGTLVTVSSAST | SQSTHVPWTFGGGTKLEIKRTVAAPSVFIFP |
| | KGPSVFPLAPSSKSTSGGTAALGCLVKDYFP | PSDEQLKSGTASVVCLLNNFYPREAKVQWKV |
| | EPVTVSWNSGALTSGVHTFPAVLQSSGLYSL | DNALQSGNSQESVTEQDSKDSTYSLSSTLTL |
| | SSVVTVPSSSLGTQTYICNVNHKPSNTKVDK | SKADYEKHKVYACEVTHQGLSSPVTKSENRG |
| | KVEPKSCDKTHTCPPCPAPEAAGGPSVFLFP | EC (SEQ ID NO: 113) |
| | PKPKDTLYITREPEVTCVVVDVSHEDPEVKE | |
| | NWYVDGVEVHNAKTKPREEQYNSTYRVVSVL | |
| | TVLHQDWLNGKEYKCKVSNKALAAPIEKTIS | |
| | KAKGQPREPQVYTLPPSRDELTKNQVSLTCL | |
| | VKGFYPSDIAVEWESNGQPENNYKTTPPVLD | |
| | SDGSFFLYSKLTVDKSRWQQGNVFSCSVMHE | |
| | ALHNHYTQKSLSLSPG (SEQ ID NO: | |
| | 114) | |
| Ab26 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSNRFTG |
| | EFRDRATLTVDKSTSTAYMELSSLRSEDTAV | VPDRFSGSGSGTDFTLKISRVEAEDVGVYFC |
| | YYCARGGYFGEEWYHDVWGQGTLVTVSSAST | AQSTQLPYTFGGGTKLEIKRTVAAPSVFIFP |
| | KGPSVFPLAPSSKSTSGGTAALGCLVKDYFP | PSDEQLKSGTASVVCLLNNFYPREAKVQWKV |
| | EPVTVSWNSGALTSGVHTFPAVLQSSGLYSL | DNALQSGNSQESVTEQDSKDSTYSLSSTLTL |
| | SSVVTVPSSSLGTQTYICNVNHKPSNTKVDK | SKADYEKHKVYACEVTHQGLSSPVTKSENRG |
| | KVEPKSCDKTHTCPPCPAPEAAGGPSVFLFP | EC (SEQ ID NO: 116) |
| | PKPKDTLMISRTPEVTCVVVDVSHEDPEVKF | |
| | NWYVDGVEVHNAKTKPREEQYNSTYRVVSVL | |
| | TVLHQDWLNGKEYKCKVSNKALAAPIEKTIS | |
| | KAKGQPREPQVYTLPPSRDELTKNQVSLTCL | |
| | VKGFYPSDIAVEWESNGQPENNYKTTPPVLD | |
| | SDGSFFLYSKLTVDKSRWQQGNVFSCSVLHE | |
| | ALHSHYTQKSLSLSPG (SEQ ID NO: | |
| | 115) | |
| Ab27 | EVQLVQSGAEVKKPGSSVKVSCKASGYSFTR | DIVMTQSPLSLPVTPGEPASISCRSSQSLVH |
| | YWMHWVRQAPGQGLEWIGKIDPSDSETRLNQ | RNGNTYLHWYLQKPGQSPQLLIYEVSNRFTG |
| | EFRDRATLTVDKSTSTAYMELSSLRSEDTAV | VPDRFSGSGSGTDFTLKISRVEAEDVGVYFC |
| | YYCARGGYFGEEWYHDVWGQGTLVTVSSAST | AQSTQLPYTFGGGTKLEIKRTVAAPSVFIFP |
| | KGPSVFPLAPSSKSTSGGTAALGCLVKDYFP | PSDEQLKSGTASVVCLLNNFYPREAKVQWKV |
| | EPVTVSWNSGALTSGVHTFPAVLQSSGLYSL | DNALQSGNSQESVTEQDSKDSTYSLSSTLTL |
| | SSVVTVPSSSLGTQTYICNVNHKPSNTKVDK | SKADYEKHKVYACEVTHQGLSSPVTKSENRG |
| | KVEPKSCDKTHTCPPCPAPEAAGGPSVFLFP | EC (SEQ ID NO: 116) |
| | PKPKDTLYITREPEVTCVVVDVSHEDPEVKF | |
| | NWYVDGVEVHNAKTKPREEQYNSTYRVVSVL | |
| | TVLHQDWLNGKEYKCKVSNKALAAPIEKTIS | |
| | KAKGQPREPQVYTLPPSRDELTKNQVSLTCL | |
| | VKGFYPSDIAVEWESNGQPENNYKTTPPVLD | |
| | SDGSFFLYSKLTVDKSRWQQGNVFSCSVMHE | |
| | ALHNHYTQKSLSLSPG (SEQ ID NO: | |
| | 117) | |

In some embodiments, the integrin α7β1 antibody comprises a heavy chain comprising an amino acid sequence at least 80% identical to SEQ ID NO: 112. In some embodiments, the integrin α7β1 antibody comprises a heavy chain comprising an amino acid sequence at least 83% identical to SEQ ID NO: 112. In some embodiments, the integrin α7β1 antibody comprises a heavy chain comprising an amino acid sequence at least 85% identical to SEQ ID NO: 112. In some embodiments, the integrin α7β1 antibody comprises a heavy chain comprising an amino acid sequence at least 87% identical to SEQ ID NO: 112. In some embodiments, the integrin α7β1 antibody comprises a heavy chain comprising an amino acid sequence at least 90% identical to SEQ ID NO: 112. In some embodiments, the integrin α7β1 antibody comprises a heavy chain comprising an amino acid sequence at least 92% identical to SEQ ID NO: 112. In some embodiments, the integrin α7β1 antibody comprises a heavy chain comprising an amino acid sequence at least 93% identical to SEQ ID NO: 112. In some embodiments, the integrin α7β1 antibody comprises a heavy chain comprising an amino acid sequence at least 94% identical to SEQ ID NO: 112. In some embodiments, the integrin α7β1 antibody comprises a heavy chain comprising an amino acid sequence at least 95% identical to SEQ ID NO: 112. In some embodiments, the integrin α7β1 antibody comprises a heavy chain comprising an amino acid sequence at least 97% identical to SEQ ID NO: 112. In some embodiments, the integrin α7β1 antibody comprises a heavy chain comprising an amino acid sequence at least 98% identical to SEQ ID NO: 112. In some embodiments, the integrin α7β1 antibody comprises a heavy chain comprising an amino acid sequence at least 99% identical to SEQ ID NO: 112. In some embodiments, the integrin α7β1 antibody comprises a heavy chain comprising an amino acid sequence identical to SEQ ID NO: 112.

In some embodiments, the integrin α7β1 antibody comprises a heavy chain comprising an amino acid sequence at least 80% identical to SEQ ID NO: 114. In some embodiments, the integrin α7β1 antibody comprises a heavy chain comprising an amino acid sequence at least 83% identical to SEQ ID NO: 114. In some embodiments, the integrin α7β1 antibody comprises a heavy chain comprising an amino acid sequence at least 85% identical to SEQ ID NO: 114. In some embodiments, the integrin α7β1 antibody comprises a heavy chain comprising an amino acid sequence at least 87% identical to SEQ ID NO: 114. In some embodiments, the integrin α7β1 antibody comprises a heavy chain comprising an amino acid sequence at least 90% identical to SEQ ID NO: 114. In some embodiments, the integrin α7β1 antibody comprises a heavy chain comprising an amino acid sequence at least 92% identical to SEQ ID NO: 114. In some embodiments, the integrin α7β1 antibody comprises a heavy chain comprising an amino acid sequence at least 93% identical to SEQ ID NO: 114. In some embodiments, the integrin α7β1 antibody comprises a heavy chain comprising an amino acid sequence at least 94% identical to SEQ ID NO: 114. In some embodiments, the integrin α7β1 antibody comprises a heavy chain comprising an amino acid sequence at least 95% identical to SEQ ID NO: 114. In some embodiments, the integrin α7β1 antibody comprises a heavy chain comprising an amino acid sequence at least 97% identical to SEQ ID NO: 114. In some embodiments, the integrin α7β1 antibody comprises a heavy chain comprising an amino acid sequence at least 98% identical to SEQ ID NO: 114. In some embodiments, the integrin α7β1 antibody comprises a heavy chain comprising an amino acid sequence at least 99% identical to SEQ ID NO: 114. In some embodiments, the integrin α7β1 antibody comprises a heavy chain comprising an amino acid sequence identical to SEQ ID NO: 114.

In some embodiments, the integrin α7β1 antibody comprises a heavy chain comprising an amino acid sequence at least 80% identical to SEQ ID NO: 115. In some embodiments, the integrin α7β1 antibody comprises a heavy chain comprising an amino acid sequence at least 83% identical to SEQ ID NO: 115. In some embodiments, the integrin α7β1 antibody comprises a heavy chain comprising an amino acid sequence at least 85% identical to SEQ ID NO: 115. In some embodiments, the integrin α7β1 antibody comprises a heavy chain comprising an amino acid sequence at least 87% identical to SEQ ID NO: 115. In some embodiments, the integrin α7β1 antibody comprises a heavy chain comprising an amino acid sequence at least 90% identical to SEQ ID NO: 115. In some embodiments, the integrin α7β1 antibody comprises a heavy chain comprising an amino acid sequence at least 92% identical to SEQ ID NO: 115. In some embodiments, the integrin α7β1 antibody comprises a heavy chain comprising an amino acid sequence at least 93% identical to SEQ ID NO: 115. In some embodiments, the integrin α7β1 antibody comprises a heavy chain comprising an amino acid sequence at least 94% identical to SEQ ID NO: 115. In some embodiments, the integrin α7β1 antibody comprises a heavy chain comprising an amino acid sequence at least 95% identical to SEQ ID NO: 115. In some embodiments, the integrin α7β1 antibody comprises a heavy chain comprising an amino acid sequence at least 97% identical to SEQ ID NO: 115. In some embodiments, the integrin α7β1 antibody comprises a heavy chain comprising an amino acid sequence at least 98% identical to SEQ ID NO: 115. In some embodiments, the integrin α7β1 antibody comprises a heavy chain comprising an amino acid sequence at least 99% identical to SEQ ID NO: 115. In some embodiments, the integrin α7β1 antibody comprises a heavy chain comprising an amino acid sequence identical to SEQ ID NO: 115.

In some embodiments, the integrin α7β1 antibody comprises a heavy chain comprising an amino acid sequence at least 80% identical to SEQ ID NO: 117. In some embodiments, the integrin α7β1 antibody comprises a heavy chain comprising an amino acid sequence at least 83% identical to SEQ ID NO: 117. In some embodiments, the integrin α7β1 antibody comprises a heavy chain comprising an amino acid sequence at least 85% identical to SEQ ID NO: 117. In some embodiments, the integrin α7β1 antibody comprises a heavy chain comprising an amino acid sequence at least 87% identical to SEQ ID NO: 117. In some embodiments, the integrin α7β1 antibody comprises a heavy chain comprising an amino acid sequence at least 90% identical to SEQ ID NO: 117. In some embodiments, the integrin α7β1 antibody comprises a heavy chain comprising an amino acid sequence at least 92% identical to SEQ ID NO: 117. In some embodiments, the integrin α7β1 antibody comprises a heavy chain comprising an amino acid sequence at least 93% identical to SEQ ID NO: 117. In some embodiments, the integrin α7β1 antibody comprises a heavy chain comprising an amino acid sequence at least 94% identical to SEQ ID NO: 117. In some embodiments, the integrin α7β1 antibody comprises a heavy chain comprising an amino acid sequence at least 95% identical to SEQ ID NO: 117. In some embodiments, the integrin α7β1 antibody comprises a heavy chain comprising an amino acid sequence at least 97% identical to SEQ ID NO: 117. In some embodiments, the integrin α7β1 antibody comprises a heavy chain comprising an amino acid sequence at least 98% identical to SEQ ID NO: 117. In some embodiments, the integrin α7β1 antibody comprises a heavy chain comprising an amino acid sequence at least 99% identical to SEQ ID NO: 117. In some embodiments, the integrin α7β1 antibody comprises a heavy chain comprising an amino acid sequence identical to SEQ ID NO: 117.

In some embodiments, the integrin α7β1 antibody comprises a light chain comprising an amino acid sequence at least 80% identical to SEQ ID NO: 113. In some embodiments, the integrin α7β1 antibody comprises a light chain comprising an amino acid sequence at least 83% identical to SEQ ID NO: 113. In some embodiments, the integrin α7β1 antibody comprises a light chain comprising an amino acid sequence at least 85% identical to SEQ ID NO: 113. In some embodiments, the integrin α7β1 antibody comprises a light chain comprising an amino acid sequence at least 87% identical to SEQ ID NO: 113. In some embodiments, the integrin α7β1 antibody comprises a light chain comprising an amino acid sequence at least 90% identical to SEQ ID NO: 113. In some embodiments, the integrin α7β1 antibody comprises a light chain comprising an amino acid sequence at least 92% identical to SEQ ID NO: 113. In some embodiments, the integrin α7β1 antibody comprises a light chain comprising an amino acid sequence at least 93% identical to SEQ ID NO: 113. In some embodiments, the integrin α7β1 antibody comprises a light chain comprising an amino acid sequence at least 94% identical to SEQ ID NO: 113. In some embodiments, the integrin α7β1 antibody comprises a light chain comprising an amino acid sequence at least 95% identical to SEQ ID NO: 113. In some embodiments, the integrin α7β1 antibody comprises a light chain comprising an amino acid sequence at least 97% identical to SEQ ID NO: 113. In some embodiments, the integrin α7β1 antibody comprises a light chain comprising an amino acid sequence at least 98% identical to SEQ ID NO: 113. In some embodiments, the integrin α7β1 antibody comprises a light chain comprising an amino acid sequence at least 99% identical to SEQ ID NO: 113. In some embodiments, the integrin α7β1 antibody comprises a light chain comprising an amino acid sequence identical to SEQ ID NO: 113.

In some embodiments, the integrin α7β1 antibody comprises a light chain comprising an amino acid sequence at least 80% identical to SEQ ID NO: 116. In some embodiments, the integrin α7β1 antibody comprises a light chain comprising an amino acid sequence at least 83% identical to SEQ ID NO: 116. In some embodiments, the integrin α7β1 antibody comprises a light chain comprising an amino acid sequence at least 85% identical to SEQ ID NO: 116. In some embodiments, the integrin α7β1 antibody comprises a light chain comprising an amino acid sequence at least 87% identical to SEQ ID NO: 116. In some embodiments, the integrin α7β1 antibody comprises a light chain comprising an amino acid sequence at least 90% identical to SEQ ID NO: 116. In some embodiments, the integrin α7β1 antibody comprises a light chain comprising an amino acid sequence at least 92% identical to SEQ ID NO: 116. In some embodiments, the integrin α7β1 antibody comprises a light chain comprising an amino acid sequence at least 93% identical to SEQ ID NO: 116. In some embodiments, the integrin α7β1 antibody comprises a light chain comprising an amino acid sequence at least 94% identical to SEQ ID NO: 116. In some embodiments, the integrin α7β1 antibody comprises a light chain comprising an amino acid sequence at least 95% identical to SEQ ID NO: 116. In some embodiments, the integrin α7β1 antibody comprises a light chain comprising an amino acid sequence at least 97% identical to SEQ ID NO: 116. In some embodiments, the integrin α7β1 antibody comprises a light chain comprising an amino acid sequence at least 98% identical to SEQ ID NO: 116. In some embodiments, the integrin α7β1 antibody comprises a light chain comprising an amino acid sequence at least 99% identical to SEQ ID NO: 116. In some embodiments, the integrin α7β1 antibody comprises a light chain comprising an amino acid sequence identical to SEQ ID NO: 116.

In some embodiments, the integrin α7β1 antibody or antigen binding fragment thereof comprises a heavy chain comprising an amino acid sequence of SEQ ID NO: 112 and a light chain comprising an amino acid sequence of SEQ ID NO: 113.

In some embodiments, the integrin α7β1 antibody or antigen binding fragment thereof comprises a heavy chain comprising an amino acid sequence of SEQ ID NO: 114 and a light chain comprising an amino acid sequence of SEQ ID NO: 113.

In some embodiments, the integrin α7β1 antibody or antigen binding fragment thereof comprises a heavy chain comprising an amino acid sequence of SEQ ID NO: 115 and a light chain comprising an amino acid sequence of SEQ ID NO: 116.

In some embodiments, the integrin α7β1 antibody or antigen binding fragment thereof comprises a heavy chain comprising an amino acid sequence of SEQ ID NO: 117 and a light chain comprising an amino acid sequence of SEQ ID NO: 116.

In some embodiments, the antibody or antigen binding fragment thereof is selected from an antibody described in Table A. In some embodiments, the antibody or antigen binding fragment thereof competes for integrin binding and/or binds the same epitope as an anti-α7β1 antibody described in Table A. In some embodiments, the antibody or antigen binding fragment thereof is selected from an antibody described in Table B. In some embodiments, the antibody or antigen binding fragment thereof competes for integrin binding and/or binds the same epitope as an anti-α7β1 antibody described in Table B. In some embodiments, the antibody or antigen binding fragment thereof is selected from an antibody described in Table C. In some embodiments, the antibody or antigen binding fragment thereof competes for integrin binding and/or binds the same epitope as an anti-α7β1 antibody described in Table C. In some embodiments, the antibody or antigen binding fragment thereof is selected from an antibody described in Table D. In some embodiments, the antibody or antigen binding fragment thereof competes for integrin binding and/or binds the same epitope as an anti-α7β1 antibody described in Table D. In some embodiments, the antibody or antigen binding fragment thereof is selected from an antibody described in Table E. In some embodiments, the antibody or antigen binding fragment thereof competes for integrin binding and/or binds the same epitope as an anti-α7β1 antibody described in Table E. In some embodiments, the antibody or antigen binding fragment thereof is selected from an antibody described in Table F. In some embodiments, the antibody or antigen binding fragment thereof competes for integrin binding and/or binds the same epitope as an anti-α7β1 antibody described in Table F. In some embodiments, the antibody or antigen binding fragment thereof is selected from an antibody described in Table G. In some embodiments, the antibody or antigen binding fragment thereof competes for integrin binding and/or binds the same epitope as an anti-α7β1 antibody described in Table G.

In some embodiments, the anti-α7β1 integrin antibody or an antigen binding fragment is a Fab, a single chain Fc (scFv), a single domain antibody (VHH), one or more CDRs, a variable heavy chain (VH), a variable light chain (VL), a Fab-like bispecific antibody (bsFab), a single-domain antibody-linked Fab (s-Fab), an antibody, or a combination thereof. In some embodiments, the α7β1 integrin activator is an Fab. In some embodiments, the anti-α7β1 integrin antibody or an antigen binding fragment is a single chain Fc (scFv). In some embodiments, the anti-α7β1 integrin antibody or an antigen binding fragment is a single domain antibody (VHH). In some embodiments, the anti-α7β1 integrin antibody or an antigen binding fragment is one or more CDRs. In some embodiments, the anti-α7β1 integrin antibody or an antigen binding fragment activator is a variable heavy chain (VH). In some embodiments, the anti-α7β1 integrin antibody or an antigen binding fragment is a variable light chain (VL). In some embodiments, the anti-α7β1 integrin antibody or an antigen binding fragment is a Fab-like bispecific antibody (bsFab). In some embodiments, the anti-α7β1 integrin antibody or an antigen binding fragment is a single-domain antibody-linked Fab (s-Fab).

In some embodiments, the antibody or antigen-binding fragment thereof comprises IgG, F(ab′)₂, F(ab)₂, Fab′, Fab, scFv, diabody, triabody, tetrabody, or single chain camelid antibody. In some embodiments, the antibody or antigen-binding fragment thereof comprises an IgG. In some embodiments, the antibody or antigen-binding fragment thereof comprises an IgG4. In some embodiments, the antibody or antigen-binding fragment thereof comprises a F(ab′)₂. In some embodiments, the antibody or antigen-binding fragment thereof comprises a F(ab)₂. In some embodiments, the antibody or antigen-binding fragment thereof comprises a Fab′. In some embodiments, the antibody or antigen-binding fragment thereof comprises a Fab. In some embodiments, the antibody or antigen-binding fragment thereof comprises an scFv. In some embodiments, the antibody or antigen-binding fragment thereof comprises a diabody. In some embodiments, the antibody or antigen-binding fragment thereof comprises a triabody. In some embodiments, the antibody or antigen-binding fragment thereof comprises a tetrabody. In some embodiments, the antibody or antigen-binding fragment thereof comprises a single chain camelid antibody.

In some embodiments, the anti-α7β1 integrin antibody comprises an Fc domain. In some embodiments, an Fc domain is derived from IgG1. In some embodiments, an Fc domain is derived from IgG2. In some embodiments, an Fc domain is derived from IgG3. In some embodiments, an Fc domain is derived from IgG4. In some embodiments, an Fc domain is modified.

In some embodiments, the antibody or antigen-binding fragment thereof comprises a monoclonal antibody. In some embodiments, the antibody or antigen-binding fragment thereof is a human monoclonal antibody. In some embodiments, the antibody or antigen-binding fragment thereof comprises a mouse monoclonal antibody. In some embodiments, the antibody or antigen-binding fragment thereof is a humanized monoclonal antibody. In some embodiments, the antibody or antigen-binding fragment thereof is a chimeric monoclonal antibody.

In some embodiments, the antibody or antigen-binding fragment thereof comprises a human Fc region. In some embodiments, the Fc region comprises one or more mutations that increase the in vivo half-life of the antibody. In some embodiments, the Fc region comprises a LALAPA sequence (i.e., an effector-null variant). In some embodiments, the Fc region comprises a LS or YTE sequence (i.e., half-life extension variants). In some embodiments, the Fc region comprises a LALAPA sequence and LS sequence. In some embodiments, the Fc region comprises a LALAPA sequence and a YTE sequence.

In some embodiments, the antibody or antigen-binding fragment thereof has an affinity (Kd) of between about 0.1 to 6 nM. In some embodiments, the antibody or antigen-binding fragment thereof has an affinity (Kd) of between about 0.3 to 6 nM. In some embodiments, the antibody or antigen-binding fragment thereof has an affinity (Kd) of between about 0.5 to 6 nM. In some embodiments, the antibody or antigen-binding fragment thereof has an affinity of about 0.5 nM. In some embodiments, the antibody or antigen-binding fragment thereof has an affinity of about 1 nM. In some embodiments, the antibody or antigen-binding fragment thereof has an affinity of about 2 nM. In some embodiments, the antibody or antigen-binding fragment thereof has an affinity of about 3 nM. In some embodiments, the antibody or antigen-binding fragment thereof has an affinity of about 4 nM. In some embodiments, the antibody or antigen-binding fragment thereof has an affinity of about 5 nM. In some embodiments, the antibody or antigen-binding fragment thereof has an affinity of about 6 nM. In some embodiments, the antibody or antigen-binding fragment thereof has an affinity less than 0.5 nM. In some embodiments, the antibody or antigen-binding fragment thereof has an affinity greater than 6 nM.

In some embodiments, the antibody or antigen-binding fragment thereof activates α7β1 integrin with a maximal efficacy or Emax of between 65% to 150%. In some embodiments, the antibody or antigen-binding fragment thereof activates α7β1 integrin with a maximal efficacy or Emax less than 65%. In some embodiments, the antibody or antigen-binding fragment thereof activates α7β1 integrin with a maximal efficacy or Emax of 65%. In some embodiments, the antibody or antigen-binding fragment thereof activates α7β1 integrin with a maximal efficacy or Emax of 70%. In some embodiments, the antibody or antigen-binding fragment thereof activates α7β1 integrin with a maximal efficacy or Emax of 75%. In some embodiments, the antibody or antigen-binding fragment thereof activates α7β1 integrin with a maximal efficacy or Emax of 80%. In some embodiments, the antibody or antigen-binding fragment thereof activates α7β1 integrin with a maximal efficacy or Emax of 85%. In some embodiments, the antibody or antigen-binding fragment thereof activates α7β1 integrin with a maximal efficacy or Emax of 90%. In some embodiments, the antibody or antigen-binding fragment thereof activates α7β1 integrin with a maximal efficacy or Emax of 95%. In some embodiments, the antibody or antigen-binding fragment thereof activates α7β1 integrin with a maximal efficacy or Emax of 100%.

In some embodiments, the antibody or antigen-binding fragment thereof activates α7β1 integrin with a maximal efficacy or Emax of 105%. In some embodiments, the antibody or antigen-binding fragment thereof activates α7β1 integrin with a maximal efficacy or Emax of 110%. In some embodiments, the antibody or antigen-binding fragment thereof activates α7β1 integrin with a maximal efficacy or Emax of 115%. In some embodiments, the antibody or antigen-binding fragment thereof activates α7β1 integrin with a maximal efficacy or Emax of 120%. In some embodiments, the antibody or antigen-binding fragment thereof activates α7β1 integrin with a maximal efficacy or Emax of 125%. In some embodiments, the antibody or antigen-binding fragment thereof activates α7β1 integrin with a maximal efficacy or Emax of 130%. In some embodiments, the antibody or antigen-binding fragment thereof activates α7β1 integrin with a maximal efficacy or Emax of 135%. In some embodiments, the antibody or antigen-binding fragment thereof activates α7β1 integrin with a maximal efficacy or Emax of 140%. In some embodiments, the antibody or antigen-binding fragment thereof activates α7β1 integrin with a maximal efficacy or Emax of 145%. In some embodiments, the antibody or antigen-binding fragment thereof activates α7β1 integrin with a maximal efficacy or Emax of 150%. In some embodiments, the antibody or antigen-binding fragment thereof activates α7β1 integrin with a maximal efficacy or Emax greater than 150%.

In some embodiments, the antibody or antigen-binding fragment thereof has a potency as measured by EC50 of between 0.05 and 20 nM. In some embodiments, the antibody or antigen-binding fragment thereof has a potency as measured by EC50 of between 0.5 and 1.7 nM. In some embodiments, the antibody or antigen-binding fragment thereof has a potency as measured by EC50 of 1.7 nM.

In some embodiments, the antibody or antigen-binding fragment thereof has a potency as measured by EC50 of less than 0.05 nM. In some embodiments, the antibody or antigen-binding fragment thereof has a potency as measured by EC50 of about 0.05 nM. In some embodiments, the antibody or antigen-binding fragment thereof has a potency as measured by EC50 of about 1 nM. In some embodiments, the antibody or antigen-binding fragment thereof has a potency as measured by EC50 of about 2 nM. In some embodiments, the antibody or antigen-binding fragment thereof has a potency as measured by EC50 of about 3 nM. In some embodiments, the antibody or antigen-binding fragment thereof has a potency as measured by EC50 of about 4 nM. In some embodiments, the antibody or antigen-binding fragment thereof has a potency as measured by EC50 of about 5 nM.

In some embodiments, the antibody or antigen-binding fragment thereof has a potency as measured by EC50 of about 6 nM. In some embodiments, the antibody or antigen-binding fragment thereof has a potency as measured by EC50 of about 7 nM. In some embodiments, the antibody or antigen-binding fragment thereof has a potency as measured by EC50 of about 8 nM. In some embodiments, the antibody or antigen-binding fragment thereof has a potency as measured by EC50 of about 9 nM. In some embodiments, the antibody or antigen-binding fragment thereof has a potency as measured by EC50 of about 10 nM.

In some embodiments, the antibody or antigen-binding fragment thereof has a potency as measured by EC50 of about 11 nM. In some embodiments, the antibody or antigen-binding fragment thereof has a potency as measured by EC50 of about 12 nM. In some embodiments, the antibody or antigen-binding fragment thereof has a potency as measured by EC50 of about 13 nM. In some embodiments, the antibody or antigen-binding fragment thereof has a potency as measured by EC50 of about 14 nM. In some embodiments, the antibody or antigen-binding fragment thereof has a potency as measured by EC50 of about 15 nM.

In some embodiments, the antibody or antigen-binding fragment thereof has a potency as measured by EC50 of about 16 nM. In some embodiments, the antibody or antigen-binding fragment thereof has a potency as measured by EC50 of about 17 nM. In some embodiments, the antibody or antigen-binding fragment thereof has a potency as measured by EC50 of about 18 nM. In some embodiments, the antibody or antigen-binding fragment thereof has a potency as measured by EC50 of about 19 nM. In some embodiments, the antibody or antigen-binding fragment thereof has a potency as measured by EC50 of about 20 nM. In some embodiments, the antibody or antigen-binding fragment thereof has a potency as measured by EC50 of greater than 20 nM.

### Methods of Treatment

The present disclosure provides a method of treating a muscular dystrophy in a subject, comprising administering an anti-α7β1 integrin antibody. In some embodiments, the present disclosure provides a method of treating a disease associated with decreased expression or activity of dystrophin, comprising administering an anti-α7β1 integrin antibody.

In some embodiments, the disease is muscular dystrophy. In some embodiments, the disease is a dystrophinopathy. In some embodiments, the disease is Duchenne muscular dystrophy.

In one aspect, the present disclosure provides, among other things, a method of treating a disorder or a disease in a subject comprising administering to the subject a therapeutically effective amount of an anti-α7β1 antibody or an antibody fragment thereof, thereby treating the disorder or the disease in the subject. The disorder or the disease can be characterized by one or more of: a malfunction of α7β1 integrin in the subject; a dystrophin deficiency in the subject; a mutation in dystrophin in the subject; non-functioning dystrophin in the subject; or a muscle dysfunction other than α7β1 malfunction, or dystrophin deficiency, mutation, or non-function.

In some embodiments, the muscle dysfunction is caused by or associated with one or more of: cancer, congestive heart failure, chronic obstructive pulmonary disease, chronic kidney disease, HIV infection/AIDS, anorexia nervosa, bulimia, malnutrition, exposure, nausea, type I diabetes, type II diabetes, metabolic syndrome, cachexia, anemia, heart failure, high blood pressure, rhabdomyolysis, sepsis, sarcopenia, physical inactivity, damage due to excess physical activity, hypothermia, hyperthermia, injury, denervation, amyotrophic lateral sclerosis, multiple sclerosis, spinal muscular atrophy, alcohol-associated myopathy, bum-associated myopathy, stroke, steroid therapy or the withdrawal of steroid therapy, dermatomyositis, Guillain-Barre syndrome, neuropathy, osteoarthritis, infection, polio, polymyositis, inflammation, rheumatoid arthritis, hypocholesterolemia, electrical injury, heat stroke, prolonged immobilization, lack of blood flow to a limb, or contact with venom. The methods of treatment of the disclosure may further comprise a step of identifying a subject having a muscle dysfunction.

In some embodiments, the disease is a muscle wasting disease. In some embodiments, a muscle wasting disease is a muscular dystrophy. In certain cases, the muscular dystrophy is Duchenne muscular dystrophy (DMD), Becker muscular dystrophy, merosin-deficient congenital muscular dystrophy type 1A or limb-girdle muscular dystrophy. The methods of treatment of the disclosure may further comprise a step of identifying a subject having a muscle wasting disease.

### Muscular Dystrophy

Muscular dystrophy refers to a group of genetic disorders that lead to progressive muscle weakness. Muscular dystrophy can result in skeletal muscle weakness, leading to a variety of impaired physiological functions. No satisfactory treatment of muscular dystrophy exists. Existing treatments typically focus on ameliorating the effects of the disease and improving quality of life, such as through physical therapy or provision of orthopedic devices.

Several gene mutations are associated with muscular dystrophy and responsible for encoding a number of proteins associated with the costameric protein network. Such proteins include laminin-2, collagen, dystroglycan, integrins, caveolin-3, ankyrin, dystrophin, α-dystrobrevin, vinculin, plectin, BPAG1b, muscle LIM protein, desmin, actinin-associated LIM protein, α-actin, titin, telethonin, cypher, myotilin, and the sarcoglycan/sarcospan complex. The most common form of muscular dystrophy is Duchenne Muscular Dystrophy, affecting 1 in 3,500 live male births.

### Duchenne Muscular Dystrophy

Duchenne Muscular Dystrophy (DMD) is a disease characterized by the progressive deterioration of muscles and loss of muscle related functions throughout the body. It is contemplated that the present disclosure provides methods and compositions for treating symptoms and features associates with DMD and other dystrophinopathies.

DMD is a recessive X-linked form of muscular dystrophy which results in muscle degeneration and eventual death. DMD is characterized by weakness in the proximal muscles, abnormal gait, pseudohypertrophy in the gastroenemius (calf) muscles, and elevated creatine kinase (CK). Many DMD patients are diagnosed around the age of 5, when symptoms/signs typically become more obvious. Affected individuals typically stop walking around age 10-13 and die in or before their mid to late 20's due to cardiorespiratory dysfunction.

The disorder DMD is caused by a mutation in the dystrophin gene, located on the human X chromosome, which codes for the protein dystrophin, an important structural component within the muscle tissue that provides structural stability to the dystroglycan complex (DGC) of the cell membrane. While persons of both sexes can carry the mutation, females rarely exhibit severe signs of the disease. Dystrophin links the internal cytoplasmic actin filament network and extracellular matrix, providing physical strength to muscle fibers. Accordingly, alteration or absence of dystrophin results in abnormal sarcolemmal membrane tearing and necrosis of muscle fibers.

A main symptom of DMD is muscle weakness associated with muscle wasting with the voluntary muscles being first affected, especially affecting the muscles of the hips, pelvic area, thighs, shoulders, and calf muscles. Muscle weakness also occurs in the arms, neck, and other areas. Calves are often enlarged.

Also provided is a method for the treatment of a subject suffering from fibrosis or a fibrosis related disorder, comprising administering to said subject a therapeutically effective amount of α7β1 integrin activator according to the disclosure. The term “fibrosis” as used herein refers to a condition characterized by a deposition of extracellular matrix components in the skin or organs, including lungs, kidneys, heart, liver, skin and joints, resulting in scar tissue. The term also refers to the process of formation of scar tissue.

In some embodiments, the fibrosis-related disorder is a disorder or condition which may occur as a result of fibrosis, or which is associated with fibrosis. In some embodiments, fibrosis and/or a fibrosis-related disorders is a disease or condition selected from the group consisting of kidney fibrosis, liver fibrosis, liver cirrhosis, pulmonary fibrosis, skin fibrosis, biliary fibrosis, peritoneal fibrosis, myocardial fibrosis, pancreatic fibrosis, bone marrow and/or myelofibrosis, reperfusion injury after hepatic or kidney transplantation, Interstitial Lung Disease (ILD), cystic fibrosis (CF), atherosclerosis, systemic sclerosis, osteosclerosis, spinal disc herniation and other spinal cord injuries, fibromatosis, fibromyalgia, arthritis, restenosis. Pulmonary fibrosis includes idiopathic pulmonary fibrosis and scleroderma lung fibrosis.

### Muscle Atrophy

Muscle atrophy is the wasting (thinning) or loss of muscle tissue. Muscle atrophy (also known as muscular atrophy) is decrease in size and wasting of muscle tissue. Muscles that lose their nerve supply can atrophy and simply waste away. People may lose 20 to 40 percent of their muscle and, along with it, their strength as they age.

Provided herein is a method for the treatment of a subject suffering from muscle atrophy, comprising administering to said subject a therapeutically effective amount of α7β1 integrin activating antibody according to the disclosure.

In some embodiments, the method for the treatment of a subject suffering from muscle atrophy comprises administering to said subject a therapeutic agent to increase α7 expression. In some embodiments, the therapeutic agent comprises a small molecule. In some embodiments, the therapeutic agent comprises a biological agent such as, for example and without limitation, a nucleic acid or a protein. In some embodiments, the therapeutic agent comprises an antibody or antigen binding fragment. In some embodiments, the therapeutic agent comprises an antibody or antigen binding fragment of the instant disclosure.

In some embodiments, the method of treatment of a subject suffering from muscle atrophy comprises administering to said subject an antibody or antigen binding fragment of the instant disclosure, wherein administration results in an increase in α7 expression.

In some embodiments, the subject has reduced muscle loss as compared to a control upon administration of the antibody or the binding fragment thereof. In some embodiments, the subject has reduced loss of muscle function as compared to a control upon administration of the antibody or the binding fragment thereof. In some embodiments, the subject has reduced loss of muscle force as compared to a control upon administration of the antibody or the binding fragment thereof.

In some embodiments, the control is a baseline value prior to administration of the antibody or the binding fragment thereof. In some embodiments, the control is historical data. In some embodiments, the control is a comparable subject without the administration of the antibody or antigen binding fragment thereof.

In some embodiments, the subject has less than 40% muscle weight change as compared to a control upon administration of the antibody or the binding fragment thereof. In some embodiments, the subject has less than 35% muscle weight change as compared to a control upon administration of the antibody or the binding fragment thereof. In some embodiments, the subject has less than 30% muscle weight change as compared to a control upon administration of the antibody or the binding fragment thereof. In some embodiments, the subject has less than 28% muscle weight change as compared to a control upon administration of the antibody or the binding fragment thereof. In some embodiments, the subject has less than 27% muscle weight change as compared to a control upon administration of the antibody or the binding fragment thereof. In some embodiments, the subject has less than 26% muscle weight change as compared to a control upon administration of the antibody or the binding fragment thereof. In some embodiments, the subject has less than 25% muscle weight change as compared to a control upon administration of the antibody or the binding fragment thereof. In some embodiments, the subject has less than 24% muscle weight change as compared to a control upon administration of the antibody or the binding fragment thereof. In some embodiments, the subject has less than 23% muscle weight change as compared to a control upon administration of the antibody or the binding fragment thereof. In some embodiments, the subject has less than 22% muscle weight change as compared to a control upon administration of the antibody or the binding fragment thereof. In some embodiments, the subject has less than 21% muscle weight change as compared to a control upon administration of the antibody or the binding fragment thereof. In some embodiments, the subject has less than 20% muscle weight change as compared to a control upon administration of the antibody or the binding fragment thereof. In some embodiments, the subject has less than 19% muscle weight change as compared to a control upon administration of the antibody or the binding fragment thereof. In some embodiments, the subject has less than 18% muscle weight change as compared to a control upon administration of the antibody or the binding fragment thereof. In some embodiments, the subject has less than 17% muscle weight change as compared to a control upon administration of the antibody or the binding fragment thereof. In some embodiments, the subject has less than 16% muscle weight change as compared to a control upon administration of the antibody or the binding fragment thereof. In some embodiments, the subject has less than 15% muscle weight change as compared to a control upon administration of the antibody or the binding fragment thereof. In some embodiments, the subject has less than 14% muscle weight change as compared to a control upon administration of the antibody or the binding fragment thereof. In some embodiments, the subject has less than 13% muscle weight change as compared to a control upon administration of the antibody or the binding fragment thereof. In some embodiments, the subject has less than 12% muscle weight change as compared to a control upon administration of the antibody or the binding fragment thereof. In some embodiments, the subject has less than 11% muscle weight change as compared to a control upon administration of the antibody or the binding fragment thereof. In some embodiments, the subject has less than 10% muscle weight change as compared to a control upon administration of the antibody or the binding fragment thereof. In some embodiments, the subject has less than 9% muscle weight change as compared to a control upon administration of the antibody or the binding fragment thereof. In some embodiments, the subject has less than 8% muscle weight change as compared to a control upon administration of the antibody or the binding fragment thereof. In some embodiments, the subject has less than 7% muscle weight change as compared to a control upon administration of the antibody or the binding fragment thereof. In some embodiments, the subject has less than 6% muscle weight change as compared to a control upon administration of the antibody or the binding fragment thereof. In some embodiments, the subject has less than 5% muscle weight change as compared to a control upon administration of the antibody or the binding fragment thereof. In some embodiments, the subject has less than 4% muscle weight change as compared to a control upon administration of the antibody or the binding fragment thereof. In some embodiments, the subject has less than 3% muscle weight change as compared to a control upon administration of the antibody or the binding fragment thereof. In some embodiments, the subject has less than 2% muscle weight change as compared to a control upon administration of the antibody or the binding fragment thereof. In some embodiments, the subject has less than 1% muscle weight change as compared to a control upon administration of the antibody or the binding fragment thereof.

### Compositions

Compositions of the disclosure can be formulated in any suitable form, such as liquid, semi-solid and solid dosage forms, such as liquid solutions (e.g., injectable and infusible solutions), dispersions or suspensions, and the like. The optimal form for any composition depends on the intended mode of administration, the nature of the composition or combination, and therapeutic application or other intended use. A typical mode for delivery for a composition of the disclosure is by parenteral administration (e.g., intravenous administration). In one aspect, a composition of the disclosure is administered to a human patient by intravenous infusion or injection.

In some embodiments, this disclosure provides compositions, e.g., pharmaceutical compositions, which include an integrin α7β1 antibody or antigen binding fragment thereof described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion).

### EXAMPLES

### Example 1. Generation of Anti-α7β1 Integrin Antibodies

This example shows generation of α7 activating antibodies of the present disclosure.

Mouse anti-α7β1 integrin antibodies were generated by hybridoma platform and α7 knock-out (KO) mouse, using α7β1 leg protein as an antigen (mouse campaign). Human anti-α7β1 integrin antibodies were generated by hybridoma platform and human transgenic mouse, using α7β1 ecto domain or α7 leg as an antigen (human campaign), as shown in FIG. 1A.

About 26 clones were identified in the mouse campaign. All 26 antibodies bound to mouse α7β1 ecto domain, and 3 out of the 26 antibodies also bound to human α7β1 ecto domain.

About 11 clones were identified in the human campaign, and all of them bound human α7β1 ecto domain.

### Example 2. Epitope Determination of Anti-α7β1 Integrin Antibodies

In this example, the binding epitopes of integrin α7β1 activating antibodies were determined by ELISA assays, electron microscopy, and epitope mapping.

### Binding ELISA Assay

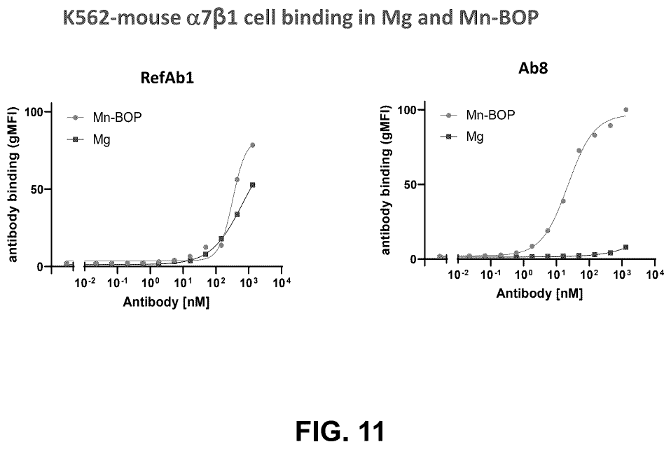

Binding ELISA assay was used to determine what domain of α7β1 integrin the exemplary antibodies bound to. All three antibodies tested, Ab2, Ab8, and RefAb1, bind to the leg domain of α7β1 as seen in FIGS. 1B-1D. Specifically, the Ab8 antibody binds to the Calf-1 domain within the leg domain of α7β1 (FIG. 1C), while the Ab2 and RefAb antibodies bind to the Calf-2 domain (FIG. 1B and FIG. 11D).

### Competitive ELISA Assays and Epitope Binning

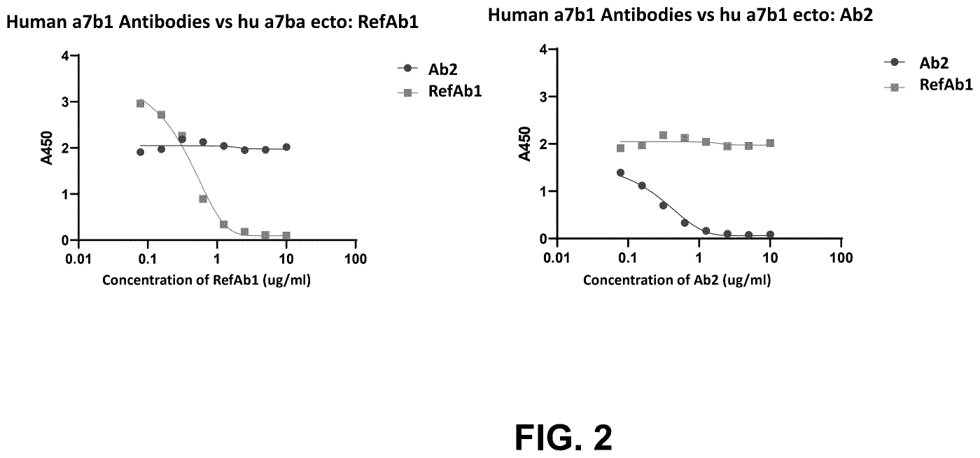

Since both RefAb1 and Ab2 bind to the Calf-2 domain, competitive ELISA assays were run to determine if the epitopes overlapped. As shown in FIG. 2, the results indicate that RefAb1 and Ab2 do not have overlapping epitopes.

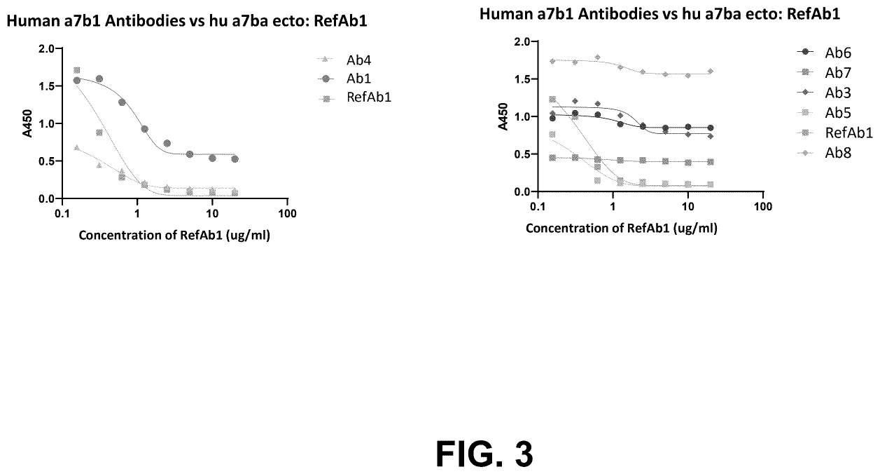

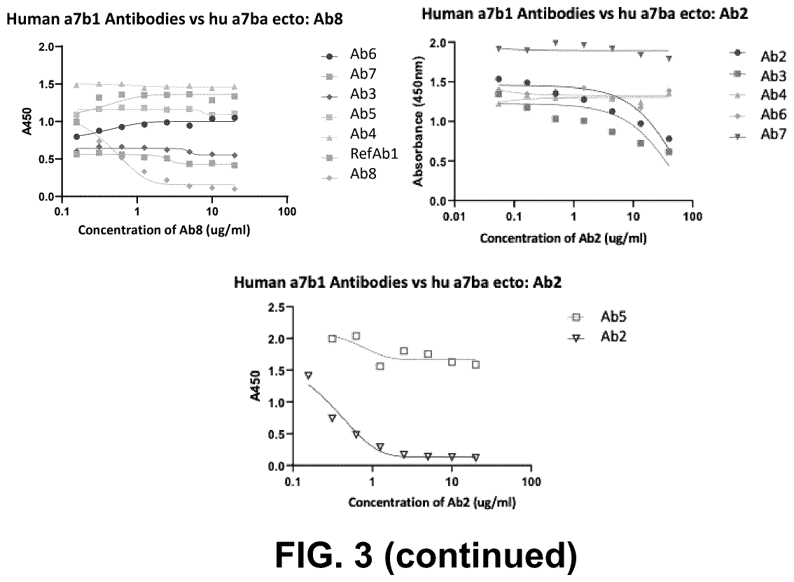

Next, several exemplary human anti-α7β1 antibodies were evaluated for competition with RefAb1, Ab2, and Ab8. Assay results are shown in FIG. 3, and the analysis is summarized in Table 1 below. Competition indicates overlapping epitopes.

TABLE 1
Epitope overlap of exemplary human anti-α7β1 antibodies
with RefAb1, Ab2, and Ab8.

| | Competes with | Competes with | Competes with |
|---|---|---|---|
| Antibody | RefAb1 | Ab2 | Ab8 |
| Ab1 | Yes | No | No |
| Ab2 | No | Yes | No |
| Ab3 | No | Yes | No |
| Ab4 | Yes | No | No |
| Ab5 | Yes | No | No |
| Ab6 | No | No | No |
| Ab7 | No | No | No |

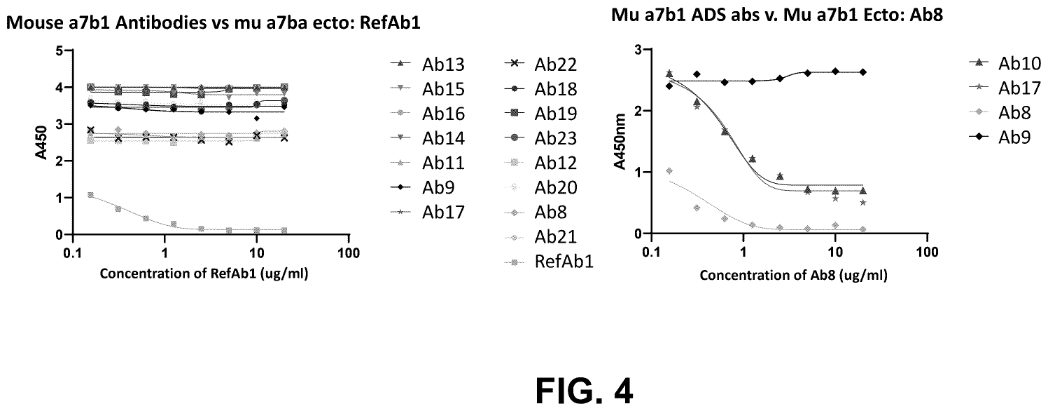

Several mouse anti-α7β1 antibodies were then similarly analyzed for competition with RefAb1 and Ab8. Assay results are shown in FIG. 4, and the analysis is summarized in Table 2 below.

TABLE 2
Epitope overlap of exemplary mouse anti-α7β1 antibodies
with RefAb1 and Ab8.

| Antibody | RefAb1 | Ab8 |
|---|---|---|
| Ab13 | No | Yes |
| Ab10 | No | Yes |
| Ab14 | No | Not Tested |
| Ab15 | No | Not Tested |
| Ab16 | No | Not Tested |
| Ab11 | No | Not Tested |
| Ab9 | No | No |
| Ab17 | No | Yes |
| Ab22 | No | Not Tested |
| Ab18 | No | Not Tested |
| Ab23 | No | Not Tested |
| Ab12 | No | Not Tested |
| Ab20 | No | Not Tested |
| Ab8 | No | Yes |
| Ab21 | No | Not Tested |

Epitope bins were determined for exemplary α7β1 integrin antibodies with more competitive ELISA assays. Results showed that Ab10, Ab17, and Ab23 have overlapping epitopes. Ab8 and Ab9 have close, but not overlapping epitopes with the group of Ab10, Ab17, and Ab23. AbA1 has another distinct epitope, and none of these tested antibodies overlap with RefAb1. A competition chart summarizing the results can be found below in Table 3. Antibody binding (Y) suggests that the antibody has a distinct epitope from the coated antibody.

TABLE 3
Competition chart for α7β1 integrin
antibodies showing antibody binding.

| | Antibody Binding | | | | |
|---|---|---|---|---|---|
| Antibody Coated | Ab10 | Ab17 | Ab8 | Ab9 | AbA1 |
| Ab10 | X | X | X | X | Y |
| Ab17 | X | X | X | X | Y |
| Ab8 | X | X | X | Y | Y |
| Ab23 | X | X | X | X | X |
| Ab9 | X | X | Y | X | Y |
| AbA1 | Y | Y | Y | Inconclusive | X |

Additionally, Ab4 and AbAb 1 have overlapping epitopes with RefAb1. The Ab2 antibody, however, was confirmed to have a distinct epitope. A competition chart comparing the epitopes of exemplary α7β1 integrin antibodies to RefAb1 can be found in Table 4. Antibody binding (Y) suggests that the antibody has a distinct epitope from the coated antibody.

TABLE 4
Competition chart for α7β1 integrin
antibodies showing antibody binding.

| Antibody Coated | RefAb1 Binding |
|---|---|
| AbA2 | Y |
| AbA3 | Y |
| Ab4 | X |
| AbA4 | Y |
| AbA5 | Y |
| Ab1 | X |
| AbA6 | Y |
| AbA7 | Y |
| AbA8 | Y |
| AbA9 | Y |
| Ab2 | Y |
| RefAb1 | X |

### Epitope Imaging and Mapping

The α7β1 integrin has different conformations that affect its interaction with laminin. At a bent-closed conformation, shown in FIG. 5A, the α7β1 integrin has a low affinity to laminin. At an extended-closed conformation, shown in FIG. 5B, the α7β1 integrin has a low affinity to laminin. The extended-open conformation, as shown in FIG. 5C however, has a high affinity to laminin. Without wishing to be bound to any theory, it is hypothesized that an antibody that favors an extended-closed or an extended-open conformation would have better therapeutic effect.

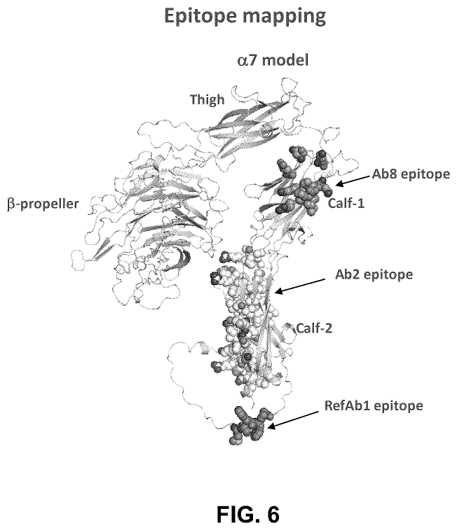

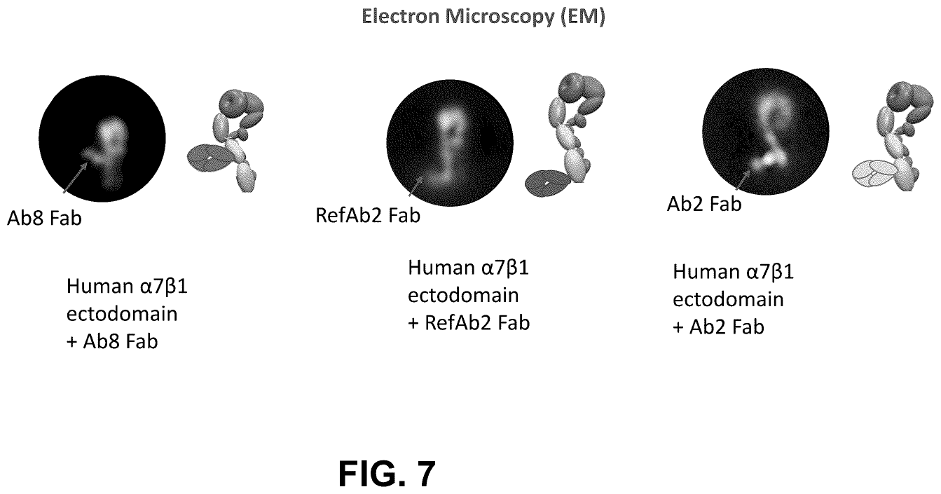

The epitopes of Ab2, Ab8, and RefAb1 were mapped onto an α7 protein model as shown in FIG. 6. Each antibody binds to a distinct epitope within the leg domain of α7β1 integrin. The binding locations of RefAb1, Ab2, and Ab8 to the ecto domain of human α7β1 were also confirmed via electron microscopy, as seen in FIG. 7.

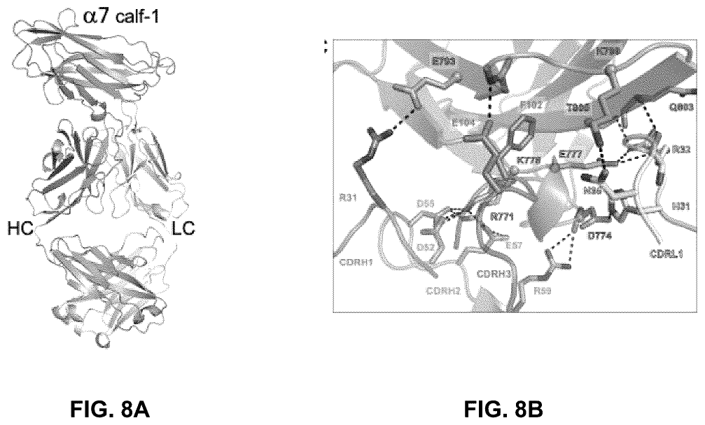

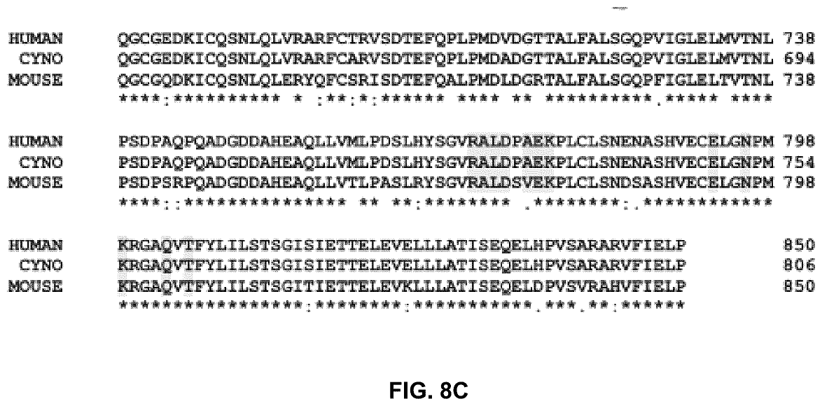

The epitope of Ab8 was then mapped using co-crystallization and analyzed. Ab8 Fab was co-crystallized with human α7 calf-1 protein. FIG. 8A shows the Ab8 heavy chain (orange) and light chain (yellow) in complex with α7 calf-1 (blue). FIG. 8B is an exemplary figure illustrating the biomolecular interactions between the Ab8 Fab and α7 calf-1 domain. The calf-1 protein is blue and is shown with the Ab8 HCDR1 (pink), HCDR2 (orange), HCDR3 (green), and LCDR1 (yellow). FIG. 8C highlights the residues of human, cynomolgus (cyno) monkey, and mouse calf-1 proteins that interact with Ab8. Ab8 is shown to interact with residues R771, A772, L773, D774, A776, E777, K778, E793, N796, K799, Q803, and T805 of human calf-1 and the corresponding residues in cyno and mouse calf-1.

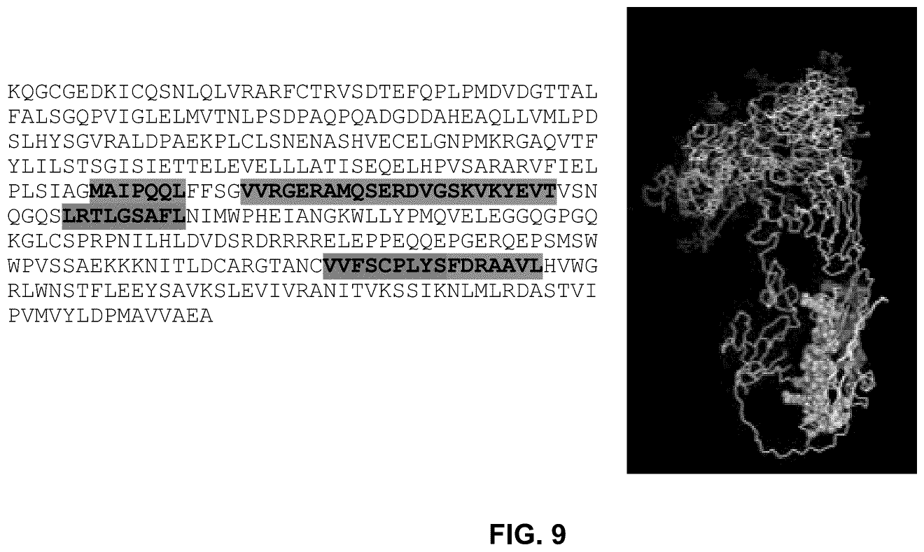

Hydrogen/Deuterium eXchange Mass Spectrometry (HDX-MS) was used to map the epitope of Ab2 on the leg domain of human α7. In HDX-MS, the protein or complex is suspended in heavy water, or water with the heavier hydrogen isotope, deuterium, and the hydrogens within the amino acids then naturally exchange with deuterium. The rate of exchange can vary based on hydrogen bonding and solvent accessibility, which causes the protein and protein-antibody complex to have different weights at fixed regions and timepoints. When analysis on specific regions of the protein or protein-antibody complex, the lack or magnitude of difference can inform where on the protein the antibody interacts or binds. Five regions of the human leg domain, highlighted and visualized in FIG. 9, displayed deuterium uptake differences when in complex with the Ab2: Positions 856-862 (MAIPQQL, SEQ ID NO: 181), 867-889 (VVRGERAMQSERDVGSKVKYEVT, SEQ ID NO: 182), 1011-1017 (FDRAAVL, SEQ ID NO: 183), 897-905 (LRTLGSAFL, SEQ ID NO: 184) and 1002-1010 (VVFSCPLYS, SEQ ID NO: 185).

Overall, this example demonstrated that Ab2, Ab8, and several other exemplary α7β1 integrin antibodies have distinct epitopes as compared to RefAb1. Further, epitope imaging and mapping was able to determine the epitope regions of Ab2 and Ab8, confirming ELISA results indicating binding to calf-2 and calf-1, respectively.

### Example 3. Integrin α7β1 Activating Antibodies Increase Laminin Interaction

In this example, the effect of exemplary α7β1 integrin antibodies on the interactions between α7β1 integrin and laminin was investigated with electric cell-substrate impedance sensing (ECIS) assays, cell adhesion assays, and solid phase assays.

### α7β1 Cell Adhesion Assay

The α7β1 cell adhesion assay was performed in 96-well assay plates in wells that were coated overnight at 4° C. with a solution of 0.625 μg/mL, 0.5 μg/mL, or 0.15 μg/mL laminin-221 for the assays against human, cynomolgus monkey, or mouse α7β1, respectively, and blocked 2 hours at 37° C. with a 1% solution of bovine serum albumin in phosphate buffered saline. Serial dilutions of antibodies and K562 cells stably expressing α7β1 were pre-incubated for 15 minutes at 37° C. in a solution of 50 mM HEPES pH 7.3, 150 mM sodium chloride, 10 mM glucose, 2 mM magnesium chloride, 2 mM calcium chloride, and 1% bovine serum albumin in a volume of 0.1 mL. The antibody and cell mixture was transferred to the assay plate and incubated for 1 hour at room temperature. Non-adherent cells were removed by inverted centrifugation, and the number of adherent cells was quantified using CellTiter-Glo (Promega #G7571) with the measurement of luminescence. EC₅₀ values were calculated using 4 parameter curve fitting for which 0% activation was set by the averaged luminescence values of an isotype control antibody and 100% activation was set by the averaged luminescence values of an activating antibody against β1 integrin.

### α7β1 Solid Phase Assay

The α7β1 solid phase assay was performed in 384-well assay plates in wells that were coated overnight at 4° C. with a solution of 10 μg/mL laminin-221, blocked 1 hour at 37° C. with a 2% solution of bovine serum albumin in phosphate buffered saline, and washed 3 times with wash buffer (0.05% Tween 20 and 1 mM magnesium chloride in phosphate buffered saline). Into each well, 6 nM purified α7β1 ectodomain protein containing a 6×His tag in a volume of 12.5 μL was added in assay buffer (100 mM HEPES pH 7.3, 150 mM sodium chloride, 1 mM magnesium chloride, 1 mM calcium chloride, 0.5% bovine serum albumin, and 0.05% Tween 20) and serial dilutions of the antibodies in assay buffer was added in a volume of 12.5 μL. The protein mixture was incubated for 1 hour at room temperature and non-adherent proteins were removed by washing 3 times with wash buffer. 25 μL of 20 ng/mL anti-6×His tag antibody conjugated to horse radish peroxidase (Abcam #ab1187) was added, incubated for 1 hour at room temperature, and washed 3 times. The amount of bound anti-6×His tag antibody was quantified using QuantaBlu (Thermo Fisher #15169) with the measurement of fluorescence. EC₅₀ values were calculated using 4 parameter curve fitting for which 0% activation was set by the averaged fluorescence values of an isotype control antibody and 100% activation was set by the averaged fluorescence values of an activating antibody against β1 integrin.

Binding of α7β1 Integrin to Laminin with α7β1Integrin Antibodies

To investigate if exemplary α7β1 integrin antibodies of the present disclosure can increase interactions between α7β1 integrin and laminin, cell adhesion assays, solid phase assays, and ECIS assays were completed with both human and mouse α7β1 integrin. Results are summarized in Table 5.

TABLE 5
Cell binding results with different α7β1 antigens.

| Assay | Antigen | Antibody | EC50 (nM) | Emax (%*) |
|---|---|---|---|---|
| Cell Adhesion | Human α7β1 | Ab8 | 3.94 | 117 |
| Assay | | Ab2 | 5.10 | 122 |
| | | RefAb1 | 1.34 | 38 |
| Cell Adhesion | Cynomolgus | Ab8 | 1.69 | 102 |
| Assay | Monkey α7β1 | Ab2 | 3.31 | 115 |
| | | RefAb1 | 0.58 | 47 |
| Cell Adhesion | Mouse α7β1 | Ab8 | 3.82 | 144 |
| Assay | | Ab2 | Undetectable | |
| | | RefAb1 | 6.2 | 48 |
| Solid Phase | Human α7β1 | Ab8 | 0.77 | 89 |
| Assay | | Ab2 | 0.40 | 97 |
| | | RefAb1 | 0.50 | 73 |
| Solid Phase | Mouse α7β1 | Ab8 | 1.55 | 148 |

| | | |
|---|---|---|
| Assay | Ab2 | Undetectable |

| | | |
|---|---|---|
| RefAb1 | 1.00 | 78 |

### ECIS Assays in DMD and Non-DMD Cell Lines

Exemplary α7β1 integrin antibodies were tested by ECIS assays in different myoblast cell lines, two of which were DMD cell lines. As seen in Table 6, exemplary α7β1 integrin antibodies have higher maximum effects than RefAb1 in DMD cells lines DMD-1 and DMD-2.

TABLE 6
ECIS binding with α7β1 integrin antibodies.

| | Non-DMD | | DMD-1 | | DMD-2 | |
|---|---|---|---|---|---|---|
| Myoblast | | Emax (% | | Emax (% | | Emax (% |
| Cells | EC50 | of | EC50 | of | EC50 | of |
| Treatment | (nM) | RefAb1) | (nM) | RefAb1) | (nM) | RefAb1) |
| Ab8 | 41.6 | 100 | 10.0 | 264 | 32.0 | 130 |
| Ab2 | 23.3 | 124 | 14.8 | 289 | 41.1 | 240 |
| Ab1 | 10.9 | 111 | 30.0 | 39 | ND | ND |
| Isotype | 8.1 | 35 | 38.1 | 59 | 8.7 | 39 |
| Control | | | | | | |
| RefAb1 | 28.8 | 100 | 20.4 | 100 | 23.9 | 100 |

### Cell Binding Under Activating Conditions

The α7β1 integrin antibodies were also tested for binding to α7β1 integrin under activating conditions in DMD-1 myoblast cells. Activating conditions, in this example, included the addition of Mn-BOP, where BOP is a pan-β1 small molecule inhibitor that stabilizes the open conformation of α7β1. The alternative condition, Mg/Ca, was considered to be more physiologically relevant and maintain of α7β1 integrin in its native, basal conformations

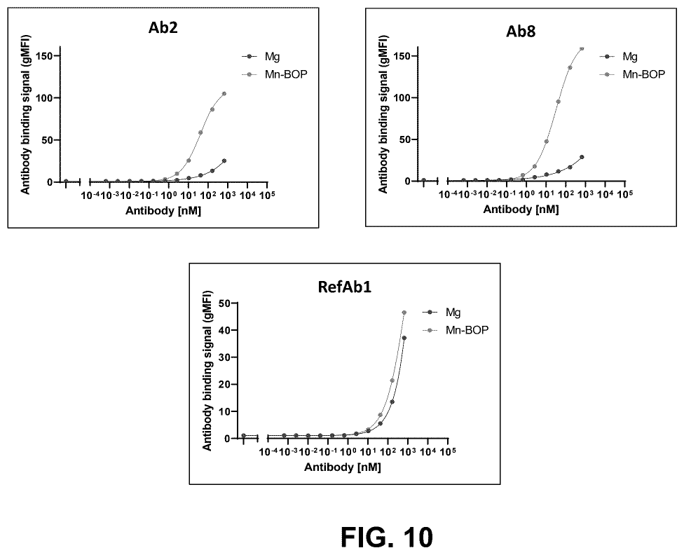

As shown in FIG. 10, the tested antibodies had significantly better binding under the activating conditions that encouraged the extended and extended-open conformations of α7β1, whereas the RefAb1 antibody could bind to α7β1 in either conformation. The Ab8 and RefAb1 antibodies were then tested against mouse α7β1 cells under activating conditions as shown in FIG. 10 with similar results. The tested antibodies had significantly lower EC50's under activating conditions as shown in Table 7.

TABLE 7
Cell binding of α7β1 integrin antibodies
under activating conditions.

| | DMD Myoblast Cells | Mouse α7β1 EC50 | |
|---|---|---|---|
| Antibody | EC50 with Mn-BOP (nM) | with Mn-BOP (nM) | LIBS |
| Ab2 | 41.1 | N/A | Yes |
| Ab8 | 32.6 | 22.6 | Yes |
| RefAb1 | >100 | >100 | No |

The results indicate that Ab2 and Ab8 have ligand-induced binding sites (LIBS), unlike RefAb1. The increased binding affinity of α7β1 with laminin can then help restore laminin interaction in cells without functional dystrophin.

Overall, this example demonstrated that exemplary antibodies exemplary antibodies have preferential binding to α7β1 in the extended-closed and extended-open conformations. Further, exemplary antibodies have higher maximum effects than RefAb1 in adhesion assays, indicating increased interaction between α7β1 and laminin when the exemplary antibodies are bound.

### Example 4. Characterization of Integrin α7β1 Activating Antibodies by Surface Plasmon Resonance

In this example, the binding affinity and characteristics of integrin α7β1 activating antibodies were determined by Surface Plasmon Resonance (SPR). Binding affinity for exemplary integrin α7β1 activating antibodies against different α7 antigens and domains can be found in Table 8. In addition, the summary of data for Ab2 and Ab8 are shown in FIG. 11.

TABLE 8
Binding Affinity of Exemplary Integrin
α7β1 Activating Antibodies.

| Antigen | Antibody | SPR Kd (nM) |
|---|---|---|
| Human α7β1 Ecto | Ab2 | 5.69 |
| Domain | Ab8 | 184 |
| | RefAb1 | 6.01 |
| Cynomolgus Monkey | Ab2 | 23.0 |
| α7β1 Ecto Domain | Ab8 | 225.0 |
| | RefAb1 | 4.50 |
| Mouse α7β1 Ecto | Ab2 | Undetectable |
| Domain | Ab8 | 5.46 |
| | RefAb1 | 44.87 |
| Human Leg α7 | Ab2 | 1.58 |
| | Ab8 | 25.01 |
| | RefAb1 | 3.35 |
| Cynomolgus Monkey | Ab2 | 19.45 |
| Leg α7 | Ab8 | 32.30 |
| | RefAb1 | 4.02 |
| Mouse Leg α7 | Ab2 | Undetectable |
| | Ab8 | 1.42 |
| | RefAb1 | 18.75 |

### Example 5. Integrin α7β1 Activating Antibodies Improve Disease Biomarkers in Acute and Chronic Studies

In this example, biomarkers for muscular dystrophy were measured with and without administration of α7β1 integrin antibodies in a mouse model of DMD. Serum and Muscle Exposure

First, the serum and muscle exposure of Ab8 and RefAb1 was analyzed. Ab8 had increased serum exposure than RefAb1 in a 4-week study. Both antibodies maintained an effective concentration in muscle throughout the study.

### CKM

A reliable mouse model for human DMD is the MDX mouse model, which contain a non-sense mutation in dystrophin exon 23 that aborts the dystrophin. Acute treadmill running is a method of causing muscle damage in MDX mice, normally leading to the leakage of muscle fiber proteins, including creatine kinase (CKM), LDHB, into the blood. It is theorized that running in MDX mice induces muscle injury and the release of muscle fiber proteins that can potentially be mitigated by the application of α7β1 integrin integrin antibodies.

In this study, B10.MDX mice were treated with 15 mg/kg on Day 1 and Day 3 of a 5-day study. On Days 2-4, mice were acclimated to the treadmill running for 3 minutes at a speed of 4 m/minute. On Day 5, the treadmill injury protocol was applied, 2 minutes at 4 m/minute, 2 minutes at 8 m/minute, and then 6 minutes at 12 m/minute. Blood was collected 1-hour post-run. This study was done repeatedly with different antibodies, and the results are compiled herein. CKM values were normalized to the rest group for each study.

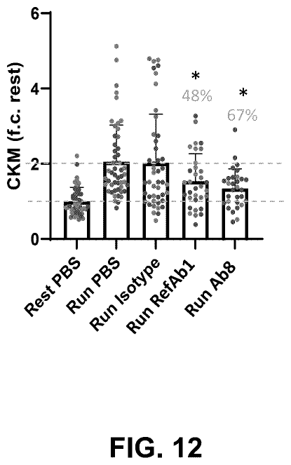

The effect of administration of Ab8 or RefAb1 was compared against several controls on the post-run CKM levels in blood. The resting condition provided a baseline value of CKM while the phosphate-buffered saline (PBS) and isotype controls with the treadmill run provided the untreated controls. As seen in FIG. 12, Ab8 resulted in a decrease in CKM in blood by 67%, significantly more than the RefAb1. The decreased CKM levels in blood suggest that the integrin antibodies protect the muscle fibers from membrane damage.

The blood was analyzed by SomaScan to determine the biomarkers most impacted by the treadmill run. PGAM1, CKM, CA3, and LDHB are among the top 15 protein increased by treadmill running in B1O.MDX mice. PGAM1 and CA3 have previously been reported as disease markers in DMD while CKM and LDHB are the most commonly-used muscle injury biomarkers.

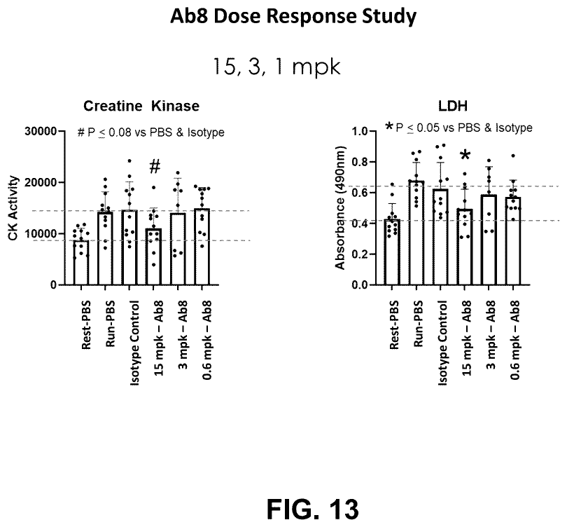

In a similar dose-response study, CKM and LDH were measured after administration of different doses of Ab8. As shown in FIG. 13, The Ab8 antibody, however, caused a dose-dependent decrease in both CKM and LDH levels.

### Chronic Efficacy Study 1

The chronic efficacy of α7β1 integrin antibodies was studied in a D2.MDX mouse model as summarized below in Table 11. Over the 9-week study, mice were dosed as described in Weeks 6-9 before a histology and gene expression profile was obtained.

TABLE 11
Chronic Efficacy Study 1 Design Summary.

| Group | Mouse | N | Treatment | Route | Dosing |
|---|---|---|---|---|---|
| 1 | WT | 5 | PBS | SC | 3x/wk |
| 2 | D2.MDX | 12 | PBS | SC | 3x/wk |
| 3 | D2.MDX | 12 | Isotype Control | SC | 3x/wk at 15 mg/kg |
| 4 | D2.MDX | 12 | RefAb2 | SC | 3x/wk at 15 mg/kg |
| 5 | D2.MDX | 12 | RefAb1 | SC | 3x/wk at 15 mg/kg |
| 6 | D2.MDX | 12 | Ab8 | SC | 3x/wk at 15 mg/kg |

Treatment with Ab8 caused intermediary changes in the transcriptome of MDX mice as compared to treatment with controls and RefAb1. Over 1800 genes were modulated by Ab8 where RefAb1 had no effect. Additionally, over 1600 genes that were dysregulated in MDX mice as compared to WT mice were reversed by treatment with Ab8.

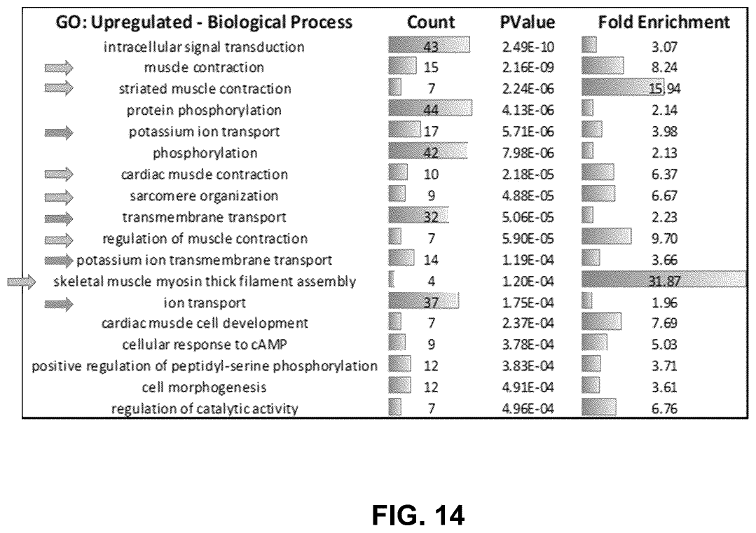

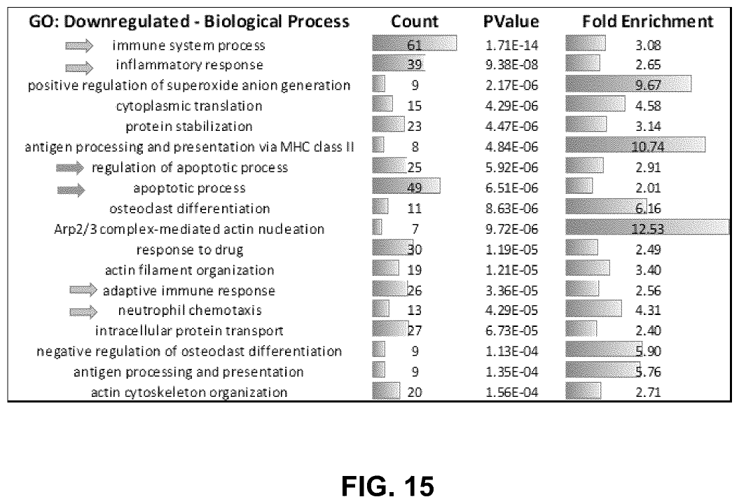

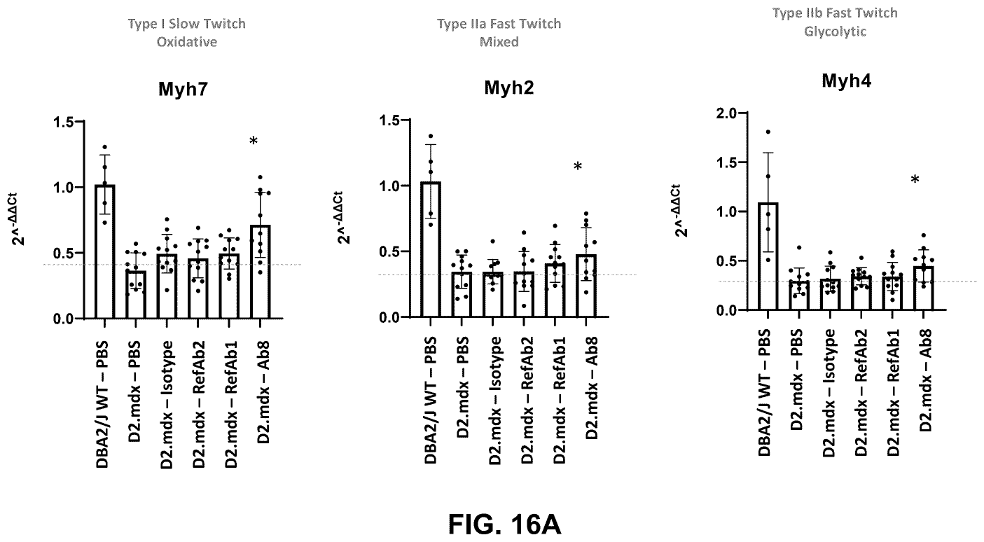

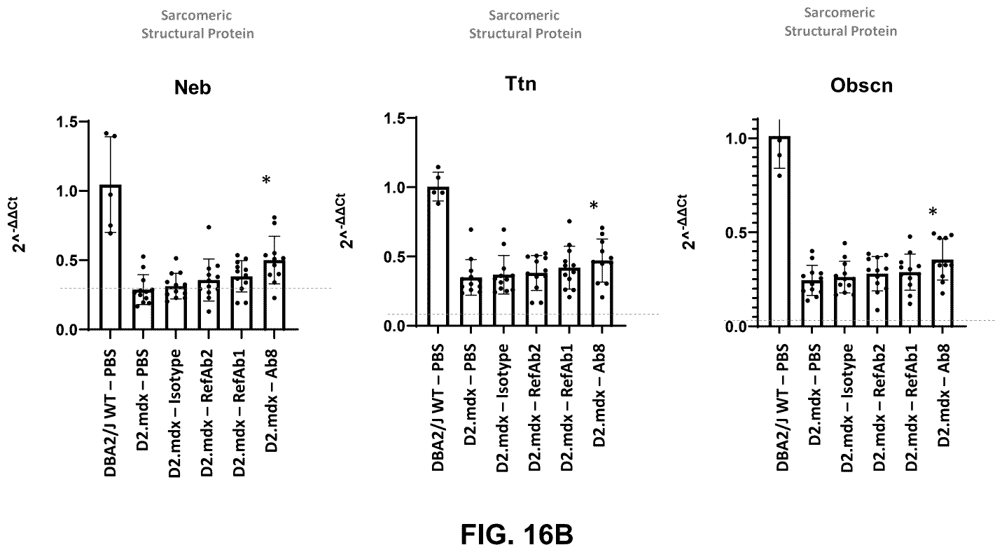

Pathway analysis on the affected genes showed that Ab8 modulated pathways associated with the pathogenesis of DMD. As shown in FIG. 14 and FIG. 15, genes related to muscle contraction, sarcomere organization, and ion handling were up-regulated, and genes related to immune response and apoptotic process were down-regulated. Increases in gene expression related to myosin, nebulin, obscurin, tropomodulin, and titin as shown in FIG. 16A and FIG. 16B, indicate that activation of α7β1 integrin by Ab8 may preserve sarcomere integrity. Ab8 was also found to increase gene expression of calcium handling genes.

Overall, this example demonstrated that the exemplary antibody, Ab8, has significant effect on gene expression related to muscle injury and disease pathology in MDX mice.

### Example 6. Increased Locomotive Ability in Muscular Dystrophy Mouse Models from Administration of Integrin α7β1 Activating Antibodies

In this example, the effect of integrin α7β1 activating antibodies on the locomotive ability of mice models with muscular dystrophy was determined through a treadmill running study and an open field activity test.

### Chronic Treadmill Running Study

A chronic treadmill study was conducted to assess the impact of exemplary α7β1 integrin antibody treatment on running capability. B10.MDX mice, starting at 6 weeks of age, were dosed as described in Table 12. After each dose, the mice were run on the treadmill following the running doses described in Table 13. After 12 sessions, or 4 weeks, the study was completed and tissue was collected for histopathology.

TABLE 12
Chronic treadmill runny study summary.

| | | | Treadmill | | | |
|---|---|---|---|---|---|---|
| Group | Mouse | N | Running | Treatment | Route | Dosing |
| 1 | B10.MDX | 12 | Yes | PBS | SC | |
| 2 | B10.MDX | 12 | Yes | Isotype | SC | 3x/wk at 15 mg/kg |
| | | | | Control | | |
| 3 | B10.MDX | 12 | Yes | RefAb1 | SC | 3x/wk at 15 mg/kg |
| 4 | B10.MDX | 12 | Yes | Ab8 | SC | 3x/wk at 15 mg/kg |
| 5 | B10.MDX | 12 | Yes | Ab8 | SC | 3x/wk at 7 mg/kg |

TABLE 13
Treadmill difficulty in chronic treadmill running study.

Running Dose

| | Speed for | Speed for | Speed for | Speed for |
|---|---|---|---|---|
| Sessions | 2 min. | 2 min. | 2 min. | 2 min. |
| 1 & 2 | 4 m/min | 8 m/min | | |
| 3-9 | 4 m/min | 8 m/min | 10 m/min | |
| 10-12 | 4 m/min | 8 m/min | 10 m/min | 12 m/min |

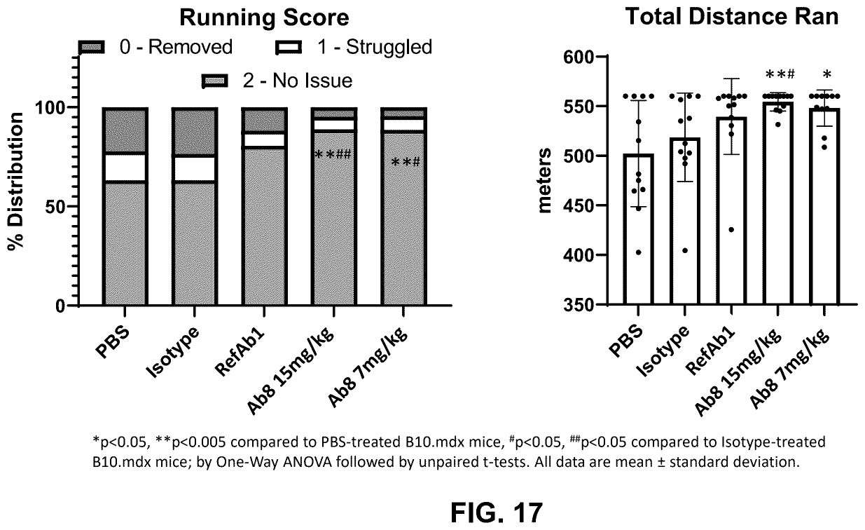

During the run, mice were scored based on the difficulty they displayed completing the run. A score of 0 was assigned if the mouse was removed, and a score of 1 was assigned if the mouse struggled but persisted. A score of 2 represents no issue while running. Scores were aggregated for each treatment group, and the distribution of scores can be seen in FIG. 17. Ab8 had a significantly higher running score than both the PBS and isotype controls, indicating increased running capability in MDX mice due to treatment.

The total distance run was also collected for each mouse as shown in FIG. 17. The 15 mg/kg dose of Ab8 showed a significantly higher average distance run than the PBS and isotype controls, while the 7 mg/kg dose was significantly higher than the PBS control. Again, this supported an increased capability to run caused by treatment with Ab8.

### Chronic Efficacy Study 2

A study on the chronic effect of treatment in MDX mice was completed as described in Table 14. Dosing began at the age of 4 weeks, and the study was completed at week 8.

TABLE 14
Chronic efficacy study 2 summary.

| Group | Mouse | N | Treatment | Route | Dose |
|---|---|---|---|---|---|
| 1 | D2.WT | 8 | PBS | SC | 0 |
| 2 | D2.MDX | 10 | PBS | SC | 0 |
| 3 | D2.MDX | 12 | Isotype Control | SC | 3x/wk at 15 mg/kg |
| 4 | D2.MDX | 12 | RefAb1 | SC | 3x/wk at 15 mg/kg |
| 5 | D2.MDX | 12 | Ab8 | SC | 3x/wk at 15 mg/kg |

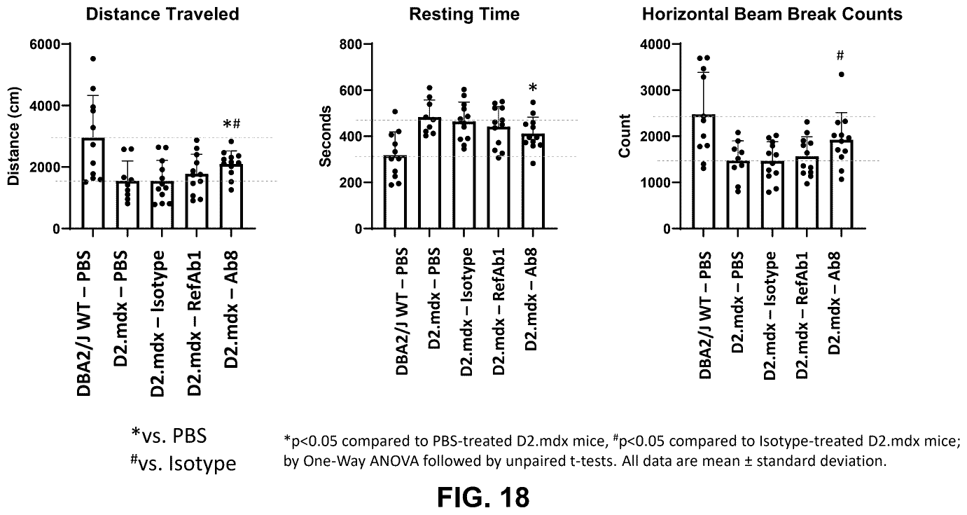

The open field activity of the mice was assessed and summarized throughout the study. The total distance run, resting time, and number of horizontal beam breaks, or the number of times the mouse broke through infrared beams that were placed in a grid over the field, were averaged for each group as shown in FIG. 18.

The group treated with Ab8 had a significantly higher average distance traveled than the PBS and isotype controls. The treatment group also had a significantly lower resting time than the PBS control, and a significantly higher number of beam breaks than the isotype control. The RefAb1 treatment group did not have any significant effect on any measure, although slight improvement can be seen.

Overall, this example demonstrated the increased therapeutic effect of exemplary α7β1 integrin antibody, Ab8, on improving the locomotive capability of MDX mice.

### Example 7. Increased Muscle Force in Muscular Dystrophy Mouse Models from Integrin α7β1 Activating Antibodies

The data in this example show that integrin α7β1 activating antibodies increased muscle force in a mouse model with muscular dystrophy.

### Chronic Efficacy Study 2

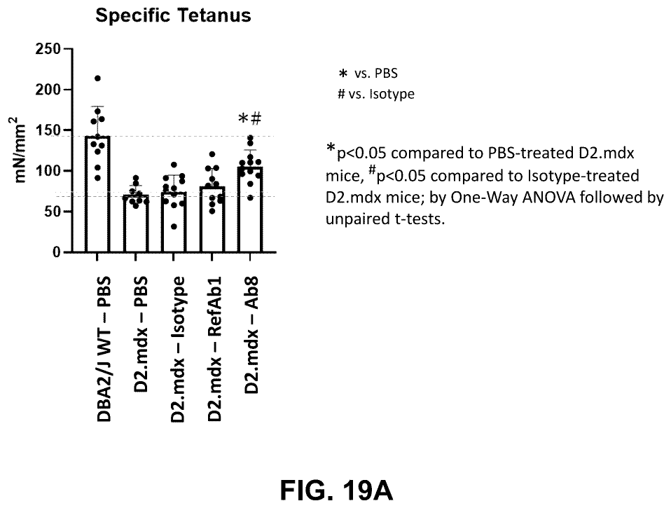

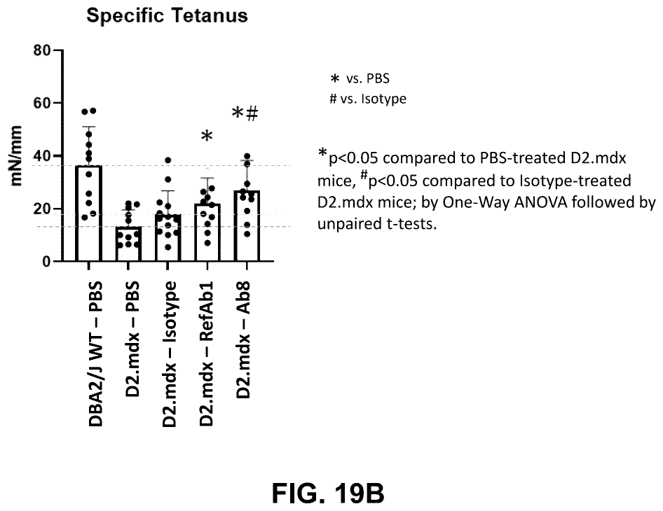

Chronic Efficacy Study 2, first described in Example 6, also assessed the impact of treatment on the physical muscle. The specific tetanus of the extensor digitorum longus and diaphragm was measured and averaged as shown in FIG. 19A and FIG. 19B. The administration of RefAb1 significantly increased the muscle force in the diaphragm as compared to the PBS control. The administration of Ab8 significantly increased the muscle force in both muscles as compared to both the PBS and isotype controls.

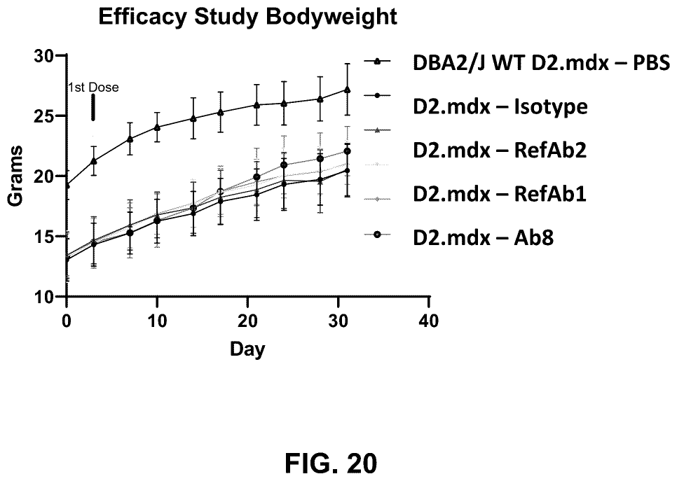

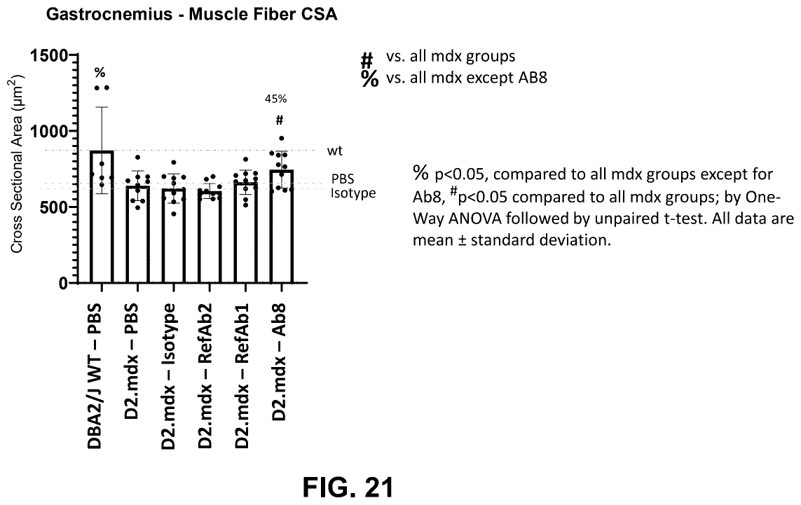

The effect of RefAb1, RefAb2, and Ab8 on muscle size and body weight was assessed. As shown in FIG. 20, treatment with Ab8 increased bodyweight 18% and 23.5% more than the PBS and isotype controls, respectively, while the reference antibodies did not significantly increase bodyweight. Additionally, as seen in FIG. 21, the administration of Ab8 significantly increased the muscle fiber size of the gastrocnemius muscle as compared to other MDX treatments, including both the controls and reference antibodies. The differences appear to be driven by the abundance of small, regenerating muscle fibers (<200 μm²).

Overall, this example demonstrated that the exemplary α7β1 integrin antibody, AbS, increases both muscle force and muscle size in MDX mice, indicating a direct therapeutic effect on the physical phenotype of DMD.

### Example 8. Reduction in Fibrosis Markers in Muscular Dystrophy Mouse Models from Integrin α7β1 Activating Antibodies

This example shows that administration of integrin α7β1 activating antibodies of the present disclosure reduced fibrosis in a muscular dystrophy mouse model.

### Chronic Efficacy Study 2

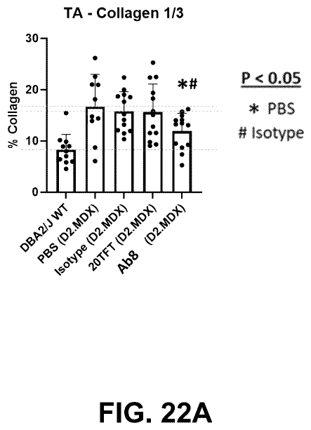

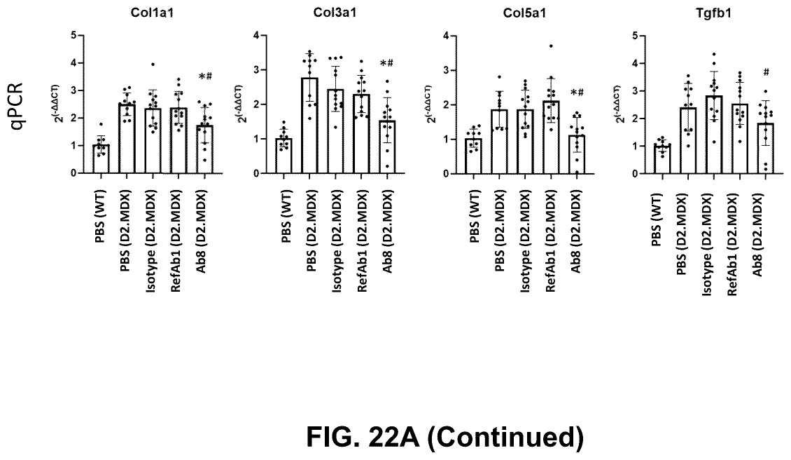

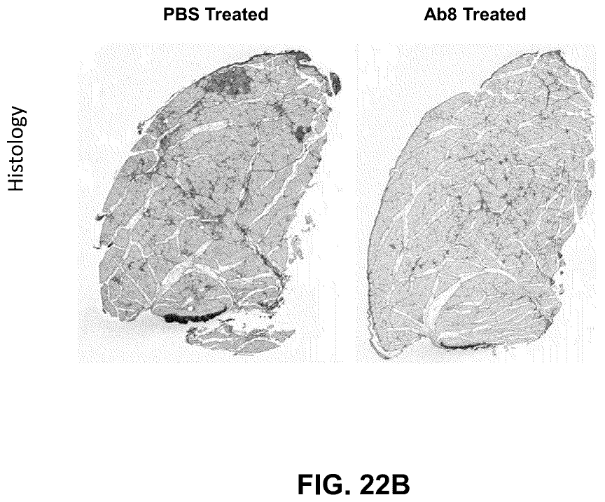

Several biomarkers for fibrosis were also assessed in the Chronic Efficacy Study 1, first described in Example 5. Administration of Ab8 significantly decreased the collagen positive area as shown in FIG. 22A. Ab8 also significantly decreased expression of Col1a1, Col3a1, Col5a1, and Tfgb1 as compared to both PBS and isotype controls as seen in. Lower collagen is sign of less scar tissue and fibrosis, a visible difference that can be seen in the histology images of FIG. 22B.

### Example 9. Prevention of Disuse Atrophy by Integrin α7Fll Activating Antibodies

This example shows that activation of α7 by an exemplary integrin α7β1 activating antibody attenuated muscle atrophy after immobilization.

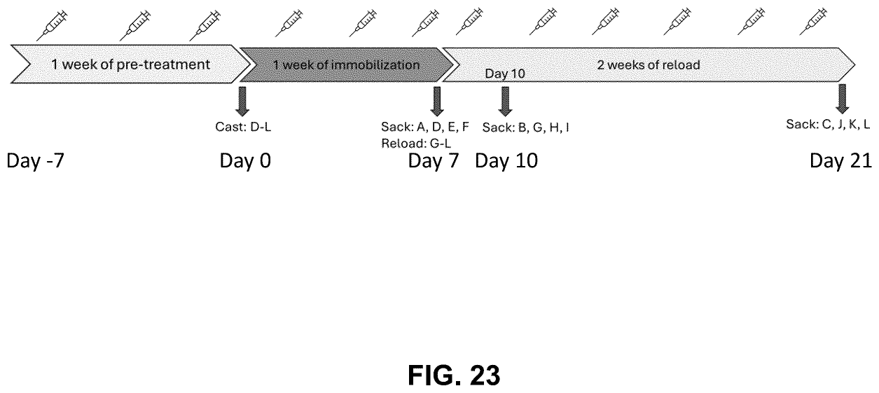

Twelve-week-old C57BL/6j male mice were subject to bilateral immobilization of the hindlimb with an elastic adhesive tape around the ankle of the hindlimbs. The Velcro tape inhibits plantar flexion and induces muscle atrophy of the gastrocnemius-soleus complex. Mice were treated with vehicle, isotype control, or an exemplary integrin α7β1 activating antibody (e.g., Ab8). As shown in FIG. 23, treatment started 1 week prior to immobilization on Day −7 and continued through the week of immobilization (Days 0-7) and the two weeks of reload (Days 8-21). Body and gastrocnemius-soleus weights were determined on Day 7 (VIM), Day 10 (3DRL), and Day 21 (14DRL). The study and testing groups are summarized in Table 15. The anti-myostatin (anti-MSTN/GDF8) antibody in Group E was added as a positive control.

TABLE 15
Summary of Study on Prevention of Disuse Atrophy.

| | | | | | Sample |
|---|---|---|---|---|---|
| Group | Condition | Duration | | Treatment | Size |
| A | Ambulatory | 7 | days | — | 3 |
| B | Immobilized | 7 | days | PBS Control | 8 |
| C | Immobilized | 7 | days | Isotype (15 mg/kg) | 8 |
| D | Immobilized | 7 | days | Ab8 (15 mg/kg) | 8 |
| E | Immobilized | 7 | days | anti-MSTN/GDF8 | 8 |
| | | | | mAb (30 mg/kg) | |
| F | Ambulatory | 7 + 3 | days | — | 3 |
| G | Immobilized/ | 7 + 3 | days | PBS Control | 8 |
| | Reloaded | | | | |
| H | Immobilized/ | 7 + 3 | days | Isotype (15 mg/kg) | 8 |
| | Reloaded | | | | |
| I | Immobilized/ | 7 + 3 | days | Ab8 (15 mg/kg) | 8 |
| | Reloaded | | | | |
| J | Ambulatory | 7 + 14 | days | — | 3 |
| K | Immobilized/ | 7 + 14 | days | PBS Control | 8 |
| | Reloaded | | | | |
| L | Immobilized/ | 7 + 14 | days | Isotype (15 mg/kg) | 8 |
| | Reloaded | | | | |
| M | Immobilized/ | 7 + 14 | days | Ab8 (15 mg/kg) | 8 |
| | Reloaded | | | | |

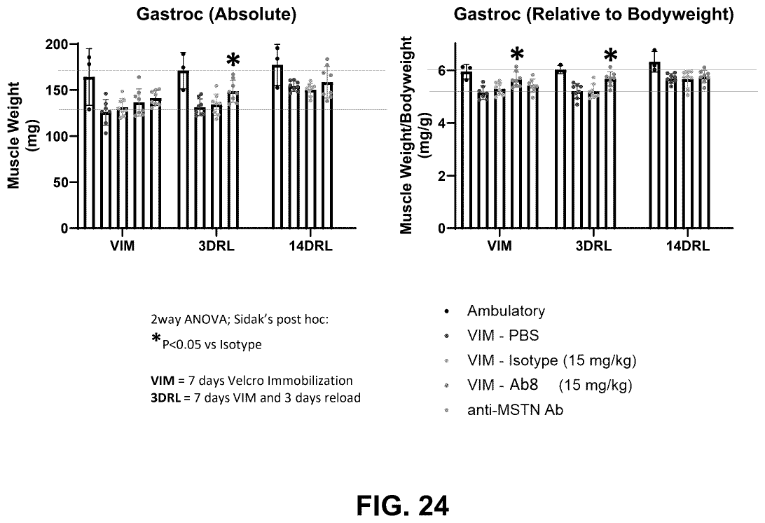

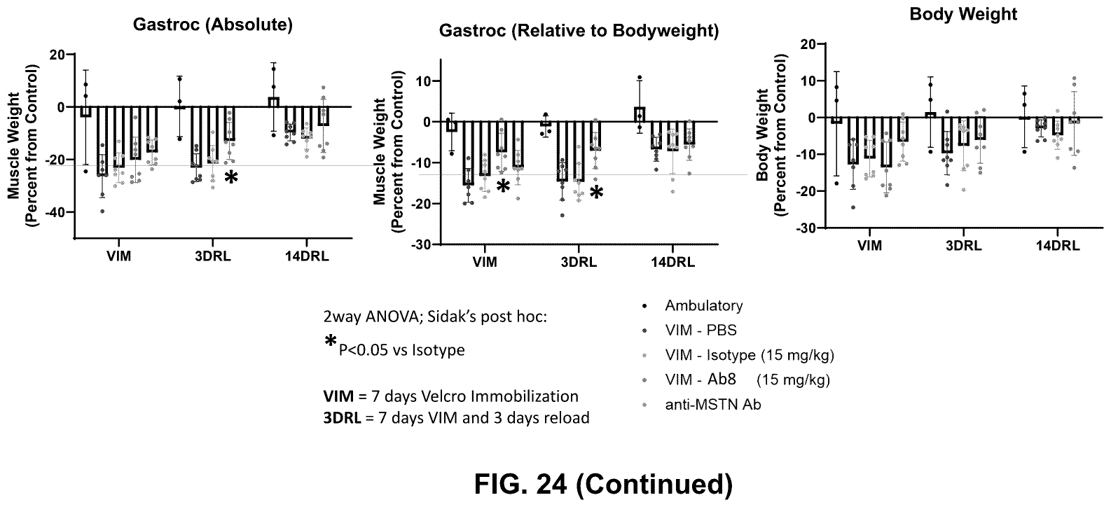

As shown in FIG. 24, exemplary integrin α7β1 activating antibody, Ab8, prevented muscle atrophy, as determined by wet muscle weight to body weight ratio, by about 50% as seen in VIM and 3DRL time points as compared to the PBS and isotype controls. By 14 days of reloading (14DRL), the control groups recovered to match the Ab8 group.

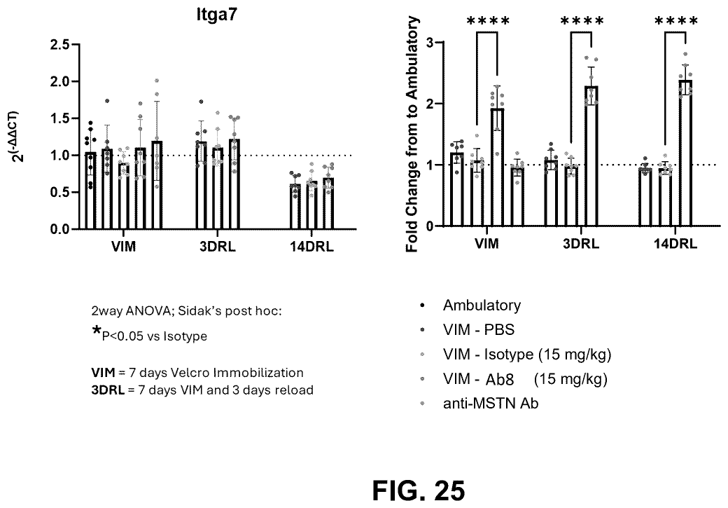

The expression level of α7β1 dimer was also measured for each testing group and compared to the ambulatory control. As shown in FIG. 25 and Table 16, administration of the exemplary integrin α7β1 activating antibody, Ab8, increased expression level of α7β1 dimer at all timepoints.

TABLE 16
Fold Change in α7β1 Expression over Ambulatory.

| | | Average Fold Change |
|---|---|---|
| Group | Days | over Ambulatory |
| Ambulatory Naïve | All | 1.00 |
| Immobilized PBS | 7 | 1.20 |
| | 10 | 1.08 |
| | 21 | 0.95 |
| Immobilized Isotype | 7 | 1.07 |
| | 10 | 0.98 |
| | 21 | 0.95 |
| Immobilized Ab8 | 7 | 1.93 |
| | 10 | 2.29 |
| | 21 | 2.39 |
| Immobilized anti-MSTN/GDF8 | 7 | 0.95 |

Overall, administration of the exemplary integrin α7β1 activating antibody, Ab8, increased α7β1 expression level and prevented loss of muscle mass in a mouse model of disuse atrophy, demonstrating potential as a therapy to attenuate muscle atrophy.

### Example 10. Generation and Characterization of Humanized, Affinity Matured Variant Antibodies

Ab8 was humanized using the CDR-grafting tool available in Bioluminate from Schrodinger (Schrödinger Release 2023-2: BioLuminate, Schrödinger, LLC, New York, NY). The tool performs a structural alignment of the original antibody structure with a collection of human antibody structures available in the Protein Data Bank (PDB). The initial structural model for Ab8 was built within Bioluminate using PDB ID 7QTA as the template with default settings. The most similar human template for the model was determined to be PDB ID 6C6Z. The CDR regions of the model were then swapped with the CDR regions of 6C6Z and energy minimized. The result was the creation a novel molecule containing the recognition elements of Ab8 in a human framework. Humanized versions of the Ab8 antibody (Ab24-27 and Ab30-42) are described in Tables E, F, and G.

Affinity maturation of humanized Ab8 was performed using a yeast platform. Mutation libraries were created in the scFv format. For LCDR1, LCDR2, LCDR3, HCDR1 and HCDR 2, CDR sequences were mined from a proprietary database based on diversities matching the humanized Ab8 V gene families. HCDR3 was selectively mutated at each amino acid position to 18 alternative residues, excluding cysteine. Oligonucleotides encoding each CDR were synthesized and fused to the appropriate family frameworks. The rest four sub-libraries (LCDR1-2, LCDR3, HCDR1-2, and HCDR3) were constructed through PCR assembly, incorporating the mutated CDR oligonucleotides while maintaining the unmutated variable regions of the antibody. Tolerated CDR sequences at each position were identified through multiple rounds of fluorescence-activated cell sorting (FACS) using human and cynomolgus proteins.

A combinatorial library was then generated by combining selected CDR sequences from LCDR1-2, LCDR3, HCDR1-2, and HCDR3 sub-libraries via assembly PCR. The library underwent magnetic-activated cell sorting (MACS) followed by FACS to isolate high-affinity clone. Sequencing of 222 clones revealed 98 unique variants, from the top 48 were selected based on their frequency and number of liabilities. These 48 sequences were cloned into expression vectors as human IgGIs and purified.

Surface plasmon resonance (SPR) was conducted to assess the kinetic binding of all 48 clones. Of these, 15 clones were exhibited increased affinity to human α7β1 ectodomain. Results for selected antibody fragments are shown in Table 17, below.

TABLE 17
Kinetic binding as measured by SPR

| | SPR KD to Human a7b1 | SPR KD to Human a7 |
|---|---|---|
| Antibody | Ecto (X2B) (nM) | Genu Calf 1-2 (nM) |
| AB-P | 63.7 | 16.4 |
| Ab24 | 2.4 | 0.1 |
| Ab25 | 3.4 | 0.3 |
| Ab26 | 2.2 | 0.3 |
| Ab30 | 3.99 | 0.57 |
| Ab31 | | 2.53 |
| Ab32 | | 1.53 |
| Ab33 | | 2.88 |
| Ab34 | | 1.35 |
| Ab35 | 4.47 | 0.54 |
| Ab36 | | 1.33 |
| Ab37 | | 1.43 |
| Ab38 | | 4.44 |
| Ab39 | | 1.57 |
| Ab40 | | 0.94 |
| Ab41 | 3.88 | 0.71 |
| Ab42 | | 5.70 |

a7b1 Activation Assay

96 well plates are coated with 1% matrigel overnight, and the following day human IPSCM (induced pluripotent cardio myocytes, FujiFilm) are plated in plating medium from Fujifilm, and 4 hours later media is changed to IPSCM growth media. Four days later, the media is changed to CyoTox Media (FujiFilm), with antibody therapeutics added to desired concentrations. After 24 hours, cells are washed in PBS, fixed in PFA for 30 minutes, stained using 9EG7 primary, and Rat IGG secondary, and imaged with the incucyte. An isotype control was included in the activation assay. This control did not increase the B1 activation response in the assay, demonstrating the biological effects of the integrin activation antibodies are indeed specific to the target rather than an IgG effect.

TABLE 18
Results of an activation assay - Ab24/Ab25

| | Ab24/Ab25 | Ab2 | TS2/16 |
|---|---|---|---|
| Emax (F.C.) | 2.29 | 2.75 | 2.34 |
| Emax (% TS2/16) | 97.87% | 117.00% | 100.00% |
| EC50 (ug/mL) | 0.28 | 0.44 | 0.31 |
| EC50 (nM) | 1.86 | 2.91 | 2.07 |

TABLE 19
Results of an activation assay - Ab26/Ab27

| | Ab26/Ab27 | Ab2 | TS2/16 |
|---|---|---|---|
| Emax (F.C.) | 1.80 | 2.09 | 2.09 |
| Emax (% TS2/16) | 86.35% | 99.95% | 100.00% |
| EC50 (ug/mL) | 0.43 | 0.42 | 0.30 |
| EC50 (nm) | 2.88 | 2.80 | 1.97 |

### Example 11. Characterization of Ab28 and Ab29

Surface plasmon resonance (SPR) was conducted to assess the kinetic binding of Ab28 and Ab29. Results for selected antibody fragments are shown in Table 20, below.

TABLE 20
Kinetic binding as measured by SPR

| | SPR KD to Human a7b1 | SPR KD to Human a7 |
|---|---|---|
| Antibody | Ecto (X2B) (nM) | Genu Calf 1-2 (nM) |
| Ab28 | 7.3 | 1.3 |
| Ab29 | 8.9 | 3.6 |

1.-97. (canceled)

98. An antibody or an antigen-binding fragment that binds α7β1 integrin, wherein the antibody comprises:
i) a HCDR1 of sequence GYTFTGYY (SEQ ID NO: 1), a HCDR2 of sequence INPNSGGT (SEQ ID NO: 15), a HCDR3 of sequence AREGANWDYYYYGMDV (SEQ ID NO: 31), a LCDR1 of sequence SSNIGSKT (SEQ ID NO: 46), a LCDR2 of sequence SNN (SEQ ID NO: 58), and a LCDR3 of sequence AAWDDSLNGPV (SEQ ID NO: 69);
ii) a HCDR1 of sequence GFTFSSYT (SEQ ID NO: 2), a HCDR2 of sequence ISIVGNYI (SEQ ID NO: 16), a HCDR3 of sequence ARRKWEHDAFDI (SEQ ID NO: 32), a LCDR1 of sequence SLRSYY (SEQ ID NO: 47), a LCDR2 of sequence GKN (SEQ ID NO: 59), and a LCDR3 of sequence TSRDNSGNHVV (SEQ ID NO: 70);
iii) a HCDR1 of sequence GFTFSSYS (SEQ ID NO: 3), a HCDR2 of sequence ISTSSSYI (SEQ ID NO: 17), a HCDR3 of sequence ARRAWESDAFAI (SEQ ID NO: 33), a LCDR1 of sequence SLRSYY (SEQ ID NO: 47), a LCDR2 of sequence GKN (SEQ ID NO: 59), and a LCDR3 of sequence NSRDSSGNHVV (SEQ ID NO: 71);
iv) a HCDR1 of sequence GFTFSSYA (SEQ ID NO: 4), a HCDR2 of sequence ISDSGGIA (SEQ ID NO: 18), a HCDR3 of sequence VRDGY (SEQ ID NO: 34), a LCDR1 of sequence QSLLDSDDGYTY (SEQ ID NO: 48), a LCDR2 of sequence TLS (SEQ ID NO: 60), and a LCDR3 of sequence MQRIEFPLT (SEQ ID NO: 72);
v) a HCDR1 of sequence GFTFSSYA (SEQ ID NO: 4), a HCDR2 of sequence IHYSGGTT (SEQ ID NO: 19), a HCDR3 of sequence VKDGY (SEQ ID NO: 35), a LCDR1 of sequence QSLLDSDDGYTY (SEQ ID NO: 48), a LCDR2 of sequence TLS (SEQ ID NO: 60), and a LCDR3 of sequence MQRIEFPLT (SEQ ID NO: 72);
vi) a HCDR1 of sequence GFTFSRFW (SEQ ID NO: 5), a HCDR2 of sequence TKQDGSEK (SEQ ID NO: 20), a HCDR3 of sequence AREGSGAYWAFDI (SEQ ID NO: 36), a LCDR1 of sequence QSLVHNDGNTY (SEQ ID NO: 49), a LCDR2 of sequence KIS (SEQ ID NO: 61), and a LCDR3 of sequence LQATQFPLT (SEQ ID NO: 73); or
vii) a HCDR1 of sequence GGSISSYY (SEQ ID NO: 6), a HCDR2 of sequence IYTRGST (SEQ ID NO: 21), a HCDR3 of sequence ARVFGGNYRFDAFHI (SEQ ID NO: 37), a LCDR1 of sequence SSNIGAGYD (SEQ ID NO: 50), a LCDR2 of sequence GNN (SEQ ID NO: 62), and a LCDR3 of sequence QSYDSSLSGYV (SEQ ID NO: 74).

99. The antibody or an antigen-binding fragment of claim 98, wherein the antibody or antigen binding fragment comprises:
i) a variable heavy region comprising an amino acid sequence of SEQ ID NO: 128, and a variable light chain region comprising an amino acid sequence of SEQ ID NO: 129;
i) a variable heavy region comprising an amino acid sequence of SEQ ID NO: 130, and a variable light chain region comprising an amino acid sequence of SEQ ID NO: 131;
ii) a variable heavy region comprising an amino acid sequence of SEQ ID NO: 132, and a variable light chain region comprising an amino acid sequence of SEQ ID NO: 133;
iii) a variable heavy region comprising an amino acid sequence of SEQ ID NO: 134, and a variable light chain region comprising an amino acid sequence of SEQ ID NO: 135;
iv) a variable heavy region comprising an amino acid sequence of SEQ ID NO: 136, and a variable light chain region comprising an amino acid sequence of SEQ ID NO: 137;
v) a variable heavy region comprising an amino acid sequence of SEQ ID NO: 138, and a variable light chain region comprising an amino acid sequence of SEQ ID NO: 139; or
vi) a variable heavy region comprising an amino acid sequence of SEQ ID NO: 140, and a variable light chain region comprising an amino acid sequence of SEQ ID NO: 141.

100. The antibody or an antigen-binding fragment of claim 98, wherein the antibody or antigen binding fragment comprises:
i) a heavy chain amino acid sequence of SEQ ID NO: 118 and a light chain amino acid sequence of SEQ ID NO: 119; or
ii) a heavy chain amino acid sequence of SEQ ID NO: 120 and a light chain amino acid sequence of SEQ ID NO: 121.

101. The antibody or antigen-binding fragment of claim 98, wherein the antibody or antigen-binding fragment stabilizes extended conformations of α7β1 integrin.

102. The antibody or antigen-binding fragment of claim 98, wherein the antibody or antigen-binding fragment has an affinity (Kd) to α7β1 integrin or a fragment thereof of between about 0.1 to 300 nM.

103. The antibody or antigen-binding fragment of claim 98, wherein the α7β1 integrin is a human α7β1 integrin.

104. The antibody or antigen-binding fragment of claim 98, wherein the antibody or antigen-binding fragment has an affinity (Kd) to a human α7β1 ectodomain of about 3 to 6 nM.

105. The antibody or antigen binding-fragment of claim 98, wherein the antibody or antigen-binding fragment has EC50 value of between 0.3 nM to 50 nM.

106. The antibody or antigen-binding of claim 98, wherein the antibody or antigen-binding fragment is a Fab, a single chain Fv (scFv), a single domain antibody (sdAb), a VHH, a variable heavy chain (VH), a variable light chain (VL), a Fab-like bispecific antibody (bsFab), a single-domain antibody-linked Fab (s-Fab), or a combination thereof.

107. The antibody or antigen-binding fragment of claim 98, wherein the antibody or antigen binding fragment is an IgG.

108. The antibody or antigen-binding fragment of claim 98, wherein the antibody or antigen-binding fragment is derived from IgG1, IgG2, IgG3, or IgG4.

109. The antibody or antigen-binding fragment of claim 98, wherein the antibody or antigen-binding fragment contains a Fc region.

110. The antibody or antigen-binding fragment claim 109, wherein the Fc region contains one or more mutations.

111. A pharmaceutical composition comprising the antibody or antigen-binding fragment of claim 98, and a pharmaceutically acceptable carrier.

112. A method of treating a disease or disorder in a subject in need thereof, the method comprising administering to the subject a therapeutically effective amount of the antibody or antigen-binding fragment of claim 98.

113. The method of claim 112, wherein the disease or disorder is cardiomyopathy, congestive heart failure, or muscular dystrophy.

114. An antibody or an antigen-binding fragment that binds α7β1 integrin, wherein the antibody comprises:
i) a HCDR1 of sequence GYSFTRYW (SEQ ID NO: 7), a HCDR2 of sequence IDPSDSET (SEQ ID NO: 22), a HCDR3 of sequence ARGGYFGEGWYFDV (SEQ ID NO: 38), a LCDR1 of sequence QSLVHRNGNTY (SEQ ID NO: 51), a LCDR2 of sequence KVS (SEQ ID NO: 63), and a LCDR3 of sequence SQSTHVPWT (SEQ ID NO: 75);
ii) a HCDR1 of sequence GYTFTSYW (SEQ ID NO: 8), a HCDR2 of sequence IYPSDSYT (SEQ ID NO: 23), a HCDR3 of sequence TRRANYVGAMDY (SEQ ID NO: 39), a LCDR1 of sequence SSVSY (SEQ ID NO: 52), a LCDR2 of sequence ATS (SEQ ID NO: 64), and a LCDR3 of sequence QQWSSNPLT (SEQ ID NO: 76);
iii) a HCDR1 of sequence GFSLTSYG (SEQ ID NO: 9), a HCDR2 of sequence IWVGGIT (SEQ ID NO: 24), a HCDR3 of sequence ARDGYYGDFDV (SEQ ID NO: 40), and a LCDR1 of sequence QDINKF (SEQ ID NO: 53), a LCDR2 of sequence YTS (SEQ ID NO: 65), and a LCDR3 of sequence LQYDNLRT (SEQ ID NO: 77);
iv) a HCDR1 of sequence GFSLTSYG (SEQ ID NO: 9), a HCDR2 of sequence IWAGGIT (SEQ ID NO: 25), a HCDR3 of sequence ARDGYYGDFDV (SEQ ID NO: 40), a LCDR1 of sequence QDINKY (SEQ ID NO: 54), a LCDR2 of sequence YTS (SEQ ID NO: 65), and a LCDR3 of sequence LQYDNLRT (SEQ ID NO: 77);
v) a HCDR1 of sequence GFSLTSYG (SEQ ID NO: 9), a HCDR2 of sequence IWAGGIT (SEQ ID NO: 25), a HCDR3 of sequence ARDGYYGHFDV (SEQ ID NO: 41), a LCDR1 of sequence QDINKY (SEQ ID NO: 54), a LCDR2 of sequence YTS (SEQ ID NO: 65), and a LCDR3 of sequence LQYDNLRA (SEQ ID NO: 78);
vi) a HCDR1 of sequence GFTFSSDA (SEQ ID NO: 10), a HCDR2 of sequence ISIGGSA (SEQ ID NO: 26), a HCDR3 of sequence ARGGDYDAMDY (SEQ ID NO: 42), and a LCDR1 of sequence QSISDL (SEQ ID NO: 55), a LCDR2 of sequence YAS (SEQ ID NO: 66), and a LCDR3 of sequence QNGHNFPFT (SEQ ID NO: 79);
vii) a HCDR1 of sequence GFTFSSSA (SEQ ID NO: 11), a HCDR2 of sequence ISVGGST (SEQ ID NO: 27), a HCDR3 of sequence ARGGDYDAMDY (SEQ ID NO: 42), a LCDR1 of sequence QSISDY (SEQ ID NO: 56), a LCDR2 of sequence YAS (SEQ ID NO: 66), and a LCDR3 of sequence QNGHSFPFT (SEQ ID NO: 80);
viii) a HCDR1 of sequence GFTFSSSA (SEQ ID NO: 11), a HCDR2 of sequence ISVGGST (SEQ ID NO: 27), a HCDR3 of sequence ARGGDYDAMDY (SEQ ID NO: 42), a LCDR1 of sequence QSISDY (SEQ ID NO: 56), a LCDR2 of sequence YSS (SEQ ID NO: 67), and a LCDR3 of sequence QNGHSFPFT (SEQ ID NO: 80);
ix) a HCDR1 of sequence GFTFSSSA (SEQ ID NO: 11), a HCDR2 of sequence ISVGGST (SEQ ID NO: 27), a HCDR3 of sequence ARGGDYDPMDY (SEQ ID NO: 43), a LCDR1 of sequence QSISDY (SEQ ID NO: 56), a LCDR2 of sequence YAS (SEQ ID NO: 66), and a LCDR3 of sequence QNGHSFPFT (SEQ ID NO: 80);
x) a HCDR1 of sequence GFTFSSDA (SEQ ID NO: 10), a HCDR2 of sequence ISIGGST (SEQ ID NO: 28), a HCDR3 of sequence ARGGDYDAMDY (SEQ ID NO: 42), a LCDR1 of sequence QSISDL (SEQ ID NO: 55), a LCDR2 of sequence YAS (SEQ ID NO: 66), and a LCDR3 of sequence QNGHNFPFT (SEQ ID NO: 79);
xi) a HCDR1 of sequence GFTFSSSA (SEQ ID NO: 11), a HCDR2 of sequence ISVGGSI (SEQ ID NO: 29), a HCDR3 of sequence ARGGDYDAMDY (SEQ ID NO: 42), a LCDR1 of sequence QSISDY (SEQ ID NO: 56), a LCDR2 of sequence YAS (SEQ ID NO: 66), and a LCDR3 of sequence QNGHSFPFT (SEQ ID NO: 80);
xii) a HCDR1 of sequence GFTFSNCA (SEQ ID NO: 12), a HCDR2 of sequence ISIGGST (SEQ ID NO: 28), a HCDR3 of sequence ARGGDYDAMDY (SEQ ID NO: 42), a LCDR1 of sequence QSISDL (SEQ ID NO: 55), a LCDR2 of sequence YAS (SEQ ID NO: 66), and a LCDR3 of sequence QNGHSFPFT (SEQ ID NO: 80);
xiii) a HCDR1 of sequence GFTFSSDA (SEQ ID NO: 10), a HCDR2 of sequence ISIGGST (SEQ ID NO: 28), a HCDR3 of sequence TRGGDYDAMDY (SEQ ID NO: 44), a LCDR1 of sequence QSISDL (SEQ ID NO: 55), a LCDR2 of sequence YAS (SEQ ID NO: 66), and a LCDR3 of sequence QNGHNFPFT (SEQ ID NO: 79);
xiv) a HCDR1 of sequence GFSLSTSDMG (SEQ ID NO: 13), a HCDR2 of sequence IYWDDDK (SEQ ID NO: 30), a HCDR3 of sequence VRGTAVVAFYWYFDV (SEQ ID NO: 45), a LCDR1 of sequence QDINNY (SEQ ID NO: 57), a LCDR2 of sequence RAN (SEQ ID NO: 68), and a LCDR3 of sequence LHYDDFPLT (SEQ ID NO: 81); or
xv) a HCDR1 of sequence GFSLSTSDLG (SEQ ID NO: 14), a HCDR2 of sequence IYWDDDK (SEQ ID NO: 30), a HCDR3 of sequence VRGTAVVAFYWYFDV (SEQ ID NO: 45), a LCDR1 of sequence QDINNY (SEQ ID NO: 57), a LCDR2 of sequence RAN (SEQ ID NO: 68), and a LCDR3 of sequence LQYDDFPLT (SEQ ID NO: 82).

115. The antibody or an antigen-binding fragment of claim 114, wherein the antibody or antigen-binding fragment comprises:
i) a variable heavy region comprising an amino acid sequence of SEQ ID NO: 142, and a variable light chain region comprising an amino acid sequence of SEQ ID NO: 143;
ii) a variable heavy region comprising an amino acid sequence of SEQ ID NO: 144, and a variable light chain region comprising an amino acid sequence of SEQ ID NO: 145;
iii) a variable heavy region comprising an amino acid sequence of SEQ ID NO: 146, and a variable light chain region comprising an amino acid sequence of SEQ ID NO: 147;
iv) a variable heavy region comprising an amino acid sequence of SEQ ID NO: 148, and a variable light chain region comprising an amino acid sequence of SEQ ID NO: 149;
v) a variable heavy region comprising an amino acid sequence of SEQ ID NO: 150, and a variable light chain region comprising an amino acid sequence of SEQ ID NO: 151;
vi) a variable heavy region comprising an amino acid sequence of SEQ ID NO: 152, and a variable light chain region comprising an amino acid sequence of SEQ ID NO: 153;
vii) a variable heavy region comprising an amino acid sequence of SEQ ID NO: 154, and a variable light chain region comprising an amino acid sequence of SEQ ID NO: 155;
viii) a variable heavy region comprising an amino acid sequence of SEQ ID NO: 156, and a variable light chain region comprising an amino acid sequence of SEQ ID NO: 157;
ix) a variable heavy region comprising an amino acid sequence of SEQ ID NO: 158, and a variable light chain region comprising an amino acid sequence of SEQ ID NO: 159;
x) a variable heavy region comprising an amino acid sequence of SEQ ID NO: 160, and a variable light chain region comprising an amino acid sequence of SEQ ID NO: 161;
xi) a variable heavy region comprising an amino acid sequence of SEQ ID NO: 162, and a variable light chain region comprising an amino acid sequence of SEQ ID NO: 163;
xii) a variable heavy region comprising an amino acid sequence of SEQ ID NO: 164, and a variable light chain region comprising an amino acid sequence of SEQ ID NO: 165;
xiii) a variable heavy region comprising an amino acid sequence of SEQ ID NO: 166, and a variable light chain region comprising an amino acid sequence of SEQ ID NO: 167;
xiv) a variable heavy region comprising an amino acid sequence of SEQ ID NO: 170, and a variable light chain region comprising an amino acid sequence of SEQ ID NO: 171; or
xv) a variable heavy region comprising an amino acid sequence of SEQ ID NO: 172, and a variable light chain region comprising an amino acid sequence of SEQ ID NO: 173.

116. The antibody or antigen-binding fragment of claim 114, wherein the antibody or antigen-binding fragment stabilizes extended conformations of α7β1 integrin.

117. The antibody or antigen-binding fragment of claim 114, wherein the antibody or antigen-binding fragment has an affinity (Kd) to α7β1 integrin or a fragment thereof of between about 0.1 to 300 nM.

118. The antibody or antigen-binding fragment of claim 114, wherein the α7β1 integrin is a human α7β1 integrin.

119. The antibody or antigen-binding fragment of claim 114, wherein the antibody or antigen-binding fragment has an affinity (Kd) to a human α7β1 ectodomain of about 3 to 6 nM.

120. The antibody or antigen binding-fragment of claim 114, wherein the antibody or antigen-binding fragment has EC50 value of between 0.3 nM to 50 nM.

121. The antibody or antigen-binding of claim 114, wherein the antibody or antigen-binding fragment is a Fab, a single chain Fv (scFv), a single domain antibody (sdAb), a VHH, a variable heavy chain (VH), a variable light chain (VL), a Fab-like bispecific antibody (bsFab), a single-domain antibody-linked Fab (s-Fab), or a combination thereof.

122. The antibody or antigen-binding fragment of claim 114, wherein the antibody or antigen binding fragment thereof is an IgG.

123. The antibody or antigen-binding fragment of claim 114, wherein the antibody or antigen-binding fragment is derived from IgG1, IgG2, IgG3, or IgG4.

124. The antibody or antigen-binding fragment of claim 114, wherein the antibody or antigen-binding fragment contains a Fc region.

125. The antibody or antigen-binding fragment claim 124, wherein the Fc region contains one or more mutations.

126. A pharmaceutical composition comprising the antibody or antigen-binding fragment of claim 114, and a pharmaceutically acceptable carrier.

127. A method of treating a disease or disorder in a subject in need thereof, the method comprising administering to the subject a therapeutically effective amount of the antibody or antigen-binding fragment of claim 114.

128. The method of claim 127, wherein the disease or disorder is cardiomyopathy, congestive heart failure, or muscular dystrophy.

129. An antibody or an antigen-binding fragment that binds α7β1 integrin, wherein the antibody comprises:
i) a HCDR1 of sequence GYSFTRYW (SEQ ID NO: 101), a HCDR2 of sequence IDPSDSET (SEQ ID NO: 27), a HCDR3 of sequence ARGGYFGEGWYFDV (SEQ ID NO: 38), a LCDR1 of sequence QSLVHRNGNTY (SEQ ID NO: 105), and a LCDR2 of sequence KVS (SEQ ID NO: 63), and a LCDR3 of sequence SQSTHVPWT (SEQ ID NO: 75);
ii) a HCDR1 of sequence GYSFTRYW (SEQ ID NO: 101), a HCDR2 of sequence IDPSDSET (SEQ ID NO: 102), a HCDR3 of sequence ARGGYFGREWYYDV (SEQ ID NO: 103), a LCDR1 of sequence QSLVHRNGNTY (SEQ ID NO: 105), a LCDR2 of sequence EVS (SEQ ID NO: 106), and a LCDR3 of sequence SQSTHVPWT (SEQ ID NO:75);
iii) a HCDR1 of sequence GYSFTRYW (SEQ ID NO: 101), a HCDR2 of sequence IDPSDSET (SEQ ID NO: 102), a HCDR3 of sequence ARGGYFGEEWYHDV (SEQ ID NO: 104), a LCDR1 of sequence QSLVHRNGNTY (SEQ ID NO: 105), a LCDR2 of sequence EVS (SEQ ID NO: 106), and a LCDR3 of sequence AQSTQLPYT (SEQ ID NO: 107);
iv) a HCDR1 of sequence GYSFTRYW (SEQ ID NO: 101), a HCDR2 of sequence IDPSDSET (SEQ ID NO: 102), a HCDR3 of sequence ARGGYFGEGWYHDV (SEQ ID NO: 188), a LCDR1 of sequence QSLVHRNGNTY (SEQ ID NO: 105), a LCDR2 of sequence EVS (SEQ ID NO: 106), and a LCDR3 of sequence AQSIQVPWT (SEQ ID NO: 198);
v) a HCDR1 of sequence GYSFTRYW (SEQ ID NO: 101), a HCDR2 of sequence IDPSDSET (SEQ ID NO: 102), a HCDR3 of sequence ARGGYFGRQWYFDV (SEQ ID NO: 189), a LCDR1 of sequence QSLVHRNGNTY (SEQ ID NO: 105), a LCDR2 of sequence EVS (SEQ ID NO: 106), and a LCDR3 of sequence SQSTHVPWT (SEQ ID NO: 75);
vi) a HCDR1 of sequence GYSFTRYW (SEQ ID NO: 101), a HCDR2 of sequence IDPSDSET (SEQ ID NO: 102), a HCDR3 of sequence ARGGYFGEQWYHDV (SEQ ID NO: 190), a LCDR1 of sequence QSLVHRNGNTY (SEQ ID NO: 105), a LCDR2 of sequence EVS (SEQ ID NO: 106), and a LCDR3 of sequence SQSTHVPWT (SEQ ID NO: 75);
vii) a HCDR1 of sequence GYSFTRYW (SEQ ID NO: 101), a HCDR2 of sequence IDPSDSET (SEQ ID NO: 102), a HCDR3 of sequence ARGGYFGVGWYNDV (SEQ ID NO: 191), a LCDR1 of sequence ARGGYFGVGWYNDV (SEQ ID NO: 105), a LCDR2 of sequence EVS (SEQ ID NO: 106), and a LCDR3 of sequence SQSTHVPWT (SEQ ID NO: 75);
viii) a HCDR1 of sequence GYSFTRYW (SEQ ID NO: 101), a HCDR2 of sequence IDPSDSET (SEQ ID NO: 102), a HCDR3 of sequence ARGGYFGETWYHDV (SEQ ID NO: 192), a LCDR1 of sequence QSLVHRNGNTY (SEQ ID NO: 105), a LCDR2 of sequence EVS (SEQ ID NO: 106), and a LCDR3 of sequence SQSTHVPWT (SEQ ID NO: 75);
ix) a HCDR1 of sequence GYSFTRYW (SEQ ID NO: 101), a HCDR2 of sequence IDPSDSET (SEQ ID NO: 102), a HCDR3 of sequence ARGGYFGENWYHDV (SEQ ID NO: 193), a LCDR1 of sequence QSLVHRNGNTY (SEQ ID NO: 105), a LCDR2 of sequence EVS (SEQ ID NO: 106), and a LCDR3 of sequence AQSIQVPWT (SEQ ID NO: 198);
x) a HCDR1 of sequence GYSFTRYW (SEQ ID NO: 101), a HCDR2 of sequence IDPSDSET (SEQ ID NO: 102), a HCDR3 of sequence ARGGYFGEKWYHDV (SEQ ID NO: 194), a LCDR1 of sequence QSLVHRNGNTY (SEQ ID NO: 105), a LCDR2 of sequence EVS (SEQ ID NO: 106), and a LCDR3 of sequence AQSTQLPYT (SEQ ID NO: 107);
xi) a HCDR1 of sequence GYSFTRYW (SEQ ID NO: 101), a HCDR2 of sequence IDPSDSET (SEQ ID NO: 102), a HCDR3 of sequence ARGGYFGEKWYHDV (SEQ ID NO: 194), a LCDR1 of sequence QSLVHRNGNTY (SEQ ID NO: 105), a LCDR2 of sequence GVS (SEQ ID NO: 196), and a LCDR3 of sequence AQSTQLPYT (SEQ ID NO: 107);
xii) a HCDR1 of sequence GYSFTRYW (SEQ ID NO: 101), a HCDR2 of sequence IDPSDSET (SEQ ID NO: 102), a HCDR3 of sequence ARGGYFGEEWYNDV (SEQ ID NO: 195), a LCDR1 of sequence QSLVHRNGNTY (SEQ ID NO: 105), a LCDR2 of sequence KVS (SEQ ID NO: 63), and a LCDR3 of sequence SQSTHVPWT (SEQ ID NO: 75);
xiii) a HCDR1 of sequence GYSFTRYW (SEQ ID NO: 101), a HCDR2 of sequence IDPSDSET (SEQ ID NO: 102), a HCDR3 of sequence ARGGYFGEEWYHDV (SEQ ID NO: 104), a LCDR1 of sequence QSLVHRNGNTY (SEQ ID NO: 105), a LCDR2 of sequence KVS (SEQ ID NO: 63), and a LCDR3 of sequence SQSTHVPWT (SEQ ID NO: 75);
xiv) a HCDR1 of sequence GYSFTRYW (SEQ ID NO: 101), a HCDR2 of sequence IDPSDSET (SEQ ID NO: 102), a HCDR3 of sequence ARGGYFGEEWYHDV (SEQ ID NO: 104), a LCDR1 of sequence QSLVHRNGNTY (SEQ ID NO: 105), a LCDR2 of sequence EGS (SEQ ID NO: 197), and a LCDR3 of sequence SQSTHVPWT (SEQ ID NO: 75);
xv) a HCDR1 of sequence GYSFTRYW (SEQ ID NO: 101), a HCDR2 of sequence IDPSDSET (SEQ ID NO: 102), a HCDR3 of sequence ARGGYFGEQWYHDV (SEQ ID NO: 190), a LCDR1 of sequence QSLVHRNGNTY (SEQ ID NO: 105), a LCDR2 of sequence EVS (SEQ ID NO: 106), and a LCDR3 of sequence AQSIQVPWT (SEQ ID NO: 198);
xvi) a HCDR1 of sequence GYSFTRYW (SEQ ID NO: 101), a HCDR2 of sequence IDPSDSET (SEQ ID NO: 102), a HCDR3 of sequence ARGGYFGRQWYFDV (SEQ ID NO: 189), a LCDR1 of sequence QSLVHRNGNTY (SEQ ID NO: 105), a LCDR2 of sequence EGS (SEQ ID NO: 197), and a LCDR3 of sequence AQSTQLPYT (SEQ ID NO: 107).

130. The antibody or an antigen-binding fragment of claim 129, wherein the antibody or antigen binding fragment comprises:
i) a variable heavy region comprising an amino acid sequence of SEQ ID NO: 186, and a variable light chain region comprising an amino acid sequence of SEQ ID NO: 187;
ii) a variable heavy region comprising an amino acid sequence of SEQ ID NO: 108, and a variable light chain region comprising an amino acid sequence of SEQ ID NO: 109;
iii) a variable heavy region comprising an amino acid sequence of SEQ ID NO: 110, and a variable light chain region comprising an amino acid sequence of SEQ ID NO: 111;
iv) a variable heavy region comprising an amino acid sequence of SEQ ID NO: 199, and a variable light chain region comprising an amino acid sequence of SEQ ID NO: 200;
v) a variable heavy region comprising an amino acid sequence of SEQ ID NO: 201, and a variable light chain region comprising an amino acid sequence of SEQ ID NO: 202;
vi) a variable heavy region comprising an amino acid sequence of SEQ ID NO: 203, and a variable light chain region comprising an amino acid sequence of SEQ ID NO: 204;
vii) a variable heavy region comprising an amino acid sequence of SEQ ID NO: 205, and a variable light chain region comprising an amino acid sequence of SEQ ID NO: 206;
viii) a variable heavy region comprising an amino acid sequence of SEQ ID NO: 207, and a variable light chain region comprising an amino acid sequence of SEQ ID NO: 208;
ix) a variable heavy region comprising an amino acid sequence of SEQ ID NO: 209, and a variable light chain region comprising an amino acid sequence of SEQ ID NO: 210;
x) a variable heavy region comprising an amino acid sequence of SEQ ID NO: 211, and a variable light chain region comprising an amino acid sequence of SEQ ID NO: 212;
xi) a variable heavy region comprising an amino acid sequence of SEQ ID NO: 213, and a variable light chain region comprising an amino acid sequence of SEQ ID NO: 214;
xii) a variable heavy region comprising an amino acid sequence of SEQ ID NO: 215, and a variable light chain region comprising an amino acid sequence of SEQ ID NO: 216;
xiii) a variable heavy region comprising an amino acid sequence of SEQ ID NO: 217, and a variable light chain region comprising an amino acid sequence of SEQ ID NO: 218;
xiv) a variable heavy region comprising an amino acid sequence of SEQ ID NO: 219, and a variable light chain region comprising an amino acid sequence of SEQ ID NO: 220;
xv) a variable heavy region comprising an amino acid sequence of SEQ ID NO: 221, and a variable light chain region comprising an amino acid sequence of SEQ ID NO: 222; or
xvi) a variable heavy region comprising an amino acid sequence of SEQ ID NO: 223, and a variable light chain region comprising an amino acid sequence of SEQ ID NO: 224.

131. The antibody or an antigen-binding fragment of claim 129, wherein the antibody or antigen-binding fragment comprises:
i) a heavy chain amino acid sequence of SEQ ID NO: 112 and a light chain amino acid sequence of SEQ ID NO: 113;
ii) a heavy chain amino acid sequence of SEQ ID NO: 114 and a light chain amino acid sequence of SEQ ID NO: 113;
iii) a heavy chain amino acid sequence of SEQ ID NO: 115 and a light chain amino acid sequence of SEQ ID NO: 116; or
iv) a heavy chain amino acid sequence of SEQ ID NO: 117 and a light chain amino acid sequence of SEQ ID NO: 116.

132. The antibody or antigen-binding fragment of claim 129, wherein the antibody or antigen-binding fragment stabilizes extended conformations of α7β1 integrin.

133. The antibody or antigen-binding fragment of claim 129, wherein the antibody or antigen-binding fragment has an affinity (Kd) to α7β1 integrin or a fragment thereof of between about 0.1 to 300 nM.

134. The antibody or antigen-binding fragment of claim 129, wherein the α7β1 integrin is a human α7β1 integrin.

135. The antibody or antigen-binding fragment of claim 129, wherein the antibody or antigen-binding fragment has an affinity (Kd) to a human α7β1 ectodomain of about 3 to 6 nM.

136. The antibody or antigen binding-fragment of claim 129, wherein the antibody or antigen-binding fragment has EC50 value of between 0.3 nM to 50 nM.

137. The antibody or antigen-binding of claim 129, wherein the antibody or antigen-binding fragment is a Fab, a single chain Fv (scFv), a single domain antibody (sdAb), a VHH, a variable heavy chain (VH), a variable light chain (VL), a Fab-like bispecific antibody (bsFab), a single-domain antibody-linked Fab (s-Fab), or a combination thereof.

138. The antibody or antigen-binding fragment of claim 129, wherein the antibody or antigen binding fragment thereof is an IgG.

139. The antibody or antigen-binding fragment of claim 129, wherein the antibody or antigen-binding fragment is derived from IgG1, IgG2, IgG3, or IgG4.

140. The antibody or antigen-binding fragment of claim 129, wherein the antibody or antigen-binding fragment contains a Fc region.

141. The antibody or antigen-binding fragment claim 140, wherein the Fc region contains one or more mutations.

142. A pharmaceutical composition comprising the antibody or antigen-binding fragment of claim 129, and a pharmaceutically acceptable carrier.

143. A method of treating a disease or disorder in a subject in need thereof, the method comprising administering to the subject a therapeutically effective amount of the antibody or antigen-binding fragment of claim 129.

144. The method of claim 143, wherein the disease or disorder is cardiomyopathy, congestive heart failure, or muscular dystrophy.
